(19) United States
(12) Patent Application Publication
(10) Pub. No.: US 2024/0076297 A1
(43) Pub. Date: Mar. 7, 2024

(54) ADENOSINE A2A AND A2B RECEPTOR DUAL ANTAGONISTS FOR IMMUNO-ONCOLOGY

(71) Applicant: Merck Sharp & Dohme LLC, Rahway, NJ (US)

(72) Inventors: Amjad Ali, Freehold, NJ (US); Jared N. Cumming, Winchester, MA (US); Manuel De Lera Ruiz, Perkasie, PA (US); Duane DeMong, Hanover, MA (US); Thomas H. Graham, Somerville, MA (US); Elisabeth T. Hennessy, Weston, MA (US); Joseph M. Kelly, Parlin, NJ (US); Rongze Kuang, Green Brook, NJ (US); Michael Man-Chu Lo, Bedminster, NJ (US); Umar Faruk Mansoor, Hopkinton, MA (US); Jesus Moreno, San Diego, CA (US); Uma Swaminathan, Auburndale, MA (US); Heping Wu, Edison, NJ (US); Yingchun Ye, Belmont, MA (US); Younong Yu, East Brunswick, NJ (US)

(73) Assignee: Merck Sharp & Dohme LLC, Rahway, NJ (US)

(21) Appl. No.: 18/015,364

(22) PCT Filed: Jul. 22, 2021
(86) PCT No.: PCT/US2021/042708

§ 371 Date: Jan. 10, 2023

Related U.S. Application Data
(60) Provisional application No. 63/056,248, filed on Jul. 24, 2020.

(51) Int. Cl. C07D 487/04; A61K 45/06; C07D 519/00; C07K 16/28

(52) U.S. Cl. CPC C07D 487/04; A61K 45/06; C07D 519/00; C07K 16/2818

(57) ABSTRACT

The present invention provides compounds of the structural Formula (I), and pharmaceutically acceptable salts thereof, wherein, are as defined herein, pharmaceutical compositions comprising one or more such compounds (alone and in combination with one or more other therapeutically active agents), and methods for their preparation and use, alone and in combination with other therapeutic agents, as antagonists of A2a and/or A2b receptors, and in the treatment of a variety of diseases, conditions, or disorders that are mediated, at least in part, by the adenosine A2a receptor and/or the adenosine A2b receptor.

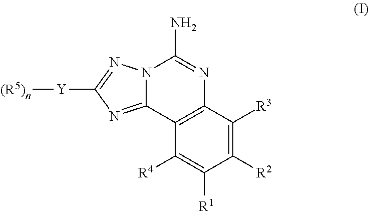

### FIELD OF THE INVENTION

The present invention relates to novel compounds that inhibit at least one of the A2a and A2b adenosine receptors, and pharmaceutically acceptable salts thereof, and compositions comprising such compound(s) and salts, methods for the synthesis of such compounds, and their use in the treatment of a variety of diseases, conditions, or disorders that are mediated, at least in part, by the adenosine A2a receptor and/or the adenosine A2b receptor. Such diseases, conditions, and disorders include but are not limited to cancer and immune-related disorders. The invention further relates to combination therapies, including but not limited to a combination comprising a compound of the invention and a PD-1 antagonist.

### BACKGROUND OF THE INVENTION

Adenosine is a purine nucleoside compound comprised of adenine and ribofuranose, a ribose sugar molecule. Adenosine occurs naturally in mammals and plays important roles in various biochemical processes, including energy transfer (as adenosine triphosphate and adenosine monophosphate) and signal transduction (as cyclic adenosine monophosphate). Adenosine also plays a causative role in processes associated with vasodilation, including cardiac vasodilation. It also acts as a neuromodulator (e.g., it is thought to be involved in promoting sleep). In addition to its involvement in these biochemical processes, adenosine is used as a therapeutic antiarrhythmic agent to treat supraventricular tachycardia and other indications.

The adenosine receptors are a class of purinergic G protein-coupled receptors with adenosine as the endogenous ligand. The four types of adenosine receptors in humans are referred to as A1, A2a, A2b, and A3. Modulation of A1 has been proposed for the management and treatment of neurological disorders, asthma, and heart and renal failure, among others. Modulation of A3 has been proposed for the management and treatment of asthma and chronic obstructive pulmonary diseases, glaucoma, cancer, stroke, and other indications. Modulation of the A2a and A2b receptors are also believed to be of potential therapeutic use.

In the central nervous system, A2a antagonists are believed to exhibit antidepressant properties and to stimulate cognitive functions. A2a receptors are present in high density in the basal ganglia, known to be important in the control of movement. Hence, A2a receptor antagonists are believed to be useful in the treatment of depression and to improve motor impairment due to neurodegenerative diseases such as Parkinson's disease, senile dementia (as in Alzheimer's disease), and in various psychoses of organic origin.

In the immune system, adenosine signaling through A2a receptors and A2b receptors, expressed on a variety of immune cells and endothelial cells, has been established as having an important role in protecting tissues during inflammatory responses. In this way (and others), tumors have been shown to evade host responses by inhibiting immune function and promoting tolerance. (See, e.g., Fishman, P., et al., Handb. Exp. Pharmacol. (2009) 193:399-441). Moreover, A2a and A2b cell surface adenosine receptors have been found to be upregulated in various tumor cells. Thus, antagonists of the A2a and/or A2b adenosine receptors represent a new class of promising oncology therapeutics. For example, activation of A2a adenosine receptors results in the inhibition of the immune response to tumors by a variety of cell types, including but not limited to: the inhibition of natural killer cell cytotoxicity, the inhibition of tumor-specific CD4+/CD8+ activity, promoting the generation of LAG-3 and Foxp3+ regulatory T-cells, and mediating the inhibition of regulatory T-cells. Adenosine A2a receptor inhibition has also been shown to increase the efficacy of PD-1 inhibitors through enhanced anti-tumor T cell responses. As each of these immunosuppressive pathways has been identified as a mechanism by which tumors evade host responses, a cancer immunotherapeutic regimen that includes an antagonist of the A2a and/or A2b receptors, alone or together with one or more other therapeutic agents designed to mitigate immune suppression, may result in enhanced tumor immunotherapy. (See, e.g., P. Beavis, et al., Cancer Immunol. Res. DOI: 10.1158/2326-6066. CIR-14-0211, Feb. 11, 2015; Willingham, S B., et al., Cancer Immunol. Res., 6(10), 1136-49; and Leone R D, et al., Cancer Immunol. Immunother., August 2018, Vol. 67, Issue 8, 1271-1284).

Cancer cells release ATP into the tumor microenvironment when treated with chemotherapy and radiation therapy, which is subsequently converted to adenosine. (See Martins, I., et al., Cell Cycle, vol. 8, issue 22, pp. 3723 to 3728.) The adenosine can then bind to A2a receptors and blunt the anti-tumor immune response through mechanisms such as those described above. The administration of A2a receptor antagonists during chemotherapy or radiation therapy has been proposed to lead to the expansion of the tumor-specific T-cells while simultaneously preventing the induction of tumor-specific regulatory T-cells. (Young, A., et al., Cancer Discovery (2014) 4:879-888).

The combination of an A2a receptor antagonist with anti-tumor vaccines is believed to provide at least an additive therapeutic effect in view of their different mechanisms of action. Further, A2a receptor antagonists may be useful in combination with checkpoint blockers. By way of example, the combination of a PD-1 inhibitor and an adenosine A2a receptor inhibitor is thought to mitigate the ability of tumors to inhibit the activity of tumor-specific effector T-cells. (See, e.g., Willingham, S B., et al., Cancer Immunol. Res.; 6(10), 1136-49; Leone, R D., et al., Cancer Immunol. Immunother., Aug. 2018, Vol. 67, Issue 8, pp. 1271-1284; Fishman, P., et al., Handb. Exp. Pharmacol. (2009) 193:399-441; and Sitkovsky, M V., et al., (2014) Cancer Immunol. Res 2:598-605.)

The A2b receptor is a G protein-coupled receptor found in various cell types. A2b receptors require higher concentrations of adenosine for activation than the other adenosine receptor subtypes, including A2a. (Fredholm, B B., et al., Biochem. Pharmacol. (2001) 61:443-448). Conditions which activate A2b have been seen, for example, in tumors where hypoxia is observed. The A2b receptor may thus play an important role in pathophysiological conditions associated with massive adenosine release. While the pathway(s) associated with A2b receptor-mediated inhibition are not well understood, it is believed that the inhibition of A2b receptors (alone or together with A2a receptors) may block pro-tumorigenic functions of adenosine in the tumor microenvironment, including suppression of T-cell function and angiogenesis, and thus expand the types of cancers treatable by the inhibition of these receptors.

A2b receptors are expressed primarily on myeloid cells. The engagement of A2b receptors on myeloid derived suppressor cells (MDSCs) results in their expansion in vitro (Ryzhov, S. et al., J. Immunol. 2011, 187:6120-6129). MDSCs suppress T-cell proliferation and anti-tumor immune responses. Selective inhibitors of A2b receptors and A2b receptor knockouts have been shown to inhibit tumor growth in mouse models by increasing MDSCs in the tumor microenvironment (Iannone, R., et al., Neoplasia Vol. 13 No. 12, (2013) pp. 1400-1409; Ryzhov, S., et al., Neoplasia (2008) 10: 987-995). Thus, A2b receptor inhibition has become an attractive biological target for the treatment of a variety of cancers involving myeloid cells. Examples of cancers that express A2b receptors can be readily obtained through analysis of the publicly available TCGA database. Such cancers include lung, colorectal, head and neck, and cervical cancer, among others, and are discussed in further detail below.

Angiogenesis plays an important role in tumor growth. The angiogenesis process is highly regulated by a variety of factors and is triggered by adenosine under particular circumstances that are associated with hypoxia. The A2b receptor is expressed in human microvascular endothelial cells, where it plays an important role in the regulation of the expression of angiogenic factors such as the vascular endothelial growth factor (VEGF). In certain tumor types, hypoxia has been observed to cause an upregulation of the A2b receptors, suggesting that inhibition of A2b receptors may limit tumor growth by limiting the oxygen supply to the tumor cells. Furthermore, experiments involving adenylate cyclase activation indicate that A2b receptors are the sole adenosine receptor subtype in certain tumor cells, suggesting that A2b receptor antagonists may exhibit effects on particular tumor types. (See, e.g., Feoktistov, I., et al., (2003) Circ. Res. 92:485-492; and P. Fishman, P., et al., Handb. Exp.

Pharmacol. (2009) 193:399-441). In view of their promising and varied therapeutic potential, there remains a need in the art for potent and selective inhibitors of the A2a and/or A2b adenosine receptors, for use alone or in combination with other therapeutic agents. The present invention addresses this and other needs.

### SUMMARY OF THE INVENTION

In one aspect, the present invention provides compounds (hereinafter referred to as compounds of the invention) which, surprisingly and advantageously, have been found to be inhibitors of the adenosine A2a receptor and/or the adenosine A2b receptor. The compounds of the invention have a structure in accordance with the structural Formula (I):

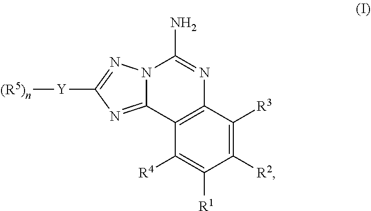

or a pharmaceutically acceptable salt thereof, wherein Y, R¹, R², R³, R⁴, R⁵ and n are as defined below.

In another aspect, the present invention provides pharmaceutical compositions comprising at least one compound of the invention, or a pharmaceutically acceptable salt thereof, and a pharmaceutically acceptable carrier or diluent. Such compositions according to the invention may optionally further include one or more additional therapeutic agents as described herein.

In another aspect, the present invention provides a method for treating or preventing a disease, condition, or disorder that is mediated, at least in part, by the adenosine A2a receptor and/or the adenosine A2b receptor in a subject (e.g., an animal or human) in need thereof, said method comprising administering to the subject a therapeutically effective amount of at least one compound of the invention, or a pharmaceutically acceptable salt thereof, alone or in combination with one or more additional therapeutic agents. These and other aspects and embodiments of the invention are described more fully below.

### DETAILED DESCRIPTION OF THE INVENTION

For each of the following embodiments, any variable not explicitly defined in the embodiment is as defined in Formula (I). In each of the embodiments described herein, each variable is selected independently of the other unless otherwise noted.

In one embodiment, the compounds of the invention have the structural Formula (I):

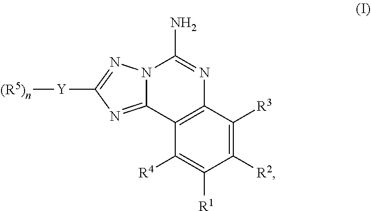

or a pharmaceutically acceptable salt thereof, wherein:
R¹ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl;
R² is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl;
R³ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl;
R⁴ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl;
Y is a straight or branched (C₁-C₅)alkyl or (C₃-C₆)cycloalkyl(C₁-C₅)alkyl, wherein one or more —CH₂— groups in Y are optionally and independently replaced with a moiety selected from the group consisting of S, O and SO₂;
each occurrence of R⁵ is independently selected from hydrogen, halogen, aryl, cycloheteroalkyl, heteroaryl, (C₁-C₆)alkylOH, OH, (C₁-C₆)haloalkyl, (C₁-C₆)alkyl, (C₁-C₆)alkynyl, SO₂R⁶, SO(═NH)R⁶, SO(═NCH₃)R⁶ and COO(C₁-C₆)alkyl, wherein the aryl, cycloheteroalkyl or heteroaryl are optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂;
R⁶ is selected from the group consisting of OH, NH₂, (C₁-C₆)alkyl, aryl, (C₁-C₆)haloalkyl, (C₃-C₆)cycloalkyl and haloaryl;
each occurrence of R⁷ is independently selected from the group consisting of H, (C₁-C₆)alkyl, (C₃-C₆)cycloalkyl, or when two R⁷ substituents are taken together with the nitrogen they are attached, form a cycloheteroalkyl;
n is 1, 2 or 3.

In another embodiment, the compounds of the invention comprise those compounds identified herein as examples in the tables below, and pharmaceutically acceptable salts thereof.

In another embodiment, the compounds described herein have a structure in accordance with the structural Formula (II):

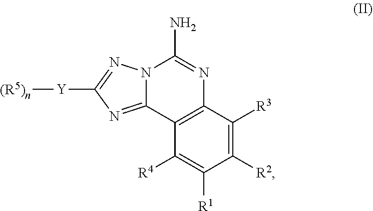

or a pharmaceutically acceptable salt thereof, wherein Y, R¹, R², R³, R⁴ and R⁵ are as defined below.

With regard to the compounds described herein, R¹ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl.

In certain embodiments, R¹ is hydrogen.

In certain embodiments, R¹ is halogen. Suitable halogens include, but are not limited to, fluorine, chlorine, bromine or iodine. In certain embodiments, R¹ is fluorine.

In certain embodiments, R¹ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R¹ is O(C₁-C₆)alkyl. Suitable alkoxys include, but are not limited to, methoxy, ethoxy, n-propoxy, isopropoxy and n-butoxy.

In certain embodiments, R¹ is OH.

In certain embodiments, R¹ is O(C₁-C₆)haloalkyl. Suitable examples of haloalkoxys include, but are not limited to, fluoromethoxy, difluoromethoxy, trifluoromethoxy, 2-fluoroethoxy, 1,2-difluoroethoxy and 2,2-difluoroethoxy.

In certain embodiments, R¹ is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R¹ is CN.

In certain embodiments, R¹ is (C₃-C₆)cycloalkyl. In certain embodiments, R¹ is a monocyclic cycloalkyl. In other embodiments, R¹ is a bicyclic cycloalkyl. In other embodiments, R¹ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R¹ is

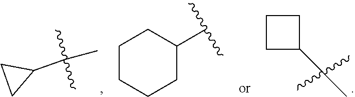

In certain embodiments, R¹ is cycloheteroalkyl. In certain embodiments, R¹ is a monocyclic cycloheteroalkyl. In other embodiments, R¹ is a bicyclic cycloheteroalkyl. In other embodiments, R¹ is a multicyclic cycloheteroalkyl. In other embodiments, R is a nitrogen-containing cycloheteroalkyl. In other embodiments, R¹ is an oxygen-containing cycloheteroalkyl. In certain embodiments the cycloheteroalkyl is

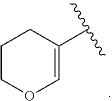 [SMILES: *C1=COCCC1]

In other embodiments, R¹ is a sulfur-containing cycloheteroalkyl.

In certain embodiments, R¹ is hydrogen or fluorine.

With regard to the compounds described herein, R² is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl.

In certain embodiments, R² is hydrogen.

In certain embodiments, R² is halogen. Suitable halogens include, but are not limited to, fluorine, chlorine, bromine and iodine.

In certain embodiments, R² is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R² is O(C₁-C₆)alkyl. Suitable alkoxys include, but are not limited to, methoxy, ethoxy, n-propoxy, isopropoxy and n-butoxy. In certain embodiments, R² is methoxy.

In certain embodiments, R² is OH.

In certain embodiments, R² is O(C₁-C₆)haloalkyl. Suitable examples of haloalkoxys include, but are not limited to, fluoromethoxys, difluoromethoxy, trifluoromethoxy, 2-fluoroethoxy, 1,2-difluoroethoxy and 2,2-difluoroethoxy.

In certain embodiments, R² is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R² is CN.

In certain embodiments, R² is (C₃-C₆)cycloalkyl. In certain embodiments, R² is a monocyclic cycloalkyl. In other embodiments, R² is a bicyclic cycloalkyl. In other embodiments, R² is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R² is

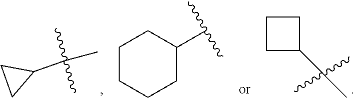

In certain embodiments, R² is cycloheteroalkyl. In certain embodiments, R² is a monocyclic cycloheteroalkyl. In other embodiments, R² is a bicyclic cycloheteroalkyl. In other embodiments, R² is a multicyclic cycloheteroalkyl. In other embodiments, R² is a nitrogen-containing cycloheteroalkyl. In other embodiments, R² is an oxygen-containing cycloheteroalkyl. In certain embodiments the cycloheteroalkyl is

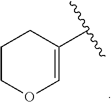 [SMILES: *C1=COCCC1]

In other embodiments, R² is a sulfur-containing cycloheteroalkyl.

In certain embodiments, R² is hydrogen or methoxy.

With regard to the compounds described herein, R³ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl.

In certain embodiments, R³ is hydrogen.

In certain embodiments, R³ is halogen. Suitable halogens include, but are not limited to, fluorine, chlorine, bromine and iodine. In certain embodiments, R³ is fluorine.

In certain embodiments, R³ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R³ is O(C₁-C₆)alkyl. Suitable alkoxys include, but are not limited to, methoxy, ethoxy, n-propoxy, isopropoxy and n-butoxy. In certain embodiments, R³ is methoxy.

In certain embodiments, R³ is OH.

In certain embodiments, R³ is O(C₁-C₆)haloalkyl. Suitable examples of haloalkoxy include, but are not limited to, fluoromethoxy, difluoromethoxy, trifluoromethoxy, 2-fluoroethoxy, 1,2-difluoroethoxy and 2,2-difluoroethoxy.

In certain embodiments, R³ is (C₁-C₆)haloalkyl. Suitable examples of haloalkyl include, but are not limited to, fluoromethyl, difluoromethyl, trifluoromethyl, 2-fluoroethyl, 1,2-difluoroethyl and 2,2-difluoroethyl.

In certain embodiments, R³ is CN.

In certain embodiments, R³ is (C₃-C₆)cycloalkyl. In certain embodiments, R³ is a monocyclic cycloalkyl. In other embodiments, R³ is a bicyclic cycloalkyl. In other embodiments, R³ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R³ is

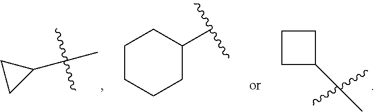

In certain embodiments, R³ is cycloheteroalkyl. In certain embodiments, R³ is a monocyclic cycloheteroalkyl. In other embodiments, R³ is a bicyclic cycloheteroalkyl. In other embodiments, R³ is a multicyclic cycloheteroalkyl. In other embodiments, R³ is a nitrogen-containing cycloheteroalkyl. In other embodiments, R³ is an oxygen-containing cycloheteroalkyl. In certain embodiments the cycloheteroalkyl is

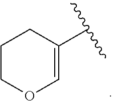 [SMILES: *C1=COCCC1]

In other embodiments, R³ is a sulfur-containing cycloheteroalkyl.

In certain embodiments, R³ is hydrogen, methoxy or fluorine.

With regard to the compounds described herein, R⁴ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl.

In certain embodiments, R⁴ is hydrogen.

In certain embodiments, R⁴ is halogen. Suitable halogens include, but are not limited to, fluorine, chlorine, bromine and iodine. In certain embodiments, R⁴ is fluorine.

In certain embodiments, R⁴ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl.

In certain embodiments, R⁴ is O(C₁-C₆)alkyl. Suitable alkoxys include, but are not limited to, methoxy, ethoxy, n-propoxy, isopropoxy and n-butoxy. In certain embodiments, R⁴ is methoxy.

In certain embodiments, R⁴ is OH.

In certain embodiments, R⁴ is O(C₁-C₆)haloalkyl. Suitable examples of haloalkoxys include, but are not limited to, fluoromethoxys, difluoromethoxy, trifluoromethoxy, 2-fluoroethoxy, 1,2-difluoroethoxy and 2,2-difluoroethoxy.

In certain embodiments, R⁴ is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R⁴ is CN.

In certain embodiments, R⁴ is (C₃-C₆)cycloalkyl. In certain embodiments, R⁴ is a monocyclic cycloalkyl. In other embodiments, R⁴ is a bicyclic cycloalkyl. In other embodiments, R⁴ is a multicyclic cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁴ is

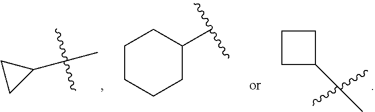

In certain embodiments, R⁴ is cycloheteroalkyl. In certain embodiments, R⁴ is a monocyclic cycloheteroalkyl. In other embodiments, R⁴ is a bicyclic cycloheteroalkyl. In other embodiments, R⁴ is a multicyclic cycloheteroalkyl. In other embodiments, R⁴ is a nitrogen-containing cycloheteroalkyl. In other embodiments, R⁴ is an oxygen-containing cycloheteroalkyl. In certain embodiments the cycloheteroalkyl is

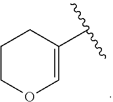 [SMILES: *C1=COCCC1]

In other embodiments, R⁴ is a sulfur-containing cycloheteroalkyl.

In certain embodiments, R⁴ is hydrogen or

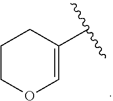 [SMILES: *C1=COCCC1]

In certain embodiments, R¹, R², R³, R⁴ are not simultaneously hydrogen.

With regard to the compounds described herein, Y is a straight or branched (C₁-C₅)alkyl or (C₃-C₆)cycloalkyl(C₁-C₅)alkyl, wherein one or more —CH₂— groups in Y are optionally and independently replaced with a moiety selected from the group consisting of S, O and SO₂. In certain embodiments, Y is a straight or branched (C₁-C₅)alkyl or (C₃-C₆)cycloalkyl(C₁-C₅)alkyl, wherein one or more —CH₂— groups in Y are independently replaced with a moiety selected from the group consisting of S, O and SO₂.

In certain embodiments described herein, Y is a straight or branched (C₁-C₅)alkyl or (C₃-C₆)cycloalkyl(C₁-C₅)alkyl, wherein one or more non-adjacent —CH₂— groups in Y are optionally and independently replaced with a moiety selected from the group consisting of S, O and SO₂.

In certain embodiments, Y is a straight or branched (C₁-C₅)alkyl or (C₃-C₆)cycloalkyl(C₁-C₅)alkyl, wherein one or more non-adjacent —CH₂— groups in Y are independently replaced with a moiety selected from the group consisting of S, O and SO₂.

In certain embodiments, Y is a straight or branched (C₂-C₅)alkyl or (C₃-C₆)cycloalkyl(C₁-C₅)alkyl, wherein one or more —CH₂— groups in Y are optionally and independently replaced with a moiety selected from the group consisting of S, O and SO₂. In certain embodiments, Y is a straight or branched (C₃-C₅)alkyl or (C₃-C₆)cycloalkyl(C₁-C₅)alkyl, wherein one or more —CH₂— groups in Y are optionally and independently replaced with a moiety selected from the group consisting of S, O and SO₂. In certain embodiments, Y is a straight or branched (C₄-C₅)alkyl or (C₃-C₆)cycloalkyl(C₁-C₅)alkyl, wherein one or more —CH₂— groups in Y are optionally and independently replaced with a moiety selected from the group consisting of S, O and SO₂. In certain embodiments, Y is a straight or branched (C₂-C₄)alkyl or (C₃-C₆)cycloalkyl(C₁-C₅)alkyl, wherein one or more —CH₂— groups in Y are optionally and independently replaced with a moiety selected from the group consisting of S, O and SO₂.

In certain embodiments, Y is C₂-C₅alkyl, wherein one or more —CH₂— groups in Y are optionally and independently replaced with a moiety selected from the group consisting of S, O and SO₂. In certain embodiments, Y is (C₃-C₆)cycloalkyl(C₁-C₅)alkyl wherein one or more —CH₂— groups in Y are optionally and independently replaced with a moiety selected from the group consisting of S, O and SO₂. In certain embodiments, Y is C₂-C₅alkyl, wherein one or more non-adjacent —CH₂— groups in Y are optionally and independently replaced with a moiety selected from the group consisting of S, O and SO₂. In certain embodiments, Y is (C₃-C₆)cycloalkyl(C₁-C₅)alkyl wherein one or more non-adjacent —CH₂— groups in Y are optionally and independently replaced with a moiety selected from the group consisting of S, O and SO₂.

In certain embodiments, Y is a straight or branched (C₂-C₄)alkyl or (C₃-C₆)cycloalkyl(C₁-C₅)alkyl, wherein one —CH₂— group in Y is optionally and independently replaced with a moiety selected from the group consisting of S, O and SO₂.

In certain embodiments, Y is a straight or branched (C₂-C₄)alkyl or (C₃-C₆)cycloalkyl(C₁-C₅)alkyl, wherein one —CH₂— group in Y is optionally and independently replaced with a moiety selected from the group consisting of S and O.

In certain embodiments, Y is a straight or branched (C₂-C₄)alkyl or (C₃-C₆)cycloalkyl(C₁-C₅)alkyl, wherein one —CH₂— group in Y is optionally and independently replaced with an SO₂.

In certain embodiments, Y is a straight (C₁-C₅)alkyl. In certain embodiments, Y is a branched (C₁-C₅)alkyl. In another embodiment, Y is a (C₃-C₆)cycloalkyl(C₁-C₅)alkyl.

In certain embodiments, Y is a straight (C₁-C₅)alkyl, wherein one or more —CH₂— groups in Y are independently replaced with a moiety selected from the group consisting of S, O and SO₂. In other embodiments, Y is a branched (C₁-C₅)alkyl, wherein one or more —CH₂— groups in Y are independently replaced with a moiety selected from the group consisting of S, O and SO₂.

In certain embodiments, Y is (C₃-C₆)cycloalkyl(C₁-C₅)alkyl, wherein one or more —CH₂— groups in Y are independently replaced with a moiety selected from the group consisting of S, O and SO₂.

In certain embodiments, Y is a straight (C₁-C₅)alkyl, wherein one or more non-adjacent —CH₂— groups in Y are independently replaced with a moiety selected from the group consisting of S, O and SO₂. In other embodiments, Y is a branched (C₁-C₅)alkyl, wherein one or more non-adjacent —CH₂— groups in Y are independently replaced with a moiety selected from the group consisting of S, O and SO₂. In certain embodiments, Y is (C₃-C₆)cycloalkyl(C₁-C₅)alkyl, wherein one or more non-adjacent —CH₂— groups in Y are independently replaced with a moiety selected from the group consisting of S, O and SO₂.

In certain embodiments, Y is a straight or branched (C₁-C₅)alkyl, wherein Y is

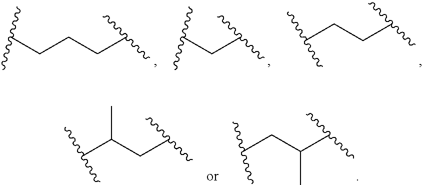

In certain embodiments, Y is a straight (C₁-C₅)alkyl, wherein Y is

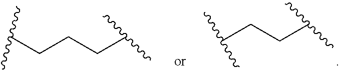

In another embodiment, Y is a (C₃-C₆)cycloalkyl(C₁-C₅)alkyl, wherein Y is

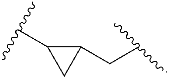 [SMILES: *CC1CC1*]

In certain embodiments, Y is a straight or branched (C₁-C₅)alkyl, wherein one or more —CH₂— groups in Y are independently replaced with a moiety selected from the group consisting of S, wherein Y is or

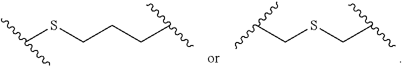 [SMILES: *CCCS* ; *CSC*]

In certain embodiments, Y is a straight or branched (C₁-C₅)alkyl, wherein one or more —CH₂— groups in Y are independently replaced with a moiety selected from the group consisting of O, wherein Y is

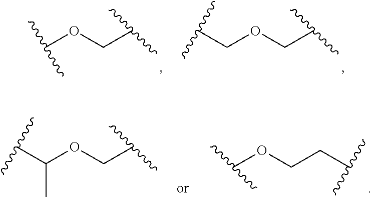

In certain embodiments, Y is a straight or branched (C₁-C₅)alkyl, wherein one or more —CH₂— groups in Y are independently replaced with a moiety selected from the group consisting of SO₂, wherein Y is

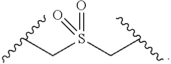 [SMILES: *CS(=O)(=O)C*]

In certain embodiments, Y is

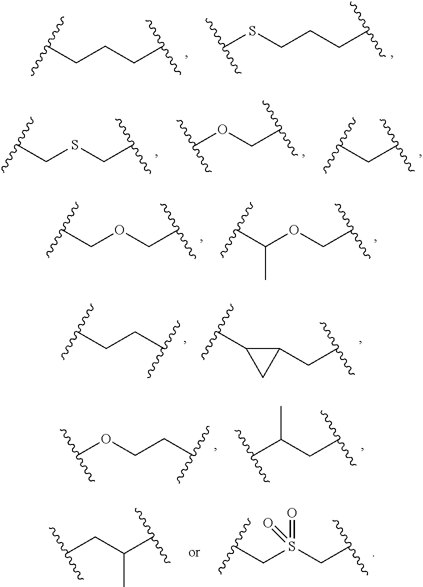

With regard to the compounds described herein, each occurrence of R⁵ is independently selected from hydrogen, halogen, aryl, cycloheteroalkyl, heteroaryl, (C₁-C₆)alkylOH, OH, (C₁-C₆)haloalkyl, (C₁-C₆)alkyl, (C₁-C₆)alkynyl, SO₂R⁶, SO(═NH)R⁶, SO(═NCH₃)R⁶ and COO(C₁-C₆)alkyl, wherein the aryl, cycloheteroalkyl or heteroaryl are optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂.

In certain embodiments, when R⁵ is attached to Y, wherein Y is a straight or branched (C₁-C₅)alkyl or (C₃-C₆)cycloalkyl(C₁-C₅)alkyl, wherein one or more —CH₂— groups in Y are optionally and independently replaced with a moiety selected from the group consisting of S, O and SO₂, R⁵ can be attached to any carbon in (C₁-C₅)alkyl.

In certain embodiments, R⁵ is hydrogen.

In certain embodiments, R⁵ is halogen. Suitable halogens include, but are not limited to, a fluorine, a chlorine, a bromine or an iodine. In certain embodiments, R⁵ is chlorine or fluorine.

In other embodiments, R⁵ is chlorine.

In certain embodiments, R⁵ is aryl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl, (C₁-C₆)alkylC(O)O(C₁-C₆)alkyl or (C₁-C₆)alkylN(R⁷)₂. In certain embodiments, R⁵ is aryl. In certain embodiments, R⁵ is phenyl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In other embodiments, R⁵ is naphthyl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl, (C₁-C₆)alkylC(O)O(C₁-C₆)alkyl or (C₁-C₆)alkylN(R⁷)₂. In other embodiments, R⁵ is a multicyclic aryl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, R⁵ is phenyl, para-substituted with a substituent selected from halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂. In certain embodiments, R⁵ is phenyl, substituted with one to three substituents selected from CF₃, flourine, C(CF₃)₂OH, C(CH₃)₂OH, or C(CH₃)₂NH₂. In certain embodiments, R⁵ is phenyl, para-substituted with CF₃, flourine, C(CF₃)₂OH, C(CH₃)₂OH, or C(CH₃)₂NH₂. In other embodiments, R⁵ is naphthyl optionally substituted with one to three substituents selected from CF₃, flourine, C(CF₃)₂OH, C(CH₃)₂OH, or C(CH₃)₂NH₂.

In certain embodiments, R⁵ is para-substituted phenyl substituted with CF₃. In certain embodiments, R⁵ is para-substituted phenyl substituted with fluorine. In certain embodiments, R⁵ is para-substituted phenyl substituted with C(CF₃)₂OH. In certain embodiments, R⁵ is para-substituted phenyl substituted with C(CH₃)₂OH. In certain embodiments, R⁵ is para-substituted phenyl substituted with C(CH₃)₂NH₂).

In certain embodiments, R⁵ is cycloheteroalkyl optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, R⁵ is a nitrogen-containing cycloheteroalkyl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, R⁵ is a sulfur-containing cycloheteroalkyl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, R⁵ is a monocyclic cycloheteroalkyl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In other embodiments, R⁵ is a bicyclic cycloheteroalkyl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In other embodiments, R⁵ is a multicyclic cycloheteroalkyl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, R⁵ is

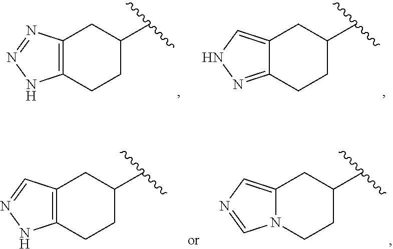 [SMILES: *C1CCc2[nH]nnc2C1 ; *C1CCc2n[nH]cc2C1 ; *C1CCc2[nH]ncc2C1 ; *C1CCn2cncc2C1]

optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂.

In certain embodiments, R⁵ is

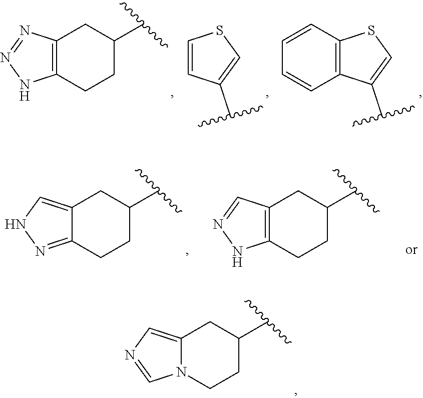

optionally substituted with (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂.

In certain embodiments, R⁵ is heteroaryl optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂. In certain embodiments, R⁵ is heteroaryl, optionally substituted with one to three substituents selected from (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂. In certain embodiments, R⁵ is a sulfur- or nitrogen-containing heteroaryl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂. In certain embodiments, R⁵ is a sulfur-containing heteroaryl.

In certain embodiments, R⁵ is a nitrogen-containing heteroaryl, optionally substituted with one, two or three substituents selected from (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂. In certain embodiments, R⁵ is a monocyclic heteroaryl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂. In other embodiments, R⁵ is a bicyclic heteroaryl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂. In other embodiments, R⁵ is a multicyclic heteroaryl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl, optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂. In certain embodiments, R⁵ is is

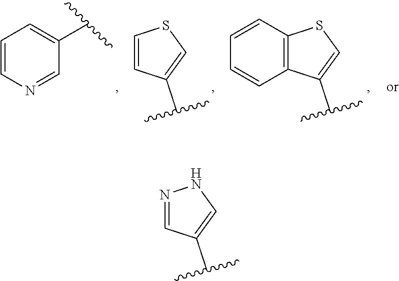 [SMILES: *c1cccnc1 ; *c1ccsc1 ; *c1csc2ccccc12 ; *c1cn[nH]c1]

optionally substituted with (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, R⁵ is

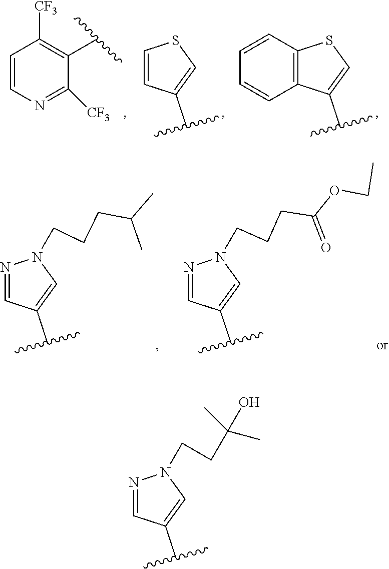 [SMILES: *c1c(C(F)(F)F)ccnc1C(F)(F)F ; *c1ccsc1 ; *c1csc2ccccc12 ; *c1cnn(CCCC(C)C)c1 ; *c1cnn(CCCC(=O)OCC)c1 ; *c1cnn(CCC(C)(C)O)c1]

optionally substituted with (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂.

In certain embodiments, R⁵ is (C₁-C₆)alkylOH. Suitable alcohols include, but are not limited to, methanol, ethanol, propanol and butanol. In certain embodiments, R⁵ is

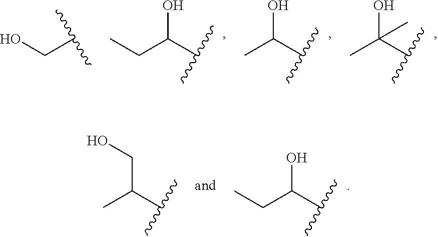

In certain embodiments, R⁵ is

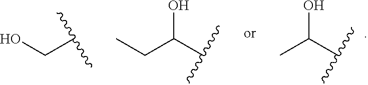

In certain embodiments, R⁵ is OH.

In certain embodiments, R⁵ is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁵ is difluoromethyl or trifluoromethyl.

In certain embodiments, R⁵ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is methyl or isopropyl.

In certain embodiments, R⁵ is SO₂R⁶, wherein R⁶ is discussed below.

In certain embodiments, R⁵ is SO(═NH)R⁶, wherein R⁶ is discussed below. In certain embodiments, R⁵ is

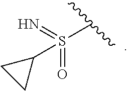 [SMILES: *S(=N)(=O)C1CC1]

In certain embodiments, R⁵ is SO(═NCH₃)R⁶, wherein R⁶ is discussed below.

In certain embodiments, R⁵ is COO(C₁-C₆)alkyl. In certain embodiments, R⁶ is —COOCH₂CH₃.

In certain embodiments, R⁵ is unsubstituted.

In other embodiments, R⁵ is substituted. In certain embodiments, when R⁵ is aryl, cycloheteroalkyl or heteroaryl, the aryl, cycloheteroalkyl or heteroaryl are optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, when R⁵ is aryl, cycloheteroalkyl or heteroaryl, the aryl, cycloheteroalkyl or heteroaryl are substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, when R⁵ is aryl, cycloheteroalkyl or heteroaryl, the aryl, cycloheteroalkyl or heteroaryl are substituted with one substituent selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, when R⁵ is aryl, cycloheteroalkyl or heteroaryl, the aryl, cycloheteroalkyl or heteroaryl are optionally substituted with two substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂. In certain embodiments, when R⁵ is aryl, cycloheteroalkyl or heteroaryl, the aryl, cycloheteroalkyl or heteroaryl are substituted with three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylN(R⁷)₂.

In certain embodiments, when R⁵ is aryl, cycloheteroalkyl, or heteroaryl, R⁵ is substituted with halogen. Suitable halogens include, but are not limited to, fluorine, chlorine, bromine or an iodine. In certain embodiments, R⁵ is substituted with fluorine.

In certain embodiments, when R⁵ is aryl, cycloheteroalkyl, or heteroaryl, R⁵ is substituted with (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is substituted with methyl or isopropyl.

In certain embodiments, when R⁵ is aryl, cycloheteroalkyl or heteroaryl, R⁵ is substituted with (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁵ is substituted with trifluoromethyl.

In certain embodiments, when R⁵ is aryl, cycloheteroalkyl or heteroaryl, R⁵ is substituted with (C₃-C₆)cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁵ is aryl, cycloheteroalkyl, or heteroaryl substituted with:

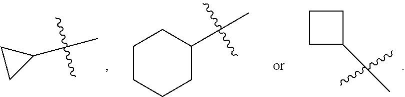

In certain embodiments, when R⁵ is aryl, cycloheteroalkyl or heteroaryl, R⁵ is substituted with (C₃-C₆)halocycloalkyl. In certain embodiments, when R⁵ is aryl, cycloheteroalkyl or heteroaryl, R⁵ is substituted with

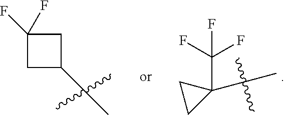 [SMILES: *C1CC(F)(F)C1 ; *C1(C(F)(F)F)CC1]

In certain embodiments, when R⁵ is aryl, cycloheteroalkyl or heteroaryl, R⁵ is substituted with (C₁-C₆)haloalkyl(C₃-C₆)cycloalkyl. In certain embodiments, R⁵ is substituted with

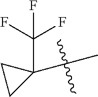 [SMILES: *C1(C(F)(F)F)CC1]

In certain embodiments, when R⁵ is aryl, cycloheteroalkyl or heteroaryl, R⁵ is substituted with (C₁-C₆)haloalkylOH. In certain embodiments, R⁵ is substituted with

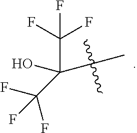 [SMILES: *C(O)(C(F)(F)F)C(F)(F)F]

In certain embodiments, when R⁵ is aryl, cycloheteroalkyl or heteroaryl, R⁵ is substituted with (C₁-C₆)alkylOH. In certain embodiments, R⁵ is substituted with

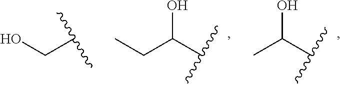

CH₂CH(OH)CH₂CH₃, CH(CH₃)(CH₂OH), CH₂C(CH₃)₂OH. In certain embodiments, R⁵ is substituted with

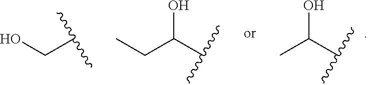

In certain embodiments, R⁵ is substituted with

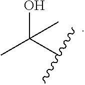 [SMILES: *C(C)(C)O]

In certain embodiments, when R⁵ is aryl, cycloheteroalkyl or heteroaryl, R⁵ is substituted with (C₁-C₆)alkylC(O)O(C₁-C₆)alkyl. In certain embodiments, when R⁵ is aryl, cycloheteroalkyl or heteroaryl, R⁵ is substituted with

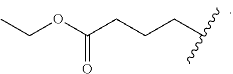 [SMILES: *CCCC(=O)OCC]

In certain embodiments, when R⁵ is aryl, cycloheteroalkyl or heteroaryl, R⁵ is substituted with (C₁-C₆)alkylN(R⁷)₂, wherein R⁷ is described below. In certain embodiments, when R⁵ is aryl, cycloheteroalkyl or heteroaryl, R⁵ is substituted with

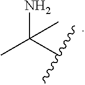 [SMILES: *C(C)(C)N]

With regard to the compounds described herein, R⁶ is selected from the group consisting of OH, NH₂, (C₁-C₆)alkyl, aryl, (C₁-C₆)haloalkyl, (C₃-C₆)cycloalkyl and haloaryl.

In certain embodiments, R⁶ is OH.

In certain embodiments, R⁶ is NH₂.

In certain embodiments, R⁶ is (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁶ is methyl, cyclopropyl, ethyl, iso-butyl or iso-propyl.

In certain embodiments, R⁶ is aryl. In certain embodiments, R⁶ is a monocyclic aryl. In other embodiments, R⁶ is a bicyclic aryl. In other embodiments, R⁶ is a multicyclic aryl. Suitable aryls include, but are not limited to, phenyl and naphthyl. In certain embodiments, R⁶ is phenyl. In certain embodiments, R⁶ naphthyl.

In certain embodiments, R⁶ is (C₁-C₆)haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁶ is trifluoromethyl.

In certain embodiments, R⁶ is (C₃-C₆)cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁶ is

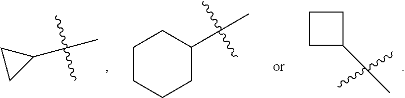

In certain embodiments, R⁶ is

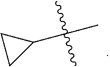 [SMILES: *C1CC1]

In certain embodiments, R⁶ is haloaryl. In certain embodiments, R⁶ is fluorophenyl.

In certain embodiments, R⁶ is methyl, NH₂, phenyl, cyclopropyl, fluorophenyl, trifluoromethyl, ethyl, iso-butyl or iso-propyl.

With regard to the compounds described herein, each occurrence of R⁷ is independently selected from the group consisting of hydrogen, (C₁-C₆)alkyl, (C₃-C₆)cycloalkyl, or when two R⁷ substituents are taken together with the nitrogen they are attached, form a cycloheteroalkyl.

In certain embodiments, R⁷ is hydrogen.

In certain embodiments, R⁷ (C₁-C₆)alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁷ is methyl, cyclopropyl, ethyl, iso-butyl or iso-propyl.

In certain embodiments, R⁷ (C₃-C₆)cycloalkyl. Suitable cycloalkyls include, but are not limited to, cyclopropyl, cyclobutyl, cyclopentyl, cyclohexyl, cycloheptyl, tetrahydronaphthyl, decahydronaphthyl, indanyl. In certain embodiments, R⁷ is

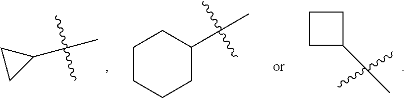

In certain embodiments, R⁷ when two R⁷ substituents are taken together with the nitrogen they are attached, form a cycloheteroalkyl.

With regard to the compounds described herein, n is 1, 2 or 3. In certain embodiments, n is 1. In certain embodiments, n is 2. In certain embodiments, n is 3.

In certain embodiments, R⁵ is chlorine, methyl, fluromethyl, difluoromethyl, trifluoromethyl, OH, propyl, phenyl, SO₂R⁶, —COOCH₂CH₃,

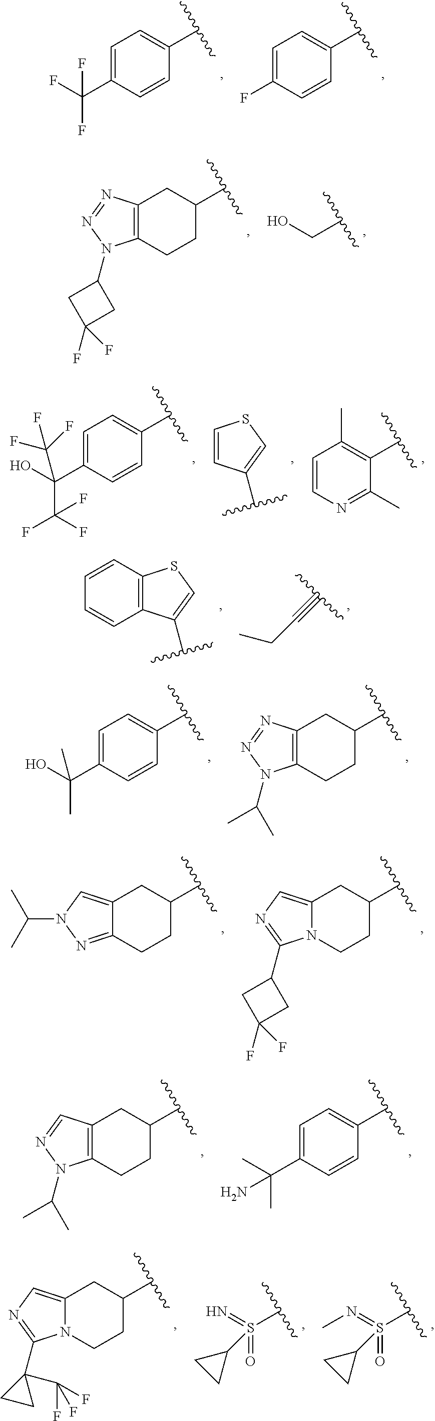

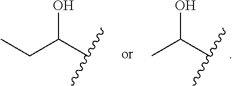

Also described herein are compounds having Formula (I):

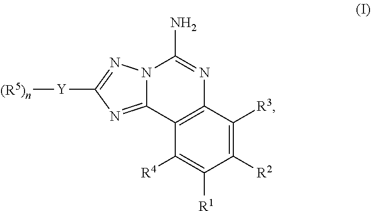

or a pharmaceutically acceptable salt thereof, wherein:
R¹ is selected from the group consisting of hydrogen, halogen, (C₁-C₆)alkyl, O(C₁-C₆)alkyl and cycloheteroalkyl;
R² is selected from the group consisting of hydrogen, halogen, (C₁-C₆)alkyl, O(C₁-C₆)alkyl and cycloheteroalkyl;
R³ is selected from the group consisting of hydrogen, halogen, (C₁-C₆)alkyl, O(C₁-C₆)alkyl and cycloheteroalkyl;
R⁴ is selected from the group consisting of hydrogen, halogen, (C₁-C₆)alkyl, O(C₁-C₆)alkyl and cycloheteroalkyl; Y is a straight or branched (C₁-C₅)alkyl or cycloalkyl(C₁-C₅)alkyl, wherein one or more —CH₂— groups in Y are optionally and independently replaced with a moiety selected from the group consisting of S and O;
R⁵ is hydrogen, halogen, aryl, cycloheteroalkyl, heteroaryl, (C₁-C₆)alkylOH, OH, (C₁-C₆)haloalkyl, (C₁-C₆)alkyl, (C₁-C₆)alkynyl, SO₂R⁶, SO(═NH)R⁶, SO(═NCH₃)R⁶ and COO(C₁-C₆)alkyl, wherein the aryl, cycloheteroalkyl or heteroaryl are optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl or (C₁-C₆)alkylNH₂;
R⁶ is selected from the group consisting of NH₂, (C₁-C₆)alkyl, aryl, (C₁-C₆)haloalkyl, (C₃-C₆)cycloalkyl and haloaryl;
n is 1, 2 or 3.

It should be noted that chemically unstable compounds are excluded from the embodiments contained herein.

Also described herein are compounds, or pharmaceutically acceptable salts thereof, having the following structure:

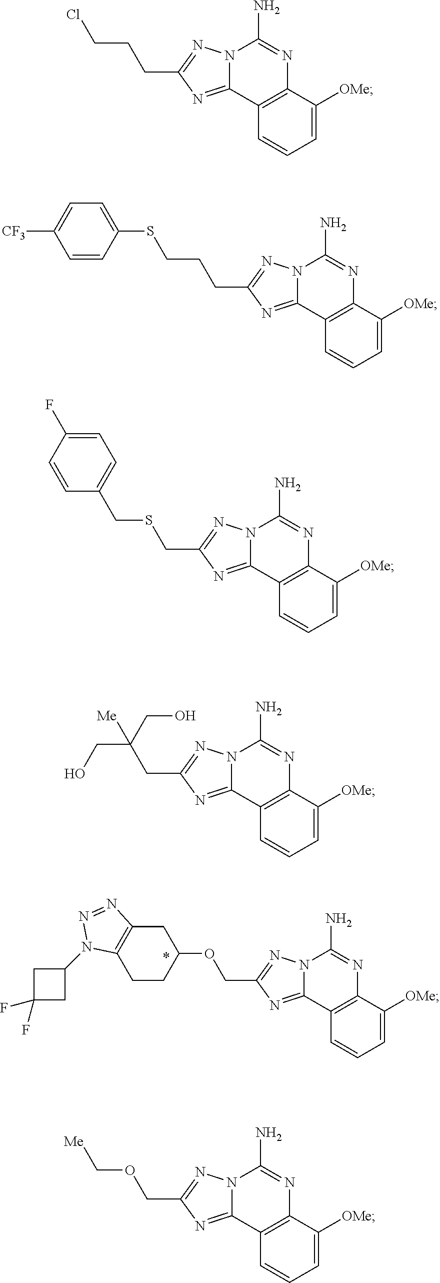 [SMILES: COc1cccc2c1nc(N)n1nc(CCCCl)nc21 ; COc1cccc2c1nc(N)n1nc(CCCSc3ccc(C(F)(F)F)cc3)nc21 ; COc1cccc2c1nc(N)n1nc(CSCc3ccc(F)cc3)nc21 ; COc1cccc2c1nc(N)n1nc(CC(C)(CO)CO)nc21 ; COc1cccc2c1nc(N)n1nc(COC3CCc4c(nnn4C4CC(F)(F)C4)C3)nc21 ; CCOCc1nc2c3cccc(OC)c3nc(N)n2n1]

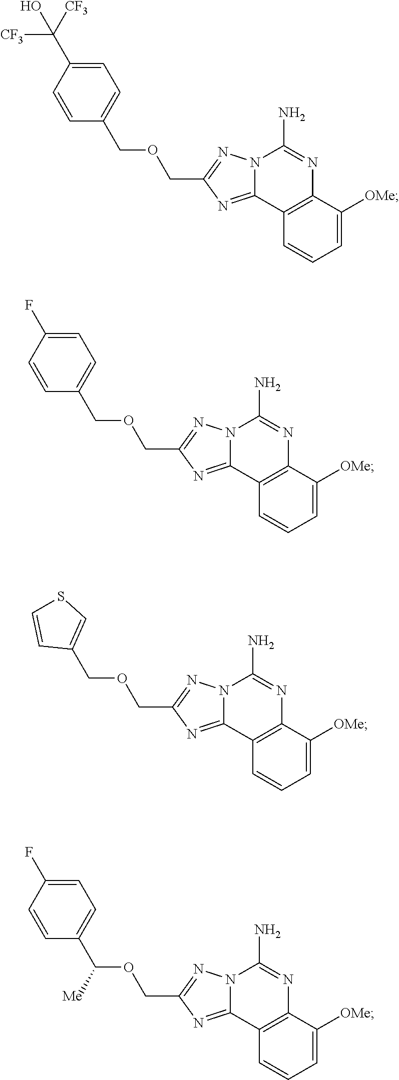 [SMILES: COc1cccc2c1nc(N)n1nc(COCc3ccc(C(O)(C(F)(F)F)C(F)(F)F)cc3)nc21 ; COc1cccc2c1nc(N)n1nc(COCc3ccc(F)cc3)nc21 ; COc1cccc2c1nc(N)n1nc(COCc3ccsc3)nc21 ; COc1cccc2c1nc(N)n1nc(CO[C@H](C)c3ccc(F)cc3)nc21]

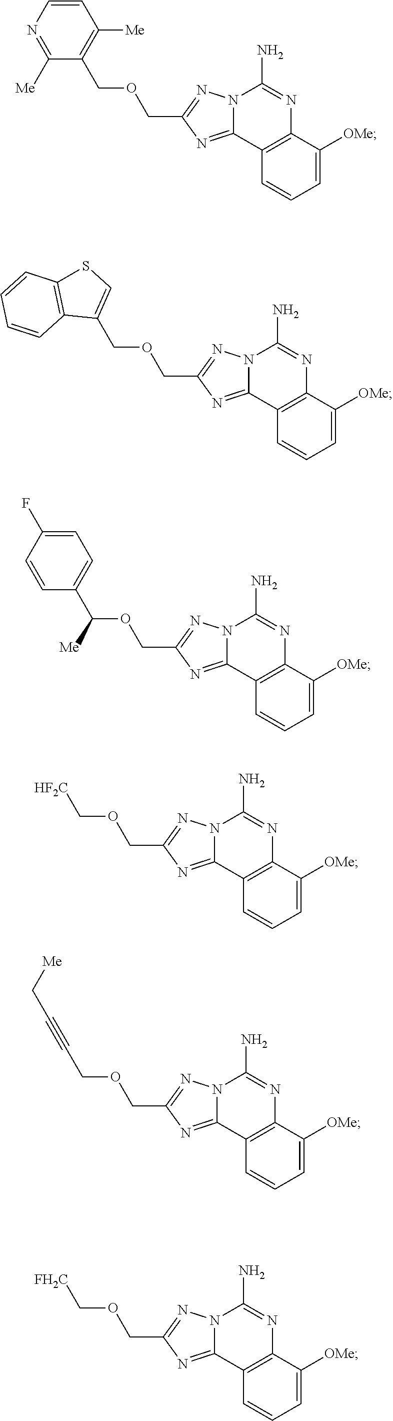

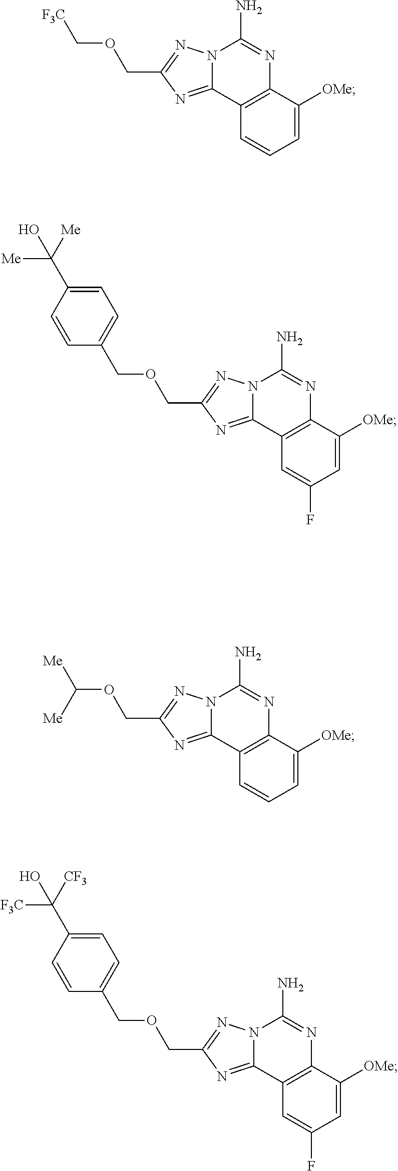 [SMILES: COc1cccc2c1nc(N)n1nc(COCC(F)(F)F)nc21 ; COc1cc(F)cc2c1nc(N)n1nc(COCc3ccc(C(C)(C)O)cc3)nc21 ; COc1cccc2c1nc(N)n1nc(COC(C)C)nc21 ; COc1cc(F)cc2c1nc(N)n1nc(COCc3ccc(C(O)(C(F)(F)F)C(F)(F)F)cc3)nc21]

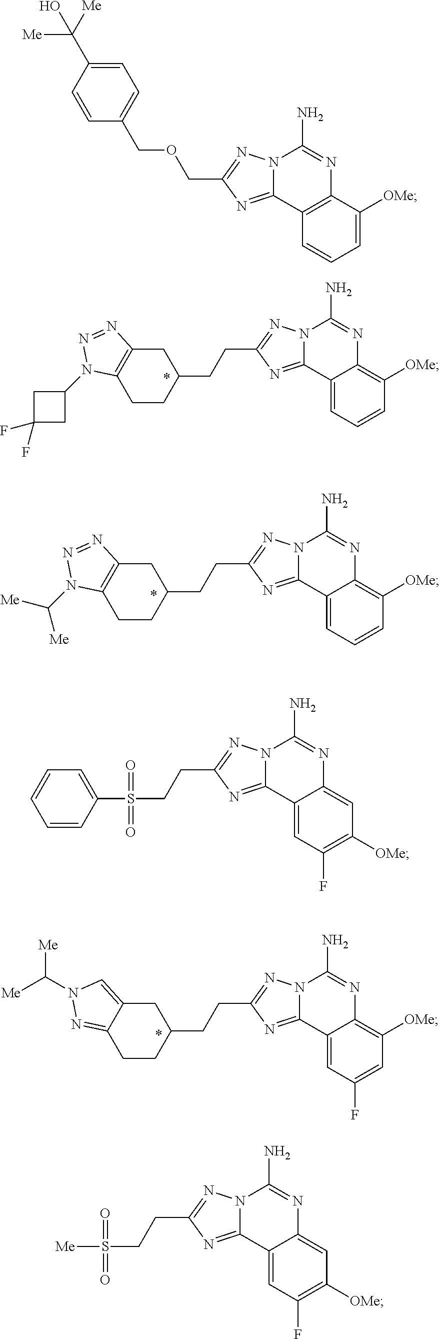

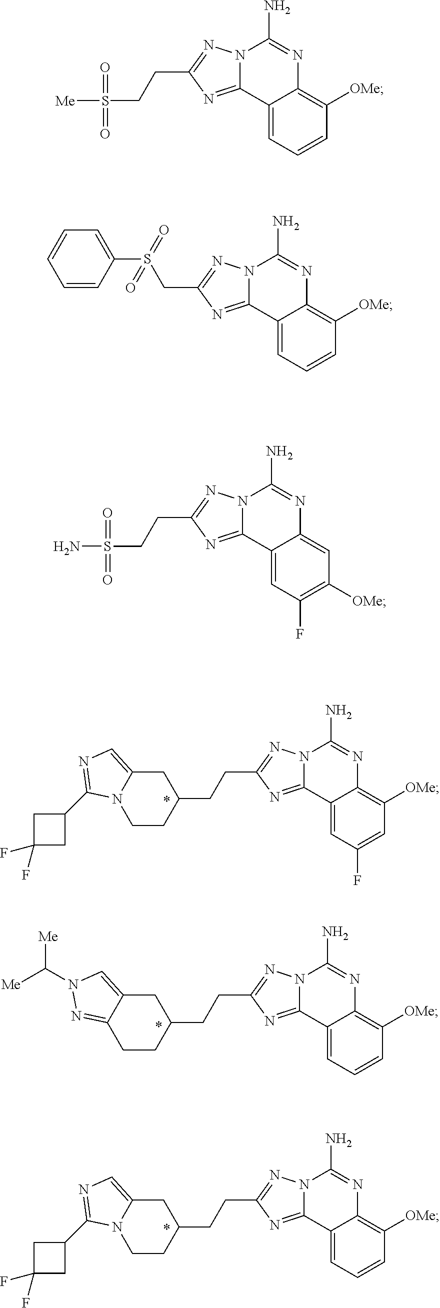 [SMILES: COc1cccc2c1nc(N)n1nc(CCS(C)(=O)=O)nc21 ; COc1cccc2c1nc(N)n1nc(CS(=O)(=O)c3ccccc3)nc21 ; COc1cc2nc(N)n3nc(CCS(N)(=O)=O)nc3c2cc1F ; COc1cc(F)cc2c1nc(N)n1nc(CCC3CCn4c(cnc4C4CC(F)(F)C4)C3)nc21 ; COc1cccc2c1nc(N)n1nc(CCC3CCc4nn(C(C)C)cc4C3)nc21 ; COc1cccc2c1nc(N)n1nc(CCC3CCn4c(cnc4C4CC(F)(F)C4)C3)nc21]

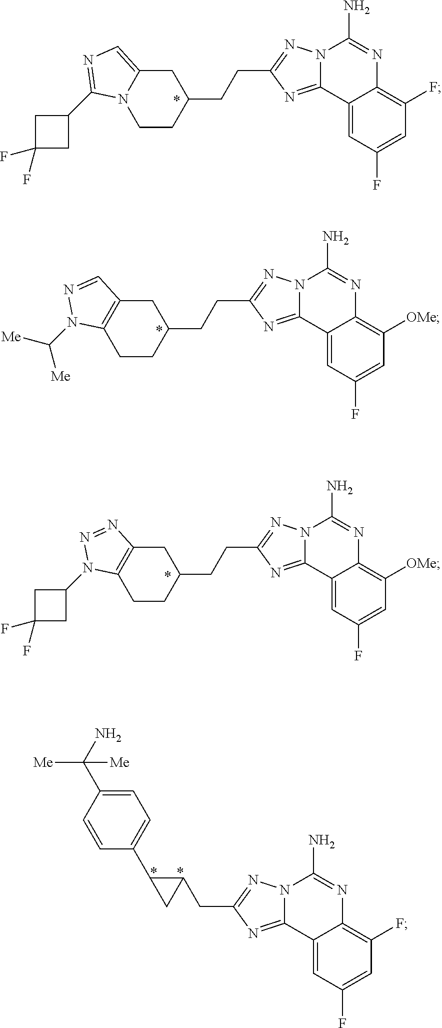 [SMILES: Nc1nc2c(F)cc(F)cc2c2nc(CCC3CCn4c(cnc4C4CC(F)(F)C4)C3)nn12 ; COc1cc(F)cc2c1nc(N)n1nc(CCC3CCc4c(cnn4C(C)C)C3)nc21 ; COc1cc(F)cc2c1nc(N)n1nc(CCC3CCc4c(nnn4C4CC(F)(F)C4)C3)nc21 ; CC(C)(N)c1ccc(C2CC2Cc2nc3c4cc(F)cc(F)c4nc(N)n3n2)cc1]

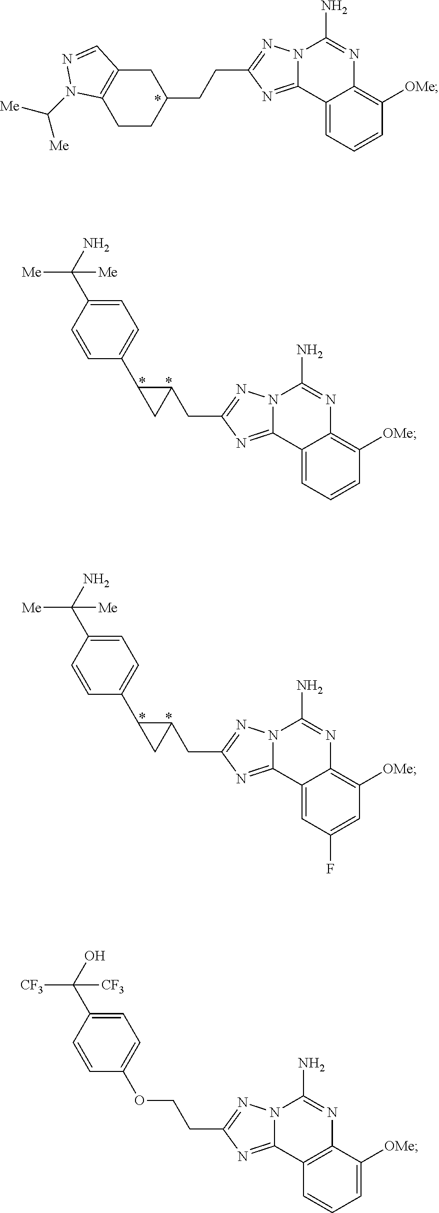 [SMILES: COc1cccc2c1nc(N)n1nc(CCC3CCc4c(cnn4C(C)C)C3)nc21 ; COc1cccc2c1nc(N)n1nc(CC3CC3c3ccc(C(C)(C)N)cc3)nc21 ; COc1cc(F)cc2c1nc(N)n1nc(CC3CC3c3ccc(C(C)(C)N)cc3)nc21 ; COc1cccc2c1nc(N)n1nc(CCOc3ccc(C(O)(C(F)(F)F)C(F)(F)F)cc3)nc21]

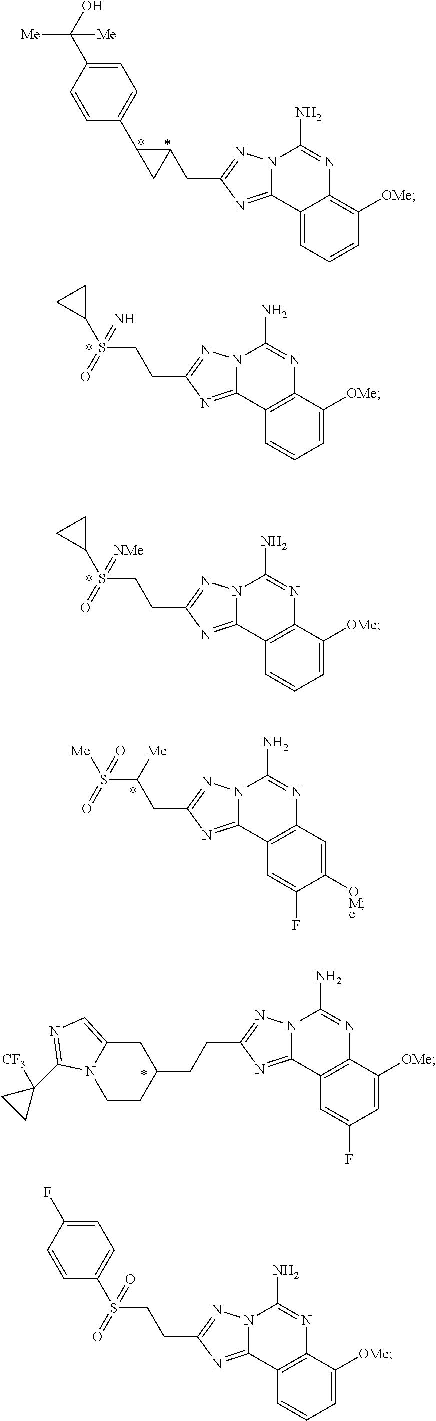

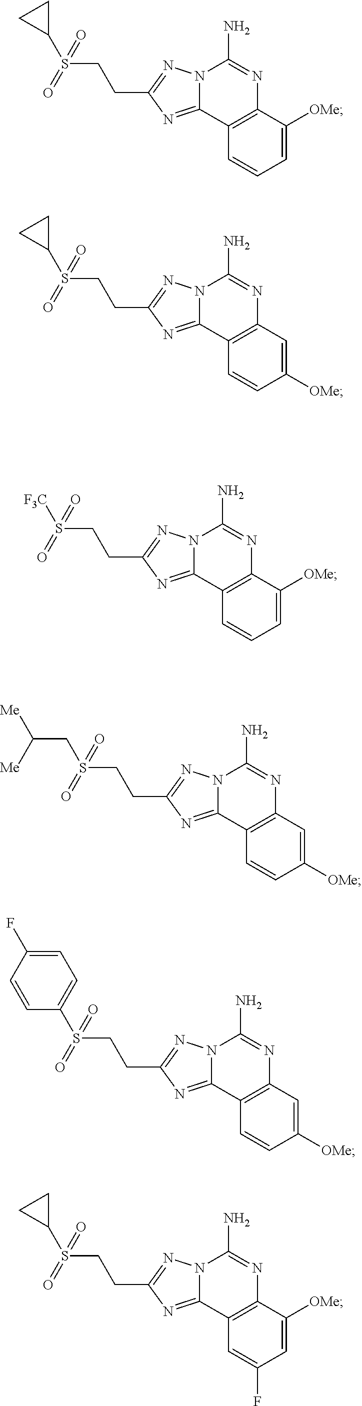 [SMILES: COc1cccc2c1nc(N)n1nc(CCS(=O)(=O)C3CC3)nc21 ; COc1ccc2c(c1)nc(N)n1nc(CCS(=O)(=O)C3CC3)nc21 ; COc1cccc2c1nc(N)n1nc(CCS(=O)(=O)C(F)(F)F)nc21 ; COc1ccc2c(c1)nc(N)n1nc(CCS(=O)(=O)CC(C)C)nc21 ; COc1ccc2c(c1)nc(N)n1nc(CCS(=O)(=O)c3ccc(F)cc3)nc21 ; COc1cc(F)cc2c1nc(N)n1nc(CCS(=O)(=O)C3CC3)nc21]

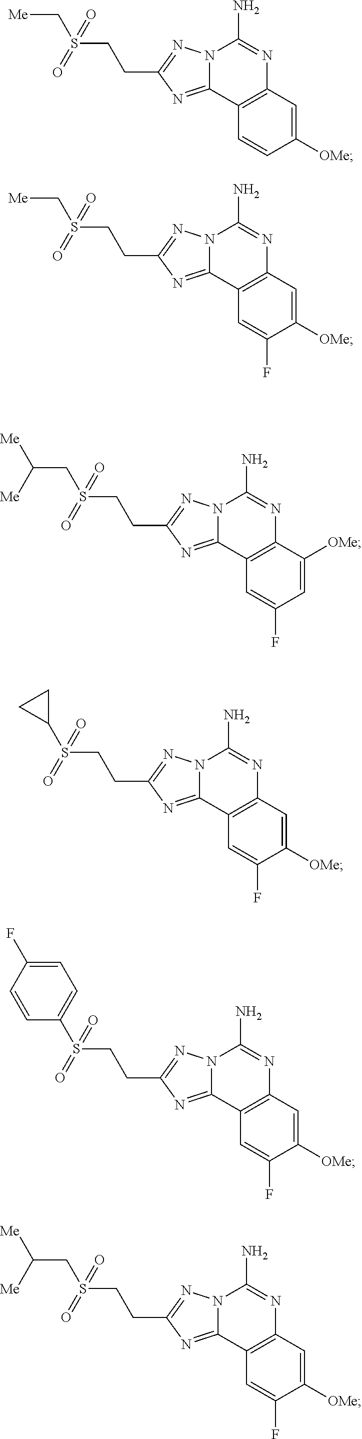

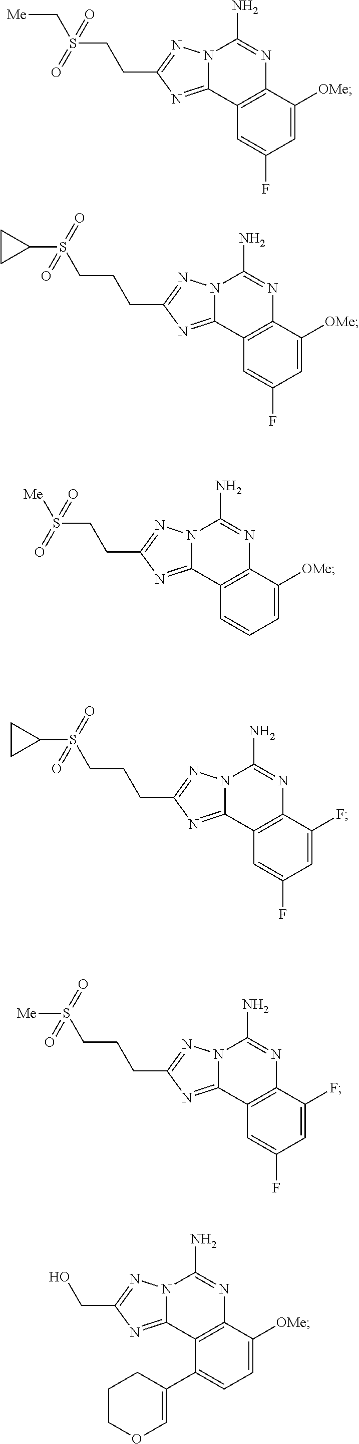 [SMILES: CCS(=O)(=O)CCc1nc2c3cc(F)cc(OC)c3nc(N)n2n1 ; COc1cc(F)cc2c1nc(N)n1nc(CCCS(=O)(=O)C3CC3)nc21 ; COc1cccc2c1nc(N)n1nc(CCS(C)(=O)=O)nc21 ; Nc1nc2c(F)cc(F)cc2c2nc(CCCS(=O)(=O)C3CC3)nn12 ; CS(=O)(=O)CCCc1nc2c3cc(F)cc(F)c3nc(N)n2n1 ; COc1ccc(C2=COCCC2)c2c1nc(N)n1nc(CO)nc21]

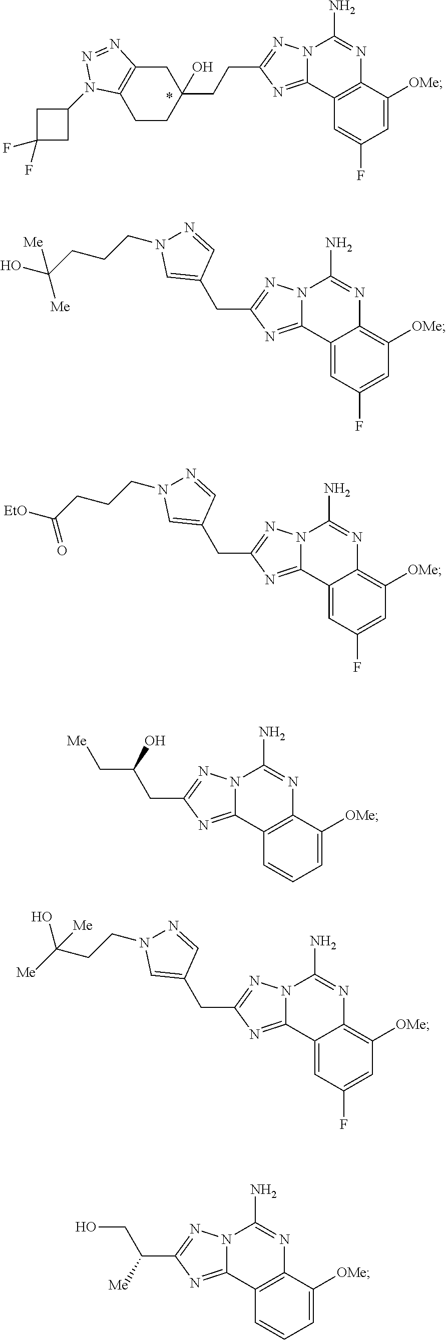 [SMILES: COc1cc(F)cc2c1nc(N)n1nc(CCC3(O)CCc4c(nnn4C4CC(F)(F)C4)C3)nc21 ; COc1cc(F)cc2c1nc(N)n1nc(Cc3cnn(CCCC(C)(C)O)c3)nc21 ; CCOC(=O)CCCn1cc(Cc2nc3c4cc(F)cc(OC)c4nc(N)n3n2)cn1 ; CC[C@@H](O)Cc1nc2c3cccc(OC)c3nc(N)n2n1 ; COc1cc(F)cc2c1nc(N)n1nc(Cc3cnn(CCC(C)(C)O)c3)nc21 ; COc1cccc2c1nc(N)n1nc([C@H](C)CO)nc21]

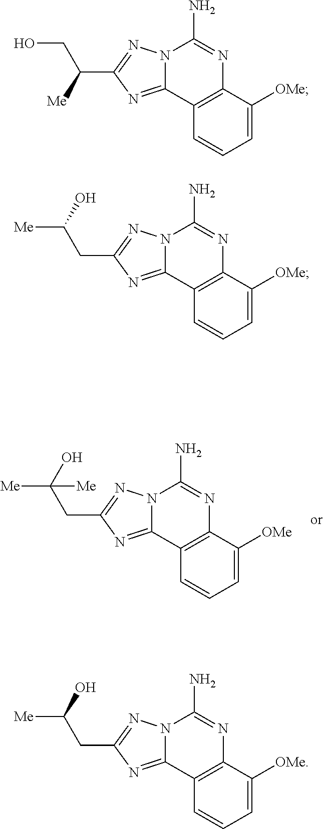

With regard to the structures described herein, Me meand methyl or CH₃.

In another aspect, the present invention provides pharmaceutical compositions comprising a pharmaceutically acceptable carrier and a compound of the invention or a pharmaceutically acceptable salt thereof. Such compositions according to the invention may optionally further include one or more additional therapeutic agents as described herein.

In another aspect, the present invention provides a method for the manufacture of a medicament or a composition which may be useful for treating diseases, conditions, or disorders that are mediated, at least in part, by the adenosine A2a receptor and/or the adenosine A2b receptor, comprising combining a compound of the invention with one or more pharmaceutically acceptable carriers.

In another aspect, the present invention provides a method for treating or preventing a disease, condition, or disorder that is mediated, at least in part, by the adenosine A2a receptor and/or the adenosine A2b receptor in a subject (e.g., an animal or human) in need thereof, said method comprising administering to the subject in need thereof a therapeutically effective amount of at least one compound of the invention, or a pharmaceutically acceptable salt thereof, alone or in combination with one or more additional therapeutic agents. Specific non-limiting examples of such diseases, conditions, and disorders are described herein.

### Oncology

In some embodiments, the disease, condition or disorder is a cancer. Any cancer for which a PD-1 antagonist and/or an A2a and/or A2b inhibitor are thought to be useful by those of ordinary skill in the art are contemplated as cancers treatable by this embodiment, either as a monotherapy or in combination with other therapeutic agents discussed below. Cancers that express high levels of A2a receptors or A2b receptors are among those cancers contemplated as treatable by the compounds of the invention. Examples of cancers that express high levels of A2a and/or A2b receptors may be discerned by those of ordinary skill in the art by reference to the Cancer Genome Atlas (TCGA) database. Non-limiting examples of cancers that express high levels of A2a receptors include cancers of the kidney, breast, lung, and liver. Non-limiting examples of cancers that express high levels of the A2b receptor include lung, colorectal, head & neck cancer, and cervical cancer.

Thus, one embodiment provides a method of treating cancer comprising administering an effective amount of a compound of the invention, or a pharmaceutically acceptable salt thereof, to a subject in need of such treatment, wherein said cancer is a cancer that expresses a high level of A2a receptor. A related embodiment provides a method of treating cancer comprising administering an effective amount of a compound of the invention, or a pharmaceutically acceptable salt thereof, to a subject in need of such treatment, wherein said cancer is selected from kidney (or renal) cancer, breast cancer, lung cancer, and liver cancer.

Another embodiment provides a method of treating cancer comprising administering an effective amount of a compound of the invention, or a pharmaceutically acceptable salt thereof, to a subject in need of such treatment, wherein said cancer is a cancer that expresses a high level of A2b receptor. A related embodiment provides a method of treating cancer comprising administering an effective amount of a compound of the invention, or a pharmaceutically acceptable salt thereof, to a subject in need of such treatment, wherein said cancer is selected from lung cancer, colorectal cancer, head & neck cancer, and cervical cancer.

Additional non-limiting examples of cancers which may be treatable by administration of a compound of the invention (alone or in combination with one or more additional agents described below) include cancers of the prostate (including but not limited to metastatic castration resistant prostate cancer), colon, rectum, pancreas, cervix, stomach, endometrium, brain, liver, bladder, ovary, testis, head, neck, skin (including melanoma and basal carcinoma), mesothelial lining, white blood cell (including lymphoma and leukemia) esophagus, breast, muscle, connective tissue, lung (including but not limited to small cell lung cancer, non-small cell lung cancer, and lung adenocarcinoma), adrenal gland, thyroid, kidney, or bone. Additional cancers treatable by a compound of the invention include glioblastoma, mesothelioma, renal cell carcinoma, gastric carcinoma, sarcoma, choriocarcinoma, cutaneous basocellular carcinoma, and testicular seminoma, and Kaposi's sarcoma.

### CNS and Neurological Disorders

In other embodiments, the disease, condition or disorder is a central nervous system or a neurological disorder. Non-limiting examples of such diseases, conditions or disorders include movement disorders such as tremors, bradykinesias, gait disorders, dystonias, dyskinesias, tardive dyskinesias, other extrapyramidal syndromes, Parkinson's disease, and disorders associated with Parkinson's disease. The compounds of the invention also have the potential, or are believed to have the potential, for use in preventing or reducing the effect of drugs that cause or worsen such movement disorders.

### Infections

In other embodiments, the disease, condition or disorder is an infective disorder. Non-limiting examples of such diseases, conditions or disorders include an acute or chronic viral infection, a bacterial infection, a fungal infection, or a parasitic infection. In one embodiment, the viral infection is human immunodeficiency virus. In another embodiment, the viral infection is cytomegalovirus.

### Immune Disease

In other embodiments, the disease, condition or disorder is an immune-related disease, condition or disorder. Non-limiting examples of immune-related diseases, conditions, or disorders include multiple sclerosis and bacterial infections. (See, e.g., Safarzadeh, E. et al., Inflamm Res 2016 65(7):511-20; and Antonioli, L., et al., Immunol Lett S0165-2478(18)30172-X 2018).

### Additional Indications

Other diseases, conditions, and disorders that have the potential to be treated or prevented, in whole or in part, by the inhibition of the A2a and/or A2b adenosine receptor(s) are also candidate indications for the compounds of the invention and salts thereof. Non-limiting examples of other diseases, conditions or disorders in which a compound of the invention, or a pharmaceutically acceptable salt thereof, may be useful include the treatment of hypersensitivity reaction to a tumor antigen and the amelioration of one or more complications related to bone marrow transplant or to a peripheral blood stem cell transplant. Thus, in another embodiment, the present invention provides a method for treating a subject receiving a bone marrow transplant or a peripheral blood stem cell transplant by administering to said subject a therapeutically effective amount of a compound of the invention, or a pharmaceutically acceptable salt thereof, sufficient to increase the delayed-type hypersensitivity reaction to tumor antigen, to delay the time-to-relapse of post-transplant malignancy, to increase relapse-free survival time post-transplant, and/or to increase long-term post-transplant survival.

### Combination Therapy

In another aspect, the present invention provides methods for the use of a compound of the invention, or a pharmaceutically acceptable salt thereof, (or a pharmaceutically acceptable composition comprising a compound of the invention or pharmaceutically acceptable salt thereof) in combination with one or more additional agents. Such additional agents may have some adenosine A2a and/or A2b receptor activity, or, alternatively, they may function through distinct mechanisms of action. The compounds of the invention may be used in combination with one or more other drugs in the treatment, prevention, suppression or amelioration of diseases or conditions for which the compounds of the invention or the other drugs described herein may have utility, where the combination of the drugs together are safer or more effective than either drug alone. The combination therapy may have an additive or synergistic effect. Such other drug(s) may be administered in an amount commonly used therefore, contemporaneously or sequentially with a compound of the invention or a pharmaceutically acceptable salt thereof. When a compound of the invention is used contemporaneously with one or more other drugs, the pharmaceutical composition may in specific embodiments contain such other drugs and the compound of the invention or its pharmaceutically acceptable salt in separate doses or in unit dosage form. However, the combination therapy may also include therapies in which the compound of the invention or its pharmaceutically acceptable salt and one or more other drugs are administered sequentially, on different or overlapping schedules. It is also contemplated that when used in combination with one or more other active ingredients, the compounds of the invention and the other active ingredients may be used in lower doses than when each is used singly. Accordingly, the pharmaceutical compositions comprising the compounds of the invention include those that contain one or more other active ingredients, in addition to a compound of the invention or a pharmaceutically acceptable salt thereof.

The weight ratio of the compound of the present invention to the second active ingredient may be varied and will depend upon the effective dose of each ingredient. Generally, an effective dose of each will be used. Thus, for example, when a compound of the invention is used in combination with another agent, the weight ratio of the compound of the present invention to the other agent may generally range from about 1000:1 to about 1:1000, in particular embodiments from about 200:1 to about 1:200. Combinations of a compound of the present invention and other active ingredients will generally also be within the aforementioned range, but in each case, an effective dose of each active ingredient should generally be used.

Given the immunosuppressive role of adenosine, the administration of an A2a receptor antagonist, an A2b receptor antagonist, and/or an A2a/A2b receptor dual antagonist according to the invention may enhance the efficacy of immunotherapies such as PD-1 antagonists. Thus, in one embodiment, the additional therapeutic agent comprises an anti-PD-1 antibody. In another embodiment, the additional therapeutic agent is an anti-PD-L1 antibody.

As noted above, PD-1 is recognized as having an important role in immune regulation and the maintenance of peripheral tolerance. PD-1 is moderately expressed on naive T-cells, B-cells and NKT-cells and up-regulated by T-cell and B-cell receptor signaling on lymphocytes, monocytes and myeloid cells (Sharpe et al., Nature Immunology (2007); 8:239-245).

Two known ligands for PD-1, PD-L1 (B7-H1) and PD-L2 (B7-DC) are expressed in human cancers arising in various tissues. In large sample sets of, for example, ovarian, renal, colorectal, pancreatic, and liver cancers, and in melanoma, it was shown that PD-L1 expression correlated with poor prognosis and reduced overall survival irrespective of subsequent treatment. (Dong et al., Nat Med. 8(8):793-800 (2002); Yang et al., Invest Ophthamol Vis Sci. 49: 2518-2525 (2008); Ghebeh et al., Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953; Inman et al., Cancer 109: 1499-1505 (2007); Shimauchi et al., Int. J. Cancer 121:2585-2590 (2007); Gao et al., Clin. Cancer Research 15: 971-979 (2009); Nakanishi J., Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T-cells in breast cancer and melanoma (Ghebeh et al., BMC Cancer. 2008 8:5714-15 (2008); and Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T-cells to attenuate T-cell activation and to evade immune surveillance, thereby contributing to an impaired immune response against the tumor.

Immune checkpoint therapies targeting the PD-1 axis have resulted in groundbreaking improvements in clinical response in multiple human cancers (Brahmer, et al., N Engl J Med 2012, 366: 2455-65; Garon et al., N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; and Wolchok et al., N Engl J Med 2013, 369: 122-33).

“PD-1 antagonist” means any chemical compound or biological molecule that blocks binding of PD-L1 expressed on a cancer cell to PD-1 expressed on an immune cell (T-cell, B-cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP_054862 and NP_079515, respectively.

PD-1 antagonists useful in any of the treatment methods, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments. Examples of PD-1 antagonists include, but are not limited to, pembrolizumab (KEYTRUDA®, Merck and Co., Inc., Kenilworth, NJ, USA). “Pembrolizumab” (formerly known as MK-3475, SCH 900475 and lambrolizumab and sometimes referred to as “pembro”) is a humanized IgG4 mAb with the structure described in WHO Drug Information, Vol. 27, No. 2, pages 161-162 (2013). Additional examples of PD-1 antagonists include nivolumab (OPDIVO®, Bristol-Myers Squibb Company, Princeton, NJ, USA), atezolizumab (MPDL3280A; TECENTRIQ®, Genentech, San Francisco, CA, USA), durvalumab (IMIFINZI®, Astra Zeneca Pharmaceuticals, LP, Wilmington, DE, and avelumab (BAVENCIO®, Merck KGaA, Darmstadt, Germany and Pfizer, Inc., New York, NY).

Examples of monoclonal antibodies (mAbs) that bind to human PD-1, and useful in the treatment methods, medicaments and uses of the present invention, are described in U.S. Pat. Nos. 7,488,802, 7,521,051, 8,008,449, 8,354,509, 8,168,757, WO2004/004771, WO2004/072286, WO2004/056875, and US2011/0271358.

Examples of mAbs that bind to human PD-L1, and useful in the treatment methods, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in any of the treatment methods, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010/027827 and WO2011/066342. Specific fusion proteins useful as the PD-1 antagonist in the treatment methods, medicaments and uses of the present invention include AMP-224 (also known as B7-DCIg), which is a PD-L2-FC fusion protein that binds to human PD-1.

Thus, one embodiment provides for a method of treating cancer comprising administering an effective amount of a compound of the invention, or a pharmaceutically acceptable salt thereof, in combination with a PD-1 antagonist to a subject in need thereof. In such embodiments, the compounds of the invention, or a pharmaceutically acceptable salt thereof, and PD-1 antagonist are administered concurrently or sequentially.

Specific non-limiting examples of such cancers in accordance with this embodiment include melanoma (including unresectable or metastatic melanoma), head & neck cancer (including recurrent or metastatic head and neck squamous cell cancer (HNSCC)), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, hepatocellular carcinoma, clear cell kidney cancer, colorectal cancer, breast cancer, squamous cell lung cancer, basal carcinoma, sarcoma, bladder cancer, endometrial cancer, pancreatic cancer, liver cancer, gastrointestinal cancer, multiple myeloma, renal cancer, mesothelioma, ovarian cancer, anal cancer, biliary tract cancer, esophageal cancer, and salivary cancer.

In one embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from unresectable or metastatic melanoma, recurrent or metastatic head and neck squamous cell cancer (HNSCC), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, and hepatocellular carcinoma. In one such embodiment, the agent is a PD-1 antagonist. In one such embodiment, the agent is pembrolizumab. In another such embodiment, the agent is nivolumab. In another such embodiment, the agent is atezolizumab.

Pembrolizumab is approved by the U.S. FDA for the treatment of patients with unresectable or metastatic melanoma and for the treatment of certain patients with recurrent or metastatic head and neck squamous cell cancer (HNSCC), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, and hepatocellular carcinoma, as described in the Prescribing Information for KEYTRUDA™ (Merck & Co., Inc., Whitehouse Station, NJ USA; initial U.S. approval 2014, updated November 2018). In another embodiment, there is provided a method of treating cancer comprising administering an effective amount of a compound of the invention, or a pharmaceutically acceptable salt thereof, to a person in need thereof, in combination with pembrolizumab, wherein said cancer is selected from unresectable or metastatic melanoma, recurrent or metastatic head and neck squamous cell cancer (HNSCC), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, and hepatocellular carcinoma.

In another embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from melanoma, non-small cell lung cancer, head and neck squamous cell cancer (HNSCC), Hodgkin lymphoma, primary mediastinal large B-cell lymphoma, urothelial carcinoma, microsatellite instability-high cancer, gastric cancer, Merkel cell carcinoma, hepatocellular carcinoma, esophageal cancer and cervical cancer. In one such embodiment, the agent is a PD-1 antagonist. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab.

In another embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from melanoma, non-small cell lung cancer, small cell lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, and salivary cancer. In one such embodiment, the agent is a PD-1 antagonist. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab.

In one embodiment, there is provided a method of treating unresectable or metastatic melanoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating recurrent or metastatic head and neck squamous cell cancer (HNSCC) comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating classical Hodgkin lymphoma (cHL) comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating urothelial carcinoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating gastric cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating cervical cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating primary mediastinal large-B-cell lymphoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating microsatellite instability-high (MSI-H) cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating non-small cell lung cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In one embodiment, there is provided a method of treating hepatocellular carcinoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab.

In another embodiment, the additional therapeutic agent is at least one immunomodulator other than an A2a or A2b receptor inhibitor. Non-limiting examples of immunomodulators include CD40L, B7, B7RP1, anti-CD40, anti-CD38, anti-ICOS, 4-IBB ligand, dendritic cell cancer vaccine, IL2, IL12, ELC/CCL19, SLC/CCL21, MCP-1, IL-4, IL-18, TNF, IL-15, MDC, IFN-a/-13, M-CSF, IL-3, GM-CSF, IL-13, anti-IL-10 and indolamine 2,3-dioxygenase 1 (IDO1) inhibitors.

In another embodiment, the additional therapeutic agent comprises radiation. Such radiation includes localized radiation therapy and total body radiation therapy.

In another embodiment, the additional therapeutic agent is at least one chemotherapeutic agent. Non-limiting examples of chemotherapeutic agents contemplated for use in combination with the compounds of the invention include: pemetrexed, alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nucleoside analogs (e.g., gemcitabine); nitroso ureas such as carmustine, lomustine, and streptozocin; topoisomerase 1 inhibitors (e.g., irinotecan); platinum complexes such as cisplatin, carboplatin and oxaliplatin; bioreductive alkylators such as mitomycin, procarbazine, dacarbazine and altretamine); anthracycline-based therapies (e.g., doxorubicin, daunorubicin, epirubicin and idarubicin); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, estramustine, vinblastine, docetaxol, epothilone derivatives, and paclitaxel); hormonal agents (e.g., estrogens; conjugated estrogens; ethynyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); luteinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

In another embodiment, the additional therapeutic agent is at least one signal transduction inhibitor (STI). Non-limiting examples of signal transduction inhibitors include BCR/ABL kinase inhibitors, epidermal growth factor (EGF) receptor inhibitors, HER-2/neu receptor inhibitors, and farnesyl transferase inhibitors (FTIs).

In another embodiment, the additional therapeutic agent is at least one anti-infective agent. Non-limiting examples of anti-infective agents include cytokines, non-limiting examples of which include granulocyte-macrophage colony stimulating factor (GM-CSF) and an flt3-ligand.

In another embodiment, the present invention provides a method for treating or preventing a viral infection (e.g., a chronic viral infection) including, but not limited to, hepatitis C virus (HCV), human papilloma virus (HPV), cytomegalovirus (CMV), Epstein-Barr virus (EBV), varicella zoster virus, coxsackievirus, and human immunodeficiency virus (HIV).

In another embodiment, the present invention provides a method for the treatment of an infective disorder, said method comprising administering to a subject in need thereof an effective amount of a compound of the invention, or a pharmaceutically acceptable salt thereof, in combination with a vaccine. In some embodiments, the vaccine is an anti-viral vaccine, including, for example, an anti-HTV vaccine. Other antiviral agents contemplated for use include an anti-HIV, anti-HPV, anti HCV, anti HSV agents and the like. In other embodiments, the vaccine is effective against tuberculosis or malaria. In still other embodiments, the vaccine is a tumor vaccine (e.g., a vaccine effective against melanoma); the tumor vaccine may comprise genetically modified tumor cells or a genetically modified cell line, including genetically modified tumor cells or a genetically modified cell line that has been transfected to express granulocyte-macrophage stimulating factor (GM-CSF). In another embodiment, the vaccine includes one or more immunogenic peptides and/or dendritic cells.

In another embodiment, the present invention provides for the treatment of an infection by administering a compound of the invention, or a pharmaceutically acceptable salt thereof, and at least one additional therapeutic agent, wherein a symptom of the infection observed after administering both the compound of the invention (or a pharmaceutically acceptable salt thereof) and the additional therapeutic agent is improved over the same symptom of infection observed after administering either alone. In some embodiments, the symptom of infection observed can be reduction in viral load, increase in CD4+ T cell count, decrease in opportunistic infections, increased survival time, eradication of chronic infection, or a combination thereof.

### Definitions

As used herein, unless otherwise specified, the following terms have the following meanings.

Unsatisfied valences in the text, schemes, examples, structural formulae, and any Tables herein are assumed to have a hydrogen atom or atoms of sufficient number to satisfy the valences.

When a variable appears more than once in any moiety or in any compound of the invention (e.g., aryl, cycloheteroalkyl, N(R)₂), the selection of moieties defining that variable for each occurrence is independent of its definition at every other occurrence unless specified otherwise in the local variable definition.

As used herein, unless otherwise specified, the term “A2a receptor antagonist” (equivalently, A2a antagonist) and/or “A2b receptor antagonist” (equivalently, A2b antagonist) means a compound exhibiting a potency (IC₅₀) of less than about 1 μM with respect to the A2a and/or A2b receptors, respectively, when assayed in accordance with the procedures described herein. Preferred compounds exhibit at least 10-fold selectivity for antagonizing the A2a receptor and/or the A2b receptor over any other adenosine receptor (e.g., A1 or A3).

As described herein, unless otherwise indicated, the use of a compound in treatment means that an amount of the compound, generally presented as a component of a formulation that comprises other excipients, is administered in aliquots of an amount, and at time intervals, which provides and maintains at least a therapeutic serum level of at least one pharmaceutically active form of the compound over the time interval between dose administrations.

The phrase “at least one” used in reference to the number of components comprising a composition, for example, “at least one pharmaceutical excipient” means that one member of the specified group is present in the composition, and more than one may additionally be present. Components of a composition are typically aliquots of isolated pure material added to the composition, where the purity level of the isolated material added into the composition is the normally accepted purity level for a reagent of the type.

Whether used in reference to a substituent on a compound or a component of a pharmaceutical composition the phrase “one or more”, means the same as “at least one”.

“Concurrently” and “contemporaneously” both include in their meaning (1) simultaneously in time (e.g., at the same time); and (2) at different times but within the course of a common treatment schedule.

“Consecutively” means one following the other.

“Sequentially” refers to a series administration of therapeutic agents that awaits a period of efficacy to transpire between administering each additional agent; this is to say that after administration of one component, the next component is administered after an effective time period after the first component; the effective time period is the amount of time given for realization of a benefit from the administration of the first component.

“Effective amount” or “therapeutically effective amount” is meant to describe the provision of an amount of at least one compound of the invention or of a composition comprising at least one compound of the invention which is effective in treating or inhibiting a disease or condition described herein, and thus produce the desired therapeutic, ameliorative, inhibitory or preventative effect. For example, in treating a cancer as described herein with one or more of the compounds of the invention optionally in combination with one or more additional agents, “effective amount” (or “therapeutically effective amount”) means, for example, providing the amount of at least one compound of the invention that results in a therapeutic response in a patient afflicted with the disease, condition, or disorder, including a response suitable to manage, alleviate, ameliorate, or treat the condition or alleviate, ameliorate, reduce, or eradicate one or more symptoms attributed to the condition and/or long-term stabilization of the condition, for example, as may be determined by the analysis of pharmacodynamic markers or clinical evaluation of patients afflicted with the condition.

“Patient” and “subject” means an animal, such as a mammal (e.g., a human being) and is preferably a human being.

“Prodrug” means compounds that are rapidly transformed, for example, by hydrolysis in blood, in vivo to the parent compound, e.g., conversion of a prodrug of a compound of the invention to a compound of the invention, or to a salt thereof. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this invention includes prodrugs of the novel compounds of this invention.

The term “substituted” means that one or more of the moieties enumerated as substituents (or, where a list of substituents are not specifically enumerated, the substituents specified elsewhere in this application) for the particular type of substrate to which said substituent is appended, provided that such substitution does not exceed the normal valence rules for the atom in the bonding configuration presented in the substrate, and that the substitution ultimate provides a stable compound, which is to say that such substitution does not provide compounds with mutually reactive substituents located geminal or vicinal to each other; and wherein the substitution provides a compound sufficiently robust to survive isolation to a useful degree of purity from a reaction mixture.

Where optional substitution by a moiety is described (e.g. “optionally substituted”) the term means that if substituents are present, one or more of the enumerated (or default) moieties listed as optional substituents for the specified substrate can be present on the substrate in a bonding position normally occupied by the default substituent, for example, a hydrogen atom on an alkyl chain can be substituted by one of the optional substituents, in accordance with the definition of “substituted” presented herein.

“Alkyl” means an aliphatic hydrocarbon group, which may be straight or branched, comprising 1 to 10 carbon atoms. “(C₁-C₆)alkyl” means an aliphatic hydrocarbon group, which may be straight or branched, comprising 1 to 6 carbon atoms. Branched means that one or more lower alkyl groups such as methyl, ethyl or propyl, are attached to a linear alkyl chain. Non-limiting examples of alkyl groups include methyl, ethyl, n-propyl, isopropyl, n-butyl, i-butyl, and t-butyl.

“Haloalkyl” means an alkyl as defined above wherein one or more hydrogen atoms on the alkyl (up to and including each available hydrogen group) is replaced by a halogen atom. As appreciated by those of skill in the art, “halo” or “halogen” as used herein is intended to include chloro (Cl), fluoro (F), bromo (Br) and iodo (I). Chloro (Cl) and fluoro(F) halogens are generally preferred.

“Alkoxy” means an alkyl-O— group in which the alkyl group encompasses straight alkyl having a carbon number of 1 to 10 and branched alkyl having a carbon number of 3 to 10. Non-limiting examples of suitable alkoxy groups include methoxy, ethoxy, n-propoxy, isopropoxy and n-butoxy. The bond to the parent moiety is through the ether oxygen.

“Halogen” includes fluorine, chlorine, bromine or iodine.

“Aryl” means an aromatic monocyclic or multicyclic ring system comprising 6 to 14 carbon atoms, preferably 6 to 10 carbon atoms. The aryl group can be optionally substituted with one or more “ring system substituents” which may be the same or different, and are as defined herein. Non-limiting examples of suitable aryl groups include phenyl and naphthyl. “Monocyclic aryl” means phenyl.

“Heteroaryl” means an aromatic monocyclic or multicyclic ring system comprising 5 to 14 ring atoms, preferably 5 to 10 ring atoms, in which one or more of the ring atoms is an element other than carbon, for example nitrogen, oxygen or sulfur, alone or in combination. Preferred heteroaryls contain 5 to 6 ring atoms. The “heteroaryl” can be optionally substituted by one or more substituents, which may be the same or different, as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl (which alternatively may be referred to as thiophenyl), pyrimidinyl, pyridone (including N-substituted pyridones), isoxazolyl, isothiazolyl, oxazolyl, oxadiazolyl, thiazolyl, thiadiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. The term “monocyclic heteroaryl” refers to monocyclic versions of heteroaryl as described above and includes 4- to 7-membered monocyclic heteroaryl groups comprising from 1 to 4 ring heteroatoms, said ring heteroatoms being independently selected from the group consisting of N, O, and S, and oxides thereof. The point of attachment to the parent moiety is to any available ring carbon or ring heteroatom. Non-limiting examples of monocyclic heteroaryl moieties include pyridyl, pyrazinyl, furanyl, thienyl, pyrimidinyl, pyridazinyl, pyridinyl, thiazolyl, isothiazolyl, oxazolyl, oxadiazolyl, isoxazolyl, pyrazolyl, furazanyl, pyrrolyl, pyrazolyl, triazolyl, thiadiazolyl (e.g., 1,2,4-thiadiazolyl), imidazolyl, and triazinyl (e.g., 1,2,4-triazinyl), and oxides thereof.

“Cycloalkyl” means a non-aromatic fully saturated monocyclic or multicyclic ring system comprising 3 to 10 carbon atoms, preferably 3 to 6 carbon atoms. The cycloalkyl can be optionally substituted with one or more substituents, which may be the same or different, as described herein. Monocyclic cycloalkyl refers to monocyclic versions of the cycloalkyl moieties described herein. Non-limiting examples of suitable monocyclic cycloalkyls include cyclopropyl, cyclopentyl, cyclohexyl, cycloheptyl and the like. Non-limiting examples of multicyclic cycloalkyls include [1.1.1]-bicyclopentane, 1-decalinyl, norbornyl, adamantyl and the like.

“Cycloheteroalkyl” (or “heterocyclyl”) means a non-aromatic saturated or partially saturated monocyclic or multicyclic ring system comprising 3 to 10 ring atoms, preferably 5 to 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred cycloheteroalkyl groups contain 4, 5 or 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more substituents, which may be the same or different, as described herein. The nitrogen or sulfur atom of the heterocyclyl can be optionally oxidized to the corresponding N-oxide, S-oxide or S,S-dioxide. Thus, the term “oxide,” when it appears in a definition of a variable in a general structure described herein, refers to the corresponding N-oxide, S-oxide, or S,S-dioxide. “Heterocyclyl” also includes rings wherein ═O replaces two available hydrogens on the same carbon atom (i.e., heterocyclyl includes rings having a carbonyl group in the ring). Such ═O groups may be referred to herein as “oxo.” An example of such a moiety is pyrrolidinone (or pyrrolidone):

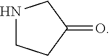

As used herein, the term “monocyclic heterocycloalkyl” refers to monocyclic versions of the heterocycloalkyl moieties described herein and include a 4- to 7-membered monocyclic heterocycloalkyl groups comprising from 1 to 4 ring heteroatoms, said ring heteroatoms being independently selected from the group consisting of N, N-oxide, O, S, S-oxide, S(O), and S(O)₂. The point of attachment to the parent moiety is to any available ring carbon or ring heteroatom. Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. Non-limiting examples of lower alkyl-substituted oxetanyl include the moiety:

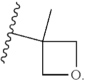 [SMILES: *C1(C)COC1]

It is noted that in hetero-atom containing ring systems of this invention, there are no hydroxyl groups on carbon atoms adjacent to a N, O or S, and there are no N or S groups on carbon adjacent to another heteroatom.

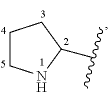 [SMILES: *C1CCCN1]

there is no —OH attached directly to carbons marked 2 and 5.

The line —, as a bond generally indicates a mixture of, or either of, the possible isomers, e.g., containing (R)- and (S)-stereochemistry. For example:

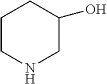

means containing both

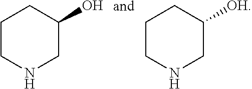

The wavy line [symbol] , as used herein, indicates a point of attachment to the rest of the compound. Lines drawn into the ring systems, such as, for example:

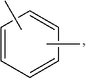

indicate that the indicated line (bond) may be attached to any of the substitutable ring atoms.

“Oxo” is defined as an oxygen atom that is double bonded to a ring carbon in a cycloalkyl, cycloalkenyl, heterocyclyl, heterocyclenyl, or other ring described herein, e.g.,

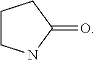

As well known in the art, a bond drawn from a particular atom wherein no moiety is depicted at the terminal end of the bond indicates a methyl group bound through that bond to the atom, unless stated otherwise. For example:

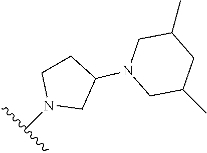 [SMILES: *N1CCC(N2CC(C)CC(C)C2)C1]

represents

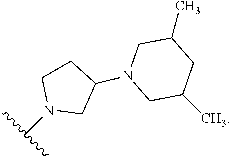 [SMILES: *N1CCC(N2CC(C)CC(C)C2)C1]

One or more compounds of the invention may also exist as, or optionally be converted to, a solvate. Preparation of solvates is generally known. Thus, for example, M. Caira et al., J. Pharmaceutical Sci., 93(3), 601-611 (2004) describe the preparation of the solvates of the antifungal fluconazole in ethyl acetate as well as from water. Similar preparations of solvates, and hemisolvate, including hydrates (where the solvent is water or aqueous-based) and the like are described by E. C. van Tonder et al., AAPS PharmSciTech., 5(1), article 12 (2004); and A. L. Bingham et al., Chem. Commun., 603-604 (2001). A typical, non-limiting, process involves dissolving the inventive compound in desired amounts of the desired solvent (for example, an organic solvent, an aqueous solvent, water or mixtures of two or more thereof) at a higher than ambient temperature, and cooling the solution, with or without an antisolvent present, at a rate sufficient to form crystals which are then isolated by standard methods. Analytical techniques such as, for example I.R. spectroscopy, show the presence of the solvent (including water) in the crystals as a solvate (or hydrate in the case where water is incorporated into the crystalline form).

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, and in sufficient purity to be characterized by standard analytical techniques described herein or well known to the skilled artisan.

This invention also includes the compounds of the invention in isolated and purified form obtained by routine techniques. Polymorphic forms of the compounds of the invention, and of the salts, solvates and prodrugs of the thereof, are intended to be included in the present invention. Certain compounds of the invention may exist in different isomeric forms (e.g., enantiomers, diastereoisomers, atropisomers). The inventive compounds include all isomeric forms thereof, both in pure form and admixtures of two or more, including racemic mixtures.

In similar manner, unless indicated otherwise, presenting a structural representation of any tautomeric form of a compound which exhibits tautomerism is meant to include all such tautomeric forms of the compound. Accordingly, where compounds of the invention, their salts, and solvates and prodrugs thereof, may exist in different tautomeric forms or in equilibrium among such forms, all such forms of the compound are embraced by, and included within the scope of the invention. Examples of such tautomers include, but are not limited to, ketone/enol tautomeric forms, imine-enamine tautomeric forms, and for example heteroaromatic forms such as the following moieties:

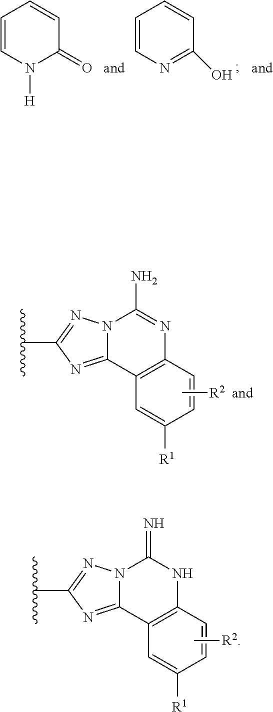

Where a reaction scheme appearing in an example employs a compound having one or more stereocenters, the stereocenters are indicated with an asterisk, as shown below:

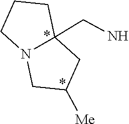 [SMILES: CC1CN2CCCC2(CN)C1]

Accordingly, the above depiction consists of the following pairs of isomers: (i) Trans-isomers ((2R,7aS)-2-methylhexahydro-1H-pyrrolizin-7a-yl)methanamine (Compound ABC-1) and ((2S,7aR)-2-methylhexahydro-1H-pyrrolizin-7a-yl)methanamine (Compound ABC-2); and (ii) Cis-isomers ((2R,7aR)-2-methylhexahydro-1H-pyrrolizin-7a-yl)methanamine (Compound ABC-3) and ((2S,7aS)-2-methylhexahydro-1H-pyrrolizin-7a-yl)methanamine (Compound ABC-4).

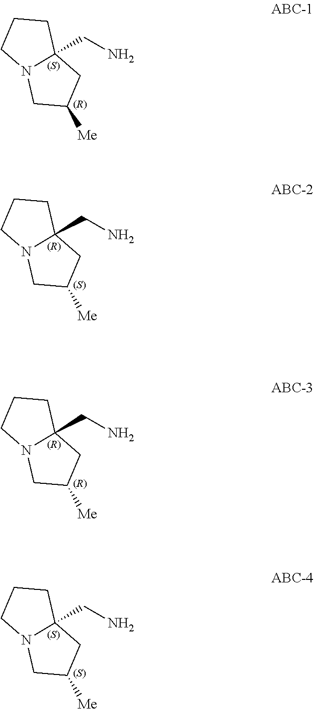

All stereoisomers of the compounds of the invention (including salts and solvates of the inventive compounds and their prodrugs), such as those which may exist due to asymmetric carbons present in a compound of the invention, and including enantiomeric forms (which may exist even in the absence of asymmetric carbons), rotameric forms, atropisomers, and diastereomeric forms, are contemplated within the scope of this invention. Individual stereoisomers of the compounds of the invention may be isolated in a pure form, for example, substantially free of other isomers, or may be isolated as an admixture of two or more stereoisomers or as a racemate. The chiral centers of the present invention can have the S or R configuration as defined by the IUPAC 1974 Recommendations. The use of the terms “salt”, “solvate” “prodrug” and the like, is intended to equally apply to salts, solvates and prodrugs of isolated enantiomers, stereoisomer pairs or groups, rotamers, tautomers, or racemates of the inventive compounds.

Where diastereomeric mixtures can be separated into their individual diastereomers on the basis of their physical chemical differences by known methods, for example, by chiral chromatography and/or fractional crystallization, simple structural representation of the compound contemplates all diastereomers of the compound. As is known, enantiomers may also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individually isolated diastereomers to the corresponding purified enantiomers.

As the term is employed herein, salts of the inventive compounds, whether acidic salts formed with inorganic and/or organic acids, basic salts formed with inorganic and/or organic bases, salts formed which include zwitterionic character, for example, where a compound contains both a basic moiety, for example, but not limited to, a nitrogen atom, for example, an amine, pyridine or imidazole, and an acidic moiety, for example, but not limited to a carboxylic acid, are included in the scope of the inventive compounds described herein. The formation of pharmaceutically useful salts from basic (or acidic) pharmaceutical compounds are discussed, for example, by S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference.

The present invention contemplates all available salts, including salts which are generally recognized as safe for use in preparing pharmaceutical formulations and those which may be formed presently within the ordinary skill in the art and are later classified as being “generally recognized as safe” for use in the preparation of pharmaceutical formulations, termed herein as “pharmaceutically acceptable salts”. Examples of pharmaceutically acceptable acid addition salts include, but are not limited to, acetates, including trifluoroacetate salts, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Examples of pharmaceutically acceptable basic salts include, but are not limited to, ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexyl-amine, choline, tromethamine, and salts with amino acids such as arginine, lysine and the like. Basic nitrogen-containing groups may be converted to an ammonium ion or quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), arylalkyl halides (e.g. benzyl and phenethyl bromides), and others.

All such acid and base salts are intended to be pharmaceutically acceptable salts within the scope of the invention and all acid and base salts are considered equivalent to the free forms of the corresponding compounds for purposes of the scope of the invention.

A functional group in a compound termed “protected” means that the group is in modified form to preclude undesired side reactions at the protected site when the protected compound is subjected to particular reaction conditions aimed at modifying another region of the molecule. Suitable protecting groups are known, for example, as by reference to standard textbooks, for example, T. W. Greene et al., Protective Groups in organic Synthesis (1991), Wiley, New York.

In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of the invention can be prepared without undue experimentation by conventional techniques well known to those skilled in the art or by processes analogous to those described in the Schemes and Examples herein using appropriate isotopically-enriched reagents and/or intermediates.

The present invention also embraces isotopically-labeled compounds of the present invention which are structurally identical to those recited herein, but for the fact that a statistically significant percentage of one or more atoms in that form of the compound are replaced by an atom having an atomic mass or mass number different from the atomic mass or mass number of the most abundant isotope usually found in nature, thus altering the naturally occurring abundance of that isotope present in a compound of the invention. Examples of isotopes that can be preferentially incorporated into compounds of the invention include isotopes of hydrogen, carbon, nitrogen, oxygen, phosphorus, iodine, fluorine and chlorine, for example, but not limited to: ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, and ³⁶Cl, ¹²³I and ¹²⁵. It will be appreciated that other isotopes also may be incorporated by known means.

Certain isotopically-labeled compounds of the invention (e.g., those labeled with ³H, ¹¹C and ¹⁴C) are recognized as being particularly useful in compound and/or substrate tissue distribution assays using a variety of known techniques. Tritiated (i.e., ³H) and carbon-14 (i.e., ¹⁴C) isotopes are particularly preferred for their ease of preparation and detection. Further, substitution of a naturally abundant isotope with a heavier isotope, for example, substitution of protium with deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in the reaction Schemes and/or in the Examples herein below, by substituting an appropriate isotopically labeled reagent for a non-isotopically labeled reagent, or by well-known reactions of an appropriately prepared precursor to the compound of the invention which is specifically prepared for such a “labeling” reaction. Such compounds are included also in the present invention.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

The term “composition” is intended to encompass a product comprising the specified ingredients in the specified amounts, and any product which results, directly or indirectly, from combination of the specified ingredients in the specified amounts.

The term “pharmaceutical composition” as used herein encompasses both the bulk composition and individual dosage units comprised of one, or more than one (e.g., two), pharmaceutically active agents such as, for example, a compound of the present invention (optionally together with an additional agent as described herein), along with any pharmaceutically inactive excipients. As will be appreciated by those of ordinary skill in the art, excipients are any constituent which adapts the composition to a particular route of administration or aids the processing of a composition into a dosage form without itself exerting an active pharmaceutical effect. The bulk composition and each individual dosage unit can contain fixed amounts of the aforesaid one, or more than one, pharmaceutically active agents. The bulk composition is material that has not yet been formed into individual dosage units.

It will be appreciated that pharmaceutical formulations of the invention may comprise more than one compound of the invention (or a pharmaceutically acceptable salt thereof), for example, the combination of two or three compounds of the invention, each present in such a composition by adding to the formulation the desired amount of the compound in a pharmaceutically acceptably pure form. It will be appreciated also that in formulating compositions of the invention, a composition may comprise, in addition to one or more of compounds of the invention, one or more other agents which also have pharmacological activity, as described herein.

While formulations of the invention may be employed in bulk form, it will be appreciated that for most applications the inventive formulations will be incorporated into a dosage form suitable for administration to a patient, each dosage form comprising an amount of the selected formulation which contains an effective amount of one or more compounds of the invention. Examples of suitable dosage forms include, but are not limited to, dosage forms adapted for: (i) oral administration, e.g., a liquid, gel, powder, solid or semi-solid pharmaceutical composition which is loaded into a capsule or pressed into a tablet and may comprise additionally one or more coatings which modify its release properties, for example, coatings which impart delayed release or formulations which have extended release properties; (ii) a dosage form adapted for intramuscular administration (IM), for example, an injectable solution or suspension, and which may be adapted to form a depot having extended release properties; (iii) a dosage form adapted for intravenous administration (IV), for example, a solution or suspension, for example, as an IV solution or a concentrate to be injected into a saline IV bag; (iv) a dosage form adapted for administration through tissues of the oral cavity, for example, a rapidly dissolving tablet, a lozenge, a solution, a gel, a sachets or a needle array suitable for providing intramucosal administration; (v) a dosage form adapted for administration via the mucosa of the nasal or upper respiratory cavity, for example a solution, suspension or emulsion formulation for dispersion in the nose or airway; (vi) a dosage form adapted for transdermal administration, for example, a patch, cream or gel; (vii) a dosage form adapted for intradermal administration, for example, a microneedle array; and (viii) a dosage form adapted for delivery via rectal or vaginal mucosa, for example, a suppository.

For preparing pharmaceutical compositions comprising compounds of the invention, generally the compounds of the invention will be combined with one or more pharmaceutically acceptable excipients. These excipients impart to the composition properties which make it easier to handle or process, for example, lubricants or pressing aids in powdered medicaments intended to be tableted, or adapt the formulation to a desired route of administration, for example, excipients which provide a formulation for oral administration, for example, via absorption from the gastrointestinal tract, transdermal or transmucosal administration, for example, via adhesive skin “patch” or buccal administration, or injection, for example, intramuscular or intravenous, routes of administration. These excipients are collectively termed herein “a carrier”. Typically formulations may comprise up to about 95 percent active ingredient, although formulations with greater amounts may be prepared.

Pharmaceutical compositions can be solid, semi-solid or liquid. Solid form preparations can be adapted to a variety of modes of administration, examples of which include, but are not limited to, powders, dispersible granules, mini-tablets, beads, which can be used, for example, for tableting, encapsulation, or direct administration. Liquid form preparations include, but are not limited to, solutions, suspensions and emulsions which for example, but not exclusively, can be employed in the preparation of formulations intended for parenteral injection, for intranasal administration, or for administration to some other mucosal membrane. Formulations prepared for administration to various mucosal membranes may also include additional components adapting them for such administration, for example, viscosity modifiers.

Aerosol preparations, for example, suitable for administration via inhalation or via nasal mucosa, may include solutions and solids in powder form, which may be in combination with a pharmaceutically acceptable propellant, for example, an inert compressed gas, e.g. nitrogen. Also included are solid form preparations which are intended to be converted, shortly before use, to a suspension or a solution, for example, for oral or parenteral administration. Examples of such solid forms include, but are not limited to, freeze dried formulations and liquid formulations adsorbed into a solid absorbent medium.

The compounds of the invention may also be deliverable transdermally or transmucosally, for example, from a liquid, suppository, cream, foam, gel, or rapidly dissolving solid form. It will be appreciated that transdermal compositions can take also the form of creams, lotions, aerosols and/or emulsions and can be provided in a unit dosage form which includes a transdermal patch of any know in the art, for example, a patch which incorporates either a matrix comprising the pharmaceutically active compound or a reservoir which comprises a solid or liquid form of the pharmaceutically active compound.

Examples of pharmaceutically acceptable carriers and methods of manufacture for various compositions mentioned above may be found in A. Gennaro (ed.), Remington: The Science and Practice of Pharmacy, 20^{th} Edition, (2000), Lippincott Williams & Wilkins, Baltimore, MD.

Preferably, the pharmaceutical preparation is in a unit dosage form. In such form, the preparations subdivided into suitably sized unit doses containing appropriate quantities of the active component, e.g., an effective amount to achieve the desired purpose.

The actual dosage employed may be varied depending upon the requirements of the patient and the severity of the condition being treated. Determination of the proper dosage regimen for a particular situation is within the skill in the art. For convenience, the total daily dosage may be divided and administered in portions during the day as required.

In accordance with the present invention, antagonism of adenosine A2a and/or A2b receptors is accomplished by administering to a patient in need of such therapy an effective amount of one or more compounds of the invention, or a pharmaceutically acceptable salt thereof.

In some embodiments it is preferred for the compound to be administered in the form of a pharmaceutical composition comprising the compound of the invention, or a salt thereof, and at least one pharmaceutically acceptable carrier (described herein). It will be appreciated that pharmaceutically formulations of the invention may comprise more than one compound of the invention, or a salt thereof, for example, the combination of two or three compounds of the invention, or, additionally or alternatively, another active agent such as those described herein, each present by adding to the formulation the desired amount of the compound or a salt thereof (or agent, where applicable) which has been isolated in a pharmaceutically acceptably pure form.

As mentioned above, administration of a compound of the invention to effect antagonism of A2a and/or A2b receptors is preferably accomplished by incorporating the compound into a pharmaceutical formulation incorporated into a dosage form, for example, one of the above-described dosage forms comprising an effective amount of at least one compound of the invention (e.g., 1, 2 or 3, or 1 or 2, or 1, and usually 1 compound of the invention), or a pharmaceutically acceptable salt thereof. Methods for determining safe and effective administration of compounds which are pharmaceutically active, for example, a compound of the invention, are known to those skilled in the art, for example, as described in the standard literature, for example, as described in the “Physicians' Desk Reference” (PDR), e.g., 1996 edition (Medical Economics Company, Montvale, NJ 07645-1742, USA), the Physician's Desk Reference, 56^{th} Edition, 2002 (published by Medical Economics company, Inc. Montvale, NJ 07645-1742), or the Physician's Desk Reference, 57^{th} Edition, 2003 (published by Thompson PDR, Montvale, NJ 07645-1742); the disclosures of which is incorporated herein by reference thereto. The amount and frequency of administration of the compounds of the invention and/or the pharmaceutically acceptable salts thereof will be regulated according to the judgment of the attending clinician considering such factors as age, condition and size of the patient as well as severity of the symptoms being treated. Compounds of the invention can be administered at a total daily dosage of up to 1,000 mg, which can be administered in one daily dose or can be divided into multiple doses per 24 hour period, for example, two to four doses per day.

As those of ordinary skill in the art will appreciate, an appropriate dosage level for a compound (or compounds) of the invention will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, or may be administered once or twice per day.

Those skilled in the art will appreciate that treatment protocols utilizing at least one compound of the invention can be varied according to the needs of the patient. Thus, compounds of the invention used in the methods of the invention can be administered in variations of the protocols described above. For example, compounds of the invention can be administered discontinuously rather than continuously during a treatment cycle.

In general, in whatever form administered, the dosage form administered will contain an amount of at least one compound of the invention, or a salt thereof, which will provide a therapeutically effective serum level of the compound in some form for a suitable period of time such as at least 2 hours, more preferably at least four hours or longer. In general, as is known in the art, dosages of a pharmaceutical composition providing a therapeutically effective serum level of a compound of the invention can be spaced in time to provide serum level meeting or exceeding the minimum therapeutically effective serum level on a continuous basis throughout the period during which treatment is administered. As will be appreciated the dosage form administered may also be in a form providing an extended release period for the pharmaceutically active compound which will provide a therapeutic serum level for a longer period, necessitating less frequent dosage intervals. As mentioned above, a composition of the invention can incorporate additional pharmaceutically active components or be administered simultaneously, contemporaneously, or sequentially with other pharmaceutically active agents as may be additionally needed or desired in the course of providing treatment. As will be appreciated, the dosage form administered may also be in a form providing an extended release period for the pharmaceutically active compound which will provide a therapeutic serum level for a longer period, necessitating less frequent dosage intervals.

### PREPARATIVE EXAMPLES

The compounds of the present invention can be prepared readily according to the following schemes and specific examples, or modifications thereof, using readily available starting materials, reagents and conventional synthetic procedures. In these reactions, it is also possible to make use of variants which are themselves known to those of ordinary skill in this art but are not mentioned in detail. The general procedures for making the compounds claimed in this invention can be readily understood and appreciated by one skilled in the art from viewing the following Schemes and descriptions.

Abbreviations used herein have the following meaning:

| | |
|---|---|
| ° C. | Degrees Celsius |
| μL | Microliter |
| μmol | Micromolar |
| Ac | Acetyl |
| Ac₂O | Acetic anhydride |
| AcOH | Acetic acid |
| aq. | Aqueous |
| B₂Pin₂ | Bis(pinacolato)diboron or 4,4,4',4',5,5,5',5'- |
| | Octamethyl-2,2'-bi-1,3,2-dioxaborolane |
| Boc | Tert-butoxycarbonyl |
| Boc₂0 | Di-tert-butyl dicarbonate |
| BSA | N,O-Bis(trimethylsily1)acetamide |
| Celite ® | Diatomaceous earth |
| COMU | (1-Cyano-2-ethoxy-2- |
| | oxoethylidenaminooxy)dimethylamino- |
| | morpholino-carbenium hexafluorophosphate |
| DBU | 1,8-Diazabicyclo[5.4.0]undec-7-ene |
| DCE | Dichloroethane |
| DCM | Dichloromethane |
| DDQ | 2,3-Dichloro-5,6-dicyano-1,4-benzoquinone |
| DIPEA | N,N-Diisopropylethylamine |
| DMAP | 4-Dimethylaminopyridine |
| DMF | Dimethylformamide |
| DMP | Dess-Martin periodinane or 1,1,1- |
| | Tris(acetyloxy)-1,1-dihydro-1,2-benziodoxol- |
| | 3-(1H)-one |
| DMSO | Dimethyl Sulfoxide |
| DMSO-d₆ | Deuterated Dimethyl Sulfoxide |
| Dppf | Bis(diphenylphosphino)ferrocene |
| ESI | Electrospray Ionization |
| Et₂NH | Diethylamine |
| Et₃N | Triethylamine |
| EtOAc | Ethyl Acetate |
| EtOH | Ethanol |
| G | Grams |
| h | Hour/Hours |
| HBPin | Pinacolborane or 4,4,5,5-Tetramethyl-1,3,2- |
| | dioxaborolane |
| Hoveyda-Grubbs Catalyst © 2^{nd} Generation | 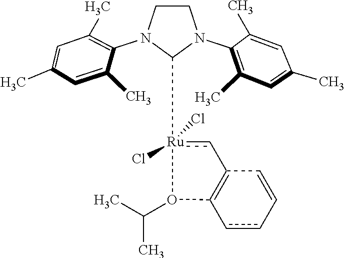 |
| | Dichloro[1,3-bis(2,4,6-trimethylphenyl)-2- |
| | imidazolidinylidene](2- |
| | isopropoxyphenylmethylene)ruthenium(II) |
| | CAS# 301224-40-8 |
| HPLC | High Performance Liquid Chromatography |
| i-PrBr | Isopropyl bromide |
| i-PrOH | Isopropyl alcohol or 2-propanol |
| M | Molar |
| MeCN | Acetonitrile |
| MeMgBr | Methylmagnesium bromide |
| MeOD-d₄ | Deuterated Methanol-d4 |
| MeOH | Methanol |
| Mg | Milligrams |
| MHz | Megahertz |
| Min | Minutes |
| mL | Milliliters |
| Mmol | Millimoles |
| MS | Mass Spectroscopy |
| Ms | Methanesulfonyl |
| nM | Nanomolar |
| NMM | N-methylmorpholine |
| NMR | Nuclear Magnetic Resonance |
| OAc | Acetate or acetoxy |
| OMe | Methoxide or methoxy |
| Oms | Mesylate or methanesulfonate |
| Oxone ® | Potassium peroxymonosulfate triple salt, |
| | 2KHSO₅•KHSO₄•K₂SO₄ |
| P(t-Bu)₃ Pd G2 | 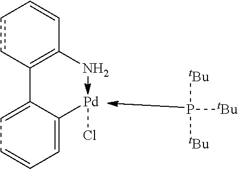 |
| | Chloro[(tri-tert-butylphosphine)-2-(2- |
| | aminobiphenyl)] palladium(II) |
| | CAS# 1375325-71-5 |
| Pd/C | Palladium on Carbon |
| Pd(PPh₃)₄ | Tetrakis(triphenylphosphine)palladium(0) |
| Ph | Phenyl |
| PIDA | (Diacetoxyiodo)benzene |
| Psi | Pounds per square inch |
| Py | Pyridine or pyridyl |
| RockPhos Pd G3 | 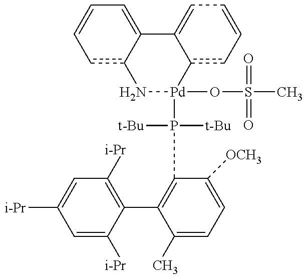 |
| | [(2-Di-tert-butylphosphino-3-methoxy-6- |
| | methyl-2',4',6'-triisopropyl-1,1'-biphenyl)-2- |
| | (2-aminobiphenyl)]palladium(II) |
| | methanesulfonate |
| | CAS# 2009020-38-4 |
| sat. | Saturated |
| SFC | Supercritical fluid chromatography (CO₂) |
| T3P ® | 1-Propanephosphonic anhydride |
| TBSCI | Tert-butyldimethylsilyl chloride |
| t-BuXPhos Pd G3 | 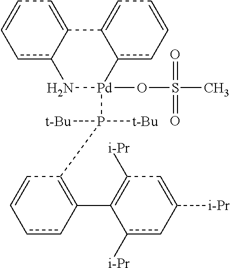 |
| | [(2-Di-tert-butylphosphino-2',4',6'- |
| | triisopropyl-1,1'-biphenyl)-2-(2'-amino-1,1'- |
| | biphenyl)] palladium(II) methanesulfonate |
| | CAS# 1447963-75-8 |
| t-BuXPhos | 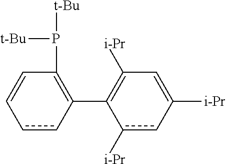 |
| | 2-Di-tert-butylphosphino-2',4',6'- |
| | triisopropylbiphenyl |
| | CAS# 564483-19-8 |
| TFA | Trifluoroacetic acid |
| THF | Tetrahydrofuran |
| TLC | Thin Layer Chromatography |
| TMS | Trimethylsilyl |
| Ts | Toluenesulfonyl |

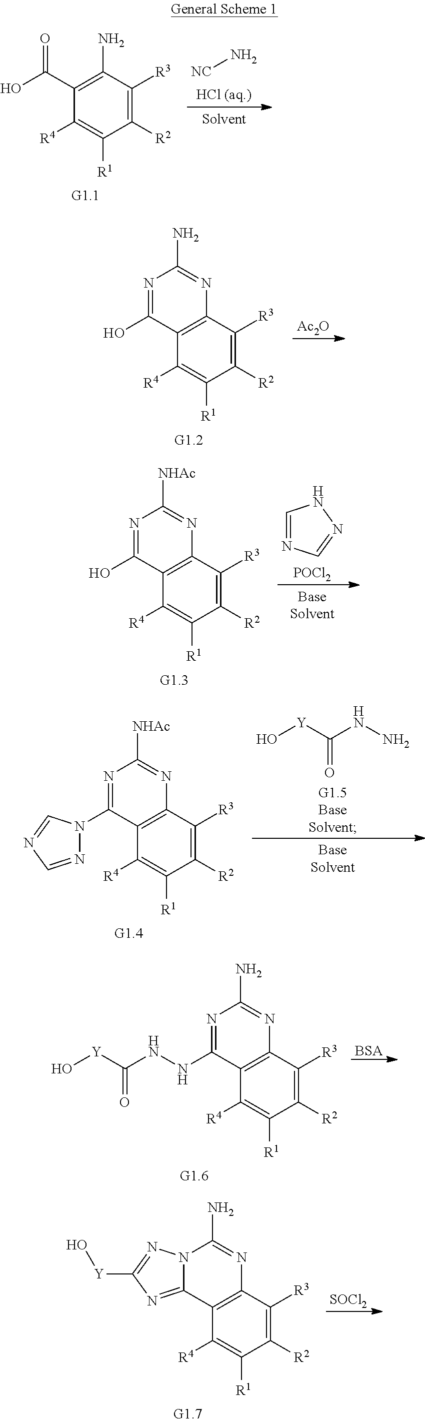

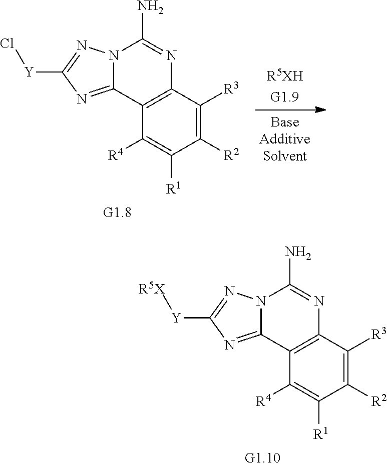

One general strategy for the synthesis of compounds of type G1.10 is via a seven-step procedure shown in General Scheme 1, wherein Y, R¹, R², R³, R⁴ and R⁵ are defined in Formula (I). In the first two steps, amino benzoic acids G1.1 can be converted into amino quinazolines G1.3 via treatment with cyanamide in the presence of aqueous HCl in a solvent such as EtOH, followed by subsequent acetylation with Ac₂O. In the third step, intermediates of type G1.3 can be converted into intermediates of type G1.4 through coupling with 1,2,4-triazole, following treatment with POCl₃ in a solvent such as MeCN, and a base such as DIPEA. In the fourth step, intermediates of type G1.4 can be treated with hydrazides G1.5 in a solvent such as THF, and a base such as DIPEA, followed by deprotection with a base such as K₂CO₃ and a solvent such as MeOH to provide products of type G1.6. In the fifth step, intermediates of type G1.6 can undergo a rearrangement upon heating in neat BSA to form products of type G1.7. In the sixth step, intermediates of type G1.7 can be converted into intermediates of type G1.8 upon heating in neat SOCl₂. In the seventh and final step, intermediates of type G1.8 can be converted into intermediates of type G1.10 through a displacement reaction with nucleophiles G1.9 wherein additives such as KI, bases such as NaH, K₂CO₃, and Cs₂CO₃, and solvents such as DMF and MeCN can be used. Products of type G1.10 can be purified by trituration, filtering, and washing with an appropriate solvent, preparative TLC, silica gel chromatography, preparative reversed-phase HPLC, and/or chiral SFC. In addition, subsequent manipulations can be performed on G1.10 to provide further elaborated products.

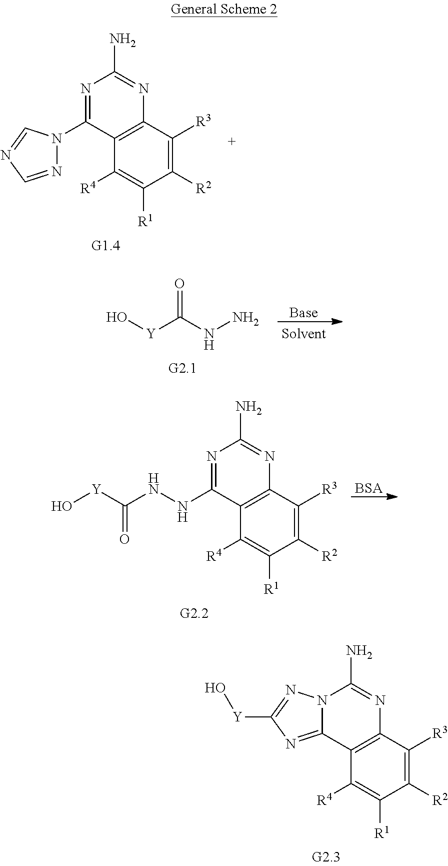

Analogous to Steps 4 and 5 in General Scheme 1, one general strategy for the synthesis of compounds of type G2.3 is via a two-step procedure shown in General Scheme 2, wherein Y, R¹, R², R³, and R⁴ are defined in Formula (I). In the first step, intermediates of type G1.4 can be treated with hydrazides G2.1 in a solvent such as dioxane, and a base such as DIPEA, to provide products of type G2.2. In the second and final step, intermediates of type G2.2 can undergo a rearrangement upon heating in neat BSA to form products of type G2.3.

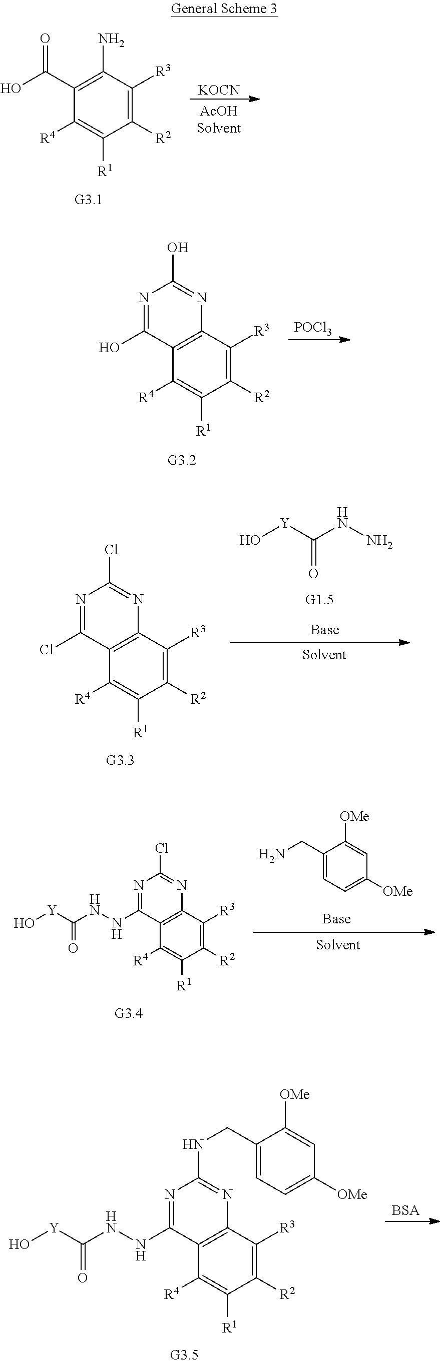

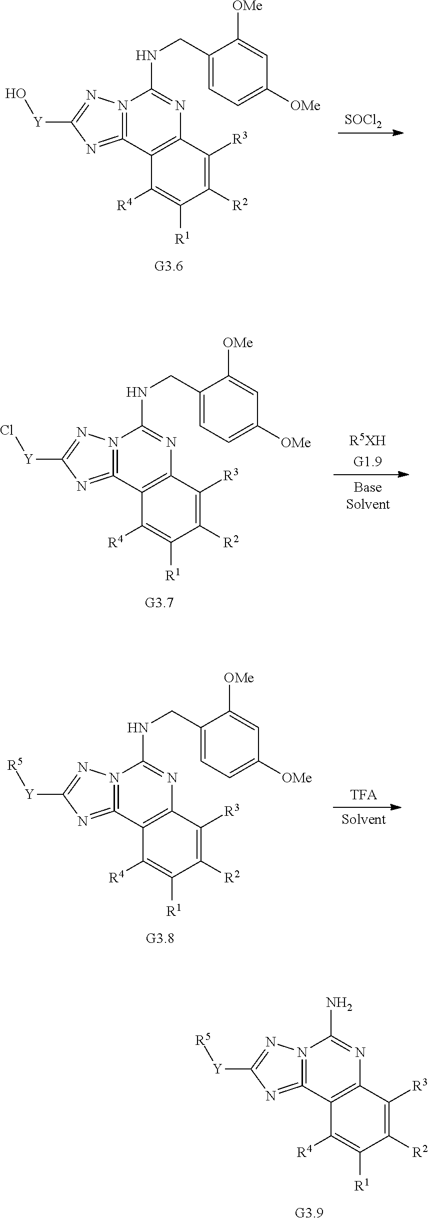

One general strategy for the synthesis of compounds of type G3.9 is via an eight-step procedure shown in General Scheme 3, wherein Y, R¹, R², R³, R⁴ and R⁵ are defined in Formula (I). In the first step, amino benzoic acids G3.1 can be converted into quinazolines G3.2 via treatment with KOCN in the presence of AcOH in a solvent such as water. In the second step, intermediates of type G3.2 can be converted into intermediates of type G3.3, following treatment with POCl₃ in a solvent such as MeCN, and a base such as DIPEA. In the third step, intermediates of type G3.3 can be treated with hydrazides G1.5 in a solvent such as THF, and a base such as DIPEA, to provide products of type G3.4. In the fourth step, 2,4-dimethoxybenzyl amine is added in along with a base such as DIPEA in a solvent such as dioxane to generate intermediates of type G3.5. In the fifth step, intermediates of type G3.5 can undergo a rearrangement upon heating in neat BSA to form products of type G3.6. In the sixth step, intermediates of type G3.6 can be converted into intermediates of type G3.7 upon heating in neat SOCl₂. In the seventh step, intermediates of type G3.7 can be converted into intermediates of type G3.8 through a displacement reaction with nucleophiles G1.9 (XH═OH, SH, SO₂H) wherein bases such as NaH, and solvents such as DMF can be used. In the eighth and final step, the 2,4-dimethoxybenzyl group of G3.8 is removed via treatment with TFA, either neat or in the presence of a solvent like DCM, to provide products of type G3.9. Products of type G3.9 can be purified by trituration, filtering, and washing with an appropriate solvent, preparative TLC, silica gel chromatography, preparative reversed-phase HPLC, and/or chiral SFC.

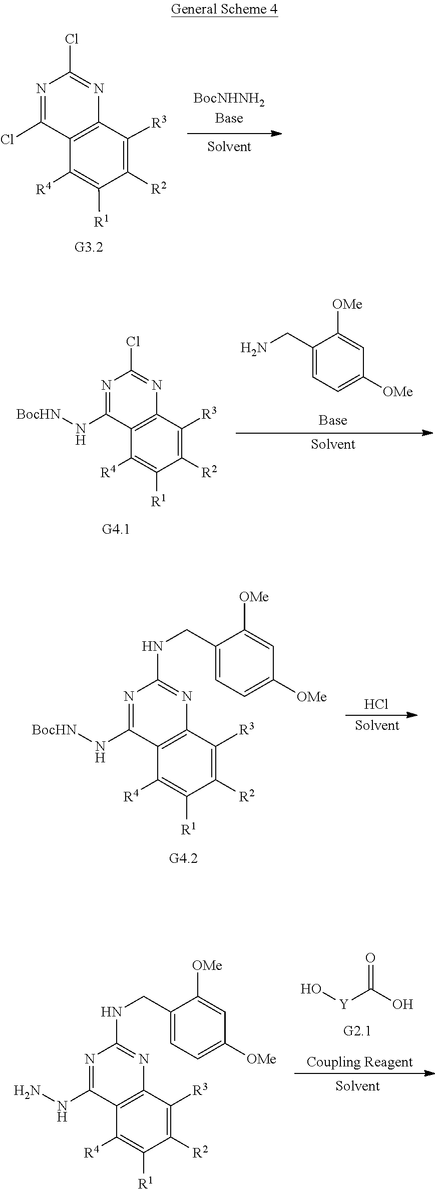

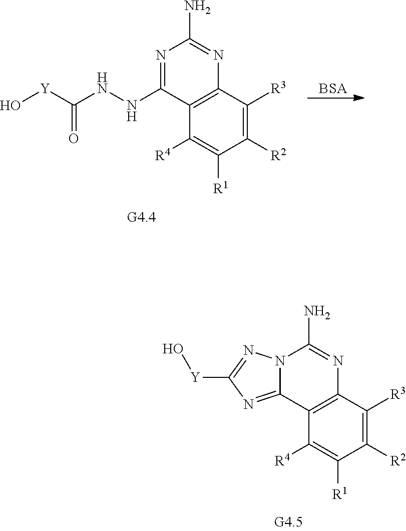

As an extension to the utility of intermediates like G3.2, one general strategy for the synthesis of compounds of type G4.5 is outlined in General Scheme 4, wherein Y, R¹, R², R³, and R⁴ are defined in Formula (I). In the first step, analogous to Step 3 in General Scheme 3, intermediates of type G3.2 can be treated with tert-butyl hydrazinecarboxylate in a solvent such as THF, and a base such as DIPEA, to provide products of type G4.1. In the second step, analogous to Step 4 in General Scheme 3, 2,4-dimethoxybenzyl amine is added in along with a base such as DIPEA in a solvent such as dioxane to generate intermediates of type G4.2. In the third step, the Boc group of G4.2 is removed via treatment with dilute HCl, in the presence of a solvent like MeOH, to provide products of type G4.3. In the fourth step, intermediates of type G4.3 can then be combined with acids G2.1 in the presence of a coupling reagent such as COMU in the presence of a base such as DIPEA, and a solvent such as DMA to produce the coupled products G4.4. In the second and final step, intermediates of type G4.4 can undergo a rearrangement upon heating in neat BSA to form products of type G4.5.

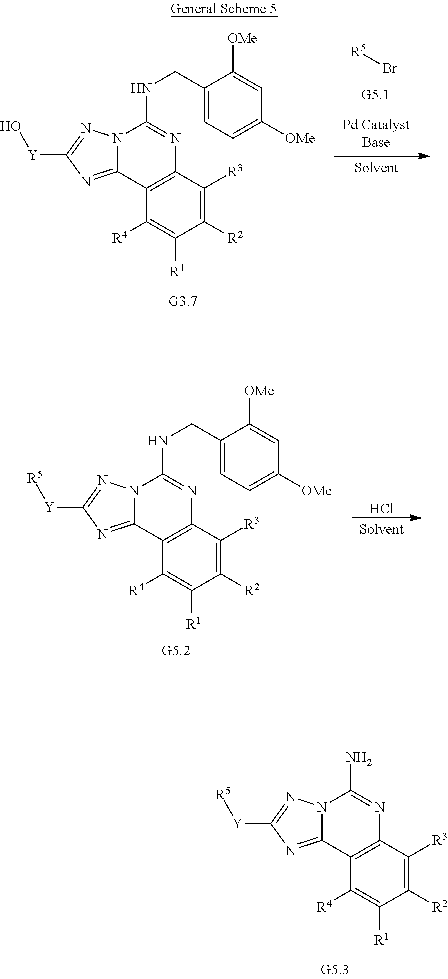

As an extension to the utility of intermediates like G3.7, one general strategy for the synthesis of compounds of type G5.3 is via a two-step procedure outlined in General Scheme 5, wherein Y, R¹, R², R³, R⁴ and R⁵ are defined in Formula (I). In the first step, intermediates of type G3.7 can be converted into intermediates of type G5.2, wherein one CH₂ in Y is replaced with an oxygen, through a palladium-catalyzed C—C coupling reaction with bromides G5.1. The reaction is performed under deoxygenated conditions at the appropriate temperature with palladium catalysts such as RockPhos Pd G3, a base such as Cs₂CO₃, and a solvent such as toluene. In the second and final step, the 2,4-dimethoxybenzyl group of G3.10 is removed via treatment with HCl in the presence of a solvent like water, to provide products of type G5.3. Products of type G5.3 can be purified by trituration, filtering, and washing with an appropriate solvent, preparative TLC, silica gel chromatography, preparative reversed-phase HPLC, and/or chiral SFC.

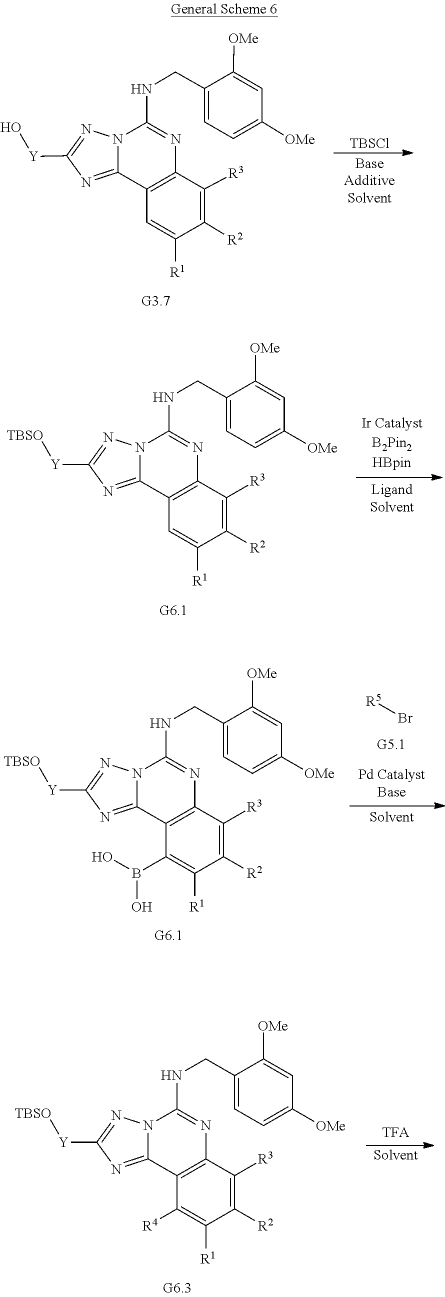

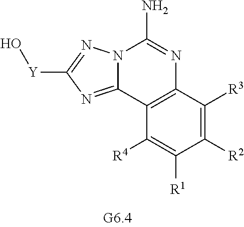

As another extension to the utility of intermediates like G3.7, one general strategy for the synthesis of compounds of type G6.4 is via a four-step procedure outlined in General Scheme 6, wherein Y, R¹, R², R³, and R⁴ are defined in Formula (I). In the first step, intermediates of type G3.7 can be converted into intermediates of type G6.1 through a protection reaction with TBSCl, wherein bases such as DIPEA, additives such as DMAP, and solvents such as DMF can be used. In the second step, intermediates of type G6.1 can be converted into intermediates of type G6.2 through an iridium-catalyzed C—H functionalization reaction with B₂Pin₂ and HBpin. The reaction is performed under deoxygenated conditions at the appropriate temperature with iridium catalysts such as [(COD)IrOMe]₂, a ligand such as P(C₆F₅)₃, and a solvent such as Me-THF. In the third step, intermediates of type G6.2 can be converted into intermediates of type G6.3 through a palladium-catalyzed C—C coupling reaction with bromides G5.1. The reaction is performed under deoxygenated conditions at the appropriate temperature with palladium catalysts such as PdCl₂(dppf)·CH₂Cl₂, a base such as K₂CO₃, and a solvent such as dioxane. In the fourth and final step, the 2,4-dimethoxybenzyl group of G6.3 is removed via treatment with TFA, either neat or in the presence of a solvent like DCM, to provide products of type G6.4. Products of type G6.4 can be purified by trituration, filtering, and washing with an appropriate solvent, preparative TLC, silica gel chromatography, preparative reversed-phase HPLC, and/or chiral SFC.

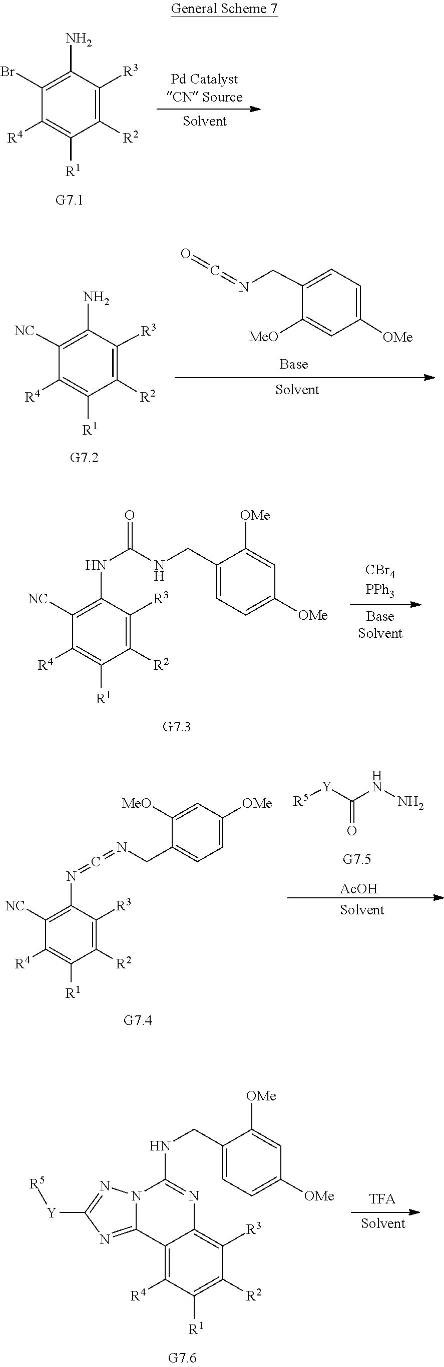

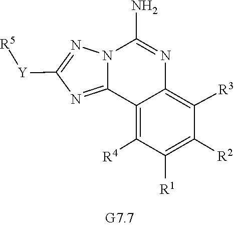

One general strategy for the synthesis of compounds of type G7.6 is via a five-step procedure shown in General Scheme 7, wherein Y, R¹, R², R³, R⁴ and R⁵ are defined in Formula (I). In the first step, intermediates of type G7.1 can be converted into intermediates of type G7.2 through a palladium-catalyzed cyanation reaction. The reaction is performed under deoxygenated conditions at the appropriate temperature with palladium catalysts such as Pd(PPh₃)₄, a “CN” source such as Zn(CN)₂, and a solvent such as DMF. In the second step, amino benzonitriles G7.2 can be treated with 1-(isocyanatomethyl)-2,4-dimethoxybenzene in a solvent such as dichloromethane, and a base such as pyridine to form intermediate ureas G7.3. In the third step, ureas G7.3 can be dehydrated to the corresponding carbodiimides G7.4 in the presence of PPh₃, CBr₄, a base such as Et₃N, and a solvent such as DCM. In the fourth step, treatment of carbodiimides G7.4 with a hydrazide of the type G7.5 in the presence of AcOH in a solvent such as DCM, DMF, or dioxane, produces products of the type G7.6. In addition, subsequent manipulations can be done on R⁴ to provide further elaborated products. In the fifth and final step, the 2,4-dimethoxybenzyl group of G7.6 is removed via treatment with TFA, either neat or in the presence of a solvent like DCM, to provide products of type G7.7. Products of type G7.7 can be purified by trituration, filtering, and washing with an appropriate solvent, preparative TLC, silica gel chromatography, preparative reversed-phase HPLC, and/or chiral SFC.

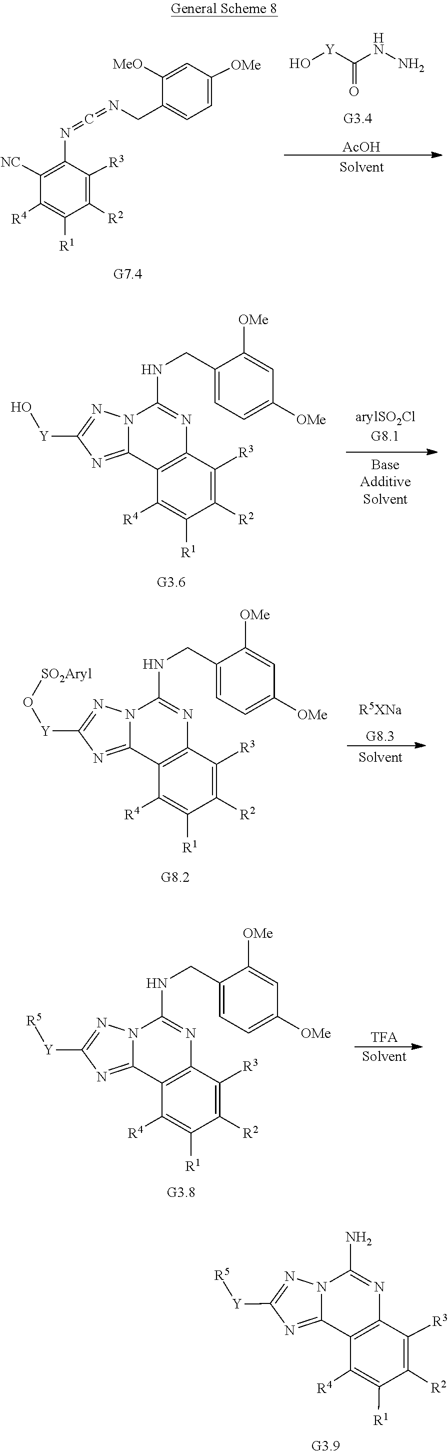

As an extension to the utility of intermediates like G7.4, another general strategy for the synthesis of compounds of type G3.9 is via a four-step procedure shown in General Scheme 8, wherein Y, R¹, R², R³, R⁴ and R⁵ are defined in Formula (I). In the first step, analogous to Step 4 of General Scheme 7, treatment of carbodiimides G7.4 with a hydrazide of the type G3.4 in the presence of AcOH in a solvent such as DCM, DMF, or dioxane, produces products of the type G3.6, illustrating another route to access these intermediates. In the second step, intermediates of type G3.6 can be converted into intermediates of type G8.2 upon treatment with sulfonyl chlorides G8.1 in the presence of a solvent such as THE or DCM, a base such as Et₃N, and an additive such as DMAP. In the third step, intermediates of type G8.2 can be treated with sodium salts G8.3 (X═O, S, or SO₂) in the presence of solvents such as EtOH, DMF, or MeCN to provide products of the type G3.8, illustrating another route to access these intermediates. In the fourth and final step, the 2,4-dimethoxybenzyl group of G3.8 is removed via treatment with TFA, either neat or in the presence of a solvent like DCM, to provide products of type G3.9, illustrating another route to access these products. Products of type G3.9 can be purified by trituration, filtering, and washing with an appropriate solvent, preparative TLC, silica gel chromatography, preparative reversed-phase HPLC, and/or chiral SFC.

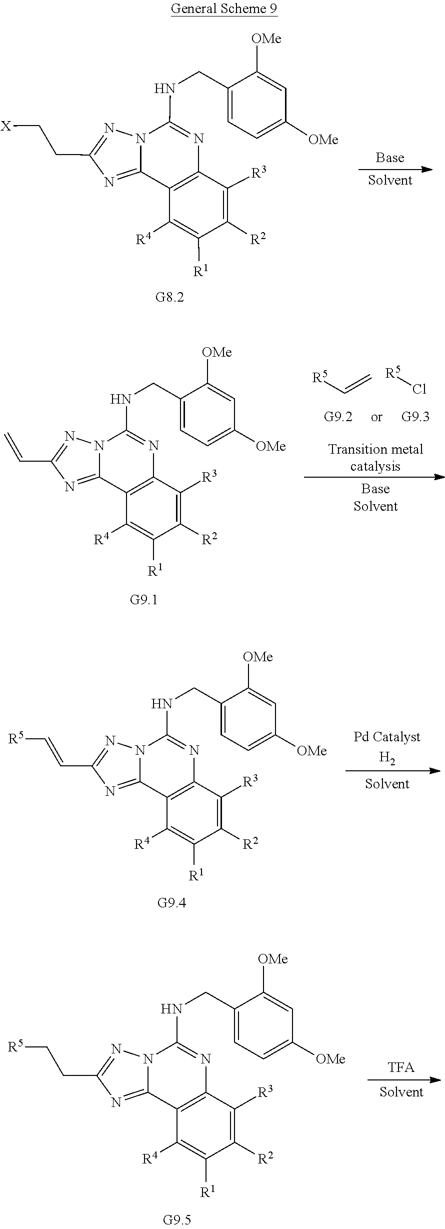

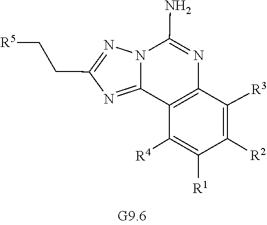

As an extension to the utility of intermediates like G8.2 (when n=2), another general strategy for the synthesis of compounds of type G9.6 is via a four-step procedure shown in General Scheme 9, wherein Y, R¹, R², R³, R⁴ and R⁵ are defined in Formula (I). In the first step, intermediates of type G8.2 (when n=2) can be converted into intermediates of type G9.1 upon treatment with a base such as DBU, and a solvent such as DCE. In the second step, intermediates of type G9.1 can be converted into intermediates of type G9.4 through a transition-metal catalyzed coupling reaction. When using olefins G9.2, the reaction is performed at the appropriate temperature with Hoveyda-Grubbs 2^{nd} Generation Catalyst© 2^{nd} generation catalysts and a solvent such as DCM. When using bromides G9.3, the reaction is performed under deoxygenated conditions at the appropriate temperature with palladium catalysts such as P(t-Bu)₃ Pd G2, a base such as DIPEA, and a solvent such as DMF. In the second step, intermediates of type G9.4 can be converted into intermediates of type G9.5 through a palladium-catalyzed hydrogenation reaction. The reaction is performed under an atmosphere of H2 at the appropriate temperature and pressure with palladium catalysts such as Pd/C or Pd(OH)₂, and a solvent such as MeOH. In the fourth and final step, the 2,4-dimethoxybenzyl group of G9.5 is removed via treatment with TFA, either neat or in the presence of a solvent like DCM, to provide products of type G9.6. Products of type G9.6 can be purified by trituration, filtering, and washing with an appropriate solvent, preparative TLC, silica gel chromatography, preparative reversed-phase HPLC, and/or chiral SFC.

### General Experimental Information:

Unless otherwise noted, all reactions were magnetically stirred and performed under an inert atmosphere such as nitrogen or argon.

Unless otherwise noted, diethyl ether used in the experiments described below was Fisher ACS certified material and stabilized with BHT.

Unless otherwise noted, “degassed” refers to a solvent from which oxygen has been removed, generally by bubbling an inert gas such as nitrogen or argon through the solution for 10 to 15 minutes with an outlet needle to normalize pressure.

Unless otherwise noted, “concentrated” means evaporating the solvent from a solution or mixture using a rotary evaporator or vacuum pump.

Unless otherwise noted, flash chromatography was carried out on an ISCO®, Analogix®, or Biotage® automated chromatography system using a commercially available cartridge as the column. Columns were usually filled with silica gel as the stationary phase. Reversed-phase preparative HPLC conditions can be found at the end of the experimental section. Aqueous solutions were concentrated on a Genevac® evaporator or were lyophilized.

Unless otherwise noted, proton nuclear magnetic resonance (H NMR) spectra and proton-decoupled carbon nuclear magnetic resonance (¹³C{¹H} NMR) spectra were recorded on 400, 500, or 600 MHz Bruker or Varian NMR spectrometers at ambient temperature. All chemical shifts (δ) were reported in parts per million (ppm). Proton resonances were referenced to residual protium in the NMR solvent, which can include, but is not limited to, CDCl₃, DMSO-d₆, and MeOD-d₄. Carbon resonances are referenced to the carbon resonances of the NMR solvent. Data are represented as follows: chemical shift, multiplicity (br=broad, br s=broad singlet, s=singlet, d=doublet, dd=doublet of doublets, ddd=doublet of doublet of doublets, t=triplet, q=quartet, m=multiplet), coupling constants (J) in Hertz (Hz), integration.

### Preparation of Intermediate A.1, 2-(4-(hydroxymethyl)phenyl)propan-2-ol

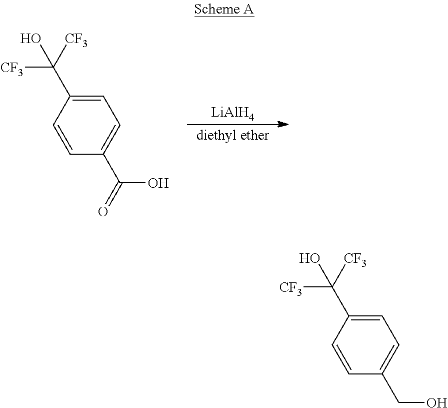 [SMILES: O=C(O)c1ccc(C(O)(C(F)(F)F)C(F)(F)F)cc1 ; "LiAlH4" ; "diethyl ether" ; OCc1ccc(C(O)(C(F)(F)F)C(F)(F)F)cc1]

LiAlH₄ (2 M in THF, 2.60 mL, 5.21 mmol) was added dropwise to a stirred solution of 4-(1,1,1,3,3,3-hexafluoro-2-hydroxypropan-2-yl)benzoic acid (500 mg, 1.74 mmol) in diethyl ether (8.7 mL) over 1 minute at 0° C. The reaction was allowed to warm to room temperature. After 2 h, the reaction was quenched by the dropwise addition of water (5 mL). The resulting slurry was filtered through Celite©. The resulting filtrate was diluted with water (25 mL) and extracted with diethyl ether (2×25 mL). The combined organic layers were dried over anhydrous MgSO₄, filtered, and concentrated under reduced pressure. The resulting crude residue was purified by silica gel chromatography (gradient elution: 0-50% EtOAc/hexanes) to provide 2-(4-(hydroxymethyl)phenyl)propan-2-ol. MS (ESI) m/z calc'd for C₁₀H₇F₆O [M]⁺ (—OH fragment) 257.0. found 257.1.

### Preparation of Intermediate B.1, 2-(4-(hydroxymethyl)phenyl)propan-2-ol

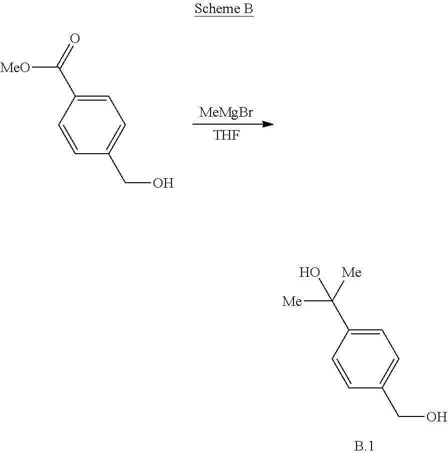 [SMILES: "MeMgBr" ; COC(=O)c1ccc(CO)cc1 ; CC(C)(O)c1ccc(CO)cc1 ; "THF"]

MeMgBr (3.4 M in 2-MeTHF, 1.55 mL, 5.27 mmol) was added dropwise to a stirred solution of methyl 4-(hydroxymethyl)benzoate (250 mg, 1.50 mmol) in THE (9.29 mL) over 1 minute at 0° C. The reaction was allowed to warm to room temperature. After 1.5 h, the reaction was quenched with sat. aq. NH₄Cl (5 mL), diluted with water (25 mL), and extracted with EtOAc (3×10 mL). The combined organic layers were dried over anhydrous MgSO₄, filtered, and concentrated under reduced pressure to provide 2-(4-(hydroxymethyl)phenyl)propan-2-ol, which was used in subsequent steps without further purification. MS (ESI) m/z calc'd for C₁₀H₁₃O [M]⁺ (—OH fragment) 149.1. found 149.2.

### Preparation of Intermediate C.6, 1-(3,3-difluorocyclobutyl)-5-vinyl-4,5,6,7-tetrahydro-1H-benzo[d][1,2,3]triazol-5-ol

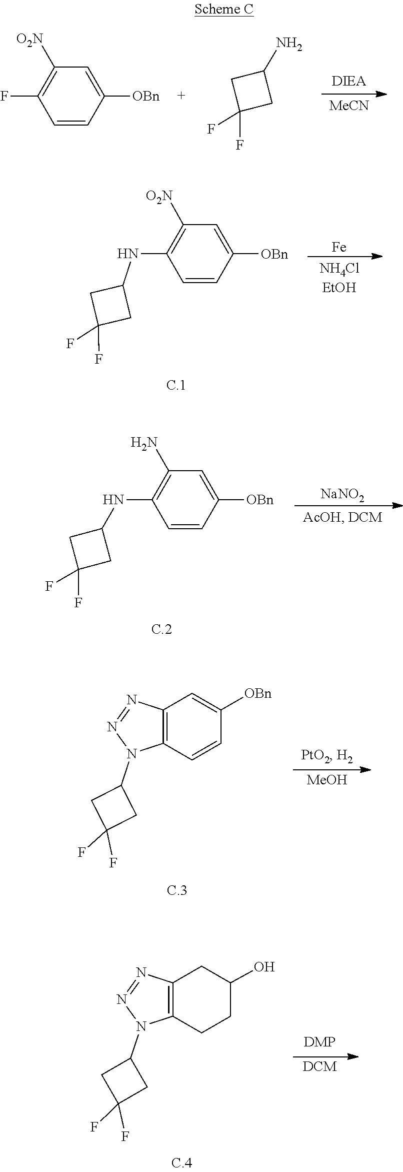

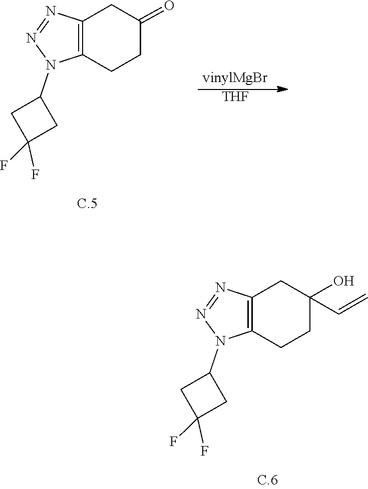 [SMILES: O=C1CCc2c(nnn2C2CC(F)(F)C2)C1 ; C=CC1(O)CCc2c(nnn2C2CC(F)(F)C2)C1 ; "THF"]

### Step 1—Synthesis of Intermediate C.1, of 4-(benzyloxy)-N-(3,3-difluorocyclobutyl)-2-nitroaniline

3,3-difluorocyclobutanamine hydrochloride (9.87 g, 68.8 mmol) and DIPEA (31.7 mL, 172 mmol) were added to a stirred solution 4-(benzyloxy)-1-fluoro-2-nitrobenzene (8.5 g, 34.4 mmol) in MeCN (50 mL). The reaction was stirred vigorously at 80° C. for 48 h. The reaction was cooled to room temperature and concentrated under reduced pressure. The resulting crude residue was purified by silica gel chromatography (gradient elution: 0-30% EtOAc/petroleum ether) to provide 4-(benzyloxy)-N-(3,3-difluorocyclobutyl)-2-nitroaniline. MS (ESI) m/z calc'd for C₁₇H₁₆F₂N₂O₃ [M+H]+ 335.1. found 335.1.

### Step 2—Synthesis of Intermediate C.2, 4-(benzyloxy)-N^{I}-(3,3-difluorocyclobutyl)benzene-1,2-diamine

Iron (6.82 g, 122 mmol) and NH₄Cl (6.53 g, 122 mmol) were added to a stirred solution of 4-(benzyloxy)-N-(3,3-difluorocyclobutyl)-2-nitroaniline (10.2 g, 30.5 mmol) in EtOH (30 mL) and water (10 mL) at 80° C. The reaction was heated at 80° C. for 1 h. The reaction was cooled to room temperature, filtered, and concentrated under reduced pressure to provide 4-(benzyloxy)-N^{I}-(3,3-difluorocyclobutyl)benzene-1,2-diamine, which was used in the subsequent step without further purification. MS (ESI) m/z calc'd for C₁₇H₁₈F₂N₂O [M+H]+ 305.1. found 305.1.

### Step 3—Synthesis of Intermediate C.3, 5-(benzyloxy)-1-(3,3-difluorocyclobutyl)-1H-benzo[d][1,2,3]triazole

NaNO₂ (3.85 g, 55.9 mmol) was added to a stirred solution of 4-(benzyloxy)-N^{I}-(3,3-difluorocyclobutyl)benzene-1,2-diamine (8.5 g, 27.9 mmol) in DCM (80 mL) and AcOH (80 mL) at 0° C. The reaction was stirred vigorously at 0° C. for 5 h. While still at 0° C., the reaction was diluted with water (50 mL) and extracted with DCM (2×50 mL). The combined organic layers were dried over anhydrous Na₂SO₄, filtered, and concentrated under reduced pressure. The resulting crude residue was purified by silica gel chromatography (gradient elution: 0-50% EtOAc/petroleum ether) to provide 5-(benzyloxy)-1-(3,3-difluorocyclobutyl)-1H-benzo[d][1,2,3]triazole. MS (ESI) m/z calc'd for C₁₇H₁₅F₂N₃O [M+H]+ 316.2. found 316.0.

### Step 4—Synthesis of Intermediate C.4, 1-(3,3-difluorocyclobutyl)-4,5,6,7-tetrahydro-1H-benzo[d][1,2,3]triazol-5-ol

PtO₂ (2.16 g, 9.51 mmol) was added to a stirred solution 5-(benzyloxy)-1-(3,3-difluorocyclobutyl)-1H-benzo[d][1,2,3]triazole (1.5 g, 4.76 mmol) in MeOH (100 mL) under an atmosphere of H₂. The reaction was stirred vigorously at 50° C. for 48 h under an atmosphere of H₂ at 50 psi. The reaction was cooled to room temperature, filtered, and concentrated under reduced pressure to provide 1-(3,3-difluorocyclobutyl)-4,5,6,7-tetrahydro-1H-benzo[d][1,2,3]triazol-5-ol, which was used in subsequent steps without further purification. MS (ESI) m/z calc'd for C₁₀H₁₃F₂N₃O [M+H]+ 230.1. found 230.1.

### Step 5—Synthesis of Intermediate C.5, of 1-(3,3-difluorocyclobutyl)-6,7-dihydro-1H-benzo[d][1,2,3]triazol-5(4H)-one

DMP (1.48 g, 3.49 mmol) was added to a stirred solution of 1-(3,3-difluorocyclobutyl)-4,5,6,7-tetrahydro-1H-benzo[d][1,2,3]triazol-5-ol (400 mg, 1.745 mmol) in DCM (5 mL) at 15° C. The reaction was stirred vigorously at 15° C. for 15 h. The reaction was warmed to room temperature, filtered, and concentrated under reduced pressure. The resulting crude residue was purified by preparative TLC (35% EtOAc/petroleum ether) to provide 1-(3,3-difluorocyclobutyl)-6,7-dihydro-1H-benzo[d][1,2,3]triazol-5(4H)-one. MS (ESI) m/z calc'd for C₁₀H₁₁F₂N₃O [M+H]⁺ 228.0. found 228.1.

### Step 6—Synthesis of Intermediate C.6, 1-(3,3-difluorocyclobutyl)-5-vinyl-4,5,6,7-tetrahydro-1H-benzo[d][1,2,3]triazol-5-ol

Vinylmagnesium bromide (1 M in THF, 3.98 mL, 3.98 mmol) was added to a stirred solution of 1-(3,3-difluorocyclobutyl)-6,7-dihydro-1H-benzo[d][1,2,3]triazol-5(4H)-one (362 mg, 1.593 mmol) in THE (5 mL) at 0° C. The reaction was stirred vigorously at 0° C. for 2 h.

While still at 0° C., the reaction was quenched with water (1 mL) and extracted with EtOAc (3×10 mL). The combined organic layers were dried over anhydrous Na₂SO₄, filtered, and concentrated under reduced pressure. The resulting crude residue was purified by preparative TLC (65% EtOAc/petroleum ether) to provide 1-(3,3-difluorocyclobutyl)-5-vinyl-4,5,6,7-tetrahydro-1H-benzo[d][1,2,3]triazol-5-ol. MS (ESI) m/z calc'd for C₁₂H₁₅F₂N₃O [M+H]⁺ 256.1. found 256.1.

### Preparation of Intermediate D.2, 4-bromo-N^{I}-(3,3-difluorocyclobutyl)benzene-1,2-diamine

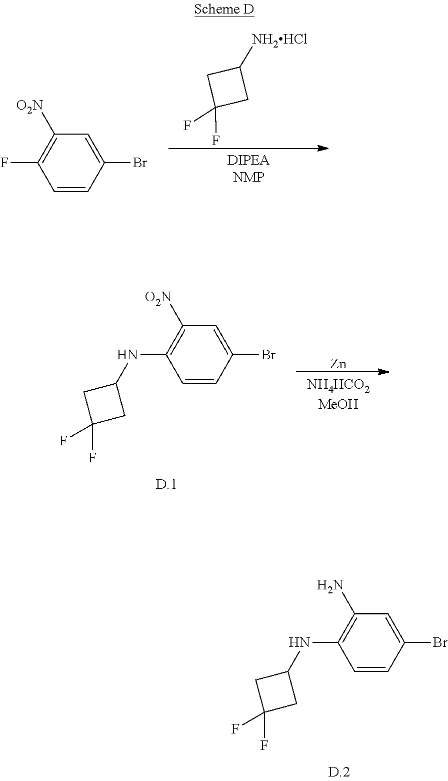

### Step 1—Synthesis of Intermediate D.1, 4-bromo-N-(3,3-difluorocyclobutyl)-2-nitroaniline

DIPEA (30.4 mL, 174 mmol) was added dropwise to a stirred solution of 3,3-difluorocyclobutan-1-amine hydrochloride (10.0 g, 69.7 mmol) and 4-bromo-1-fluoro-2-nitrobenzene (15.3 g, 69.7 mmol) in NMP (100 mL). The reaction was stirred vigorously at 65° C. for 16 h. The reaction was cooled to room temperature, diluted with sat. aq. K₂CO₃ and stirred for 30 min. The reaction mixture was extracted with diethyl ether (3×100 mL), and the combined organic layers were dried over anhydrous MgSO₄, filtered, and concentrated under reduced pressure to provide 4-bromo-N-(3,3-difluorocyclobutyl)-2-nitroaniline, which was used in the subsequent step without further purification. MS (ESI) m/z calc'd for C₁₀H₁₀BrF₂N₂O₂ [M+H]⁺ 307.0. found 307.0, 309.0.

### Step 2—Synthesis of Intermediate D.2, 4-bromo-N^{I}-(3,3-difluorocyclobutyl)benzene-1,2-diamine

NH₄HCO₂ (4.83 g, 77.0 mmol) was added to a stirred solution of 4-bromo-N-(3,3-difluorocyclobutyl)-2-nitroaniline (5.00 g, 16.3 mmol) and Zn (3.51 g, 53.7 mmol) in MeOH (109 mL). The reaction was stirred vigorously at room temperature for 15 min. The reaction was diluted with sat. aq. NaHCO₃ (200 mL) and EtOAc (200 mL), and stirred vigorously for 30 min. The combined organic layers were dried over anhydrous Na₂SO₄, filtered, and concentrated under reduced pressure to provide 4-bromo-N^{I}-(3,3-difluorocyclobutyl)benzene-1,2-diamine, which was used in subsequent steps without further purification. MS (ESI) m/z calc'd for C₁₀H₁₂BrF₂N₂ [M+H]⁺ 277.1. found 277.0, 279.1.

### Preparation of Intermediate E.3, ethyl 3-(1-isopropyl-4,5,6,7-tetrahydro-1H-benzo[d][1,2,3]triazol-5-yl)propanoate

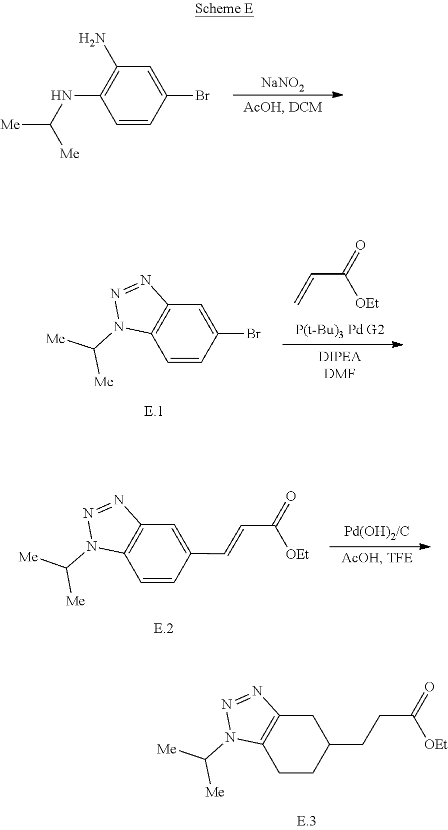

### Step 1—Synthesis of Intermediate E.1, 5-bromo-1-isopropyl-1H-benzo[d][1,2,3]triazole

NaNO₂ (241 mg, 3.49 mmol) was added to a stirred solution of 4-bromo-N^{I}-isopropylbenzene-1,2-diamine (500 mg, 2.18 mmol) in AcOH (5.5 mL) and DCM (5.5 mL) at 0° C. The reaction was stirred vigorously at 0° C. for 30 min. While still at 0° C., the reaction was diluted with DCM (10 mL), carefully quenched with sat. aq. NaHCO₃, extracted with DCM (4×100 mL). The combined organic layers were dried over anhydrous MgSO₄, filtered, and concentrated under reduced pressure. The resulting crude residue was purified by silica gel chromatography (gradient elution: 0-30% EtOAc/hexanes) to provide 5-bromo-1-isopropyl-1H-benzo[d][1,2,3]triazole. MS (ESI) m/z calc'd for C₉H₁₁BrN₃ [M+H]⁺ 240.0. found 240.1, 242.0.

### Step 2—Synthesis of Intermediate E.2, ethyl (E)-3-(1-isopropyl-1H-benzo[d][1,2,3]triazol-5-yl)acrylate

5-bromo-1-isopropyl-1H-benzo[d][1,2,3]triazole (2.72 g, 11.3 mmol), P(t-Bu)₃ Pd G2 (1.16 g, 2.27 mmol), ethyl acrylate (1.81 mL, 17.0 mmol), and DIPEA (3.96 mL, 22.7 mmol) were combined. DMF (45.3 mL) was added, and the reaction was degassed by sparging with argon for 3 min. The reaction was heated to 100° C. for 16 h. The reaction was cooled to room temperature, diluted with water (100 mL) and extracted with DCM (3×100 mL). The combined organic layers were dried over anhydrous MgSO₄, filtered, and concentrated under reduced pressure. The resulting crude residue was purified by silica gel chromatography (gradient elution: 0-60% EtOAc/hexanes) to provide ethyl (E)-3-(1-isopropyl-1H-benzo[d][1,2,3]triazol-5-yl)acrylate. MS (ESI) m/z calc'd for C₁₄H₁₈N₃O₂ [M+H]⁺ 260.1. found 260.1.

### Step 3—Synthesis of Intermediate E.3, ethyl 3-(1-isopropyl-4,5,6,7-tetrahydro-1H-benzo[d][1,2,3]triazol-5-yl)propanoate

Pd(OH)₂/C (50%, 14 mg, 0.05 mmol) was added to a stirred solution of ethyl (E)-3-(1-isopropyl-1H-benzo[d][1,2,3]triazol-5-yl)acrylate (133 mg, 0.5 mmol) in TFE (3.6 mL) and AcOH (0.25 mL). The reaction was stirred vigorously at 60° C. for 18 h under an atmosphere of H₂ at 150 psi. The reaction was cooled to room temperature, filtered, and concentrated under reduced pressure to provide ethyl 3-(1-isopropyl-4,5,6,7-tetrahydro-1H-benzo[d][1,2,3]triazol-5-yl)propanoate, which was used in subsequent steps without further purification. MS (ESI) m/z calc'd for C₁₄H₂₄N₃O₂ [M+H]⁺ 266.2. found 266.2.

The following compound in Table 1 was prepared according to Scheme E starting from Intermediate D.2.

TABLE 1
Intermediate Componds Prepared According to Scheme E

| En- | Structure | Observed m/z |
|---|---|---|
| try | Name | [M + H ]⁺ |
| E.4 | 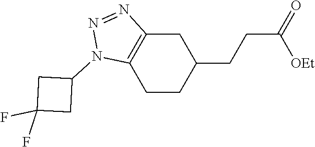 [SMILES: CCOC(=O)CCC1CCc2c(nnn2C2CC(F)(F)C2)C1] | 314 |
| | ethyl 3-(1-(3,3-difluorocyclobutyl)-4,5,6,7-tetrahydro- | |
| | 1H-benzo[d][1,2,3]triazol-5-yl)propanoate | |

### Preparation of Intermediate F.2, 7-chloro-3-(3,3-difluorocyclobutyl)imidazo[1,5-a]pyridine

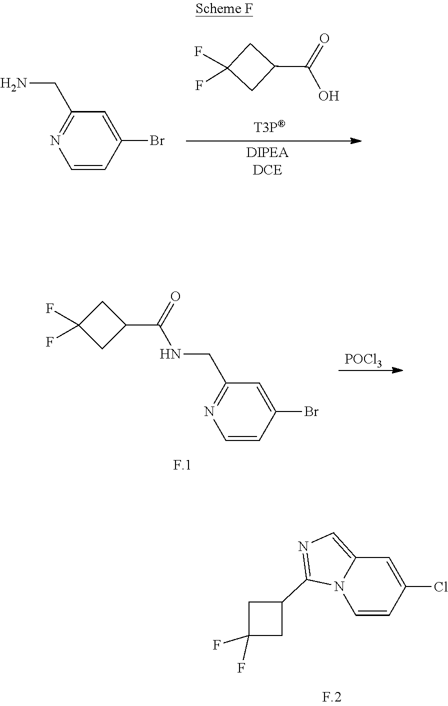 [SMILES: NCc1cc(Br)ccn1 ; O=C(O)C1CC(F)(F)C1 ; O=C(NCc1cc(Br)ccn1)C1CC(F)(F)C1 ; "POCl3" ; FC1(F)CC(c2ncc3cc(Cl)ccn23)C1]

### Step 1—Synthesis of Intermediate F.1, N-((4-bromopyridin-2-yl)methyl)-3,3-difluorocyclobutane-1-carboxamide

3,3-Difluorocyclobutane-1-carboxylic acid (469 mg, 3.44 mmol), DIPEA (1.57 mL, 8.98 mmol) and T3P© (>50 wt % in EtOAc, 2.53 mL, 3.59 mmol) were sequentially added to a stirred solution of (4-bromopyridin-2-yl)methanamine (560 mg, 2.99 mmol) in DCE (15.0 mL). The reaction was stirred vigorously at room temperature 1 h. The reaction was diluted with DCM and washed with 1 N aq. NaOH. The combined organic layers were dried over anhydrous MgSO₄, filtered, and concentrated under reduced pressure to provide N-((4-bromopyridin-2-yl)methyl)-3,3-difluorocyclobutane-1-carboxamide. MS (ESI) m/z calc'd for C₁H₁₂BrF₂N₂O [M+H]+ 305.0. found 305.0, 307.0.

### Step 2—Synthesis of Intermediate F.2, 7-chloro-3-(3,3-difluorocyclobutyl)imidazo[1,5-a]pyridine

POCl₃ (4.26 mL, 45.7 mmol) was added to N-((4-bromopyridin-2-yl)methyl)-3,3-difluorocyclobutane-1-carboxamide (930 mg, 3.05 mmol). The reaction was stirred vigorously at 80° C. for 1.5 h. The reaction was quenched with sat. aq. K₂CO₃ at 0° C. until pH 7-8 (30 mL) and extracted with DCM (3×30 mL). The combined organic layers were dried over anhydrous MgSO₄, filtered, and concentrated under reduced pressure. The resulting crude residue was purified by silica gel chromatography (gradient elution: 5-30% EtOAc/hexanes) to provide 7-chloro-3-(3,3-difluorocyclobutyl)imidazo[1,5-a]pyridine. MS (ESI) m/z calc'd for C₁₁H₁₀ClF₂N₂ [M+H]⁺ 243.0. found 243.1.

The following compound in Table 2 was prepared according to Scheme F starting from (4-bromopyridin-2-yl)methanamine and the commercially available acid.

TABLE 2
Intermediate Compounds Prepared According to Scheme F

| | Structure | Observed m/z |
|---|---|---|
| Entry | Name | [M + H ]⁺ |
| F.3 | 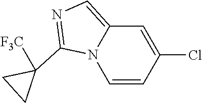 [SMILES: FC(F)(F)C1(c2ncc3cc(Cl)ccn23)CC1] | 261.0 |
| | 7-chloro-3-(1-(trifluoromethyl)cyclopropyl)imidazo[1,5- | |
| | a]pyridine | |

### Preparation of Intermediate G.2, ethyl 3-(3-(3,3-difluorocyclobutyl)-5,6,7,8-tetrahydroimidazo[1,5-a]pyridin-7-yl)propanoate

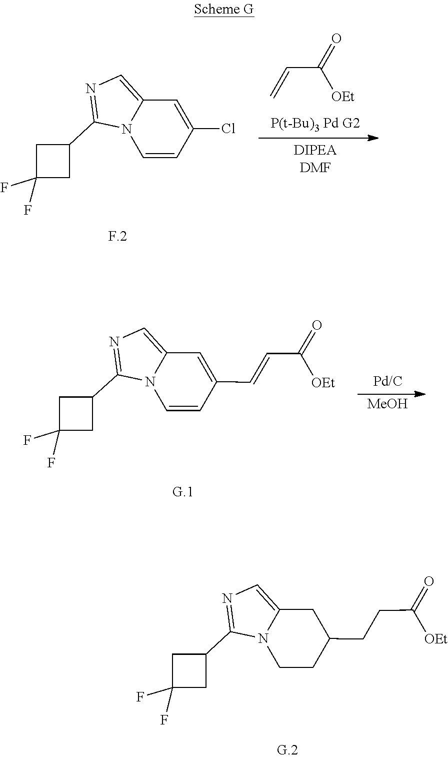

### Step 1—Synthesis of Intermediate G.1, ethyl (E)-3-(3-(3,3-difluorocyclobutyl)imidazo[1,5-a]pyridin-7-yl)acrylate

7-Chloro-3-(3,3-difluorocyclobutyl)imidazo[1,5-a]pyridine (375 mg, 1.55 mmol), P(t-Bu)₃ Pd G2 (79 mg, 0.155 mmol), ethyl acrylate (0.33 mL, 3.09 mmol), and DIPEA (0.54 mL, 3.09 mmol) were combined. DMF (7.7 mL) was added, and the reaction was degassed by sparging with argon for 3 min. The reaction was heated to 110° C. for 16 h. The reaction was cooled to room temperature, diluted with EtOAc (10 mL), filtered, and concentrated under reduced pressure. The resulting crude residue was purified by silica gel chromatography (gradient elution: 0-40% [3:1 EtOAc/i-PrOH]/hexanes) to provide ethyl (E)-3-(3-(3,3-difluorocyclobutyl)imidazo[1,5-a]pyridin-7-yl)acrylate. MS (ESI) m/z calc'd for C₁₆H₁₇F₂N₂O₂ [M+H]⁺ 307.1. found 307.1.

### Step 2—Synthesis of Intermediate G.2, ethyl 3-(3-(3,3-difluorocyclobutyl)-5,6,7,8-tetrahydroimidazo[1,5-a]pyridin-7-yl)propanoate

Pd/C (10%, 193 mg, 0.18 mmol) was added to a stirred solution of ethyl (E)-3-(3-(3,3-difluorocyclobutyl)imidazo[1,5-a]pyridin-7-yl)acrylate (108 mg, 0.5 mmol) in MeOH (12 mL) The reaction was stirred vigorously at room temperature for 16 h under an atmosphere of H₂. The reaction was diluted with DCM, filtered through Celite©, and concentrated under reduced pressure. The resulting crude residue was purified by silica gel chromatography (gradient elution: 5-45% [3:1 EtOAc/i-PrOH]/hexanes) to provide ethyl 3-(3-(3,3-difluorocyclobutyl)-5,6,7,8-tetrahydroimidazo[1,5-a]pyridin-7-yl)propanoate. MS (ESI) m/z calc'd for C₁₆H₂₃F₂N₂O₂ [M+H]⁺ 313.2. found 313.2.

### Preparation of Intermediates B.3 and B.4, ethyl 3-(2-isopropyl-4,5,6,7-tetrahydro-2H-indazol-5-yl)propanoate and ethyl 3-(1-isopropyl-4,5,6,7-tetrahydro-1H-indazol-5-yl)propanoate

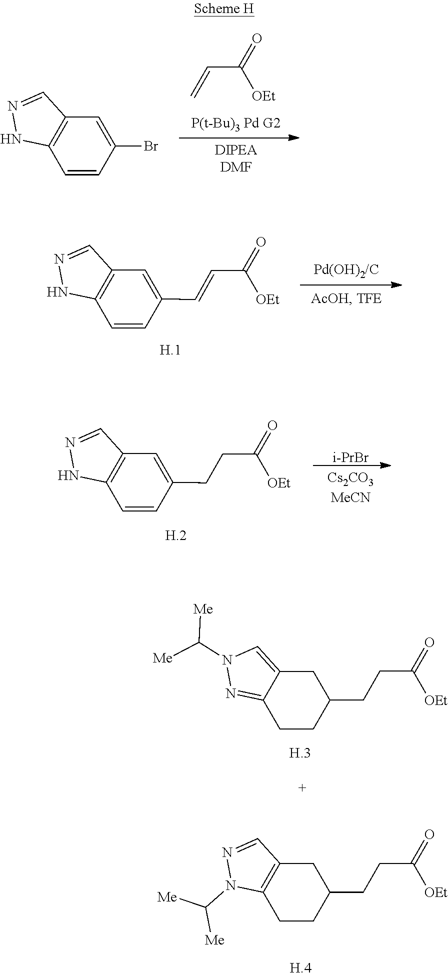

### Step 1—Synthesis of Intermediate H.1, ethyl (E)-3-(1H-indazol-5-yl)acrylate

5-bromo-1H-indazole (5.00 g, 25 mmol), P(t-Bu)₃ Pd G2 (3.00 g, 5.80 mmol), ethyl acrylate (4.00 mL, 38.2 mmol), and DIPEA (8.90 mL, 50.8 mmol) were combined. DMF (100 mL) was added, and the reaction was degassed by sparging with argon for 3 min. The reaction was heated at 100° C. for 16 h. The reaction was cooled to room temperature, diluted with water (100 mL) and extracted with DCM (3×100 mL). The combined organic layers were dried over anhydrous MgSO₄, filtered, and concentrated under reduced pressure. The resulting crude residue was purified by silica gel chromatography (gradient elution: 0-50% [3:1 EtOAc/i-PrOH]/hexanes) followed by reversed-phase HPLC [Method A] to provide ethyl (E)-3-(1H-indazol-5-yl)acrylate. MS (ESI) m/z calc'd for C₁₂H₁₃N₂O₂ [M+H]⁺ 217.1. found 217.1.

### Step 2—Synthesis of Intermediate H.2, ethyl 3-(1H-indazol-5-yl)propanoate

Pd(OH)₂/C (50%, 14 mg, 0.05 mmol) was added to a stirred solution of ethyl (E)-3-(1H-indazol-5-yl)acrylate (108 mg, 0.5 mmol) in TFE (3.6 mL) and AcOH (0.25 mL). The reaction was stirred vigorously at 80° C. for 18 h under an atmosphere of H₂ at 150 psi. The reaction was cooled to room temperature, filtered, and concentrated under reduced pressure to provide ethyl 3-(1H-indazol-5-yl)propanoate, which was used in the subsequent step without further purification. MS (ESI) m/z calc'd for C₁₂H₁₉N₂O₂ [M+H]⁺ 223.1. found 223.1.

### Step 3—Synthesis of Intermediates H.3 and H.4, ethyl 3-(2-isopropyl-4,5,6,7-tetrahydro-2H-indazol-5-yl)propanoate and ethyl 3-(1-isopropyl-4,5,6,7-tetrahydro-1H-indazol-5-yl)propanoate

i-PrBr (0.49 mL, 5.2 mmol) was added to a suspension of ethyl 3-(1H-indazol-5-yl)propanoate (526 mg, 2.37 mmol) and Cs₂CO₃ (1.54 g, 4.73 mmol) in MeCN (15 mL). The reaction was heated to 80° C. for 16 h. The reaction was cooled to room temperature, diluted with water and extracted with EtOAc. The combined organic layers were dried over anhydrous MgSO₄, filtered, and concentrated under reduced pressure. The mixture of the two regioisomers was purified by silica gel chromatography (gradient elution: 0-60% EtOAc/hexanes) to provide ethyl 3-(2-isopropyl-4,5,6,7-tetrahydro-2H-indazol-5-yl)propanoate (faster eluting) and ethyl 3-(1-isopropyl-4,5,6,7-tetrahydro-1H-indazol-5-yl)propanoate (slower eluting) as racemic mixtures. Ethyl 3-(2-isopropyl-4,5,6,7-tetrahydro-2H-indazol-5-yl)propanoate (faster eluting): MS (ESI) m/z calc'd for C₁₅H₂₅N₂O₂ [M+Na]⁺ 265.2. found 265.2. Ethyl 3-(1-isopropyl-4,5,6,7-tetrahydro-1H-indazol-5-yl)propanoate (slower eluting): MS (ESI) m/z calc'd for C₁₅H₂₅N₂O₂ [M+Na]⁺ 265.2. found 265.2.

### Preparation of Intermediate 1.3, methyl 2-(2-(4-cyanophenyl)cyclopropyl)acetate

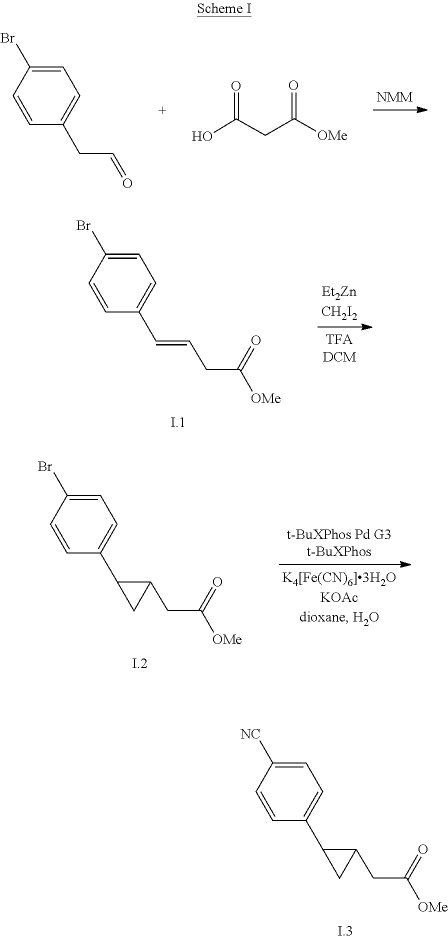 [SMILES: O=CCc1ccc(Br)cc1 ; "+" ; COC(=O)CC(=O)O ; COC(=O)C/C=C/c1ccc(Br)cc1 ; COC(=O)CC1CC1c1ccc(Br)cc1 ; COC(=O)CC1CC1c1ccc(C#N)cc1]

### Step 1—Synthesis of Intermediate 1.1, methyl (E)-4-(4-bromophenyl)but-3-enoate

2-(4-bromophenyl)acetaldehyde (1.99 g, 10.0 mmol) was added to a stirred solution of NMM (1.21 mL, 11.0 mmol) and 3-methoxy-3-oxopropanoic acid (1.15 mL, 11.0 mmol). The reaction was stirred vigorously at 80° C. for 4 h. The reaction was cooled to room temperature, diluted with water (10 mL) and extracted with DCM (3×10 mL). The combined organic layers were dried over anhydrous MgSO₄, filtered, and concentrated under reduced pressure. The resulting crude residue was purified by silica gel chromatography (gradient elution: 0-50% EtOAc/hexanes) to provide methyl (E)-4-(4-bromophenyl)but-3-enoate. MS (ESI) m/z calc'd for C₁₁H₁₂BrO₂ [M+H]⁺ 255.0. found 255.0, 257.0.

### Step 2—Synthesis of Intermediate 1.2, methyl 2-(2-(4-bromophenyl)cyclopropyl)acetate

CH₂I₂ (1.65 mL, 20.4 mmol) was added to a stirred solution of Et₂Zn (1 M in hexanes, 10.2 mL, 10.2 mmol) in DCM (15.0 mL) at −78° C. After 15 minutes, TFA (0.79 mL, 10.2 mmol) was added dropwise over 2 minutes at −78° C. After 15 minutes, a solution of methyl (E)-4-(4-bromophenyl)but-3-enoate (869 mg, 3.41 mmol) in DCM (2.0 mL) was added dropwise over 2 minutes at −78° C. The reaction was allowed to warm to room temperature. After 16 h, the reaction was quenched with sat. aq. NaHCO₃ water (5 mL) and filtered through Celite©. The resulting filtrate was diluted with water (10 mL) and extracted with DCM (3×25 mL). The combined organic layers were dried over anhydrous MgSO₄, filtered, and concentrated under reduced pressure. The resulting crude residue was purified by silica gel chromatography (gradient elution: 0-50% EtOAc/hexanes) to provide methyl 2-(2-(4-bromophenyl)cyclopropyl) acetate. MS (ESI) m/z calc'd for C₁₂H₁₄BrO₂ [M+H]⁺ 269.0. found 269.1, 271.1.

### Step 3—Synthesis of Intermediate 1.3, methyl 2-(2-(4-cyanophenyl)cyclopropyl)acetate

K₄[Fe(CN)₆]·3H₂O (466 mg, 1.27 mmol), KOAc (31 mg, 0.316 mmol), t-BuXPhos (10.7 mg, 0.025 mmol), and t-BuXPhos Pd G3 (20.1 mg, 0.025 mmol) were combined. The reaction vessel was sealed and flushed with nitrogen for 5 min, evacuated for 1 min, and backfilled with nitrogen for 1 min. A solution of methyl 2-(2-(4-bromophenyl)cyclopropyl)acetate (681 mg, 2.53 mmol) in dioxane (6.3 mL) and water (6.3 mL) were then added, and the reaction was degassed by sparging with nitrogen for 15 min, then backfilled with nitrogen for 1 min. The reaction was heated to 100° C. for 1 h in a microwave reactor. The reaction was cooled to room temperature, diluted with brine (10 mL) and extracted with EtOAc (3×10 mL). The combined organic layers were dried over anhydrous MgSO₄, filtered, and concentrated under reduced pressure. The resulting crude residue was purified by silica gel chromatography (gradient elution: 0-50% EtOAc/hexanes) to provide methyl 2-(2-(4-cyanophenyl)cyclopropyl)acetate. MS (ESI) m/z calc'd for C₁₃H₁₄NO₂ [M+H]⁺ 216.1. found 216.2.

### Preparation of Intermediate J.1, 2-(2-(4-cyanophenyl)cyclopropyl)acetohydrazide

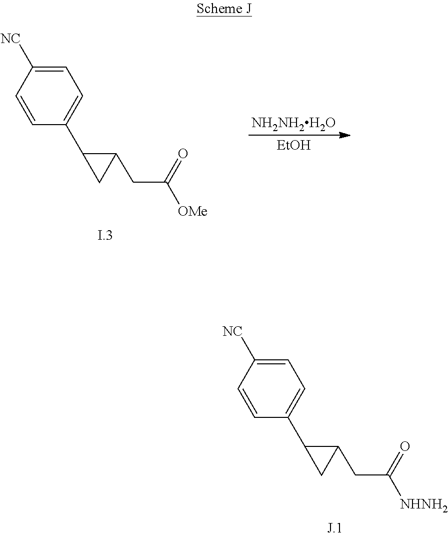 [SMILES: "EtOH" ; COC(=O)CC1CC1c1ccc(C#N)cc1 ; "NH2NH2·H2O" ; N#Cc1ccc(C2CC2CC(=O)NN)cc1]

Hydrazine monohydrate (1.08 mL, 22.3 mmol) was added to a stirred solution of methyl 2-(2-(4-cyanophenyl)cyclopropyl)acetate (408 mg, 2.23 mmol) in EtOH (4.46 mL). The reaction was stirred vigorously at 90° C. for 2 h. The reaction was cooled to room temperature and concentrated under reduced pressure to remove the volatiles. The concentrated mixture was heated to 120° C. and thoroughly dried under reduced pressure to provide 2-(2-(4-cyanophenyl)cyclopropyl)acetohydrazide, which was used in subsequent steps without further purification. MS (ESI) m/z calc'd for C₁₂H₁₄N₃O [M+H]⁺ 216.1. found 216.1.

The following intermediate compounds in Table 3 were prepared according to Scheme J starting from the appropriate ester intermediates.

TABLE 3
Intermediate Compounds Prepared According to Scheme J

| | | Observed |
|---|---|---|
| En- | Structure | m/z |
| try | Name | [M + H]⁺ |
| J.2 | 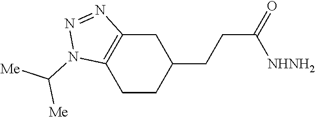 [SMILES: CC(C)n1nnc2c1CCC(CCC(=O)NN)C2] | 252.2 |
| | 3-(1-isopropyl-4,5,6,7-tetrahydro-1H-benzo[d][1,2,3] | |
| | triazol-5-yl)propanehydrazide | |
| J.3 | 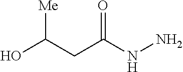 | 119.1 |
| | 3-hydroxybutanehydrazide | |
| J.4 | 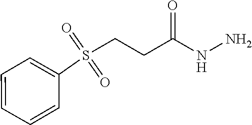 [SMILES: NNC(=O)CCS(=O)(=O)c1ccccc1] | 228.1 |
| | 3-(phenylsulfonyl)propanehydrazide | |
| J.5 | 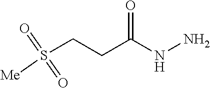 | 189.0 [M + Na]⁺ |
| | 3-(methylsulfonyl)propanehydrazide | |
| J.6 | 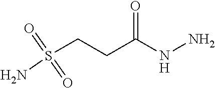 | 168.0 |
| | 3-hydrazinyl-3-oxopropane-1-sulfonamide | |
| J.7 | 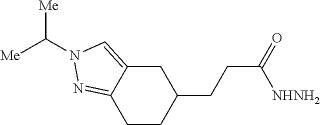 [SMILES: CC(C)n1cc2c(n1)CCC(CCC(=O)NN)C2] | 251.2 |
| | 3-(2-isopropyl-4,5,6,7-tetrahydro-2H-indazol-5- | |
| | yl)propanehydrazide | |
| J.8 | 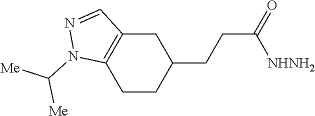 [SMILES: CC(C)n1ncc2c1CCC(CCC(=O)NN)C2] | 251.2 |
| | 3-(1-isopropyl-4,5,6,7-tetrahydro-1H-indazol-5- | |
| | yl)propanehydrazide | |
| J.9 | 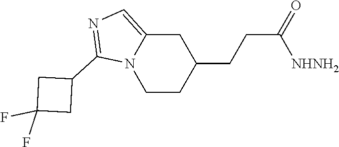 [SMILES: NNC(=O)CCC1CCn2c(cnc2C2CC(F)(F)C2)C1] | 299.2 |
| | 3-(3-(3,3-difluorocyclobutyl)-5,6,7,8-tetrahydroimidazo | |
| | [1,5-a]pyridin-7-yl)propanehydrazide | |
| J.10 | 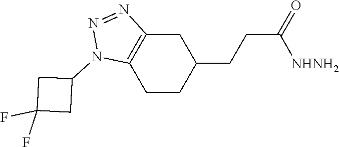 [SMILES: NNC(=O)CCC1CCc2c(nnn2C2CC(F)(F)C2)C1] | 300.3 |
| | 3-(1-(3,3-difluorocyclobutyl)-4,5,6,7-tetrahydro-1H- | |
| | benzo[d][1,2,3]triazol-5-yl)propanehydrazide | |
| J.11 | 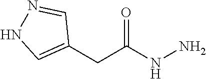 | 141.2 |
| | 2-(1H-pyrazol-4-yl)acetohydrazide | |
| J.12 | 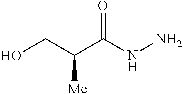 | 119 |
| | (S)-3-hydroxy-2-methylpropanehydrazide | |
| J.13 | 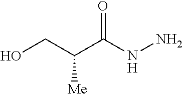 | 119 |
| | (R)-3-hydroxy-2-methylpropanehydrazide | |
| J.14 | 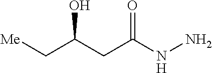 | 133 |
| | (R)-3-hydroxypentanehydrazide | |
| J.15 | 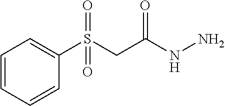 [SMILES: NNC(=O)CS(=O)(=O)c1ccccc1] | 215 |
| | 2-(phenylsulfonyl)acetohydrazide | |

### Preparation of Intermediate K.5, N-(2,4-dimethoxybenzyl)-4-hydrazinyl-8-methoxyquinazolin-2-amine

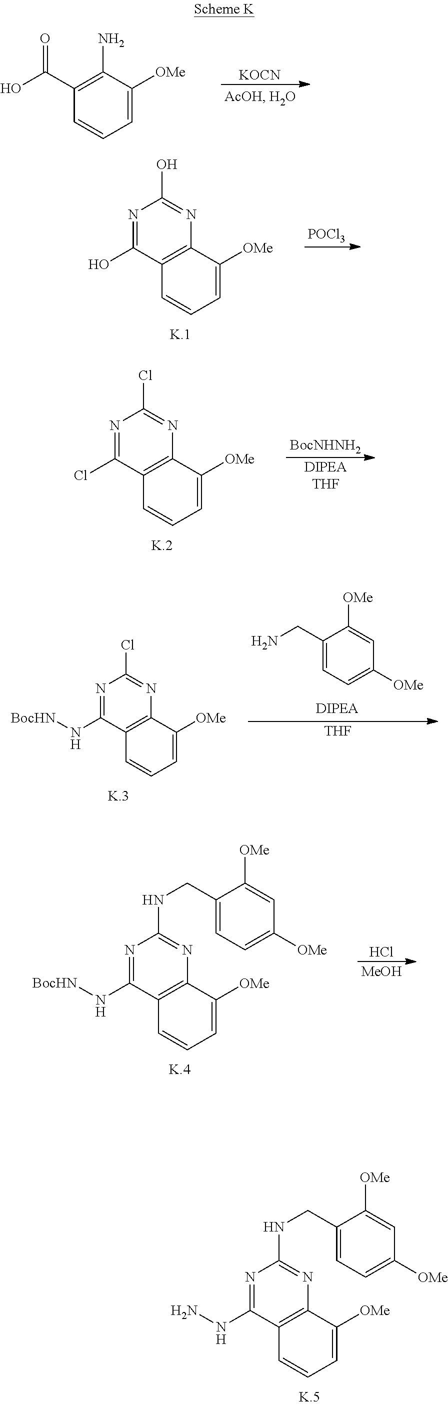

### Step 1—Synthesis of Intermediate K.1, of 8-methoxyquinazoline-2,4-diol

A solution of KOCN (175 g, 2.15 mol) in water (700 mL) was added to a suspension of 2-amino-3-methoxybenzoic acid (150 g, 0.9 mol) in water (3000 mL) and AcOH (55 mL, 0.96 mol) at 55° C. The reaction was stirred vigorously at 55° C. for 16 h. The reaction was cooled to room temperature and stirred for an additional 3 h. The reaction was cooled to 0° C. and acidified to pH 5-6 with conc. HCl (3.5 L). The resulting solids were filtered, washed with water, and dried under reduced pressure to provide 8-methoxyquinazoline-2,4-diol, which was used in the subsequent step without further purification.

### Step 2—Synthesis of Intermediate K.2, 2,4-dichloro-8-methoxyquinazoline

POCl₃ (315 mL, 3.38 mol) was added to 8-methoxyquinazoline-2,4-diol (50 g, 0.26 mol). The reaction was heated at 105° C. for 16 h. The reaction was cooled to room temperature and concentrated under reduced pressure. The reaction mixture was diluted with toluene and concentrated under reduced pressure. This process was repeated 3 times. The resulting residue was diluted with EtOAc and washed with sat. aq. NaHCO₃. The combined organic layers were washed with brine, dried over anhydrous MgSO₄, filtered, and concentrated under reduced pressure to provide 2,4-dichloro-8-methoxyquinazoline, which was used in the subsequent step without further purification.

### Step 3—Synthesis of Intermediate K.3, tert-butyl 2-(2-chloro-8-methoxyquinazolin-4-yl)hydrazine-1-carboxylate

DIPEA (9.2 mL, 0.052 mol) and tert-butyl hydrazinecarboxylate (5.8 g, 0.044 mol) were added to a stirred solution of 2,4-dichloro-8-methoxyquinazoline (10 g, 0.044 mol) in THE (200 mL). The reaction was stirred vigorously at 65° C. for 16 h. The reaction was cooled to room temperature and concentrated under reduced pressure to provide tert-butyl 2-(2-chloro-8-methoxyquinazolin-4-yl)hydrazine-1-carboxylate, which was used in the subsequent step without further purification.

### Step 4—Synthesis of Intermediate K.4, tert-butyl 2-(2-((2,4-dimethoxybenzyl)amino)-8-methoxyquinazolin-4-yl)hydrazine-1-carboxylate

DIPEA (19 mL, 0.34 mol) and (2,4-dimethoxyphenyl)methanamine (9.46 g, 0.057 mol) were added to a suspension of tert-butyl 2-(2-chloro-8-methoxyquinazolin-4-yl)hydrazine-1-carboxylate in dioxane (100 mL). The reaction was heated to 100° C. for 16 h. The reaction was cooled to room temperature, concentrated under reduced pressure, and diluted with water. The resulting mixture stirred vigorously at room temperature for 30 min. The reaction was filtered, washed with dioxane and hexanes, and dried under reduced pressure to provide tert-butyl 2-(2-((2,4-dimethoxybenzyl)amino)-8-methoxyquinazolin-4-yl)hydrazine-1-carboxylate, which was used in the subsequent step without further purification.

### Step 5—Synthesis of Intermediate K.5, N-(2,4-dimethoxybenzyl)-4-hydrazinyl-8-methoxyquinazolin-2-amine

HCl (2.5 M in MeOH, 1 L, 2.5 mol) was added to tert-butyl 2-(2-((2,4-dimethoxybenzyl)amino)-8-methoxyquinazolin-4-yl)hydrazine-1-carboxylate (60 g, 0.132 mol). The reaction was stirred vigorously at room temperature for 16 h. The reaction diluted with triethanolamine (1.5 L), and the resulting solids was filtered, washed with triethanolamine, and dried under reduced pressure to provide N-(2,4-dimethoxybenzyl)-4-hydrazinyl-8-methoxyquinazolin-2-amine, which was used in subsequent steps without further purification. ¹H-NMR (300 MHz, DMSO-d₆): δ 11.43 (s, 1H), 8.27 (s, 1H), 7.80-7.78 (d, 1H), 7.39-7.25 (m, 3H), 6.61 (d, 1H), 6.50-6.47 (d, 1H), 4.69-4.67 (d, 2H), 3.96 (s, 3H), 3.86 (s, 3H), 3.76 (s, 3H).

### Preparation of Intermediate L.3, (5-((2,4-dimethoxybenzyl)amino)-7-methoxy-[1,2,4]triazolo[1,5-c]quinazolin-2-yl)methanol

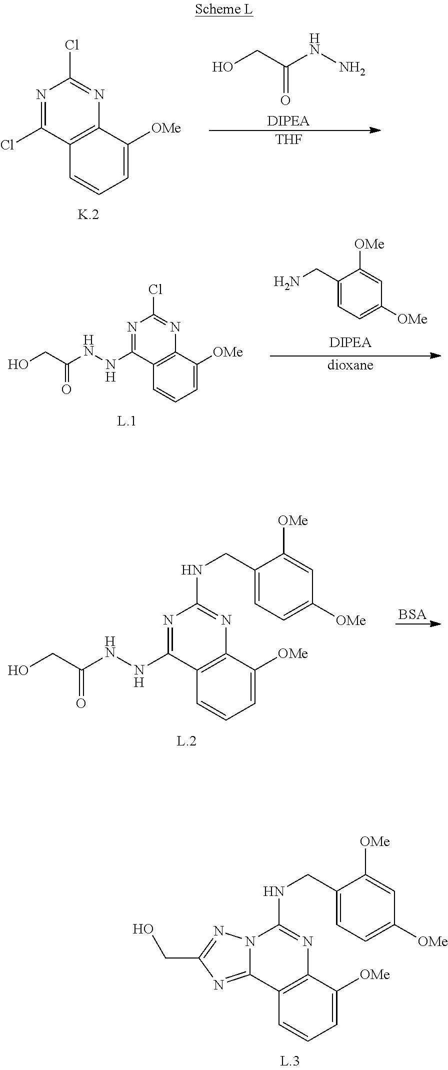

Step 1—Synthesis of Intermediate L.1, of N′-(2-chloro-8-methoxyquinazolin-4-yl)-2-hydroxyacetohydrazide.

DIPEA (13.9 mL, 80 mmol) and 2-hydroxyacetohydrazide (5.98 g, 66.4 mmol) were added to a stirred solution of 2,4-dichloro-8-methoxyquinazoline (15.2 g, 66.4 mmol) in THE (664 mL). The reaction was stirred vigorously at 65° C. for 16 h. The reaction was cooled to room temperature and concentrated under reduced pressure. The concentrated residue was diluted in DCM and stirred for 30 min. The resulting solid was filtered, washed with DCM, and dried under reduced pressure to provide N′-(2-chloro-8-methoxyquinazolin-4-yl)-2-hydroxyacetohydrazide, which was used in the subsequent step without further purification. MS (ESI) m/z calc'd for C₁₁H₁₂ClN₄O₃ [M+H]⁺ 283. found 283.

### Step 2—Synthesis of Intermediate L.2, N′-(2-((2,4-dimethoxybenzyl)amino)-8-methoxyquinazolin-4-yl)-2-hydroxyacetohydrazide

DIPEA (22.71 ml, 130 mmol) and (2,4-dimethoxyphenyl)methanamine (10.2 ml, 67.6 mmol) were added to a suspension of N′-(2-chloro-8-methoxyquinazolin-4-yl)-2-hydroxyacetohydrazide (14.7 g, 52.0 mmol) in dioxane (520 mL). The reaction was heated at 100° C. for 16 h. The reaction was cooled to room temperature, filtered, and washed with dioxane and hexanes to provide N′-(2-((2,4-dimethoxybenzyl)amino)-8-methoxyquinazolin-4-yl)-2-hydroxyacetohydrazide, which was used in the subsequent step without further purification. MS (ESI) m/z calc'd for C₂OH₂₄N₅O₅ [M+H]⁺ 414. found 414.

### Step 3—Synthesis of Intermediate L.3, (5-((2,4-dimethoxybenzyl)amino)-7-methoxy-[1,2,4]triazolo[1,5-c]quinazolin-2-yl)methanol

BSA (144 mL, 589 mmol) was added to N′-(2-((2,4-dimethoxybenzyl)amino)-8-methoxyquinazolin-4-yl)-2-hydroxyacetohydrazide (20.3 g, 49.1 mmol). The reaction was stirred vigorously at 130° C. for 16 h. The reaction was cooled to room temperature and concentrated under reduced pressure. The reaction mixture was diluted with MeOH (170 mL) and HCl (37% aq., 2.5 mL). The reaction was stirred vigorously for 10 min. The resulting solid was filtered, rinsed with water and DCM (2×50 mL), and dried under reduced pressure to provide (5-((2,4-dimethoxybenzyl)amino)-7-methoxy-[1,2,4]triazolo[1,5-c]quinazolin-2-yl)methanol, which was used in subsequent steps without further purification. MS (ESI) m/z calc'd for C₂OH₂₂N₅O₄ [M+H]⁺ 396. found 396.

The following compound in Table 4 was prepared according to Scheme L starting from K.2 and the commercially available hydrazide.

TABLE 4
Intermediate Compounds Prepared According to Scheme L

| En- | Structure | Observed m/z |
|---|---|---|
| try | Name | [M + H]⁺ |
| L.4 | 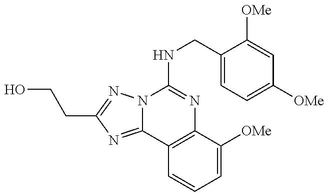 [SMILES: COc1ccc(CNc2nc3c(OC)cccc3c3nc(CCO)nn23)c(OC)c1] | 410.2 |
| | 2-(5-((2,4-dimethoxybenzyl)amino)-7-methoxy- | |
| | [1,2,4]triazolo[1,5-c]quinazolin-2-yl)ethan-1-ol | |

### Preparation of Intermediate M.5, (5-amino-7-methoxy-[1,2,4]triazolo[1,5-c]quinazolin-2-yl)methanol

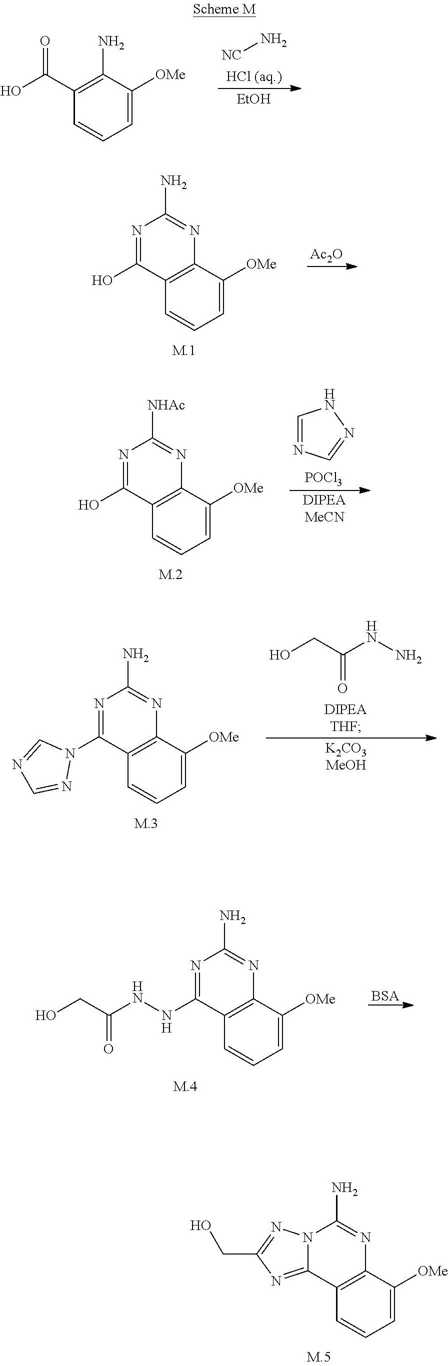

### Step 1—Synthesis of Intermediate M.1, of 2-amino-8-methoxyquinazolin-4-ol

Cyanamide (18.9 g, 449 mmol) and HCl (37% aq., 12 mL, 299 mmol) were added to a suspension of 2-amino-3,5-difluorobenzoic acid (50.0 g, 299 mmol) in EtOH (400 mL). The reaction was stirred vigorously at 80° C. for 16 h. The reaction was cooled to room temperature and the resulting solids were filtered and washed with cold EtOH to provide 2-amino-8-methoxyquinazolin-4-ol, which was used in the subsequent step without further purification.

### Step 2—Synthesis of Intermediate M.2, N-(4-hydroxy-8-methoxyquinazolin-2-yl)acetamide

Ac₂O (60 mL, 52.3 mmol) was added to 2-amino-8-methoxyquinazolin-4-ol (10 g, 52.3 mmol). The reaction was heated at 130° C. for 40 min to provide N-(4-hydroxy-8-methoxyquinazolin-2-yl)acetamide, which was used in the subsequent step without further purification.

### Step 3—Synthesis of Intermediate M.3, N-(8-methoxy-4-(1H-1,2,4-triazol-1-yl)quinazolin-2-yl)acetamide

POCl₃ (8.22 mL, 90 mmol) was added dropwise to a stirred solution of N-(4-hydroxy-8-methoxyquinazolin-2-yl)acetamide (7.0 g, 30.0 mmol), 1,2,4-triazole (20.7 g, 300 mmol), and DIPEA (15.3 mL, 90 mmol) in MeCN (300 mL). The reaction was stirred vigorously at room temperature for 16 h. The resulting solids were filtered and washed with EtOH and diethyl ether to provide N-(8-methoxy-4-(1H-1,2,4-triazol-1-yl)quinazolin-2-yl)acetamide, which was used in the subsequent step without further purification.

### Step 4—Synthesis of Intermediate M.4, N′-(2-amino-8-methoxyquinazolin-4-yl)-2-hydroxyacetohydrazide

2-hydroxyacetohydrazide (1.39 g, 15.5 mmol) and DIPEA (1.71 g, 17.9 mmol) were added to a suspension of N-(8-methoxy-4-(1H-1,2,4-triazol-1-yl)quinazolin-2-yl)acetamide (4.0 g, 14.1 mmol) in THE (300 mL). The reaction was stirred vigorously at 60° C. for 48 h. The reaction was cooled to room temperature and concentrated under reduced pressure. The concentrated residue was dissolved in MeOH (200 mL) and water (100 mL). K₂CO₃ was added, and the reaction was stirred vigorously at 65° C. for 2 h. The reaction was cooled to room temperature and concentrated under reduced pressure. The resulting solids were filtered, washed with cold water and DCM/hexanes, and dried under reduced pressure to provide N′-(2-amino-8-methoxyquinazolin-4-yl)-2-hydroxyacetohydrazide, which was used in the subsequent step without further purification. MS (ESI) m/z calc'd for C₁₁H₁₄N₅O₃ [M+H]⁺ 264. found 264.

### Step 5—Synthesis of Intermediate M.5, (5-amino-7-methoxy-[1,2,4]triazolo[1,5-c]quinazolin-2-yl)methanol

BSA (100 mL) was added to N′-(2-amino-8-methoxyquinazolin-4-yl)-2-hydroxyacetohydrazide (3.5 g, 13.3 mmol). The reaction was heated to 120° C. for 3 h. The reaction was cooled to room temperature, concentrated under reduced pressure, and diluted with MeOH. The resulting solids were suspended in MeOH, cooled, filtered, washed with MeOH, and dried under reduced pressure to provide (5-amino-7-methoxy-[1,2,4]triazolo[1,5-c]quinazolin-2-yl)methanol, which was used in subsequent steps without further purification. MS (ESI) m/z calc'd for C₁₁H₁₂N₅O₂ [M+H]⁺ 246. found 246.

The following compounds in Table 5 were prepared according to Scheme M starting from the appropriate commercially available carboxylic acid in Step 1 or the appropriate commercially available hydrazide in Step 4.

TABLE 5
Intermediate Compounds Prepared According to Scheme M

| En- | Structure | Observed m/z |
|---|---|---|
| try | Name | [M + H]⁺ |
| M.6 | 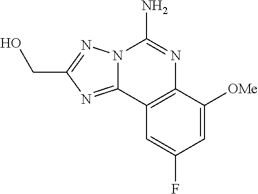 [SMILES: COc1cc(F)cc2c1nc(N)n1nc(CO)nc21] | 264.1 |
| | (5-amino-9-fluoro-7-methoxy-[1,2,4]triazolo[1,5- | |
| | c]quinazolin-2-yl)methanol | |
| M.7 | 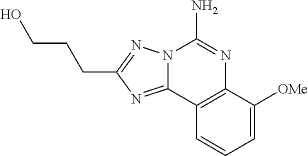 [SMILES: COc1cccc2c1nc(N)n1nc(CCCO)nc21] | 274.2 |
| | 3-(5-amino-7-methoxy-[1,2,4]triazolo[1,5-c]quinazolin- | |
| | 2-yl)propan-1-ol | |

### Preparation of Intermediate N.1, 2-(chloromethyl)-7-methoxy-[1,2,4]triazolo[1,5-c]quinazolin-5-amine

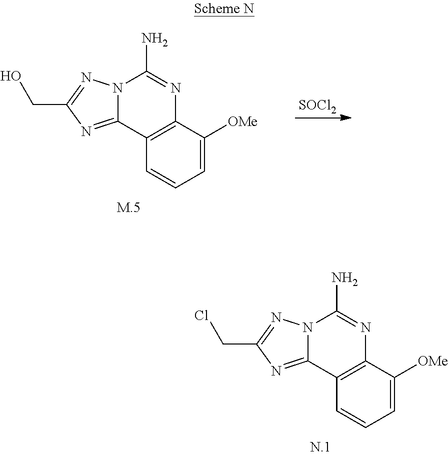 [SMILES: COc1cccc2c1nc(N)n1nc(CO)nc21 ; "SOCl2" ; COc1cccc2c1nc(N)n1nc(CCl)nc21]

SOCl₂ (10 mL) was added to (5-amino-7-methoxy-[1,2,4]triazolo[1,5-c]quinazolin-2-yl)methanol (2.8 g, 11.4 mmol). The reaction was stirred vigorously at 65° C. for 45 min. The reaction was cooled to room temperature and concentrated under reduced pressure. The resulting residue was suspended in DCM. The resulting solids were filtered and dried under reduced pressure to provide 2-(chloromethyl)-7-methoxy-[1,2,4]triazolo[1,5-c]quinazolin-5-amine, which was used in subsequent steps without further purification. MS (ESI) m/z calc'd for C₁₁H₁₁ClN₅O [M+H]⁺ 264. found 264.

The following compounds in Table 6 were prepared according to Scheme N starting from the appropriate alcohol intermediate.

TABLE 6
Intermediate Compounds Prepared According to Scheme N

| | Structure | Observed m/z |
|---|---|---|
| Entry | Name | [M + H⁺ |
| N.2 | 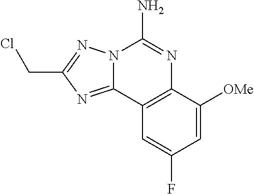 [SMILES: COc1cc(F)cc2c1nc(N)n1nc(CCl)nc21] | 282.1 |
| | 2-(chloromethyl)-9-fluoro-7-methoxy-[1,2,4]triazolo | |
| | [1,5-c]quinazolin-5-amine | |
| N.3 | 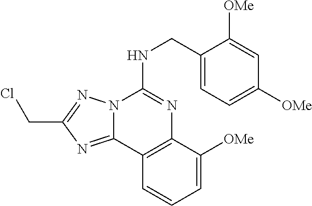 [SMILES: COc1ccc(CNc2nc3c(OC)cccc3c3nc(CCl)nn23)c(OC)c1] | 414.2 |
| | 2-(chloromethyl)-N-(2,4-dimethoxybenzyl)-7-methoxy- | |
| | [1,2,4]triazolo[1,5-c]quinazolin-5-amine | |

### Preparation of Intermediate O.3, 2-((((2,4-dimethoxybenzyl)imino)methylene)amino)-5-fluoro-4-methoxybenzonitrile

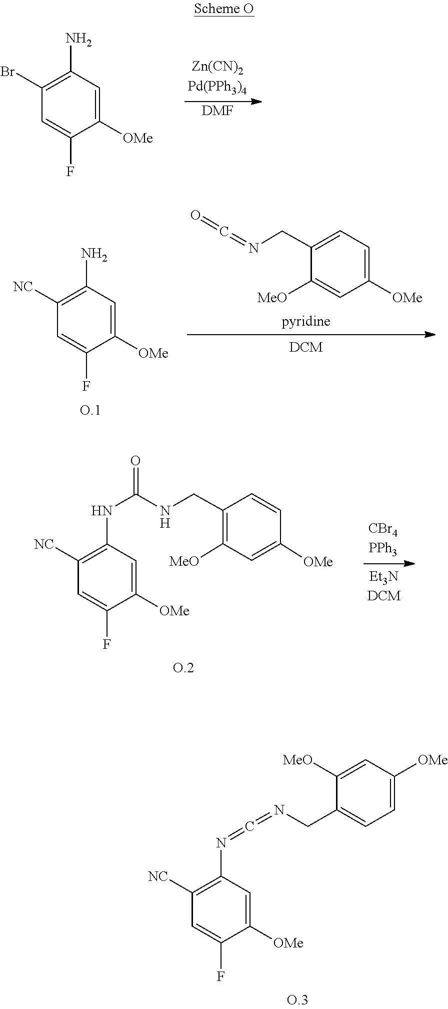

### Step 1—Synthesis of Intermediate O.1, 2-amino-5-fluoro-4-methoxybenzonitrile

Zn(CN)₂ (327 g, 2.78 mol) and Pd(PPh₃)₄ (90.0 g, 0.0778 mol) were added to a stirred solution of 2-bromo-4-fluoro-5-methoxyaniline (300 g, 1.36 mol) in DMF (2.1 L). The mixture was degassed under vacuum and purged with nitrogen. The reaction was stirred vigorously at 130° C. for 1 h. The reaction was cooled to room temperature, diluted with ice water (4 L) and extracted with EtOAc (3 L, 2 L, 1 L). The combined organic layers were washed with brine (2 L, 1.5 L), dried over anhydrous Na₂SO₄, filtered, and concentrated under reduced pressure. The resulting crude residue was purified by silica gel chromatography (gradient elution: 0-50% EtOAc/petroleum ether) to provide 2-amino-5-fluoro-4-methoxybenzonitrile. MS (ESI) m/z calc'd for C₈H₈FN₂O [M+H]⁺ 167. found 167.

### Step 2—Synthesis of Intermediate O.2, 1-(2-cyano-4-fluoro-5-methoxyphenyl)-3-(2,4-dimethoxybenzyl)urea

1-(isocyanatomethyl)-2,4-dimethoxybenzene (1425 mg, 7.380 mmol) was added to a stirred solution of 2-amino-5-fluoro-4-methoxybenzonitrile (817 mg, 4.92 mmol) and pyridine (1 mL) in DCM (6 mL). The reaction was stirred vigorously at 40° C. for 16 h. The reaction was cooled to room temperature, and the resulting solids were filtered and washed with MeOH (3×3 mL) to provide 1-(2-cyano-4-fluoro-5-methoxyphenyl)-3-(2,4-dimethoxybenzyl)urea, which was used in the subsequent step without further purification.

### Step 3—Synthesis of Intermediate O.3, 2-((((2,4-dimethoxybenzyl)imino)methylene)amino)-5-fluoro-4-methoxybenzonitrile

A solution of CBr₄ (2.14 g, 6.44 mmol) in DCM (5 mL) was added to a stirred solution of 1-(2-cyano-4-fluoro-5-methoxyphenyl)-3-(2,4-dimethoxybenzyl)urea (1.16 g, 3.22 mmol), PPh₃ (1.69 g, 6.44 mmol), and triethylamine (1.80 ml, 12.9 mmol) in DCM (25 mL) dropwise at 0° C. The reaction was stirred vigorously at 0° C. for 30 min. The reaction was concentrated, and the resulting residue was purified by silica gel chromatography (gradient elution: 0-70% EtOAc/hexanes) to provide 2-((((2,4-dimethoxybenzyl)imino)methylene)amino)-5-fluoro-4-methoxybenzonitrile. MS (ESI) m/z calc'd for C₁₈H₁₆FN₃NaO₃ [M+Na]⁺364. found 364. The following compounds in Table 7 were prepared according to Scheme O starting from the appropriate commercially available starting materials. For the preparation of O.6 and O.7, Step 1 was omitted as the appropriate nitriles were commercially available.

TABLE 7
Intermediate Compounds Prepared According to Scheme O

| | Structure | Observed m/z |
|---|---|---|
| Entry | Name | [M + Na]⁺ |
| O.4 | 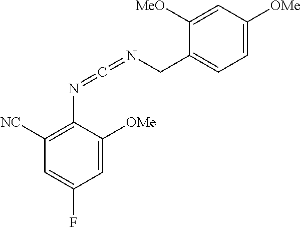 [SMILES: COc1ccc(CN=C=Nc2c(C#N)cc(F)cc2OC)c(OC)c1] | 364 |
| | 2-((((2,4-dimethoxybenzyl)imino)methylene)amino)-5- | |
| | fluoro-3-methoxybenzonitrile | |
| O.5 | 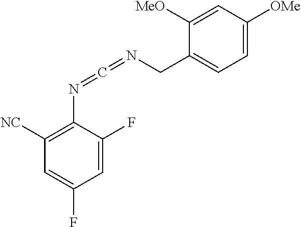 [SMILES: COc1ccc(CN=C=Nc2c(F)cc(F)cc2C#N)c(OC)c1] | 352 |
| | 2-((((2,4-dimethoxybenzyl)imino)methylene)amino)-3,5- | |
| | difluorobenzonitrile | |
| O.6 | 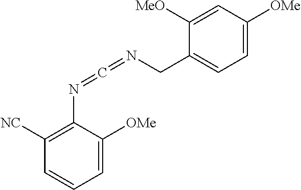 [SMILES: COc1ccc(CN=C=Nc2c(C#N)cccc2OC)c(OC)c1] | 346 |
| | 2-((((2,4-dimethoxybenzyl)imino)methylene)amino)-3- | |
| | methoxybenzonitrile | |
| O.7 | 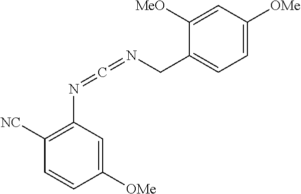 [SMILES: COc1ccc(C#N)c(N=C=NCc2ccc(OC)cc2OC)c1] | 346 |
| | 2-((((2,4-dimethoxybenzyl)imino)methylene)amino)-4- | |
| | methoxybenzonitrile | |

### Preparation of Intermediate P.1, 1-(5-((2,4-dimethoxybenzyl)amino)-9-fluoro-8-methoxy-[1,2,4]triazolo[1,5-c]quinazolin-2-yl)propan-2-ol

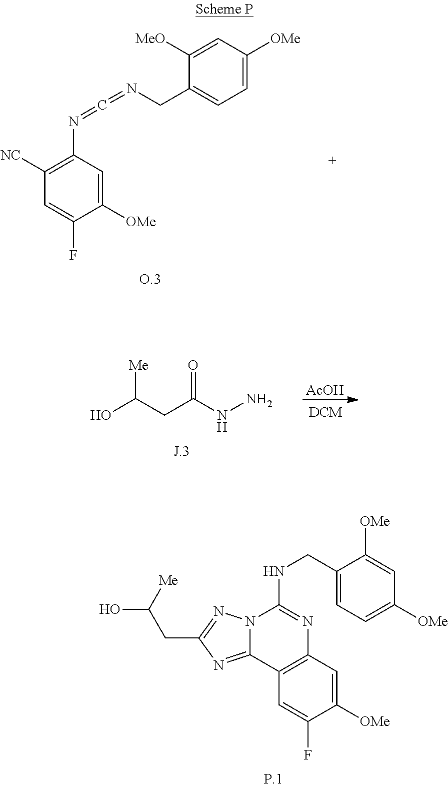 [SMILES: COc1ccc(CN=C=Nc2cc(OC)c(F)cc2C#N)c(OC)c1 ; "AcOH" ; "+" ; CC(O)CC(=O)NN ; COc1ccc(CNc2nc3cc(OC)c(F)cc3c3nc(CC(C)O)nn23)c(OC)c1]

2-((((2,4-dimethoxybenzyl)imino)methylene)amino)-5-fluoro-4-methoxybenzonitrile (650 mg, 1.904 mmol) and AcOH (60 μL, 0.952 mmol) were added to a stirred solution of 3-hydroxybutanehydrazide (270 mg, 2.285 mmol) in DCM (2 mL). The reaction was stirred vigorously at 35° C. for 16 h. The reaction was cooled to room temperature and concentrated under reduced pressure. The resulting crude residue was purified by silica gel chromatography (gradient elution: 10-50% EtOAc/petroleum ether) to 1-(5-((2,4-dimethoxybenzyl)amino)-9-fluoro-8-methoxy-[1,2,4]triazolo[1,5-c]quinazolin-2-yl)propan-2-ol. MS (ESI) m/z calc'd for C₂₂H₂₄FN₅O₄ [M+H]⁺ 442.2. found 442.3.

The following compound in Table 8 was prepared according to Scheme P starting from Intermediate O.5 and the commercially available hydrazide.

TABLE 8
Intermediate Compounds Prepared According to Scheme P

| En- | Structure | Observed m/z |
|---|---|---|
| try | Name | [M + H]⁺ |
| P.2 | 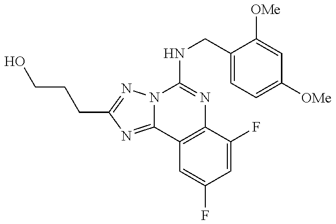 [SMILES: COc1ccc(CNc2nc3c(F)cc(F)cc3c3nc(CCCO)nn23)c(OC)c1] | 430.2 |
| | 3-(5-((2,4-dimethoxybenzyl)amino)-7,9-difluoro- | |
| | [1,2,4]triazolo[1,5-c]quinazolin-2-yl)propan-1-ol | |

### Preparation of Intermediate Q.1, 2-(5-((3,4-dimethylbenzyl)amino)-9-fluoro-8-methoxy-[1,2,4]triazolo[1,5-c]quinazolin-2-yl)ethyl 4-(trifluoromethyl)benzenesulfonate

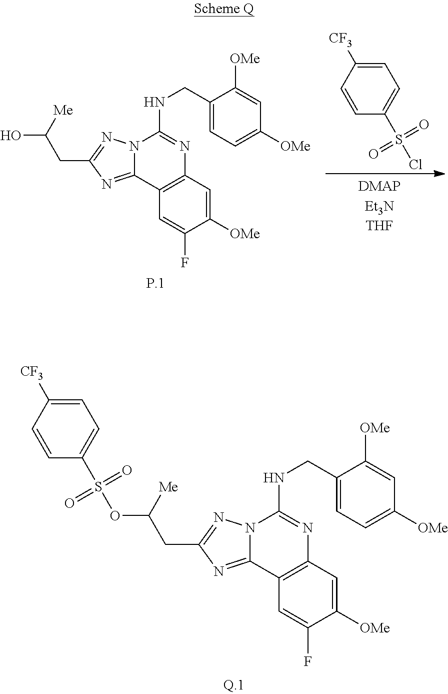 [SMILES: COc1ccc(CNc2nc3cc(OC)c(F)cc3c3nc(CC(C)O)nn23)c(OC)c1 ; O=S(=O)(Cl)c1ccc(C(F)(F)F)cc1 ; COc1ccc(CNc2nc3cc(OC)c(F)cc3c3nc(CC(C)OS(=O)(=O)c4ccc(C(F)(F)F)cc4)nn23)c(OC)c1]

Et₃N (80 mg, 0.793 mmol) was added to a stirred solution of 1-(5-((2,4-dimethoxybenzyl)amino)-9-fluoro-8-methoxy-[1,2,4]triazolo[1,5-c]quinazolin-2-yl)propan-2-ol (50 mg, 0.113 mmol), 4-(trifluoromethyl)benzene-1-sulfonyl chloride (139 mg, 0.566 mmol) and DMAP (2.8 mg, 0.023 mmol) in DCM (2 mL) at 0° C. The reaction was stirred vigorously at room temperature for 16 h. The reaction was diluted with DCM (5 mL) and washed with brine (3×5 mL). The combined organic layers were dried over anhydrous Na₂SO₄, filtered, and concentrated under reduced pressure. The resulting crude residue was purified by silica gel chromatography (gradient elution: 0-80% EtOAc/petroleum ether) to provide 2-(5-((3,4-dimethylbenzyl)amino)-9-fluoro-8-methoxy-[1,2,4]triazolo[1,5-c]quinazolin-2-yl)ethyl 4-(trifluoromethyl)benzenesulfonate. MS (ESI) m/z calc'd for C₂₉H₂₇F₄N₅O₆S [M+H]⁺ 650.2. found 650.3.

The following compounds in Table 9 were prepared according to Scheme Q starting from the appropriate alcohol intermediates.

TABLE 9
Intermediate Compounds Prepared According to Scheme Q

| | Structure | Observed m/z |
|---|---|---|
| Entry | Name | [M + H]⁺ |
| R.2 | 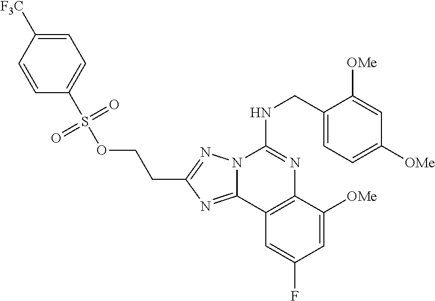 | 636.2 |
| | 2-(5-((2,4-dimethoxybenzyl)amino)-9-fluoro-7-methoxy- | |
| | [1,2,4]triazolo[1,5-c]quinazolin-2-yl)ethyl 4- | |
| | (trifluoromethyl)benzenesulfonate | |
| R.3 | 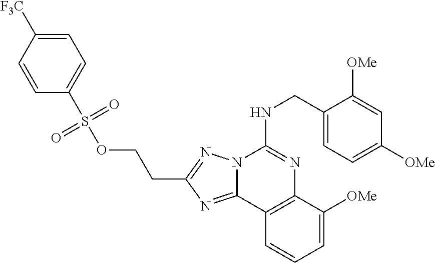 | 618.1 |
| | 2-(5-((2,4-dimethoxybenzyl)amino)-7-methoxy- | |
| | [1,2,4]triazolo[1,5-c]quinazolin-2-yl)ethyl 4- | |
| | (trifluoromethyl)benzenesulfonate | |
| R.4 | 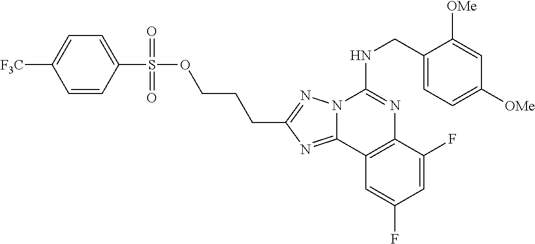 | 638.1 |
| | 3-(5-((2,4-dimethoxybenzyl)amino)-7,9-difluoro- | |
| | [1,2,4]triazolo[1,5-c]quinazolin-2-yl)propyl 4- | |
| | (trifluoromethyl)benzenesulfonate | |

### Preparation of Intermediate R.3, N-(2,4-dimethoxybenzyl)-9-fluoro-7-methoxy-2-vinyl-[1,2,4]triazolo[1,5-c]quinazolin-5-amine

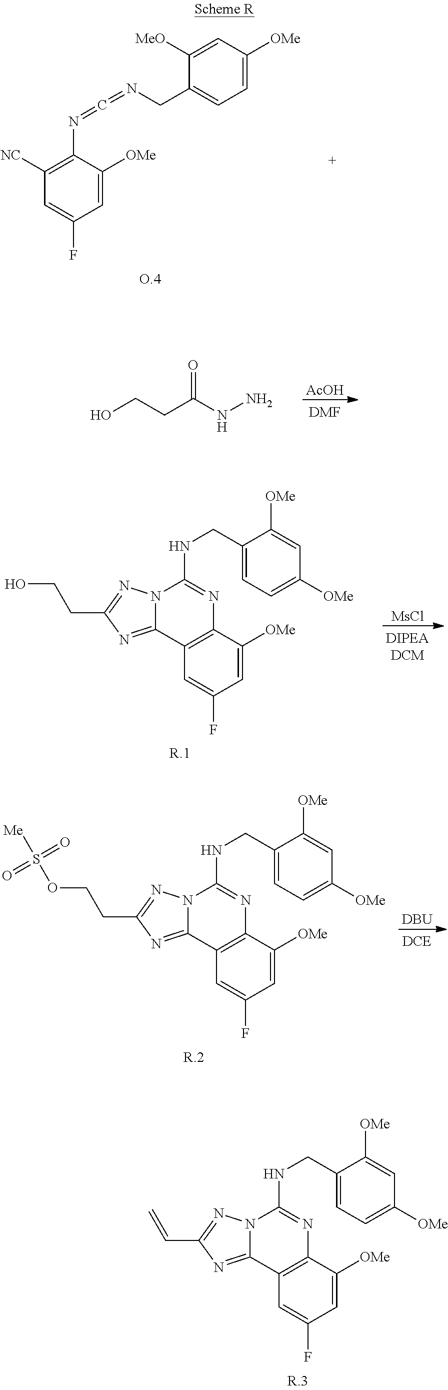

### Step 1—Synthesis of Intermediate R.1, 2-(5-((2,4-dimethoxybenzyl)amino)-9-fluoro-8-methoxy-[1,2,4]triazolo[1,5-c]quinazolin-2-yl)ethanol

2-((((2,4-dimethoxybenzyl)imino)methylene)amino)-5-fluoro-3-methoxybenzonitrile (3.20 g, 9.37 mmol) and AcOH (0.322 mL, 5.62 mmol) were added to a stirred solution of 3-hydroxypropanehydrazide (1.03 g, 9.84 mmol) in dioxane (37.5 mL). The reaction was stirred vigorously at 45° C. for 24 h. The reaction was cooled to room temperature and concentrated under reduced pressure to provide 2-(5-((2,4-dimethoxybenzyl)amino)-9-fluoro-7-methoxy-[1,2,4]triazolo[1,5-c]quinazolin-2-yl)ethanol, which was used in the subsequent step without further purification. MS (ESI) m/z calc'd for C₂₁H₂₂FN₅O₄ [M+H]⁺ 428.2. found 428.2.

### Step 2—Synthesis of Intermediate R.2, 2-(5-((2,4-dimethoxybenzyl)amino)-9-fluoro-7-methoxy-[1,2,4]triazolo[1,5-c]quinazolin-2-yl)ethyl methanesulfonate

DIPEA (1.12 mL, 6.43 mmol) and MsCl (0.220 mL, 2.83 mmol) were added to a suspension of 2-(5-((2,4-dimethoxybenzyl)amino)-9-fluoro-7-methoxy-[1,2,4]triazolo[1,5-c]quinazolin-2-yl)ethanol (1.1 g, 2.57 mmol) in DCM (25.7 mL) at 0° C. The reaction was stirred vigorously at room temperature for 3 h. The reaction was diluted with water and extracted with DCM. The combined organic layers were dried over anhydrous Na₂SO₄, filtered, and concentrated under reduced pressure to provide 2-(5-((2,4-dimethoxybenzyl)amino)-9-fluoro-7-methoxy-[1,2,4]triazolo[1,5-c]quinazolin-2-yl)ethyl methanesulfonate, which was used in the subsequent step without further purification. MS (ESI) m/z calc'd for C₂₂H₂₅FN₅O₆S [M+H]⁺ 506.2. found 506.1.

### Step 3—Synthesis of Intermediate R.3, N-(2,4-dimethoxybenzyl)-9-fluoro-7-methoxy-2-vinyl-[1,2,4]triazolo[1,5-c]quinazolin-5-amine

DBU (0.914 mL, 6.07 mmol) was added to a stirred solution of 2-(5-((2,4-dimethoxybenzyl)amino)-9-fluoro-7-methoxy-[1,2,4]triazolo[1,5-c]quinazolin-2-yl)ethyl methanesulfonate (1.46 g, 2.89 mmol) in DCE (3.85 mL). The reaction was stirred vigorously at 60° C. for 3 h. The reaction was cooled to room temperature, diluted with 1 M aq. citric acid and extracted with DCM. The combined organic layers were concentrated under reduced pressure. The resulting crude residue was purified by silica gel chromatography (gradient elution: 0-50% EtOAc/hexanes) to provide N-(2,4-dimethoxybenzyl)-9-fluoro-7-methoxy-2-vinyl-[1,2,4]triazolo[1,5-c]quinazolin-5-amine. MS (ESI) m/z calc'd for C₂₁H₂₁FN₅O₃ [M+H]⁺ 410.2. found 410.3.

### Preparation of Intermediate S.3, tert-butyl (cyclopropyl(2-(5-((2,4-dimethoxybenzyl)amino)-7-methoxy-[1,2,4]triazolo[1,5-c]quinazolin-2-yl)ethyl)(oxo)-l6-sulfanylidene)carbamate

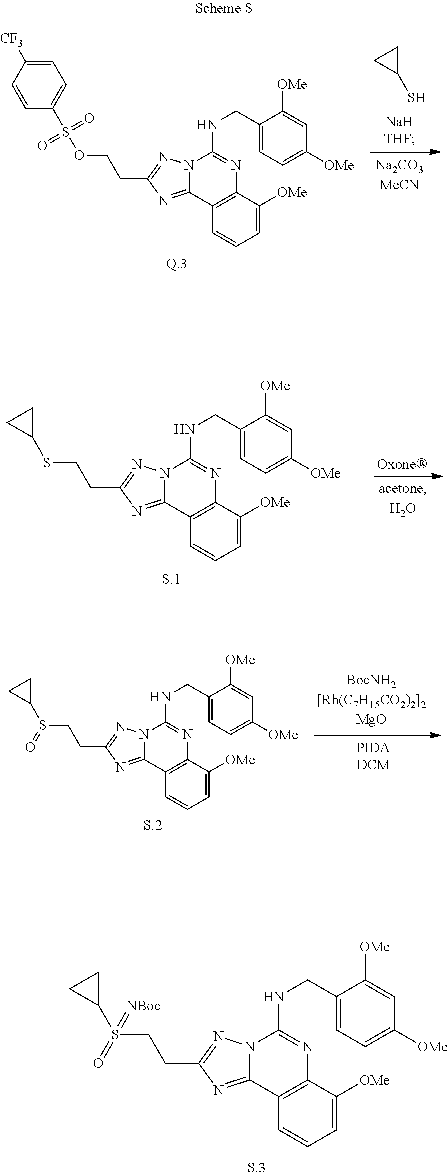

### Step 1—Synthesis of Intermediate S.1, 2-(2-(cyclopropylthio)ethyl)-N-(2,4-dimethoxybenzyl)-7-methoxy-[1,2,4]triazolo[1,5-c]quinazolin-5-amine

NaH (60%, 65 mg, 1.63 mmol) was added to a stirred solution of cyclopropanethiol (120 mg, 1.619 mmol) in THF (5 mL) at 0° C. The reaction was stirred vigorously at 0° C. for 2 h. The reaction was warmed to room temperature and concentrated under reduced pressure. The concentrated residue was re-dissolved in MeCN (5 mL), and 2-(5-((2,4-dimethoxybenzyl)amino)-7-methoxy-[1,2,4]triazolo[1,5-c]quinazolin-2-yl)ethyl 4-(trifluoromethyl)benzenesulfonate (200 mg, 0.324 mmol) and Na₂CO₃ (103 mg, 0.972 mmol) were added. The reaction was stirred vigorously at 65° C. for 16 h. The reaction was cooled to room temperature, diluted with brine (20 mL), and extracted with EtOAc (20 mL). The combined organic layers were dried over anhydrous Na₂SO₄, filtered, and concentrated under reduced pressure. The resulting crude residue was purified by silica gel chromatography (gradient elution: 0-75% EtOAc/petroleum ether) to provide 2-(2-(cyclopropylthio)ethyl)-N-(2,4-dimethoxybenzyl)-7-methoxy-[1,2,4]triazolo[1,5-c]quinazolin-5-amine. MS (ESI) m/z calc'd for C₂₄H₂₇N₅O₃S [M+H]⁺ 466.2. found 466.2.

### Step 2—Synthesis of Intermediate S.2, 2-(2-(cyclopropylsulfinyl)ethyl)-N-(2,4-dimethoxybenzyl)-7-methoxy-[1,2,4]triazolo[1,5-c]quinazolin-5-amine

A solution of Oxone© (24 mg, 0.039 mmol) in water (2 mL) was added to a stirred solution of 2-(2-(cyclopropylthio)ethyl)-N-(2,4-dimethoxybenzyl)-7-methoxy-[1,2,4]triazolo[1,5-c]quinazolin-5-amine (50 mg, 0.107 mmol) in acetone (5 mL) at 0° C. The reaction was stirred vigorously at 0° C. for 40 min. The reaction was warmed to room temperature, quenched with 5% aq. NaHSO₃, and extracted with EtOAc (2×20 mL). The combined organic layers were washed with sat. NaHCO₃ (20 mL), brine (20 mL), dried over anhydrous Na₂SO₄, filtered, and concentrated under reduced pressure to provide 2-(2-(cyclopropylsulfinyl)ethyl)-N-(2,4-dimethoxybenzyl)-7-methoxy-[1,2,4]triazolo[1,5-c]quinazolin-5-amine, which was used in the subsequent step without further purification. MS (ESI) m/z calc'd for C₂₄H₂₇N₅O_{4S} [M+H]⁺ 482.2. found 482.2.

### Step 3—Synthesis of Intermediate S.3, 2-(2-aminoethyl)-N-(2,4-dimethoxybenzyl)-9-fluoro-7-methoxy-[1,2,4]triazolo[1,5-c]quinazolin-5-amine

PIDA (50 mg, 0.155 mmol) was added to a suspension of 2-(2-(cyclopropylsulfinyl)ethyl)-N-(2,4-dimethoxybenzyl)-7-methoxy-[1,2,4]triazolo[1,5-c]quinazolin-5-amine (50 mg, 0.104 mmol), BocNH₂ (19 mg, 0.162 mmol), MgO (18 mg, 0.447 mmol) and [Rh(C₇H₁₅CO₂)]₂ (5 mg, 6.42 μmol) in DCM (4 mL). The reaction was stirred vigorously at 40° C. for 16 h. The reaction was cooled to room temperature, filtered through Celite®, and concentrated under reduced pressure to provide tert-butyl (cyclopropyl(2-(5-((2,4-dimethoxybenzyl)amino)-7-methoxy-[1,2,4]triazolo[1,5-c]quinazolin-2-yl)ethyl)(oxo)-16-sulfanylidene)carbamate, which was used in subsequent steps without further purification. MS (ESI) m/z calc'd for C₂₉H₃₇N₆O₆S [M+H]⁺ 597.2. found 597.5.

### EXAMPLES

### Preparation of Examples 1.1 and 1.2, 2-(3-chloropropyl)-7-methoxy-[1,2,4]triazolo[1,5-c]quinazolin-5-amine and 7-methoxy-2-(3-((4-(trifluoromethyl)phenyl)thio)propyl)-[1,2,4]triazolo[1,5-c]quinazolin-5-amine

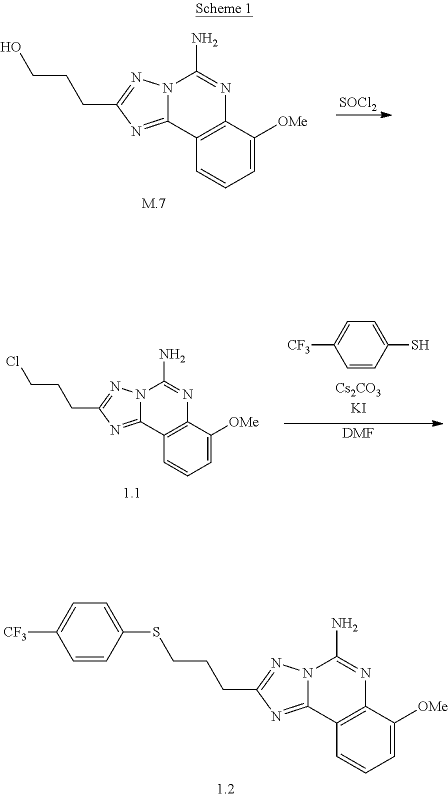

### Step 1—Synthesis of Example 1.1, 2-(3-chloropropyl)-7-methoxy-[1,2,4]triazolo[1,5-c]quinazolin-5-amine

SOCl₂ (5 mL) was added to 3-(5-amino-7-methoxy-[1,2,4]triazolo[1,5-c]quinazolin-2-yl)propan-1-ol (410 mg, 1.5 mmol). The reaction was stirred vigorously at 65° C. for 2 h. The reaction was cooled to room temperature and concentrated under reduced pressure to provide 2-(3-chloropropyl)-7-methoxy-[1,2,4]triazolo[1,5-c]quinazolin-5-amine (Example 1.1). MS (ESI) m/z calc'd for C₁₃H₁₅ClN₅O [M+H]⁺ 292.1. found 292.1. A_{2A} IC₅₀ 15.5 nM (B).

### Step 2—Synthesis of Example 1.2, 7-methoxy-2-(3-((4-(trifluoromethyl)phenyl)thio)propyl)-[1,2,4]triazolo[1,5-c]quinazolin-5-amine

Cs₂CO₃ (67.0 mg, 0.206 mmol) was added to a stirred solution of 4-(trifluoromethyl)thiophenol (17 μL, 0.123 mmol) in DMF (1 mL). The reaction was stirred vigorously at room temperature for 30 min. 2-(3-chloropropyl)-7-methoxy-[1,2,4]triazolo[1,5-c]quinazolin-5-amine (30 mg, 0.103 mmol) and KI (0.854 mg, 5.14 μmole) were added, and the reaction was stirred vigorously at 70° C. for 16 h. The reaction was cooled to room temperature, diluted with water (6 mL), and extracted with EtOAc (2×8 mL). The combined organic layers were washed with brine (8 mL), dried over anhydrous MgSO₄, filtered, and concentrated under reduced pressure. The resulting crude residue was purified by silica gel chromatography (gradient elution: 50-100% EtOAc/hexanes) to provide 7-methoxy-2-(3-((4-(trifluoromethyl)phenyl)thio)propyl)-[1,2,4]triazolo[1,5-c]quinazolin-5-amine (Example 1.2). MS (ESI) m/z calc'd for C₂₀H₁₉F₃N₅OS [M+H]⁺ 434.1. found 434.0. A_{2A} IC₅₀ 8.4 nM (B).

The following example in Table 10 was prepared according to Step 2 of Scheme 1 and General Scheme 1, using intermediate N.1 and the commercially available thiol. Asterisk (*) indicates that A2B data is not available.

TABLE 10
Examples Prepared According to Scheme 1

| | | | A2a |
|---|---|---|---|
| | | | IC₅₀ |
| | | | (nM) |
| | | Observed | A2b |
| | Structure | m/z | IC₅₀ |
| Example | Name | [M + H]⁺ | (nM) |
| 1.3 | 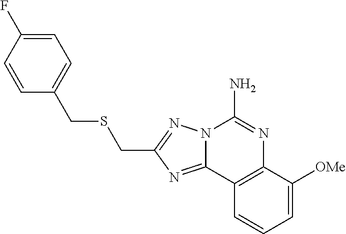 [SMILES: COc1cccc2c1nc(N)n1nc(CSCc3ccc(F)cc3)nc21] | 370.1 | 7.3 (B) |
| | 2-(((4-fluorobenzyl)thio)methyl)-7-methoxy- | | * |
| | [1,2,4]triazolo[1,5-c]quinazolin-5-amine | | |

### Preparation of Examples 2.2A and 2.2B, (S or R)-2-(((1-(3,3-difluorocyclobutyl)-4,5,6,7-tetrahydro-1H-benzo[d][1,2,3]triazol-5-yl)oxy)methyl)-7-methoxy-[1,2,4]triazolo[1,5-c]quinazolin-5-amine and (R or S)-2-(((1-(3,3-difluorocyclobutyl)-4,5,6,7-tetrahydro-1H-benzo[d][1,2,3]triazol-5-yl)oxy)methyl)-7-methoxy-[1,2,4]triazolo[1,5-c]quinazolin-5-amine

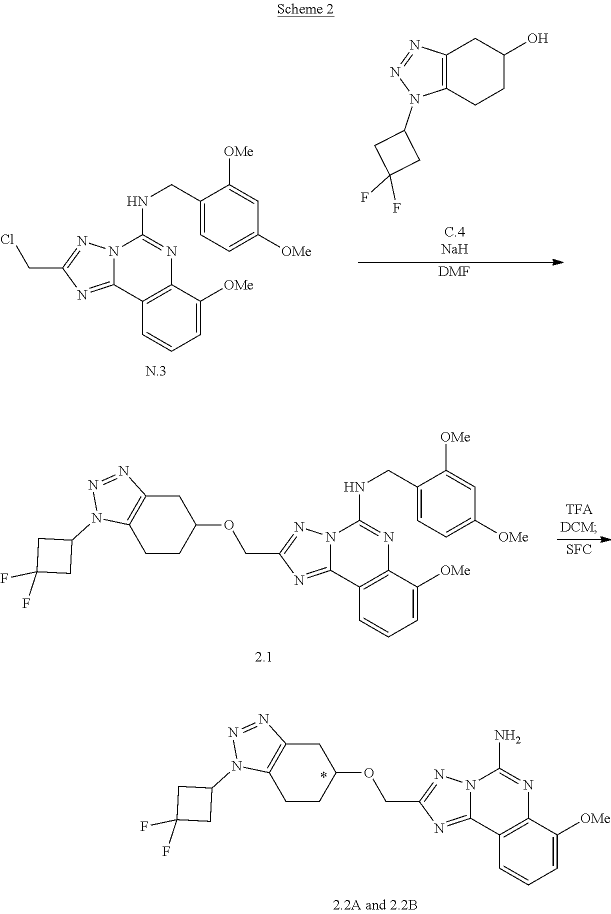

### Step 1—Synthesis of Intermediate 2.1, 2-(((1-(3,3-difluorocyclobutyl)-4,5,6,7-tetrahydro-1H-benzo[d][1,2,3]triazol-5-yl)oxy)methyl)-N-(2,4-dimethoxybenzyl)-7-methoxy-[1,2,4]triazolo[1,5-c]quinazolin-5-amine

NaH (60%, 3.93 mg, 0.098 mmol) was added to a stirred solution of 1-(3,3-difluorocyclobutyl)-4,5,6,7-tetrahydro-1H-benzo[d][1,2,3]triazol-5-ol (15 mg, 0.065 mmol) in DMF (1.5 mL) at 15° C. The reaction was stirred vigorously at 15° C. for 10 min. 2-(chloromethyl)-N-(2,4-dimethoxybenzyl)-7-methoxy-[1,2,4]triazolo[1,5-c]quinazolin-5-amine (27.1 mg, 0.065 mmol) was added, and the reaction was stirred vigorously at room temperature for 15 h. The reaction was cooled, carefully quenched with water (3 mL), and extracted with EtOAc (3×20 mL). The combined organic layers were dried over anhydrous Na₂SO₄, filtered and concentrated under reduced pressure to provide 2-(((1-(3,3-difluorocyclobutyl)-4,5,6,7-tetrahydro-1H-benzo[d][1,2,3]triazol-5-yl)oxy)methyl)-N-(2,4-dimethoxybenzyl)-7-methoxy-[1,2,4]triazolo[1,5-c]quinazolin-5-amine, which was used in the subsequent step without further purification. MS (ESI) m/z calc'd for C₃₀H₃₂F₂N₈O₄ [M+H]⁺ 607.2. found 607.3.

### Step 2—Synthesis of Examples 2.2A and 2.2B, (S or R)-2-(((1-(3,3-difluorocyclobutyl)-4,5,6,7-tetrahydro-1H-benzo[d][1,2,3]triazol-5-yl)oxy)methyl)-7-methoxy-[1,2,4]triazolo[1, 5-c]quinazolin-5-amine and (R or S)-2-(((1-(3,3-difluorocyclobutyl)-4,5,6,7-tetrahydro-1H-benzo[d][1,2,3]triazol-5-yl)oxy)methyl)-7-methoxy-[1,2,4]triazolo[1,5-c]quinazolin-5-amine

TFA (1 mL) was added to a stirred solution of 2-(((1-(3,3-difluorocyclobutyl)-4,5,6,7-tetrahydro-1H-benzo[d][1,2,3]triazol-5-yl)oxy)methyl)-N-(2,4-dimethoxybenzyl)-7-methoxy-[1,2,4]triazolo[1,5-c]quinazolin-5-amine (21 mg, 0.023 mmol) in DCM (1 mL). The reaction was stirred vigorously at 50° C. for 15 h. The reaction was cooled to room temperature and concentrated under reduced pressure. The resulting crude residue was purified by reversed-phase HPLC [Method A]. The racemic mixture was separated by chiral SFC (Column: Chiralpak AD-3, 50×4.6 mm, gradient elution: 5-40% i-PrOH [w/1% Et₂NH]/CO₂) to provide Example 2.2A (faster eluting) and Example 2.2B (slower eluting).

Fasting eluting (Example 2.2A): MS (ESI) m/z: calc'd for C₂₁H₂₃F₂N₈O₂ [M+H]⁺: 457.1. found 457.1. ¹H NMR (400 MHz, MeOD-d₄) δ 7.96 (d, J=7.0 Hz, 1H), 7.40 (t, J=8.0 Hz, 1H), 7.18 (d, J=8.2 Hz, 1H), 6.05 (br s, 2H), 4.93 (s, 2H), 4.64 (br d, J=4.3 Hz, 1H), 4.17 (s, 1H), 4.07 (s, 3H), 3.44 (br s, 2H), 3.23-2.98 (m, 4H), 2.80 (s, 2H), 2.62 (br d, J=16.0 Hz, 2H), 2.20 (s, 1H), 2.08 (br d, J=6.7 Hz, 1H). A_{2A} IC₅₀ 3.4 nM (A).

Slower eluting (Example 2.2B): MS (ESI) m/z: calc'd for C₂₁H₂₃F₂N₈O₂ [M+H]⁺: 457.1. found 457.1. ¹H NMR (400 MHz, MeOD-d₄) δ 7.96 (d, J=7.0 Hz, 1H), 7.40 (t, J=8.0 Hz, 1H), 7.18 (d, J=8.2 Hz, 1H), 6.05 (br s, 2H), 4.93 (s, 2H), 4.64 (br d, J=4.3 Hz, 1H), 4.17 (s, 1H), 4.07 (s, 3H), 3.44 (br s, 2H), 3.23-2.98 (m, 4H), 2.80 (s, 2H), 2.62 (br d, J=16.0 Hz, 2H), 2.20 (s, 1H), 2.08 (br d, J=6.7 Hz, 1H). A_{2A} IC₅₀ 0.6 nM (A).

### Preparation of Example 3.2, 2-((5-amino-7-methoxy-[1,2,4]triazolo[1,5-c]quinazolin-2-yl)methyl)-2-methylpropane-1,3-diol

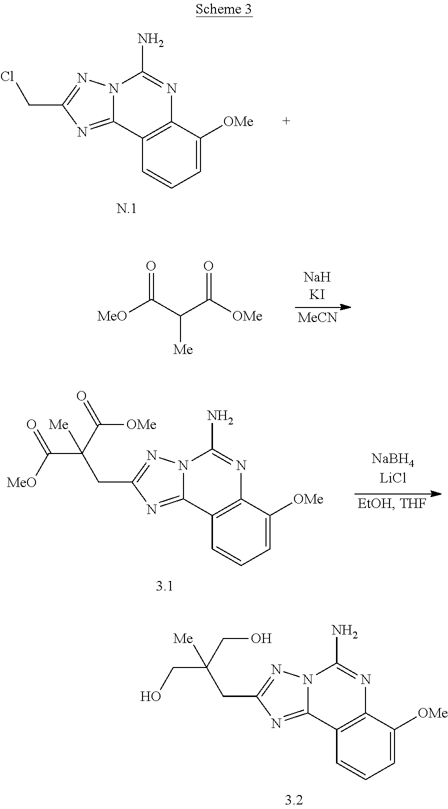

### Step 1—Synthesis of Intermediate 3.1, dimethyl 2-((5-amino-7-methoxy-[1,2,4]triazolo[1,5-c]quinazolin-2-yl)methyl)-2-methylmalonate

2-(chloromethyl)-7-methoxy-[1,2,4]triazolo[1,5-c]quinazolin-5-amine (1.00 g, 3.79 mmol) was added to a suspension of dimethyl 2-methylmalonate (1.66 g, 11.4 mmol), KI (1.89 g, 11.4 mmol), and NaH (197 mg, 4.93 mmol) in MeCN (10 ml). The reaction was stirred vigorously at 70° C. for 16 h. The reaction was cooled to room temperature and diluted with water. The resulting solid was filtered, washed with water, and dried under reduced pressure to provide dimethyl 2-((5-amino-7-methoxy-[1,2,4]triazolo[1,5-c]quinazolin-2-yl)methyl)-2-methylmalonate, which was used in the subsequent step without further purification. MS (ESI) m/z calc'd for C₁₇H₂₀N₅O₅ [M+H]⁺ 374.1. found 374.1.

### Step 2—Synthesis of Example 3.2, 2-((5-amino-7-methoxy-[1,2,4]triazolo[1,5-c]quinazolin-2-yl)methyl)-2-methylpropane-1,3-diol

Dimethyl 2-((5-amino-7-methoxy-[1,2,4]triazolo[1,5-c]quinazolin-2-yl)methyl)-2-methylmalonate (800 mg, 2.14 mmol) was added to a suspension of LiCl (18.2 mg, 0.429 mmol) and NaBH₄ (324 mg, 8.57 mmol) in THE (5 mL) and EtOH (5 mL). The reaction was stirred vigorously at room temperature for 16 h. The reaction mixture was cooled to 0° C. and carefully quenched with 1 N aq. HCl to pH 4-5. The resulting solid was filtered, washed with water, and dried under reduced pressure to provide 2-((5-amino-7-methoxy-[1,2,4]triazolo[1,5-c]quinazolin-2-yl)methyl)-2-methylpropane-1,3-diol (Example 3.2). MS (ESI) m/z calc'd for C₁₅H₂₀N₅O₃ [M+H]⁺ 318.2. found 318.2. A_{2A} IC₅₀ 10.9 nM (A).

### Preparation of Example 4.1 2-(4-(((5-amino-7-methoxy-[1,2,4]triazolo[1,5-c]quinazolin-2-yl)methoxy)methyl)phenyl)-1,1,1,3,3,3-hexafluoropropan-2-ol

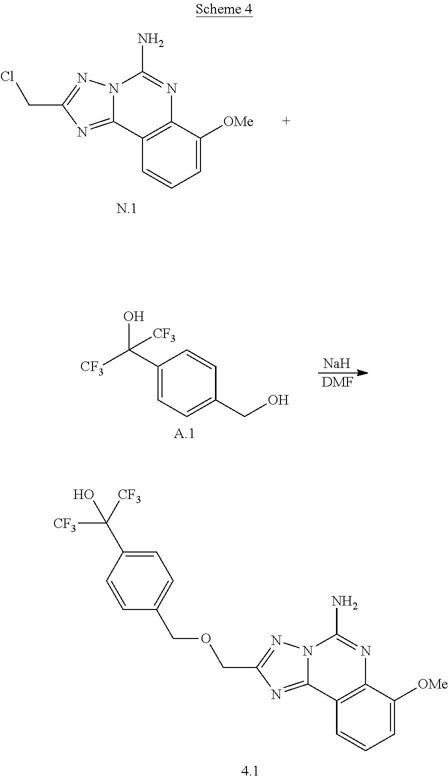

NaH (21.2 mg, 0.531 mmol) was added to a stirred solution of 1,1,1,3,3,3-hexafluoro-2-(4-(hydroxymethyl)phenyl)propan-2-ol (29.1 mg, 0.106 mmol) in DMF (0.5 mL) at 0° C. The reaction was stirred vigorously at 0° C. for 30 min, at which point, a solution of 2-(chloromethyl)-7-methoxy-[1,2,4]triazolo[1,5-c]quinazolin-5-amine (28 mg, 0.106 mmol) in DMF (1.1 mL) was added. The reaction was stirred vigorously at room temperature for 16 h. The reaction was quenched with water (1.5 mL) and MeOH (1.5 mL), filtered, and concentrated under reduced pressure. The resulting crude residue was purified by reversed-phase HPLC [Method B] to provide 2-(4-(((5-amino-7-methoxy-[1,2,4]triazolo[1,5-c]quinazolin-2-yl)methoxy)methyl)phenyl)-1,1,1,3,3,3-hexafluoropropan-2-ol (Example 4.1). MS (ESI) m/z calc'd for C₂₁H₁₈F₆N₅O₃ [M+H]⁺ 502.1. found 502.1. ¹H NMR (500 MHz, DMSO-d₆) δ 8.76 (br s, 1H), 7.90 (s, 2H), 7.77 (dd, J=7.9, 1.2 Hz, 1H), 7.69 (d, J=8.0 Hz, 2H), 7.55 (d, J=8.2 Hz, 2H), 7.33 (t, J=7.9 Hz, 1H), 7.25 (d, J=8.0 Hz, 1H), 4.83 (s, 2H), 4.73 (s, 2H), 3.92 (s, 3H). A_{2A} IC₅₀ 0.1 nM (A), A_{2B} IC₅₀ 0.6 nM (A).

The following Examples in Table 11 were prepared according to Scheme 4 and General Scheme 1, using the appropriate alcohol. Compounds were generally purified by filtering and washing with an appropriate solvent, silica gel chromatography, or reversed-phase prep-HPLC. Example 4.2 was prepared using K₂CO₃ and KI, and Example 4.3 was prepared using NaH and KI. The use of KI is as described in Step 2 of Scheme 1. Asterisk (*) indicates that A2B data is not available.

TABLE 11
Examples Prepared According to Scheme 4

| | | | A2a IC₅₀ |
|---|---|---|---|
| | | Observed | (nM) |
| | Structure | m/z | A2b IC₅₀ |
| Example | Name | [M + H]⁺ | (nM) |
| 4.2 | 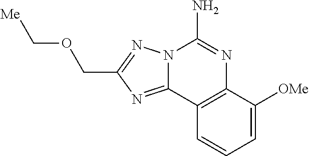 [SMILES: CCOCc1nc2c3cccc(OC)c3nc(N)n2n1] | 274.2 | 8.7 (B) |
| | 2-(ethoxymethyl)-7-methoxy-[1,2,4]triazolo[1,5- | | * |
| | c]quinazolin-5-amine | | |
| 4.3 | 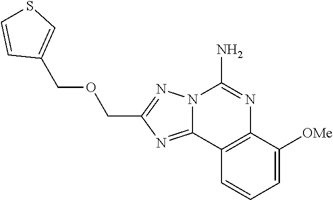 [SMILES: COc1cccc2c1nc(N)n1nc(COCc3ccsc3)nc21] | 342.2 | 2.7 (B) |
| | 7-methoxy-2-((thiophen-3-ylmethoxy)methyl)- | | * |
| | [1,2,4]triazolo[1,5-c]quinazolin-5-amine | | |
| 4.4 | 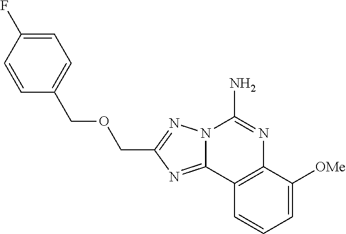 [SMILES: COc1cccc2c1nc(N)n1nc(COCc3ccc(F)cc3)nc21] | 354.0 | 4.7 (B) |
| | 2-(((4-fluorobenzyl)oxy)methyl)-7-methoxy- | | * |
| | [1,2,4]triazolo[1,5-c]quinazolin-5-amine | | |
| 4.5 | 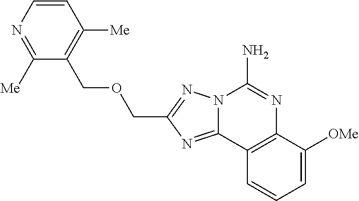 [SMILES: COc1cccc2c1nc(N)n1nc(COCc3c(C)ccnc3C)nc21] | 365.2 | 8.7 (B) |
| | 2-(((2,4-dimethylpyridin-3-yl)methoxy)methyl)-7- | | * |
| | methoxy-[1,2,4]triazolo[1,5-c]quinazolin-5-amine | | |
| 4.6 | 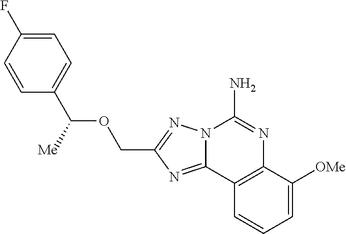 [SMILES: COc1cccc2c1nc(N)n1nc(CO[C@H](C)c3ccc(F)cc3)nc21] | 368.0 | 8.3 (B) |
| | (R)-2-((1-(4-fluorophenyl)ethoxy)methyl)-7-methoxy- | | * |
| | [1,2,4]triazolo[1,5-c]quinazolin-5-amine | | |
| 4.7 | 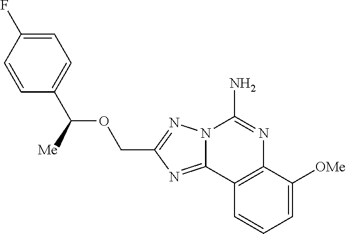 [SMILES: COc1cccc2c1nc(N)n1nc(CO[C@@H](C)c3ccc(F)cc3)nc21] | 368.0 | 4.6 (B) |
| | (S)-2-((1-(4-fluorophenyl)ethoxy)methyl)-7-methoxy- | | * |
| | [1,2,4]triazolo[1,5-c]quinazolin-5-amine | | |
| 4.8 | 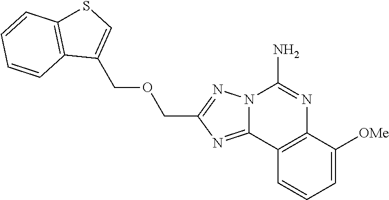 [SMILES: COc1cccc2c1nc(N)n1nc(COCc3csc4ccccc34)nc21] | 392.0 | 2.9 (B) |
| | 2-((benzo[b]thiophen-3-ylmethoxy)methyl)-7-methoxy- | | * |
| | [1,2,4]triazolo[1,5-c]quinazolin-5-amine | | |
| 4.9 | 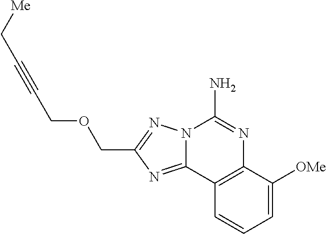 [SMILES: CCC#CCOCc1nc2c3cccc(OC)c3nc(N)n2n1] | 312.0 | 6.1 (B) |
| | 7-methoxy-2-((pent-2-yn-1-yloxy)methyl)- | | * |
| | [1,2,4]triazolo[1,5-c]quinazolin-5-amine | | |
| 4.10 | 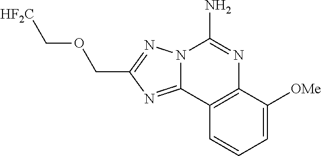 | 310.0 | 4.4 (B) |
| | 2-((2,2-difluoroethoxy)methyl)-7-methoxy- | | * |
| | [1,2,4]triazolo[1,5-c]quinazolin-5-amine | | |
| 4.11 | 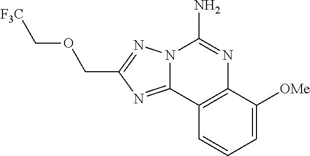 [SMILES: COc1cccc2c1nc(N)n1nc(COCC(F)(F)F)nc21] | 328.1 | 3.5 (B) |
| | 7-methoxy-2-((2,2,2-trifluoroethoxy)methyl)- | | * |
| | [1,2,4]triazolo[1,5-c]quinazolin-5-amine | | |
| 4.12 | 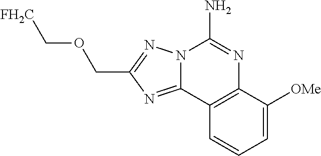 | 292.1 | 7.2 (B) |
| | 2-((2-fluoroethoxy)methyl)-7-methoxy- | | * |
| | [1,2,4]triazolo[1,5-c]quinazolin-5-amine | | |
| 4.13 | 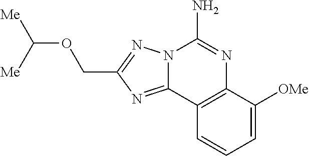 [SMILES: COc1cccc2c1nc(N)n1nc(COC(C)C)nc21] | 288.1 | 5.7 (B) |
| | 2-(isopropoxymethyl)-7-methoxy-[1,2,4]triazolo[1,5- | | * |
| | c]quinazolin-5-amine | | |
| 4.14 | 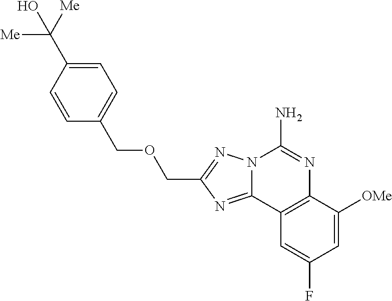 [SMILES: COc1cc(F)cc2c1nc(N)n1nc(COCc3ccc(C(C)(C)O)cc3)nc21] | 412.1 | 0.2 (A) |
| | 2-(4-(((5-amino-9-fluoro-7-methoxy-[1,2,4]triazolo[1,5- | | 9.1 (A) |
| | c]quinazolin-2-yl)methoxy)methyl)phenyl)propan-2-ol | | |
| 4.15 | 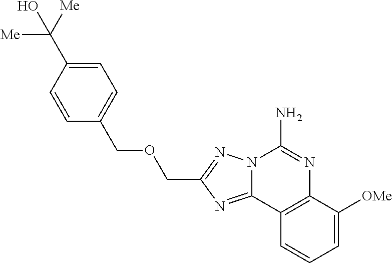 [SMILES: COc1cccc2c1nc(N)n1nc(COCc3ccc(C(C)(C)O)cc3)nc21] | 394.2 | 0.2 (A) |
| | 2-(4-(((5-amino-7-methoxy-[1,2,4]triazolo[1,5- | | 10.0 (A) |
| | c]quinazolin-2-yl)methoxy)methyl)phenyl)propan-2-ol | | |
| 4.16 | 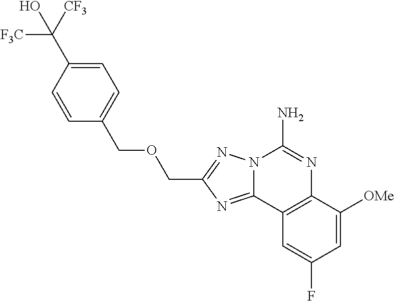 [SMILES: COc1cc(F)cc2c1nc(N)n1nc(COCc3ccc(C(O)(C(F)(F)F)C(F)(F)F)cc3)nc21] | 520.1 | 0.3 (A) |
| | 2-(4-(((5-amino-9-fluoro-7-methoxy-[1,2,4]triazolo[1,5- | | 0.9 (A) |
| | c]quinazolin-2-yl)methoxy)methyl)phenyl)-1,1,1,3,3,3- | | |
| | hexafluoropropan-2-ol | | |

### Preparation of Examples 5.2A and 5.2B, (S or R)-2-(2-(1-isopropyl-4,5,6,7-tetrahydro-1H-benzo[d][1,2,3]triazol-5-yl)ethyl)-7-methoxy-[1,2,4]triazolo[1,5-c]quinazolin-5-amine (R or S) 2-(2-(1-isopropyl-4,5,6,7-tetrahydro-1H-benzo[d][1,2,3]triazol-5-yl)ethyl)-7-methoxy-[1,2,4]triazolo[1,5-c]quinazolin-5-amine

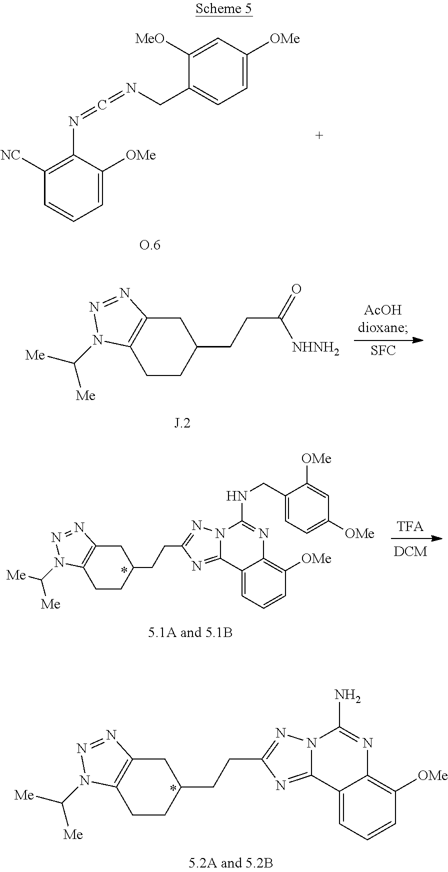 [SMILES: COc1ccc(CN=C=Nc2c(C#N)cccc2OC)c(OC)c1 ; CC(C)n1nnc2c1CCC(CCC(=O)NN)C2 ; "SFC" ; COc1ccc(CNc2nc3c(OC)cccc3c3nc(CCC4CCc5c(nnn5C(C)C)C4)nn23)c(OC)c1 ; COc1cccc2c1nc(N)n1nc(CCC3CCc4c(nnn4C(C)C)C3)nc21]

### Step 1—Synthesis of Intermediates 5.1A and 5.1B, (S or R)—N-(2,4-dimethoxybenzyl)-2-(2-(1-isopropyl-4,5,6,7-tetrahydro-1H-benzo[d][1,2,3]triazol-5-yl)ethyl)-7-methoxy-[1,2,4]triazolo[1,5-c]quinazolin-5-amine and (R or S)—N-(2,4-dimethoxybenzyl)-2-(2-(1-isopropyl-4,5,6,7-tetrahydro-1H-benzo[d][1,2,3]triazol-5-yl)ethyl)-7-methoxy-[1,2,4]triazolo[1,5-c]quinazolin-5-amine

2-((((2,4-Dimethoxybenzyl)imino)methylene)amino)-3-methoxybenzonitrile (144 mg, 0.445 mmol) and AcOH (13 μL, 0.223 mmol) were added to a stirred solution of 3-(1-isopropyl-4,5,6,7-tetrahydro-1H-benzo[d][1,2,3]triazol-5-yl)propanehydrazide (134 mg, 0.534 mmol) in dioxane (2.23 mL) at 70° C. The reaction was stirred vigorously at 70° C. for 3 h. The reaction was cooled to room temperature and concentrated under reduced pressure. The concentrated residue was diluted with water and extracted with DCM. The combined organic layers were concentrated under reduced pressure. The resulting crude residue was purified by reversed-phase HPLC [Method B]. The racemic mixture was separated by chiral SFC (Column: OJ-H, 21×250 mm, gradient elution: 80% MeOH [w/0.1% NH₄OH]/CO₂) to provide Intermediate 5.1A (faster eluting) and Intermediate 5.1B (slower eluting).

### Step 2—Synthesis of Examples 5.2A and 5.2B, (S or R)-2-(2-(1-isopropyl-4,5,6,7-tetrahydro-1H-benzo[d][1,2,3]triazol-5-yl)ethyl)-7-methoxy-[1,2,4]triazolo[1,5-c]quinazolin-5-amine and (R or S)-2-(2-(1-isopropyl-4,5,6,7-tetrahydro-1H-benzo[d][1,2,3]triazol-5-yl)ethyl)-7-methoxy-[1,2,4]triazolo[1,5-c]quinazolin-5-amine

TFA (0.30 mL) was added to a stirred solution of Intermediate 5.1A (21.4 mg, 0.038 mmol) in DCM (0.77 mL). The reaction was stirred vigorously at 50° C. for 4 h. The reaction was cooled to room temperature and concentrated under reduced pressure. The resulting crude residue was purified by reversed-phase HPLC [Method B] to provide Example 5.2A (from faster eluting Intermediate 5.1A): MS (ESI) m/z: calc'd for C₂₁H₂₇N₈O [M+H]⁺: 407.2. found 407.3. ¹H NMR (500 MHz, DMSO-d₆) δ 7.78 (s, 2H), 7.73 (dd, J=7.9, 1.2 Hz, 1H), 7.30 (t, J=7.9 Hz, 1H), 7.22 (d, J=7.1 Hz, 1H), 4.59 (hept, J=6.7 Hz, 1H), 3.90 (s, 3H), 3.01 (t, J=7.8 Hz, 2H), 2.88 (dd, J=15.3, 4.8 Hz, 1H), 2.82-2.72 (m, 1H), 2.67-2.57 (m, 2H), 2.40-2.27 (m, 1H), 2.15-1.75 (m, 4H), 1.58-1.39 (m, 7H). A_{2A} IC₅₀ 0.7 nM (A).

TFA (0.16 mL) was added to a stirred solution of Intermediate 5.1B (23.1 mg, 0.041 mmol) in DCM (0.83 mL). The reaction was stirred vigorously at 50° C. for 4 h. The reaction was cooled to room temperature and concentrated under reduced pressure. The resulting crude residue was purified by reversed-phase HPLC [Method B] to provide Example 5.2B (from slower eluting Intermediate 5.1B): MS (ESI) m/z: calc'd for C₂₁H₂₇N₈O [M+H]⁺: 407.2. found 407.2. ¹H NMR (500 MHz, DMSO-d₆) δ 7.79 (s, 1H), 7.73 (dd, J=7.9, 1.2 Hz, 1H), 7.30 (t, J=7.9 Hz, 1H), 7.26-7.19 (m, 1H), 4.59 (hept, J=6.8 Hz, 1H), 3.90 (s, 3H), 3.01 (t, J=7.8 Hz, 2H), 2.88 (dd, J=15.4, 4.8 Hz, 1H), 2.76 (dd, J=11.1, 3.3 Hz, 1H), 2.68-2.57 (m, 2H), 2.41-2.24 (m, 1H), 2.09-1.75 (m, 4H), 1.59-1.36 (m, 7H). A_{2A} IC₅₀ 0.8 nM (A), A_{2B} IC₅₀ 12.0 nM (A).

The following examples in Table 12 were prepared according to Scheme 5 and General Scheme 7, using the appropriate nitrile and hydrazide. SFC conditions for the resolution involved in Step 1 are provided, following the table. Asterisk (*) indicates that A2B data is not available.

TABLE 12
Examples Prepared According to Scheme 5

| | | | A2a IC₅₀ |
|---|---|---|---|
| | | Observed | (nM) |
| | Structure | m/z | A2b IC₅₀ |
| Example | Name | [M + H]⁺ | (nM) |
| 5.3A (from faster eluting intermediate) | 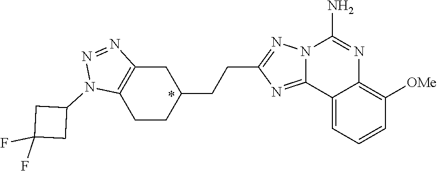 [SMILES: COc1cccc2c1nc(N)n1nc(CCC3CCc4c(nnn4C4CC(F)(F)C4)C3)nc21] | 455.2 | 0.9 (A) * |
| | (S or R)-2-(2-(1-(3,3-difluorocyclobutyl)-4,5,6,7-tetrahydro-1H- | | |
| | benzo[d][1,2,3]triazol-5-yl)ethyl)-7-methoxy-[1,2,4]triazolo- | | |
| | [1,5-c]quinazolin-5-amine | | |
| 5.3B (from slower eluting intermediate) | 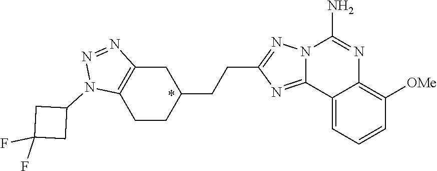 [SMILES: COc1cccc2c1nc(N)n1nc(CCC3CCc4c(nnn4C4CC(F)(F)C4)C3)nc21] | 455.2 | 0.9 (A) 3.1 (A) |
| | (R or S)-2-(2-(1-(3,3-difluorocyclobutyl)-4,5,6,7-tetrahydro-1H- | | |
| | benzo[d][1,2,3]triazol-5-yl)ethyl)-7-methoxy-[1,2,4]triazolo- | | |
| | [1,5-c]quinazolin-5-amine | | |
| 5.4A (from faster eluting intermediate) | 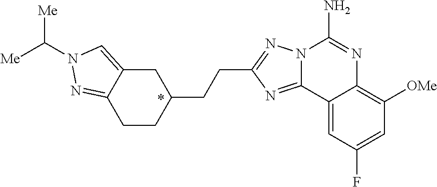 [SMILES: COc1cc(F)cc2c1nc(N)n1nc(CCC3CCc4nn(C(C)C)cc4C3)nc21] | 424.2 | 1.9 (A) 9.4 (A) |
| | (S or R)-9-fluoro-2-(2-(2-isopropyl-4,5,6,7-tetrahydro-2H-indazol-5- | | |
| | yl)ethyl)-7-methoxy-[1,2,4]triazolo[1,5-c]quinazolin-5-amine | | |
| 5.4B (from slower eluting intermediate) | 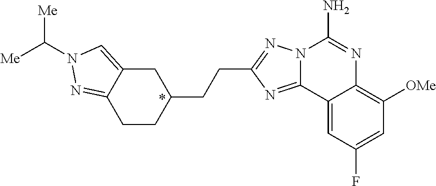 [SMILES: COc1cc(F)cc2c1nc(N)n1nc(CCC3CCc4nn(C(C)C)cc4C3)nc21] | 424.2 | 5.5 (A) * |
| | (R or S)-9-fluoro-2-(2-(2-isopropyl-4,5,6,7-tetrahydro-2H-indazol-5- | | |
| | yl)ethyl)-7-methoxy-[1,2,4]triazolo[1,5-c]quinazolin-5-amine | | |

### Examples 5.3A and 5.3B

The racemic intermediate en route to Examples 5.3A and 5.3B was separated by chiral SFC (Column: AS-H, 21×250 mm, 80% i-PrOH [w/0.1% NH₄OH]/CO₂).

### Examples 5.4A and 5.4B

The racemic intermediate en route to Examples 5.4A and 5.4B was separated by chiral SFC (Column: CCA, 21×250 mm, 70% MeOH [w/0.1% NH₄OH]/CO₂).

### Preparation of Example 6.2, 9-fluoro-8-methoxy-2-(2-(phenylsulfonyl)ethyl)-[1,2,4]triazolo[1,5-c]quinazolin-5-amine

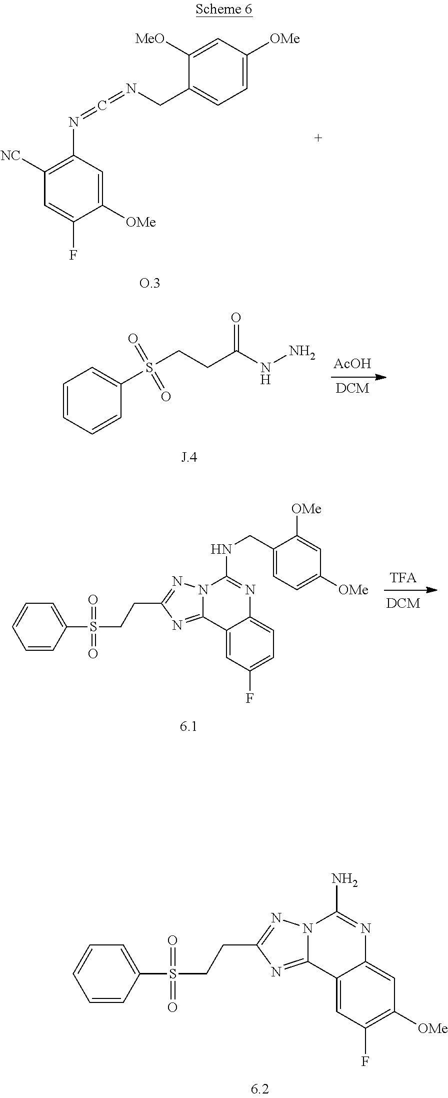 [SMILES: COc1ccc(CN=C=Nc2cc(OC)c(F)cc2C#N)c(OC)c1 ; "AcOH" ; NNC(=O)CCS(=O)(=O)c1ccccc1 ; COc1ccc(CNc2nc3ccc(F)cc3c3nc(CCS(=O)(=O)c4ccccc4)nn23)c(OC)c1 ; COc1cc2nc(N)n3nc(CCS(=O)(=O)c4ccccc4)nc3c2cc1F]

### Step 1—Synthesis of Intermediate 6.1, N-(2,4-dimethoxybenzyl)-9-fluoro-8-methoxy-2-(2-(phenylsulfonyl)ethyl)-[1,2,4]triazolo[1,5-c]quinazolin-5-amine

AcOH (19 μL, 0.329 mmol) and 2-((((2,4-dimethoxybenzyl)imino)methylene)amino)-5-fluoro-4-methoxybenzonitrile (224 mg, 0.657 mmol) were added to a stirred solution of 3-(phenylsulfonyl)propanehydrazide (150 mg, 0.657 mmol) in DCM (10 mL). The reaction was stirred vigorously at 35° C. The reaction was cooled to room temperature and concentrated under reduced pressure. The resulting crude residue was purified by silica gel chromatography (gradient elution: 20-50% EtOAc/petroleum ether) to provide N-(2,4-dimethoxybenzyl)-9-fluoro-8-methoxy-2-(2-(phenylsulfonyl)ethyl)-[1,2,4]triazolo[1,5-c]quinazolin-5-amine. MS (ESI) m/z: calc'd for C₂₇H₂₇FN₅O₅S [M+H]⁺: 552.3. found 552.3.

### Step 2—Synthesis of Example 6.2, 9-fluoro-8-methoxy-2-(2-(phenylsulfonyl)ethyl)-[1,2,4]triazolo[1,5-c]quinazolin-5-amine

TFA (3.00 mL) was added to a stirred solution of N-(2,4-dimethoxybenzyl)-9-fluoro-8-methoxy-2-(2-(phenylsulfonyl)ethyl)-[1,2,4]triazolo[1,5-c]quinazolin-5-amine (80.0 mg, 0.145 mmol) in DCM (3.00 mL). The reaction was stirred vigorously at 50° C. for 16 h. The reaction was cooled to room temperature and concentrated under reduced pressure. The resulting crude residue was purified by reversed-phase HPLC [Method A] to provide 9-fluoro-8-methoxy-2-(2-(phenylsulfonyl)ethyl)-[1,2,4]triazolo[1,5-c]quinazolin-5-amine (Example 6.2). MS (ESI) m/z: calc'd for C₁₈H₁₇FN₅O₅S [M+H]⁺: 402. ¹. found 402.1. ¹H NMR (400 MHz, DMSO-d₆) δ 7.86-7.92 (m, 2H), 7.77 (br d, J=11.0 Hz, 3H), 7.51-7.63 (m, 3H), 7.16 (d, J=7.8 Hz, 1H), 3.96 (s, 3H), 3.86 (br t, J=7.6 Hz, 3H), 3.20 (t, J=7.5 Hz, 2H). A_{2A} IC₅₀ 4.8 nM (A), A_{2B} IC₅₀ 455 nM (A).

The following examples in Table 13 were prepared according to Scheme 6 and General Scheme 7, using the appropriate nitrile and hydrazide. Asterisk (*) indicates that A2B data is not available.

TABLE 13
Examples Prepared According to Scheme 6

| | | | A2a IC₅₀ |
|---|---|---|---|
| | | Observed | (nM) |
| | Structure | m/z | A2b IC₅₀ |
| Example | Name | [M + H]⁺ | (nM) |
| 6.3 | 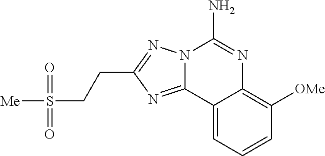 [SMILES: COc1cccc2c1nc(N)n1nc(CCS(C)(=O)=O)nc21] | 322.1 | 8.2 (A) 535 (A) |
| | 7-methoxy-2-(2-(methylsulfonyl)ethyl)-[1,2,4]- | | |
| | triazolo[1,5-c]quinazolin-5-amine | | |
| 6.4 | 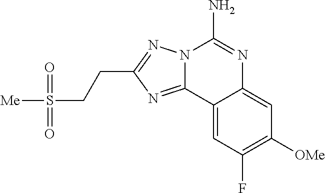 [SMILES: COc1cc2nc(N)n3nc(CCS(C)(=O)=O)nc3c2cc1F] | 340.1 | 222 (A) 1904 (A) |
| | 9-fluoro-8-methoxy-2-(2-(methylsulfonyl)ethyl)- | | |
| | [1,2,4]triazolo[1,5-c]quinazolin-5-amine | | |
| 6.5 | 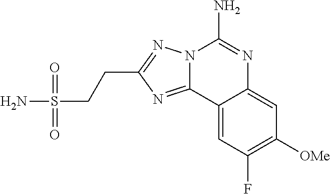 [SMILES: COc1cc2nc(N)n3nc(CCS(N)(=O)=O)nc3c2cc1F] | 341.1 | 107 (A) 113 (A) |
| | 2-(5-amino-9-fluoro-8-methoxy-[1,2,4]triazolo- | | |
| | [1,5-c]quinazolin-2-yl)ethane-1-sulfonamide | | |
| 6.6 | 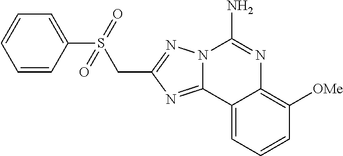 [SMILES: COc1cccc2c1nc(N)n1nc(CS(=O)(=O)c3ccccc3)nc21] | 370.1 | 4.2 (A) 3778 (B) |
| | 7-methoxy-2-((phenylsulfonyl)methyl)-[1,2,4]triazolo- | | |
| | [1,5-c]quinazolin-5-amine | | |

### Preparation of Examples 7.2A and 7.2B, (S or R)-2-(2-(2-isopropyl-4,5,6,7-tetrahydro-2H-indazol-5-yl)ethyl)-7-methoxy-[1,2,4]triazolo[1,5-c]quinazolin-5-amine and (R or S) 2(2-2 isopropyl-4,5,6,7-tetrahydro-2H-indazol-5-yl)ethyl)-7-methoxy-[1,2,4]triazolo[1,5-c]quinazolin-5-amine

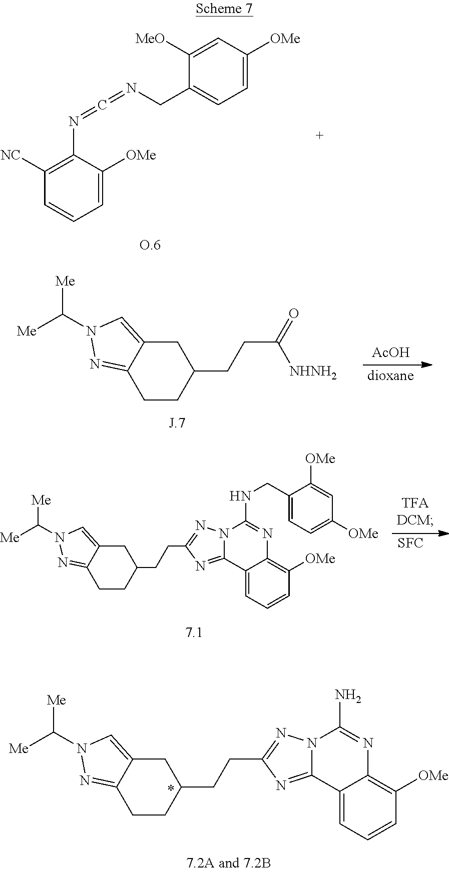

### Step 1—Synthesis of Intermediate 7.1, N-(2,4-dimethoxybenzyl)-2-(2-(2-isopropyl-4,5,6,7-tetrahydro-2H-indazol-5-yl)ethyl)-7-methoxy-[1,2,4]triazolo[1,5-c]quinazolin-5-amine

AcOH (12 μL, 0.21 mmol) and 2-((((2,4-dimethoxybenzyl)imino)methylene)amino)-3-methoxybenzonitrile (138 mg, 0.43 mmol) were added to a stirred solution of 3-(2-isopropyl-4,5,6,7-tetrahydro-2H-indazol-5-yl)propanehydrazide (128 mg, 0.51 mmol) in dioxane (2.1 mL). The reaction was stirred vigorously at 70° C. for 3 h. The reaction was cooled to room temperature and concentrated under reduced pressure. The concentrated residue was diluted with DCM (5 mL) and washed with 1 N aq. HCl (5 mL). The combined organic layers were concentrated under reduced pressure. The resulting crude residue was purified by reversed-phase HPLC [Method A] to provide N-(2,4-dimethoxybenzyl)-2-(2-(2-isopropyl-4,5,6,7-tetrahydro-2H-indazol-5-yl)ethyl)-7-methoxy-[1,2,4]triazolo[1,5-c]quinazolin-5-amine. MS (ESI) m/z: calc'd for C₃₁H₃₈N₇O₃ [M+H]⁺: 556. found 556.

### Step 2—Synthesis of Examples 7.2A and 7.2B, (S or R)-2-(2-(2-isopropyl-4,5,6,7-tetrahydro-2H-indazol-5-yl)ethyl)-7-methoxy-[1,2,4]triazolo[1,5-c]quinazolin-5-amine and (R or S)-2-(2-(2-isopropyl-4,5,6,7-tetrahydro-2H-indazol-5-yl)ethyl)-7-methoxy-[1,2,4]triazolo[1, 5-c]quinazolin-5-amine

TFA (0.60 mL) was added to N-(2,4-dimethoxybenzyl)-2-(2-(2-isopropyl-4,5,6,7-tetrahydro-2H-indazol-5-yl)ethyl)-7-methoxy-[1,2,4]triazolo[1,5-c]quinazolin-5-amine (210 mg, 0.31 mmol). The reaction was stirred vigorously at 45° C. for 1 h. The reaction was cooled to room temperature and concentrated under reduced pressure. The resulting crude residue was purified by reversed-phase HPLC [Method A]. The racemic mixture was separated by chiral SFC (Column: CCO, 21×250 mm, 75% MeOH [w/0.1% NH₄OH]/CO₂) to provide Example 7.2A (faster eluting) and Example 7.2B (slower eluting).

Fast eluting (Example 7.2A): MS (ESI) m/z: calc'd for C₂₁H₂₈N₇O [M+H]⁺: 406. found 406. ¹H NMR (600 MHz, DMSO-d₆) δ 7.78 (s, 2H), 7.73 (dd, J=8.0, 1.1 Hz, 1H), 7.37 (s, 1H), 7.29 (t, J=7.9 Hz, 1H), 7.22 (d, J=7.1 Hz, 1H), 4.37-4.31 (m, 1H), 3.90 (s, 3H), 2.97 (t, J=7.9 Hz, 2H), 2.72 (dd, J=15.1, 4.9 Hz, 1H), 2.65-2.61 (m, 1H), 2.49-2.47 (m, 1H), 2.16 (dd, J=15.1, 10.0 Hz, 1H), 2.01-1.93 (m, 1H), 1.93-1.81 (m, 2H), 1.75-1.70 (m, 1H), 1.49-1.42 (m, 1H), 1.35 (d, J=6.7 Hz, 6H). A_{2A} IC₅₀ 2.5 nM (A).

Slower eluting (Example 7.2B): MS (ESI) m/z: calc'd for C₂₁H₂₈N₇O [M+H]⁺: 406. found 406. ¹H NMR (600 MHz, DMSO-d₆) δ 7.78 (s, 2H), 7.73 (dd, J=8.0, 1.1 Hz, 1H), 7.37 (s, 1H), 7.29 (t, J=7.9 Hz, 1H), 7.22 (d, J=7.1 Hz, 1H), 4.37-4.31 (m, 1H), 3.90 (s, 3H), 2.97 (t, J=7.9 Hz, 2H), 2.72 (dd, J=15.1, 4.9 Hz, 1H), 2.65-2.61 (m, 1H), 2.49-2.47 (m, 1H), 2.16 (dd, J=15.1, 10.0 Hz, 1H), 2.01-1.93 (m, 1H), 1.93-1.81 (m, 2H), 1.75-1.70 (m, 1H), 1.49-1.42 (m, 1H), 1.35 (d, J=6.7 Hz, 6H). A_{2A} IC₅₀ 2.3 nM (A).

The following examples in Table 14 were prepared according to Scheme 7 and General Scheme 7, using the appropriate nitrile and hydrazide. SFC conditions for the resolution involved in Step 2 are provided, following the table. Asterisk (*) indicates that A2B data is not available.

TABLE 14
Examples Prepared According to Scheme 7

| | | | A2a IC₅₀ |
|---|---|---|---|
| | | Observed | (nM) |
| | Structure | m/z | A2b IC₅₀ |
| Example | Name | [M + H]⁺ | (nM) |
| 7.3A (faster eluting) | 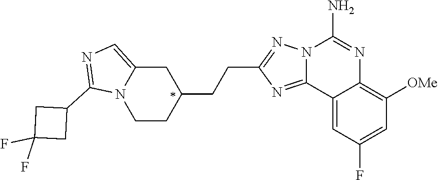 [SMILES: COc1cc(F)cc2c1nc(N)n1nc(CCC3CCn4c(cnc4C4CC(F)(F)C4)C3)nc21] | 472.2 | 2.8 (A) * |
| | (S or R)-2-(2-(3-(3,3-difluorocyclobutyl)-5,6,7,8-tetrahydroimidazo- | | |
| | [1,5-a]pyridin-7-yl)ethyl)-9-fluoro-7-methoxy-[1,2,4]triazolo[1,5-c]- | | |
| | quinazolin-5-amine | | |
| 7.3B (slower eluting) | 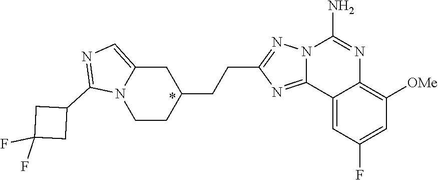 [SMILES: COc1cc(F)cc2c1nc(N)n1nc(CCC3CCn4c(cnc4C4CC(F)(F)C4)C3)nc21] | 472.2 | 4.6 (A) * |
| | (R or S)-2-(2-(3-(3,3-difluorocyclobutyl)-5,6,7,8-tetrahydroimidazo- | | |
| | [1,5-a]pyridin-7-yl)ethyl)-9-fluoro-7-methoxy-[1,2,4]triazolo[1,5-c]- | | |
| | quinazolin-5-amine | | |
| 7.4A (faster eluting) | 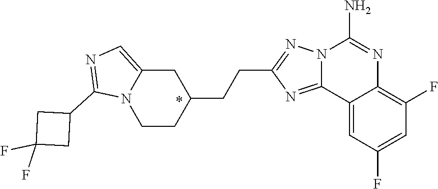 [SMILES: Nc1nc2c(F)cc(F)cc2c2nc(CCC3CCn4c(cnc4C4CC(F)(F)C4)C3)nn12] | 460.2 | 31.6 (A) * |
| | (S or R)-2-(2-(3-(3,3-difluorocyclobutyl)-5,6,7,8-tetrahydroimidazo- | | |
| | [1,5-a]pyridin-7-yl)ethyl)-7,9-difluoro-[1,2,4]triazolo[1,5-c]- | | |
| | quinazolin-5-amine | | |
| 7.5A (faster eluting) | 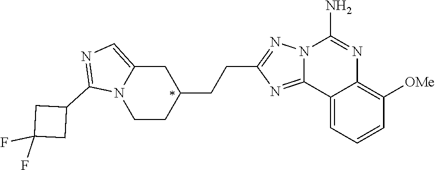 [SMILES: COc1cccc2c1nc(N)n1nc(CCC3CCn4c(cnc4C4CC(F)(F)C4)C3)nc21] | 454.2 | 2.2 (A) * |
| | (S or R)-2-(2-(3-(3,3-difluorocyclobutyl)-5,6,7,8-tetrahydroimidazo- | | |
| | [1,5-a]pyridin-7-yl)ethyl)-7-methoxy-[1,2,4]triazolo[1,5-c]- | | |
| | quinazolin-5-amine | | |
| 7.5B (slower eluting) | 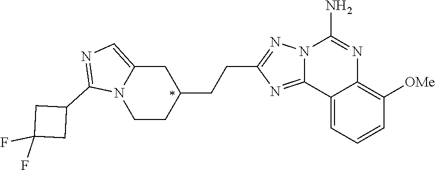 [SMILES: COc1cccc2c1nc(N)n1nc(CCC3CCn4c(cnc4C4CC(F)(F)C4)C3)nc21] | 454.2 | 0.5 (A) * |
| | (R or S)- 2-(2-(3-(3,3-difluorocyclobutyl)-5,6,7,8-tetrahydroimidazo- | | |
| | [1,5-a]pyridin-7-yl)ethyl)-7-methoxy-[1,2,4]triazolo[1,5-c]- | | |
| | quinazolin-5-amine | | |
| 7.6A (faster eluting) | 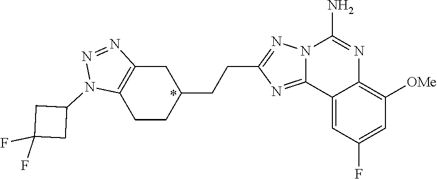 [SMILES: COc1cc(F)cc2c1nc(N)n1nc(CCC3CCc4c(nnn4C4CC(F)(F)C4)C3)nc21] | 473.3 | 1.0 (A) * |
| | (S or R)-2-(2-(1-(3,3-difluorocyclobutyl)-4,5,6,7-tetrahydro-1H- | | |
| | benzo[d][1,2,3]triazol-5-yl)ethyl)-9-fluoro-7-methoxy-[1,2,4]- | | |
| | triazolo[1,5-c]quinazolin-5-amine | | |
| 7.6B (slower eluting) | 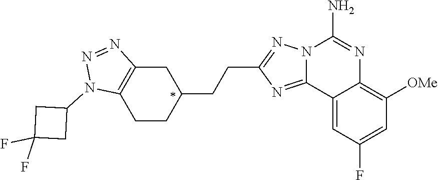 [SMILES: COc1cc(F)cc2c1nc(N)n1nc(CCC3CCc4c(nnn4C4CC(F)(F)C4)C3)nc21] | 473.2 | 4.2 (A) * |
| | (R or S)-2-(2-(1-(3,3-difluorocyclobutyl)-4,5,6,7-tetrahydro-1H- | | |
| | benzo[d][1,2,3]triazol-5-yl)ethyl)-9-fluoro-7-methoxy-[1,2,4]- | | |
| | triazolo[1,5-c]quinazolin-5-amine | | |
| 7.7A (faster eluting) | 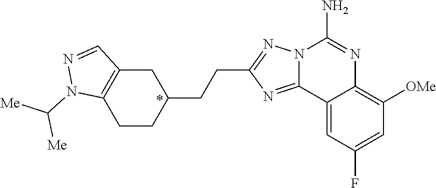 [SMILES: COc1cc(F)cc2c1nc(N)n1nc(CCC3CCc4c(cnn4C(C)C)C3)nc21] | 424.1 | 5.5 (A) * |
| | (S or R)-9-fluoro-2-(2-(1-isopropyl-4,5,6,7-tetrahydro-1H-indazol- | | |
| | 5-yl)ethyl)-7-methoxy-[1,2,4]triazolo[1,5-c]quinazolin-5-amine | | |
| 7.7B (slower eluting) | 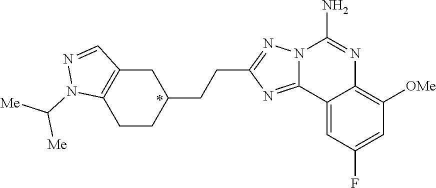 [SMILES: COc1cc(F)cc2c1nc(N)n1nc(CCC3CCc4c(cnn4C(C)C)C3)nc21] | 424.2 | 2.6 (A) * |
| | (R or S)-9-fluoro-2-(2-(1-isopropyl-4,5,6,7-tetrahydro-1H-indazol- | | |
| | 5-yl)ethyl)-7-methoxy-[1,2,4]triazolo[1,5-c]quinazolin-5-amine | | |
| 7.8A (faster eluting) | 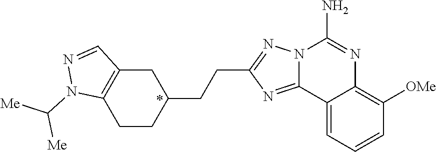 [SMILES: COc1cccc2c1nc(N)n1nc(CCC3CCc4c(cnn4C(C)C)C3)nc21] | 406.3 | 2.1 (A) * |
| | (S or R)-2-(2-(1-isopropyl-4,5,6,7-tetrahydro-1H-indazol-5-yl)- | | |
| | ethyl)-7-methoxy-[1,2,4]triazolo[1,5-c]quinazolin-5-amine | | |
| 7.8B (slower eluting) | 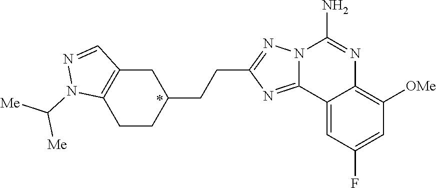 [SMILES: COc1cc(F)cc2c1nc(N)n1nc(CCC3CCc4c(cnn4C(C)C)C3)nc21] | 406.1 | 2.4 (A) * |
| | (R or S)-2-(2-(1-isopropyl-4,5,6,7-tetrahydro-1H-indazol-5-yl)ethyl)- | | |
| | 7-methoxy-[1,2,4]triazolo[1,5-c]quinazolin-5-amine | | |

### Examples 7.3A and 7.3B

The racemic mixture was separated by chiral SFC (Column: CC4, 21×250 mm, 65% MeOH [w/0.1% NH₄OH]/CO₂).

### Examples 7.4A

The racemic mixture was separated by chiral SFC (Column: CC4, 21×250 mm, 65% MeOH [w/0.1% NH₄OH]/CO₂).

### Examples 7.5A and 7.5B

The racemic mixture was separated by chiral SFC (Column: IB, 21×250 mm, 65% MeOH [w/0.1% NH₄OH]/CO₂).

### Examples 7.6A and 7.6B

The racemic mixture was separated by chiral SFC (Column: IB, 21×250 mm, 80% MeOH [w/0.1% NH₄OH]/CO₂).

### Examples 7.7A and 7.7B

The racemic mixture was separated by chiral SFC (Column: CCO, 21×250 mm, 80% MeOH [w/0.1% NH₄OH]/CO₂).

### Examples 7.8A and 7.8B

The racemic mixture was separated by chiral SFC (Column: AS-H, 21×250 mm, 85% MeOH [w/0.1% NH₄OH]/CO₂).

### Preparation of Examples 8.3A and 8.3B, 2-(((1S,2R)-2-(4-(2-aminopropan-2-yl)phenyl)cyclopropyl)methyl)-7,9-difluoro-[1,2,4]triazolo[1,5-c]quinazolin-5-amine and 2-(((1R,2S)-2-(4-(2-aminopropan-2-yl)phenyl)cyclopropyl)methyl)-7,9-difluoro-[1,2,4]triazolo[1,5-c]quinazolin-5-amine

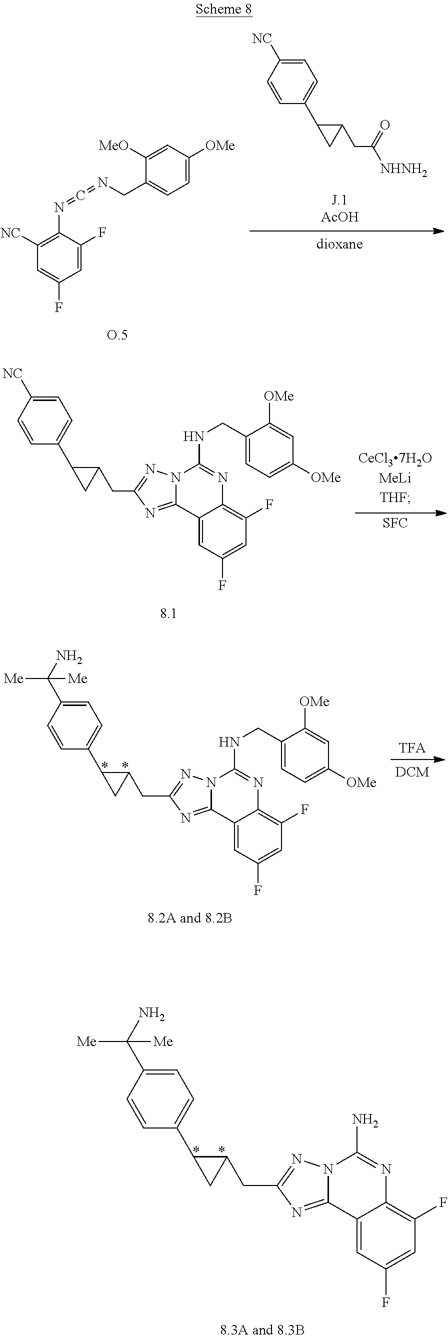 [SMILES: COc1ccc(CN=C=Nc2c(F)cc(F)cc2C#N)c(OC)c1 ; N#Cc1ccc(C2CC2CC(=O)NN)cc1 ; COc1ccc(CNc2nc3c(F)cc(F)cc3c3nc(CC4CC4c4ccc(C#N)cc4)nn23)c(OC)c1 ; "SFC" ; COc1ccc(CNc2nc3c(F)cc(F)cc3c3nc(CC4CC4c4ccc(C(C)(C)N)cc4)nn23)c(OC)c1 ; CC(C)(N)c1ccc(C2CC2Cc2nc3c4cc(F)cc(F)c4nc(N)n3n2)cc1]

### Step 1—Synthesis of Intermediate 8.1, 4-(2-((5-((2,4-dimethoxybenzyl)amino)-7,9-difluoro-[1,2,4]triazolo[1,5-c]quinazolin-2-yl)methyl)cyclopropyl)benzonitrile

AcOH (28 μL, 0.489 mmol) and 2-((((2,4-dimethoxybenzyl)imino)methylene)amino)-3,5-difluorobenzonitrile (319 mg, 0.969 mmol) were added to a stirred solution of 2-(2-(4-cyanophenyl)cyclopropyl)acetohydrazide (229 mg, 1.07 mmol) in dioxane (3.88 mL). The reaction was stirred vigorously at 65° C. for 1 h. The reaction was cooled to room temperature and concentrated under reduced pressure. The concentrated residue was purified by silica gel chromatography (gradient elution: 0-50% EtOAc/hexanes) to provide 4-(2-((5-((2,4-dimethoxybenzyl)amino)-7,9-difluoro-[1,2,4]triazolo[1,5-c]quinazolin-2-yl)methyl)cyclopropyl)benzonitrile. MS (ESI) m/z: calc'd for C₂₉H₂₅F₂N₆O₂ [M+H]⁺: 527.2. found 527.2.

### Step 2—Synthesis of Intermediates 8.2A and 8.2B, 2-(((1S,2R)-2-(4-(2-aminopropan-2-yl)phenyl)cyclopropyl)methyl)-N-(2,4-dimethoxybenzyl)-7,9-difluoro-[1,2,4]triazolo[1,5-c]quinazolin-5-amine and 2-(((1R,2S)-2-(4-(2-aminopropan-2-yl)phenyl)cyclopropyl)methyl)-N-(2,4-dimethoxybenzyl)-7,9-difluoro-[1,2,4]triazolo[1,5-c]quinazolin-5-amine

CeCl₃·7H₂O (781 mg, 2.1 mmol) was dried under reduced pressure at 150° C. for 16 h. After cooling to room temperature, THE (3 mL) was added. The reaction was stirred vigorously at room temperature for 1 h. MeLi (3.1 M, 0.68 mL, 2.1 mmol) was added dropwise over 1 min at −78° C. The reaction was stirred vigorously at −78° C. for 1 h. A solution of 4-(2-((5-((2,4-dimethoxybenzyl)amino)-7,9-difluoro-[1,2,4]triazolo[1,5-c]quinazolin-2-yl)methyl)cyclopropyl) benzonitrile (184 mg, 0.349 mmol) in THE (4 mL) was added at −78° C. The reaction was stirred vigorously at −78° C. for 3 h. The reaction was quenched with NH₄OH (1 mL), filtered, and washed with DCM (3×5 mL). The resulting filtrate was diluted with water (10 mL) and extracted with DCM (3×10 mL). The combined organic layers were dried over anhydrous MgSO₄, filtered, and concentrated under reduced pressure. The resulting crude residue was purified by silica gel chromatography (gradient elution: 0-100% [3:1 EtOAc/EtOH]/hexanes [w/1% Et₃N]). The racemic mixture was separated by chiral SFC (Column: OJ-H, 21×250 mm, 60% MeOH [w/0.1% NH₄OH]/CO₂) to provide Intermediate 8.2A (faster eluting) and Intermediate 8.2B (slower eluting). Intermediate 8.2A (faster eluting): MS (ESI) m/z: calc'd for C₃₁H₃₂F₂N₆NaO₂ [M+Na]⁺: 581.2. found 581.2. Intermediate 8.2B (slower eluting): MS (ESI) m/z: calc'd for C₃₁H₃₂F₂N₆NaO₂ [M+Na]⁺: 581.2. found 581.2.

### Step 3—Synthesis of Examples 8.3A and 8.3B, 2-(((1S,2R)-2-(4-(2-aminopropan-2-yl)phenyl)cyclopropyl)methyl)-7,9-difluoro-[1,2,4]triazolo[1,5-c]quinazolin-5-amine and 2-(((1R,2S)-2-(4-(2-aminopropan-2-yl)phenyl)cyclopropyl)methyl)-7,9-difluoro-[1,2,4]triazolo[1,5-c]quinazolin-5-amine

TFA (0.47 mL) was added to a stirred solution of Intermediate 8.2A (34 mg, 0.061 mmol) in DCM (0.61 mL). The reaction was stirred vigorously at 50° C. for 1 h. The reaction was cooled to room temperature and concentrated under reduced pressure. The resulting crude residue was purified by reversed-phase HPLC [Method B] to provide Example 8.3A (from faster eluting Intermediate 8.2A): MS (ESI) m/z: calc'd for C₂₂H₂₂F₂N₆Na [M+Na]⁺: 431.2. found 431.1. ¹H NMR (500 MHz, DMSO-d₆) δ 8.04 (s, 2H), 7.81-7.72 (m, 1H), 7.66 (ddd, J=11.8, 9.2, 2.8 Hz, 1H), 7.37 (d, J=8.3 Hz, 2H), 6.99 (d, J=8.3 Hz, 2H), 3.09-2.95 (m, 2H), 1.97-1.75 (m, 2H), 1.55-1.46 (m, 1H), 1.31 (s, 6H), 0.99 (ddd, J=19.6, 9.3, 5.0 Hz, 2H). A_{2A} IC₅₀ 38.1 nM (A).

TFA (0.48 mL) was added to a stirred solution of Intermediate 8.2B (35 mg, 0.063 mmol) in DCM (0.63 mL). The reaction was stirred vigorously at 50° C. for 1 h. The reaction was cooled to room temperature and concentrated under reduced pressure. The resulting crude residue was purified by reversed-phase HPLC [Method B] to provide Example 8.3B (from slower eluting Intermediate 8.2B): MS (ESI) m/z: calc'd for C₂₂H₂₂F₂N₆Na [M+Na]⁺: 431.2. found 431.2. ¹H NMR (500 MHz, DMSO-d₆) δ 8.04 (s, 2H), 7.81-7.72 (m, 1H), 7.66 (ddd, J=11.8, 9.2, 2.8 Hz, 1H), 7.37 (d, J=8.3 Hz, 2H), 6.99 (d, J=8.3 Hz, 2H), 3.09-2.95 (m, 2H), 1.97-1.75 (m, 2H), 1.55-1.46 (m, 1H), 1.31 (s, 6H), 0.99 (ddd, J=19.6, 9.3, 5.0 Hz, 2H). A_{2A} IC₅₀ 25.4 nM (A).

The following examples in Table 15 were prepared according to Scheme 8 and General Scheme 7, using Intermediate J.1 and the appropriate benzonitrile intermediate. Example 8.6B was prepared using MeMgBr instead of CeCl₃·7H₂O and MeLi. SFC conditions for the resolution involved in Step 2 are provided, following the table. Asterisk (*) indicates that A2B data is not available.

TABLE 15
Examples Prepared According to Scheme 8

| | | | A2a IC₅₀ |
|---|---|---|---|
| | | Observed | (nM) |
| | Structure | m/z | A2b IC₅₀ |
| Example | Name | [M + H]⁺ | (nM) |
| 8.4A (from faster eluting intermediate) | 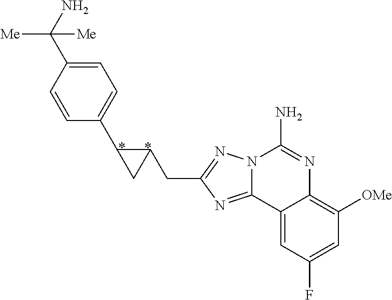 [SMILES: COc1cc(F)cc2c1nc(N)n1nc(CC3CC3c3ccc(C(C)(C)N)cc3)nc21] | 421.1 | 10.2 (A) * |
| | 2-(((1S, 2R or 1R,2S)-2-(4-(2-aminopropan-2-yl)phenyl)- | | |
| | cyclopropyl)methyl)-9-fluoro-7-methoxy-[1,2,4]triazolo- | | |
| | [1,5-c]quinazolin-5-amine | | |
| 8.4B (from slower eluting intermediate) | 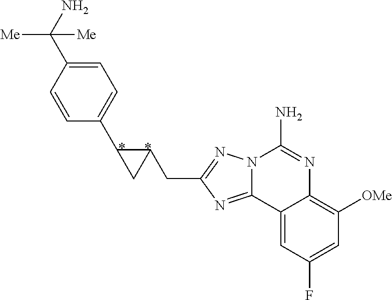 [SMILES: COc1cc(F)cc2c1nc(N)n1nc(CC3CC3c3ccc(C(C)(C)N)cc3)nc21] | 421.3 | 3.1 (A) 139 (A) |
| | 2-(((1R,2S or 1S,2R)-2-(4-(2-aminopropan-2-yl)phenyl)- | | |
| | cyclopropyl)methyl)-9-fluoro-7-methoxy-[1,2,4]triazolo- | | |
| | [1,5-c]quinazolin-5-amine | | |
| 8.5A (from faster eluting intermediate) | 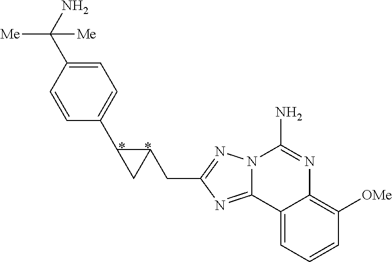 [SMILES: COc1cccc2c1nc(N)n1nc(CC3CC3c3ccc(C(C)(C)N)cc3)nc21] | 425.1 [M + Na]⁺ | 4.7 (A) * |
| | 2-(((1S,2R or 1R,2S)-2-(4-(2-aminopropan-2-yl)phenyl)- | | |
| | cyclopropyl)methyl)-7-methoxy-[1,2,4]triazolo- | | |
| | [1,5-c]quinazolin-5-amine | | |
| 8.5B (from slower eluting intermediate) | 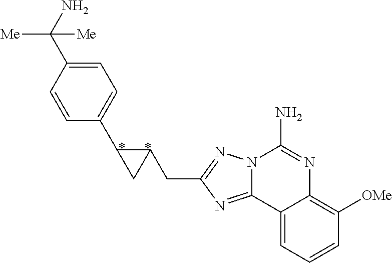 [SMILES: COc1cccc2c1nc(N)n1nc(CC3CC3c3ccc(C(C)(C)N)cc3)nc21] | 425.2 [M + Na]⁺ | 1.3 (A) 3.3 (A) |
| | 2-(((IR,2S or 1S,2R)-2-(4-(2-aminopropan-2- | | |
| | yl)phenyl)cyclopropyl)methyl)-7-methoxy- | | |
| | [1,2,4]triazolo[1,5-c]quinazolin-5-amine | | |
| 8.6B (from slower eluting intermediate) | 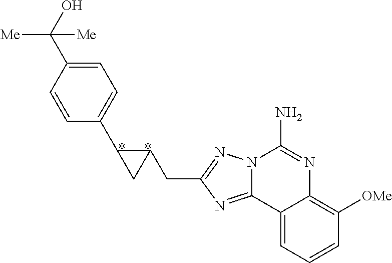 [SMILES: COc1cccc2c1nc(N)n1nc(CC3CC3c3ccc(C(C)(C)O)cc3)nc21] | 404.2 | 0.3 (A) * |
| | 2-(4-((1R,2S or 1S,2S)-2-((5-amino-7-methoxy-[1,2,4]triazolo- | | |
| | [1,5-c]quinazolin-2-yl)methyl)cyclopropyl)phenyl)propan-2-ol | | |

### Examples 8.4A and 8A4B

The racemic intermediate en route to Examples 8.4A and 8A4B was separated by chiral SFC (Column: AS-H, 21×250 mm, 600% MeOH [w/0.1% NH₄OH]/CO₂).

### Examples 8.5A and 8.5B

The racemic intermediate en route to Examples 8.5A and 8.5B was separated by chiral SFC (Column: UT, 21×250 mm, 60% o MeOH [w/0.1% NH₄OH]/CO₂).

### Example 8.6B

The racemic intermediate en route to Examples 8.6B was separated by chiral SFC (Column: Lux-3, 21×250 mm, 6500 MeOH [w/0.1% NH₄OH]/CO₂).

### Preparation of Example 9.1, 2-(4-(2-(5-amino-7-methoxy-[1,2,4]triazolo[1,5-c]quinazolin-2-yl)ethoxy)phenyl)-1,1,1,3,3,3-hexafluoropropan-2-ol

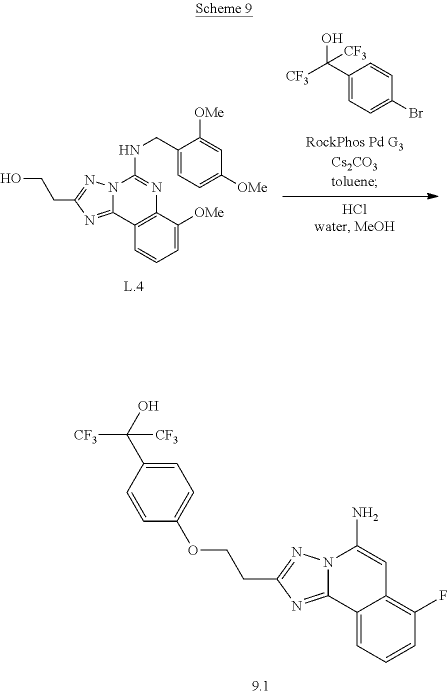 [SMILES: COc1ccc(CNc2nc3c(OC)cccc3c3nc(CCO)nn23)c(OC)c1 ; OC(c1ccc(Br)cc1)(C(F)(F)F)C(F)(F)F ; Nc1cc2c(F)cccc2c2nc(CCOc3ccc(C(O)(C(F)(F)F)C(F)(F)F)cc3)nn12]

2-(5-((2,4-Dimethoxybenzyl)amino)-7-methoxy-[1,2,4]triazolo[1,5-c]quinazolin-2-yl)ethan-1-ol (82 mg, 0.20 mmol), 2-(4-bromophenyl)-1,1,1,3,3,3-hexafluoropropan-2-ol (97 mg, 0.30 mmol), Cs₂CO₃ (131 mg, 0.40 mmol), and RockPhos Pd G3 (7.6 mg, 9.01 μmol) were combined. The reaction vessel was sealed and flushed with nitrogen for 5 min, evacuated for 1 min, and backfilled with nitrogen for 1 min. Toluene (2.0 mL) was added, and the reaction was degassed by sparging with nitrogen for 15 min, then backfilled with nitrogen for 1 min. The reaction was heated to 110° C. for 6 h in a microwave reactor. The reaction was cooled to room temperature, filtered, and concentrated under reduced pressure. The concentrated residue was dissolved in water (1 mL) and MeOH (1 mL). HCl (37% aq., 0.41 mL) was added. The reaction was stirred vigorously at 60° C. for 16 h, then cooled to room temperature, filtered, and washed with water (25 mL). The resulting filtrate was cooled to 0° C., quenched with a solution of NaOH (240 mg, 6.01 mmol) in water (5 mL), and extracted with 3:1 CHCl₃/i-PrOH (3×10 mL). The combined organic layers were dried over anhydrous MgSO₄, filtered, and concentrated under reduced pressure. The resulting crude residue was purified by reversed-phase HPLC [Method B] to provide 2-(4-(2-(5-amino-7-methoxy-[1,2,4]triazolo[1,5-c]quinazolin-2-yl)ethoxy)phenyl)-1,1,1,3,3,3-hexafluoropropan-2-ol (Example 9.1). MS (ESI) m/z calc'd for C₂₁H₁₈F₆N₅O₃ [M+H]⁺ 502.1. found 502.1. ¹H NMR (15 of 17 observed, 500 MHz, DMSO-d₆) δ 8.59 (s, 1H), 7.85 (s, 2H), 7.74 (d, J=7.9 Hz, 1H), 7.58 (d, J=8.4 Hz, 2H), 7.31 (t, J=7.9 Hz, 1H), 7.23 (d, J=7.6 Hz, 1H), 7.10 (d, J=8.9 Hz, 2H), 4.56 (t, J=6.3 Hz, 2H), 3.91 (s, 3H). A_{2A} IC₅₀ 1.7 nM (A).

### Preparation of Examples 10.3A and 10.3B, (S or R)-(2-(5-amino-7-methoxy-[1,2,4]triazolo[1,5-c]quinazolin-2-yl)ethyl)(cyclopropyl)(methylimino)-l6-sulfanone and (R or S)-(2-(5-amino-7-methoxy-[1,2,4]triazolo[1,5-c]quinazolin-2-yl)ethyl)(cyclopropyl)(methylimino)-λ⁶-sulfanone

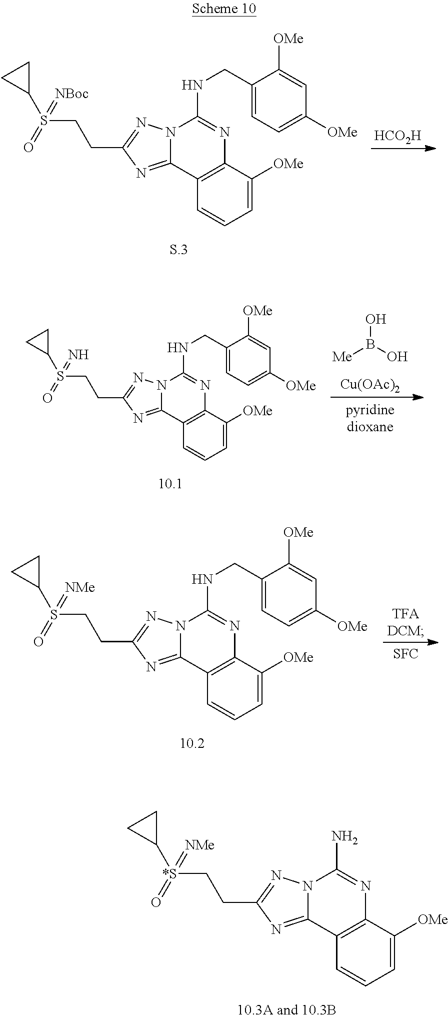

### Step 1—Synthesis of Intermediate 10.1, cyclopropyl(2-(5-((2,4-dimethoxybenzyl)amino)-7-methoxy-[1,2,4]triazolo[1,5-c]quinazolin-2-yl)ethyl)(imino)-λ⁶-sulfanone

HCO₂H (1.00 g, 21.7 mmol) was added to tert-butyl (cyclopropyl(2-(5-((2,4-dimethoxybenzyl)amino)-7-methoxy-[1,2,4]triazolo[1,5-c]quinazolin-2-yl)ethyl)(oxo)-λ⁶-sulfanylidene)carbamate (80 mg, 0.134 mmol) at 0° C. The reaction was stirred vigorously at room temperature for 2 h. The reaction was concentrated under reduced pressure. The concentrated residue was diluted with sat. aq. NaHCO₃ (10 mL) and extracted with DCM (3×5 mL). The combined organic layers were dried over anhydrous MgSO₄, filtered, and concentrated under reduced pressure to provide cyclopropyl(2-(5-((2,4-dimethoxybenzyl)amino)-7-methoxy-[1,2,4]triazolo[1,5-c]quinazolin-2-yl)ethyl)(imino)-λ⁶-sulfanone, which was used in the subsequent step without further purification. MS (ESI) m/z calc'd for C₂₄H₂₉N₆₀₄S [M+H]⁺ 497.2. found 497.2.

### Step 2—Synthesis of Intermediate 10.2, cyclopropyl(2-(5-((2,4-dimethoxybenzyl)amino)-7-methoxy-[1,2,4]triazolo[1,5-c]quinazolin-2-yl)ethyl)(methylimino)-λ⁶-sulfanone

Methylboronic acid (18.8 mg, 0.314 mmol), Cu(OAc)₂ (42.8 mg, 0.236 mmol) and pyridine (29.8 mg, 0.377 mmol) were added to a stirred solution of cyclopropyl(2-(5-((2,4-dimethoxybenzyl)amino)-7-methoxy-[1,2,4]triazolo[1,5-c]quinazolin-2-yl)ethyl)(imino)-λ⁶-sulfanone (78 mg, 0.157 mmol) in dioxane (2 mL). The reaction mixture stirred vigorously at 100° C. for 2 h. The reaction was cooled to room temperature and concentrated under reduced pressure to provide cyclopropyl(2-(5-((2,4-dimethoxybenzyl)amino)-7-methoxy-[1,2,4]triazolo[1,5-c]quinazolin-2-yl)ethyl)(methylimino)-λ⁶-sulfanone, which was used in the subsequent step without further purification. MS (ESI) m/z calc'd for C₂₅H₃₁N₆O₄S [M+H]⁺ 511.2. found 511.4.

### Step 3—Synthesis of Examples 10.3A and 10.3B, (S or R)-(2-(5-amino-7-methoxy-[1,2,4]triazolo[1,5-c]quinazolin-2-yl)ethyl)(cyclopropyl)(methylimino)-l6-sulfanone and (R or S)-(2-(5-amino-7-methoxy-[1,2,4]triazolo[1,5-c]quinazolin-2-yl)ethyl)(cyclopropyl)(methylimino)-λ⁶-sulfanone

TFA (1 mL) was added to a stirred solution of cyclopropyl(2-(5-((2,4-dimethoxybenzyl)amino)-7-methoxy-[1,2,4]triazolo[1,5-c]quinazolin-2-yl)ethyl)(methylimino)-λ⁶-sulfanone (66 mg, 0.097 mmol) in DCM (1 mL). The reaction was stirred vigorously at 40° C. for 16 h. The reaction was cooled to room temperature and concentrated under reduced pressure. The resulting crude residue was purified by reversed-phase HPLC [Method A]. The racemic mixture was separated by chiral SFC (Column: Chiralcel Amylose-C, 30×250 mm, gradient elution: 5-40% MeOH [w/0.05% Et₂NH]/CO₂) to provide Example 10.3A (faster eluting) and Example 10.3B (slower eluting).

Fasting eluting (Example 10.3A): MS (ESI) m/z: calc'd for C₁₆H₂₁N₆O₂S [M+H]⁺: 361.1. found 361.0. ¹H NMR (400 MHz, MeOD-d₄) δ 7.86 (dd, J=1.2, 8.1 Hz, 1H), 7.46-7.41 (m, 1H), 7.37-7.33 (m, 1H), 4.65-4.55 (m, 2H), 4.05 (s, 3H), 3.67 (t, J=7.2 Hz, 2H), 3.23-3.16 (m, 1H), 3.03 (s, 3H), 1.65-1.51 (m, 2H), 1.43-1.35 (m, 2H). A_{2A} IC₅₀ 1.4 nM (A).

Slower eluting (Example 10.3B): MS (ESI) m/z: calc'd for C₁₆H₂₁N₆O₂S [M+H]⁺: 361.1. found 361.2. ¹H NMR (400 MHz, MeOD-d₄) δ 7.86 (dd, J=1.0, 7.8 Hz, 1H), 7.47-7.41 (m, 1H), 7.37-7.33 (m, 1H), 4.65-4.56 (m, 2H), 4.05 (s, 3H), 3.67 (t, J=7.3 Hz, 2H), 3.24-3.16 (m, 1H), 3.03 (s, 3H), 1.66-1.52 (m, 2H), 1.44-1.36 (m, 2H). A_{2A} IC₅₀ 3.7 nM (A).

### Preparation of Examples 11.1A and 11.1B, (S or R)-(2-(5-amino-7-methoxy-[1,2,4]triazolo[1,5-c]quinazolin-2-yl)ethyl)(cyclopropyl)(imino)-26-sulfanone and (R or S)-(2-(5-amino-7-methoxy-[1,2,4]triazolo[1,5-c]quinazolin-2-yl)ethyl)(cyclopropyl)(imino)-λ⁶-sulfanone

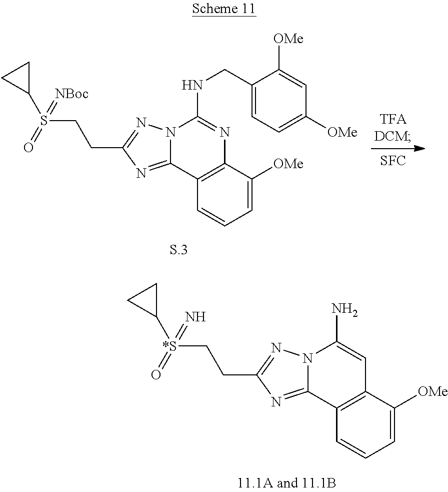

TFA (2 mL) was added to a stirred solution of tert-butyl (cyclopropyl(2-(5-((2,4-dimethoxybenzyl)amino)-7-methoxy-[1,2,4]triazolo[1,5-c]quinazolin-2-yl)ethyl)(oxo)-λ⁶-sulfanylidene)carbamate (35 mg, 0.059 mmol) in DCM (2 mL). The reaction was stirred vigorously at 45° C. for 16 h. The reaction was cooled to room temperature and concentrated under reduced pressure. The resulting crude residue was purified by reversed-phase HPLC [Method A]. The racemic mixture was separated by chiral SFC (Column: Chiralcel AS-3, 4.6×150 mm, gradient elution: 5-40% MeOH [w/0.05% Et₂NH]/CO₂) to provide Example 11.1A (faster eluting) and Example 11.1B (slower eluting).

Fasting eluting (Example 11.1A): MS (ESI) m/z: calc'd for C₁₆H₁₉N₆O₂S [M+H]⁺: 347.2. found 347.2. ¹H NMR (400 MHz, MeOD-d₄) δ 7.91 (dd, J=1.0, 8.1 Hz, 1H), 7.40 (t, J=8.1 Hz, 1H), 7.19 (d, J=7.3 Hz, 1H), 6.34 (br s, 2H), 4.07 (s, 3H), 3.79-3.73 (m, 2H), 3.61-3.56 (m, 2H), 2.57-2.49 (m, 1H), 1.24-1.18 (m, 2H), 1.05-1.01 (m, 2H). A_{2A} IC₅₀ 1.2 nM (A).

Slower eluting (Example 11.1B): MS (ESI) m/z: calc'd for C₁₆H₁₉N₆O₂S [M+H]⁺: 347.2. found 347.2. ¹H NMR (400 MHz, MeOD-d₄) δ 7.91 (dd, J=0.9, 7.9 Hz, 1H), 7.39 (t, J=8.1 Hz, 1H), 7.18 (d, J=7.8 Hz, 1H), 6.07 (br s, 2H), 4.07 (s, 3H), 3.77-3.72 (m, 2H), 3.61-3.55 (m, 2H), 2.55-2.48 (m, 1H), 1.24-1.17 (m, 2H), 1.04-1.02 (m, 2H). A_{2A} IC₅₀ 3.4 nM (A).

### Preparation of Examples 12.3A and 12.3B, (S or R)-9-fluoro-7-methoxy-2-(2-(3-(1-(trifluoromethyl)cyclopropyl)-5,6,7,8-tetrahydroimidazo[1,5-a]pyridin-7-yl)ethyl)-[1,2,4]triazolo[1,5-c]quinazolin-5-amine and (R or S)-9-fluoro-7-methoxy-2-(2-(3-(1-(trifluoromethyl)cyclopropyl)-5,6,7,8-tetrahydroimidazo[1,5-a]pyridin-7-yl)ethyl)-[1,2,4]triazolo[1,5-c]quinazolin-5-amine

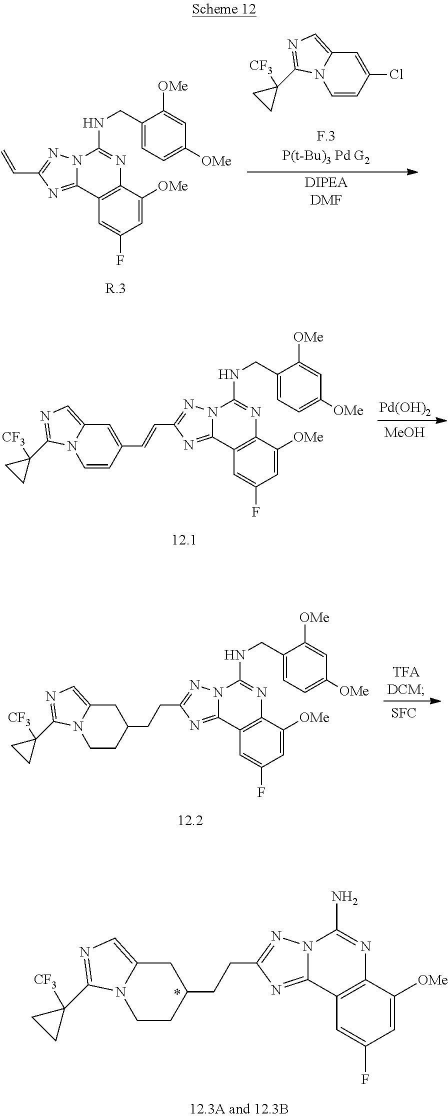

### Step 1—Synthesis of Intermediate 12.1, (E)-N-(2,4-dimethoxybenzyl)-9-fluoro-7-methoxy-2-(2-(3-(1-(trifluoromethyl)cyclopropyl)imidazo[1,5-a]pyridin-7-yl)vinyl)-[1,2,4]triazolo[1,5-c]quinazolin-5-amine

N-(2,4-dimethoxybenzyl)-9-fluoro-7-methoxy-2-vinyl-[1,2,4]triazolo[1,5-c]quinazolin-5-amine (115 mg, 0.28 mmol), P(t-Bu)₃ Pd G2 (36 mg, 0.07 mmol), 7-chloro-3-(1-(trifluoromethyl)cyclopropyl)imidazo[1,5-a]pyridine (81 mg, 0.31 mmol), and DIPEA (0.1 mL, 0.56 mmol) were combined. DMF (1.4 mL) was added, and the reaction was degassed by sparging with argon for 1 min. The reaction was heated to 115° C. for 16 h. The reaction was cooled to room temperature and concentrated under reduced pressure. The concentrated residue was diluted with DCM and washed with water. The combined organic layers were concentrated under reduced pressure. The resulting crude residue was purified by reversed-phase HPLC [Method B] to provide (E)-N-(2,4-dimethoxybenzyl)-9-fluoro-7-methoxy-2-(2-(3-(1-(trifluoromethyl)cyclopropyl)imidazo[1,5-a]pyridin-7-yl)vinyl)-[1,2,4]triazolo[1,5-c]quinazolin-5-amine. MS (ESI) m/z calc'd for C₃₂H₂₇F₄N₇O₃ [M+H]⁺ 634. found 634.

### Step 2—Synthesis of Intermediate 12.2, N-(2,4-dimethoxybenzyl)-9-fluoro-7-methoxy-2-(2-(3-(1-(trifluoromethyl)cyclopropyl)-5,6,7,8-tetrahydroimidazo[1, 5-a]pyridin-7-yl)ethyl)-[1,2,4]triazolo[1,5-c]quinazolin-5-amine

Pd(OH)₂/C (20%, 3.7 mg, 5.21 μmol) was added to a suspension of (E)-N-(2,4-dimethoxybenzyl)-9-fluoro-7-methoxy-2-(2-(3-(1-(trifluoromethyl)cyclopropyl)imidazo[1,5-a]pyridin-7-yl)vinyl)-[1,2,4]triazolo[1,5-c]quinazolin-5-amine (33 mg, 0.05 mmol) in MeOH (100 mL). The reaction was stirred vigorously at room temperature for 48 h under an atmosphere of H₂. The reaction was filtered and concentrated under reduced pressure to provide ethyl 3-(1-isopropyl-4,5,6,7-tetrahydro-1H-benzo[d][1,2,3]triazol-5-yl)propanoate, which was used in the subsequent step without further purification. MS (ESI) m/z calc'd for MS (ESI) m/z calc'd for C₃₂H₃₄F₄N₇₀₃ [M+H]⁺ 634. found 634.

### Step 3—Synthesis of Examples 12.3A and 12.3B, (S or R)-9-fluoro-7-methoxy-2-(2-(3-(1-(trifluoromethyl)cyclopropyl)-5,6,7,8-tetrahydroimidazo[1,5-a]pyridin-7-yl)ethyl)-[1,2,4]triazolo[1,5-c]quinazolin-5-amine and (R or S)-9-fluoro-7-methoxy-2-(2-(3-(1-(trifluoromethyl)cyclopropyl)-5,6,7,8-tetrahydroimidazo[1, 5-a]pyridin-7-yl)ethyl)-[1,2,4]triazolo[1,5-c]quinazolin-5-amine

TFA (0.11 mL) was added to N-(2,4-dimethoxybenzyl)-9-fluoro-7-methoxy-2-(2-(3-(1-(trifluoromethyl)cyclopropyl)-5,6,7,8-tetrahydroimidazo[1,5-a]pyridin-7-yl)ethyl)-[1,2,4]triazolo[1,5-c]quinazolin-5-amine (46 mg, 0.07 mmol). The reaction was stirred vigorously at 50° C. for 1.5 h. The reaction was cooled to room temperature and concentrated under reduced pressure. The resulting crude residue was purified by reversed-phase HPLC [Method B]. The racemic mixture was separated by chiral SFC (Column: CC4, 30×250 mm, 75% MeOH [w/0.1% NH₄OH]/CO₂) to provide Example 12.3A (faster eluting) and Example 12.3.B (slower eluting).

Fasting eluting (Example 12.3A): MS (ESI) m/z: calc'd for C₂₃H₂₄F₄N₇₀ [M+H]⁺: 490. found 490. ¹H NMR (600 MHz, DMSO-d₆) δ 7.79 (s, 2H), 7.41 (dd, J=8.4, 2.7 Hz, 1H), 7.17 (dd, J=11.1, 2.7 Hz, 1H), 6.62 (s, 1H), 4.20-4.16 (m, 1H), 3.93 (s, 3H), 3.84-3.80 (m, 1H), 3.02-2.98 (m, 2H), 2.38-2.36 (m, 1H), 2.12-2.09 (m, 1H), 1.95-1.87 (m, 2H), 1.66-1.61 (m, 1H), 1.42-1.07 (m, 4H), 0.86-0.78 (m, 2H). A_{2A} IC₅₀ 5.7 nM (A).

Slower eluting (Example 12.3B): MS (ESI) m/z: calc'd for C₂₃H₂₄F₄N₇₀ [M+H]⁺: 490. found 490. ¹H NMR (600 MHz, DMSO-d₆) δ 7.79 (s, 2H), 7.41 (dd, J=8.4, 2.7 Hz, 1H), 7.17 (dd, J=11.1, 2.7 Hz, 1H), 6.62 (s, 1H), 4.20-4.16 (m, 1H), 3.93 (s, 3H), 3.84-3.80 (m, 1H), 3.02-2.98 (m, 2H), 2.38-2.36 (m, 1H), 2.12-2.09 (m, 1H), 1.95-1.87 (m, 2H), 1.66-1.61 (m, 1H), 1.42-1.07 (m, 4H), 0.86-0.78 (m, 2H). A_{2A} IC₅₀ 6.7 nM (A).

### Preparation of Examples 13.2A and 13.2B, (S or R)-9-fluoro-8-methoxy-2-(2-(methylsulfonyl)propyl)-[1,2,4]triazolo[1,5-c]quinazolin-5-amine and (R or S)-9-fluoro-8-methoxy-2-(2-(methylsulfonyl)propyl)-[1,2,4]triazolo[1,5-c]quinazolin-5-amine

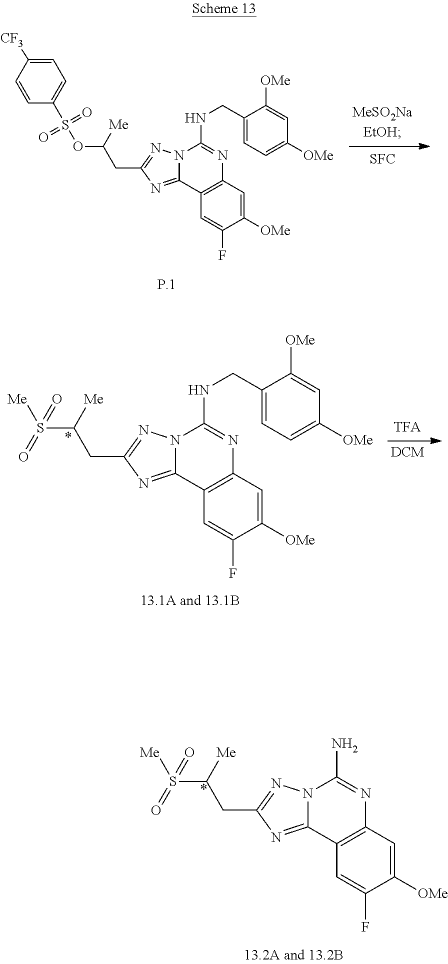

### Step 1—Synthesis of Intermediates 13.1A and 13.B, (S or R)—N-(2,4-dimethoxybenzyl)-9-fluoro-8-methoxy-2-(2-(methylsulfonyl)propyl)-[1,2,4]triazolo[1,5-c]quinazolin-5-amine and (R or S)—N-(2,4-dimethoxybenzyl)-9-fluoro-8-methoxy-2-(2-(methylsulfonyl)propyl)-[1,2,4]triazolo[1,5-c]quinazolin-5-amine

MeSO₂Na (141 mg, 1.39 mmol) was added to a stirred solution of N-(2,4-dimethoxybenzyl)-9-fluoro-8-methoxy-2-(2-(methylsulfonyl)propyl)-[1,2,4]triazolo[1,5-c]quinazolin-5-amine (180 mg, 0.277 mmol) in EtOH (5 mL). The reaction was stirred vigorously at 80° C. for 16 h. The reaction was cooled to room temperature and concentrated under reduced pressure. The resulting crude residue was purified by reversed-phase HPLC [Method A]. The racemic mixture was separated by chiral SFC (Column: AS-3, 4.6×150 mm, gradient elution: 5-40% MeOH [w/0.05% Et₂NH]/CO₂) to provide Intermediate 13.1A (faster eluting) and Intermediate 13.1B (slower eluting). Intermediate 13.1A (faster eluting): MS (ESI) m/z: calc'd for C₂₃H₂₇FN₅O₅S [M+H]⁺: 504.2. found 504.2. Intermediate 13.1B (slower eluting): MS (ESI) m/z: calc'd for C₃₁H₃₂F₂N₆NaO₂ [M+Na]⁺: 581.2. found 581.2.

### Step 2—Synthesis of Examples 13.2A and 13.2B, (S or R)-9-fluoro-8-methoxy-2-(2-(methylsulfonyl)propyl)-[1,2,4]triazolo[1,5-c]quinazolin-5-amine and (R or S)-9-fluoro-8-methoxy-2-(2-(methylsulfonyl)propyl)-[1,2,4]triazolo[1,5-c]quinazolin-5-amine

TFA (1 mL) was added to a stirred solution of Intermediate 13.1A (20 mg, 0.040 mmol) in DCM (1 mL). The reaction was stirred vigorously at 40° C. for 16 h. The reaction was cooled to room temperature and concentrated under reduced pressure. The resulting crude residue was purified by reversed-phase HPLC [Method A] to provide Example 13.2A (from faster eluting Intermediate 13.1A): MS (ESI) m/z: calc'd for C₁₄H₁₇FN₅O₃ [M+H]⁺: 354.2. found 354.2. ¹H NMR (400 MHz, MeOD-d₄) δ 7.90 (d, J=10.8 Hz, 1H), 7.23 (d, J=7.6 Hz, 1H), 4.01 (s, 3H), 3.80 (br s, 1H), 3.64 (dd, J=4.3, 15.0 Hz, 1H), 3.13 (d, J=4.9 Hz, 1H), 3.03 (s, 3H), 1.47 (d, J=6.8 Hz, 3H). A_{2A} IC₅₀ 0.7 nM (A), A_{2B} IC₅₀ 61.4 nM (A).

TFA (1 mL) was added to a stirred solution of Intermediate 13.1B (19 mg, 0.038 mmol) in DCM (1 mL). The reaction was stirred vigorously at 40° C. for 16 h. The reaction was cooled to room temperature and concentrated under reduced pressure. The resulting crude residue was purified by reversed-phase HPLC [Method A] to provide Example 13.2B (from faster eluting Intermediate 13.1B): MS (ESI) m/z: calc'd for C₁₄H₁₇FN₅O₃ [M+H]⁺: 354.2. found 354.1. ¹H NMR (400 MHz, MeOD-d₄) δ 7.90 (d, J=10.8 Hz, 1H), 7.23 (d, J=7.6 Hz, 1H), 4.01 (s, 3H), 3.81 (br d, J=7.1 Hz, 1H), 3.64 (dd, J=4.2, 14.9 Hz, 1H), 3.16-3.11 (m, 1H), 3.03 (s, 3H), 1.47 (d, J=7.1 Hz, 3H). A_{2A} IC₅₀ 8.1 nM (A), A_{2B} IC₅₀ 1055 nM (A).

The following examples in Table 16 were prepared according to Scheme 13 and General Scheme 8, using the appropriate sulfonate and sulfinate. Compounds were generally purified by filtering and washing with an appropriate solvent, silica gel chromatography, or reversed-phase prep-HPLC. Asterisk (*) indicates that A2B data is not available.

TABLE 16
Examples Prepared According to Scheme 13

| | | | A2a IC₅₀ |
|---|---|---|---|
| | | Observed | (nM) |
| | Structure | m/z | A2b IC₅₀ |
| Example | Name | [M + H]⁺ | (nM) |
| 13.3 | 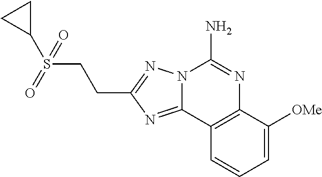 [SMILES: COc1cccc2c1nc(N)n1nc(CCS(=O)(=O)C3CC3)nc21] | 348.1 | 1.6 (A) * |
| | 2-(2-(cyclopropylsulfonyl)ethyl)-7-methoxy- | | |
| | [1,2,4]triazolo[1,5-c]quinazolin-5-amine | | |
| 13.4 | 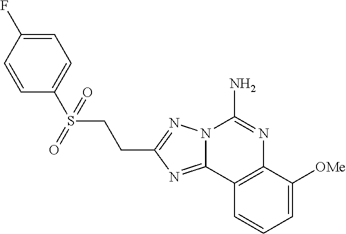 [SMILES: COc1cccc2c1nc(N)n1nc(CCS(=O)(=O)c3ccc(F)cc3)nc21] | 402.0 | 0.3 (A) 973 (A) |
| | 2-(2-((4-fluorophenyl)sulfonyl)ethyl)-7-methoxy- | | |
| | [1,2,4]triazolo[1,5-c]quinazolin-5-amine | | |
| 13.5 | 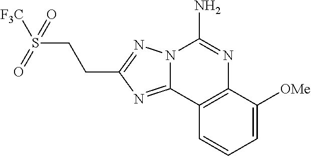 [SMILES: COc1cccc2c1nc(N)n1nc(CCS(=O)(=O)C(F)(F)F)nc21] | 354.0 | 10.9 (A) 81.3 (A) |
| | 7-methoxy-2-(2-((trifluoromethyl)sulfonyl)ethyl)- | | |
| | [1,2,4]triazolo[1,5-c]quinazolin-5-amine | | |
| 13.6 | 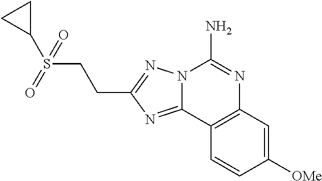 [SMILES: COc1ccc2c(c1)nc(N)n1nc(CCS(=O)(=O)C3CC3)nc21] | 348.1 | 8.2 (A) * |
| | 2-(2-(cyclopropylsulfonyl)ethyl)-8-methoxy- | | |
| | [1,2,4]triazolo[1,5-c]quinazolin-5-amine | | |
| 13.7 | 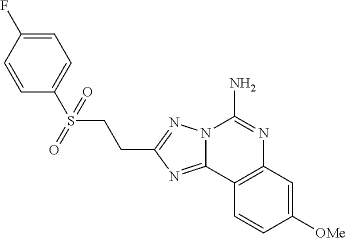 [SMILES: COc1ccc2c(c1)nc(N)n1nc(CCS(=O)(=O)c3ccc(F)cc3)nc21] | 402.1 | 170 (A) 607 (A) |
| | 2-(2-((4-fluorophenyl)sulfonyl)ethyl)-8-methoxy- | | |
| | [1,2,4]triazolo[1,5-c]quinazolin-5-amine | | |
| 13.8 | 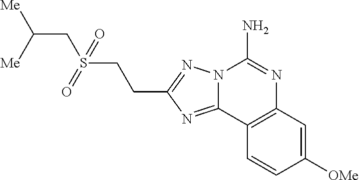 [SMILES: COc1ccc2c(c1)nc(N)n1nc(CCS(=O)(=O)CC(C)C)nc21] | 364.1 | 76.9 (A) 894 (A) |
| | 2-(2-(isobutylsulfonyl)ethyl)-8-methoxy-[1,2,4]triazolo- | | |
| | [1,5-c]quinazolin-5-amine | | |
| 13.9 | 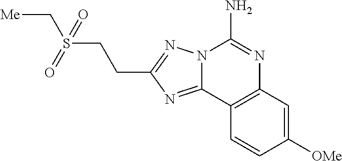 [SMILES: CCS(=O)(=O)CCc1nc2c3ccc(OC)cc3nc(N)n2n1] | 336.2 | 246 (A) 1065 (A) |
| | 2-(2-(ethylsulfonyl)ethyl)-8-methoxy-[1,2,4]triazolo- | | |
| | [1,5-c]quinazolin-5-amine | | |
| 13.10 | 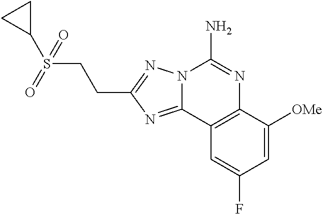 [SMILES: COc1cc(F)cc2c1nc(N)n1nc(CCS(=O)(=O)C3CC3)nc21] | 366.1 | 0.8 (A) 9.8 (A) |
| | 2-(2-(cyclopropylsulfonyl)ethyl)-9-fluoro-7-methoxy- | | |
| | [1,2,4]triazolo[1,5-c]quinazolin-5-amine | | |
| 13.11 | 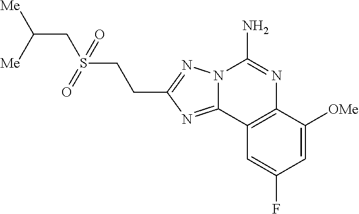 [SMILES: COc1cc(F)cc2c1nc(N)n1nc(CCS(=O)(=O)CC(C)C)nc21] | 382.1 | 28.8 (A) 666 (A) |
| | 9-fluoro-2-(2-(isobutylsulfonyl)ethyl)-7-methoxy- | | |
| | [1,2,4]triazolo[1,5-c]quinazolin-5-amine | | |
| 13.12 | 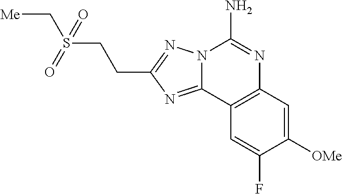 [SMILES: CCS(=O)(=O)CCc1nc2c3cc(F)c(OC)cc3nc(N)n2n1] | 354.1 | 165 (A) 1716 (A) |
| | 2-(2-(ethylsulfonyl)ethyl)-9-fluoro-8-methoxy- | | |
| | [1,2,4]triazolo[1,5-c]quinazolin-5-amine | | |
| 13.13 | 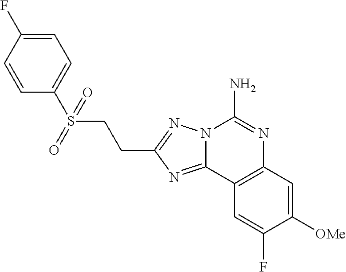 [SMILES: COc1cc2nc(N)n3nc(CCS(=O)(=O)c4ccc(F)cc4)nc3c2cc1F] | 420.1 | 57.7 (A) 1039 (A) |
| | 9-fluoro-2-(2-((4-fluorophenyl)sulfonyl)ethyl)-8- | | |
| | methoxy-[1,2,4]triazolo[1,5-c]quinazolin-5-amine | | |
| 13.14 | 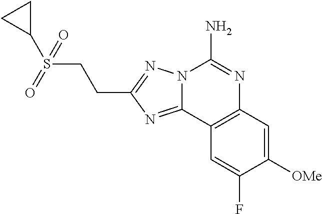 [SMILES: COc1cc2nc(N)n3nc(CCS(=O)(=O)C4CC4)nc3c2cc1F] | 366.1 | 9.1 (A) 197 (A) |
| | 2-(2-(cyclopropylsulfonyl)ethyl)-9-fluoro-8-methoxy- | | |
| | [1,2,4]triazolo[1,5-c]quinazolin-5-amine | | |
| 13.15 | 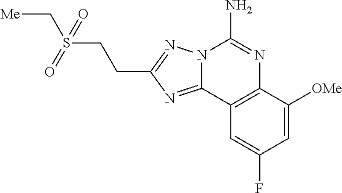 [SMILES: CCS(=O)(=O)CCc1nc2c3cc(F)cc(OC)c3nc(N)n2n1] | 354.1 | 9.8 (A) 220 (A) |
| | 2-(2-(ethylsulfonyl)ethyl)-9-fluoro-7-methoxy- | | |
| | [1,2,4]triazolo[1,5-c]quinazolin-5-amine | | |
| 13.16 | 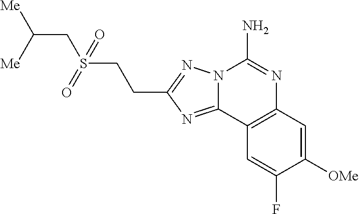 [SMILES: COc1cc2nc(N)n3nc(CCS(=O)(=O)CC(C)C)nc3c2cc1F] | 382.1 | 107 (A) 1081 (A) |
| | 9-fluoro-2-(2-(isobutylsulfonyl)ethyl)-8-methoxy- | | |
| | [1,2,4]triazolo[1,5-c]quinazolin-5-amine | | |
| 13.17 | 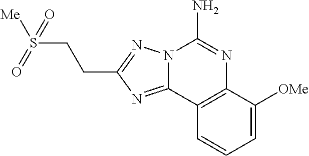 [SMILES: COc1cccc2c1nc(N)n1nc(CCS(C)(=O)=O)nc21] | 322.1 | 28.1 (A) 410 (A) |
| | 7-methoxy-2-(2-(methylsulfonyl)ethyl)- | | |
| | [1,2,4]triazolo[1,5-c]quinazolin-5-amine | | |
| 13.18 | 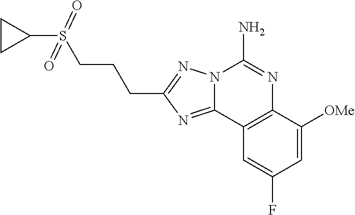 [SMILES: COc1cc(F)cc2c1nc(N)n1nc(CCCS(=O)(=O)C3CC3)nc21] | 380.1 | 1.1 (A) 112 (A) |
| | 2-(3-(cyclopropylsulfonyl)propyl)-9-fluoro-7-methoxy- | | |
| | [1,2,4]triazolo[1,5-c]quinazolin-5-amine | | |
| 13.19 | 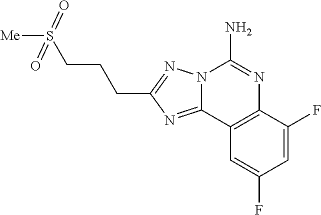 [SMILES: CS(=O)(=O)CCCc1nc2c3cc(F)cc(F)c3nc(N)n2n1] | 342.1 | 21.8 (A) * |
| | 7,9-difluoro-2-(3-(methylsulfonyl)propyl)- | | |
| | [1,2,4]triazolo[1,5-c]quinazolin-5-amine | | |
| 13.20 | 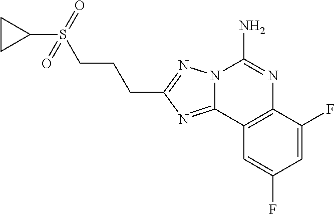 [SMILES: Nc1nc2c(F)cc(F)cc2c2nc(CCCS(=O)(=O)C3CC3)nn12] | 368.1 | 34.2 (A) * |
| | 2-(3-(cyclopropylsulfonyl)propyl)-7,9-difluoro- | | |
| | [1,2,4]triazolo[1,5-c]quinazolin-5-amine | | |

### Preparation of Examples 14.3A and 14.3B, (S or R)-5-(2-(5-amino-9-fluoro-7-methoxy-[1,2,4]triazolo[1,5-c]quinazolin-2-yl)ethyl)-1-(3,3-difluorocyclobutyl)-4,5,6,7-tetrahydro-1H-benzo[d][1,2,3]triazol-5-ol and (R or S)-5-(2-(5-amino-9-fluoro-7-methoxy-[1,2,4]triazolo[1,5-c]quinazolin-2-yl)ethyl)-1-(3,3-difluorocyclobutyl)-4,5,6, 7-tetrahydro-1H-benzo[d][1,2,3]triazol-5-ol

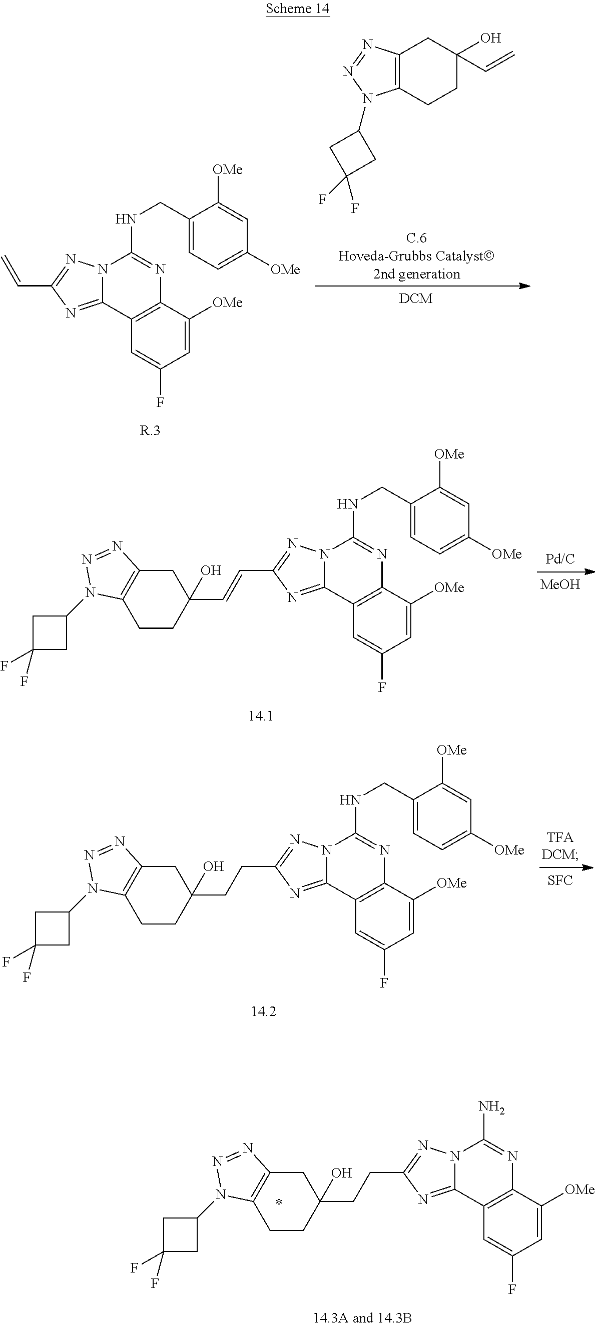

### Step 1—Synthesis of Intermediate 14.1, (E)-1-(3,3-difluorocyclobutyl)-5-(2-(5-((2,4-dimethoxybenzyl)amino)-9-fluoro-7-methoxy-[1,2,4]triazolo[1,5-c]quinazolin-2-yl)vinyl)-4,5,6,7-tetrahydro-1H-benzo[d][1,2,3]triazol-5-ol

1-(3,3-Difluorocyclobutyl)-5-vinyl-4,5,6,7-tetrahydro-1H-benzo[d][1,2,3]triazol-5-ol (70 mg, 0.274 mmol) and Hoveyda-Grubbs 2^{nd} Generation Catalyst© 2^{nd} generation (17.2 mg, 0.027 mmol) were added to a stirred solution of N-(2,4-dimethoxybenzyl)-9-fluoro-7-methoxy-2-vinyl-[1,2,4]triazolo[1,5-c]quinazolin-5-amine (112 mg, 0.274 mmol) in DCM (2 mL). The reaction was stirred vigorously at 15° C. for 15 h. The reaction was filtered and concentrated under reduced pressure. The resulting crude residue was purified by preparative TLC (10% EtOAc/DCM) to provide (E)-1-(3,3-difluorocyclobutyl)-5-(2-(5-((2,4-dimethoxybenzyl)amino)-9-fluoro-7-methoxy-[1,2,4]triazolo[1,5-c]quinazolin-2-yl)vinyl)-4,5,6,7-tetrahydro-1H-benzo[d][1,2,3]triazol-5-ol. MS (ESI) m/z calc'd for C₃₁H₃₂F₃N₈O₄ [M+H]⁺ 637.6. found 637.3.

### Step 2—Synthesis of Intermediate 14.2, 1-(3,3-difluorocyclobutyl)-5-(2-(5-((2,4-dimethoxybenzyl)amino)-9-fluoro-7-methoxy-[1,2,4]triazolo[1,5-c]quinazolin-2-yl)ethyl)-4,5,6,7-tetrahydro-1H-benzo[d][1,2,3]triazol-5-ol

Pd/C (1.8 mg, 0.017 mmol) was added to a stirred solution of (E)-1-(3,3-difluorocyclobutyl)-5-(2-(5-((2,4-dimethoxybenzyl)amino)-9-fluoro-7-methoxy-[1,2,4]triazolo[1,5-c]quinazolin-2-yl)vinyl)-4,5,6,7-tetrahydro-1H-benzo[d][1,2,3]triazol-5-ol (11 mg, 0.017 mmol) in MeOH (5 mL). The reaction was stirred vigorously at 15° C. for 3 h under an atmosphere of H₂ at 15 psi. The reaction was filtered and concentrated under reduced pressure to provide 1-(3,3-difluorocyclobutyl)-5-(2-(5-((2,4-dimethoxybenzyl)amino)-9-fluoro-7-methoxy-[1,2,4]triazolo[1,5-c]quinazolin-2-yl)ethyl)-4,5,6,7-tetrahydro-1H-benzo[d][1,2,3]triazol-5-ol, which was used in the subsequent step without further purification. MS (ESI) m/z calc'd for MS (ESI) m/z calc'd for C₃₁H₃₄F₃N₈O₄ [M+H]⁺ 639.2. found 639.3.

### Step 3—Synthesis of Examples 14.3A and 14.3B, (S or R)-5-(2-(5-amino-9-fluoro-7-methoxy-[1,2,4]triazolo[1,5-c]quinazolin-2-yl)ethyl)-1-(3,3-difluorocyclobutyl)-4,5,6,7-tetrahydro-1H-benzo[d][1,2,3]triazol-5-ol and (R or S)-5-(2-(5-amino-9-fluoro-7-methoxy-[1,2,4]triazolo[1,5-c]quinazolin-2-yl)ethyl)-1-(3,3-difluorocyclobutyl)-4,5,6,7-tetrahydro-1H-benzo[d][1,2,3]triazol-5-ol

TFA (1 mL) was added to a stirred solution of 1-(3,3-difluorocyclobutyl)-5-(2-(5-((2,4-dimethoxybenzyl)amino)-9-fluoro-7-methoxy-[1,2,4]triazolo[1,5-c]quinazolin-2-yl)ethyl)-4,5,6,7-tetrahydro-1H-benzo[d][1,2,3]triazol-5-ol (9 mg, 0.014 mmol) in DCM (1 mL). The reaction was stirred vigorously at 50° C. for 15 h. The reaction was cooled to room temperature and concentrated under reduced pressure. The resulting crude residue was purified by preparative TLC (10% MeOH/DCM). The racemic mixture was separated by chiral SFC (Column: AD-3, 4.6×50 mm, gradient elution: 5-40% i-PrOH [w/0.05% Et₂NH]/CO₂) to provide Example 14.3A (faster eluting) and Example 14.3B (slower eluting).

Fasting eluting (Example 14.3A): MS (ESI) m/z: calc'd for C₂₂H₂₄F₃N₈O₂ [M+H]⁺: 489.1. found 489.2. ¹H NMR (400 MHz, MeOD-d₄) δ 7.41 (dd, J=2.7, 8.2 Hz, 1H), 6.93 (dd, J=2.5, 10.5 Hz, 1H), 4.80-4.68 (m, 1H), 3.95 (s, 3H), 3.18-3.03 (m, 4H), 2.81 (s, 1H), 2.78-2.69 (m, 2H), 2.67-2.57 (m, 1H), 2.16-2.08 (m, 3H), 2.06-1.91 (m, 2H), 1.84-1.74 (m, 2H), 1.56-1.46 (m, 3H). A_{2A} IC₅₀ 1.1 nM (A).

Slower eluting (Example 14.3B): MS (ESI) m/z: calc'd for C₂₂H₂₄F₃N₈O₂ [M+H]⁺: 489.1. found 489.2. ¹H NMR (400 MHz, MeOD-d₄) δ 7.41 (dd, J=2.7, 8.2 Hz, 1H), 6.93 (dd, J=2.5, 10.5 Hz, 1H), 4.80-4.68 (m, 1H), 3.95 (s, 3H), 3.18-3.03 (m, 4H), 2.81 (s, 1H), 2.78-2.69 (m, 2H), 2.67-2.57 (m, 1H), 2.16-2.08 (m, 3H), 2.06-1.91 (m, 2H), 1.84-1.74 (m, 2H), 1.56-1.46 (m, 3H). A_{2A} IC₅₀ 3.4 nM (A).

### Preparation of Example 15.4, (5-amino-10-(3,4-dihydro-2H-pyran-5-yl)-7-methoxy-[1,2,4]triazolo[1,5-c]quinazolin-2-yl)methanol

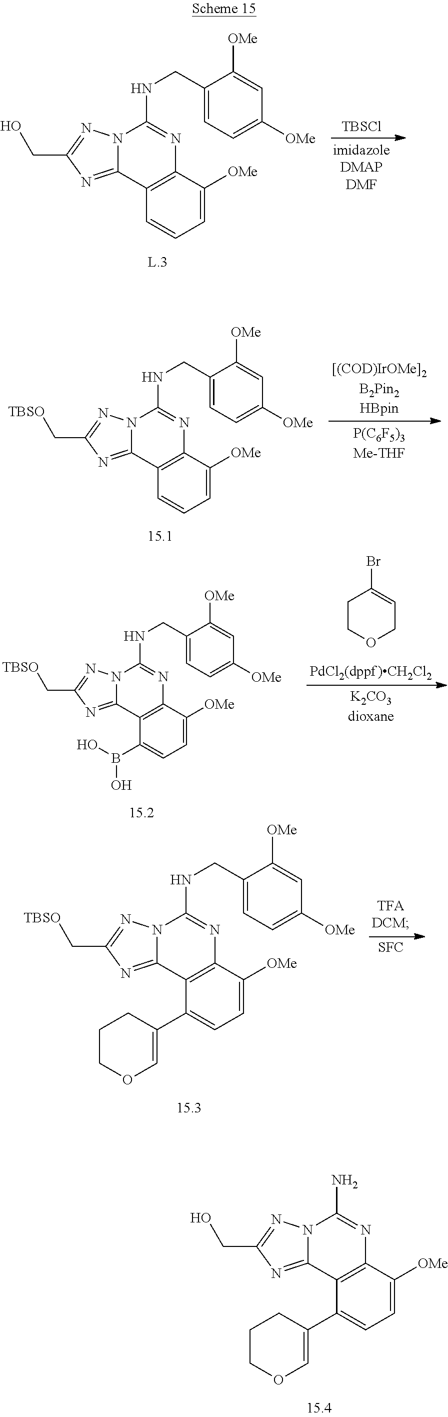

### Step 1—Synthesis of Intermediate 15.1, 2-(((tert-butyldimethylsilyl)oxy)methyl)-N-(2,4-dimethoxybenzyl)-7-methoxy-[1,2,4]triazolo[1,5-c]quinazolin-5-amine

1H-imidazole (310 mg, 4.55 mmol), DMAP (93 mg, 0.759 mmol), and TBSCl (343 mg, 2.28 mmol) were added to a stirred solution of (5-((2,4-dimethoxybenzyl)amino)-7-methoxy-[1,2,4]triazolo[1,5-c]quinazolin-2-yl)methanol (600 mg, 1.52 mmol) in DMF (10 mL). The reaction was stirred vigorously at 80° C. for 16 h. The reaction was cooled to room temperature and concentrated under reduced pressure. The resulting crude residue was purified by silica gel chromatography (gradient elution: 0-25% EtOAc/petroleum ether) to provide 2-(((tert-butyldimethylsilyl)oxy)methyl)-N-(2,4-dimethoxybenzyl)-7-methoxy-[1,2,4]triazolo[1,5-c]quinazolin-5-amine. MS (ESI) m/z calc'd for C₂₆H₃₆N₅₀₄Si [M+H]⁺ 510.4. found 510.4.

### Step 2—Synthesis of Intermediate 15.2, (2-(((tert-butyldimethylsilyl)oxy)methyl)-5-((2,4-dimethoxybenzyl)amino)-7-methoxy-[1,2,4]triazolo[1,5-c]quinazolin-10-yl)boronic acid

HBPin (13 mg, 0.102 mmol) was added to a stirred solution of P(C₆F₅)₃ (21 mg, 0.039 mmol) and [(COD)IrOMe]2 (7 mg, 10.6 μmol) in Me-THF (0.2 mL). A solution of 2-(((tert-butyldimethylsilyl)oxy)methyl)-N-(2,4-dimethoxybenzyl)-7-methoxy-[1,2,4]triazolo[1,5-c]quinazolin-5-amine (100 mg, 0.196 mmol) and B₂Pin₂ 75 mg, 0.295 mmol) in Me-THF (1 mL) was then added. The reaction was stirred vigorously at 100° C. for 16 h. The reaction was cooled to room temperature, filtered, and concentrated under reduced pressure. The resulting crude residue was purified by reversed-phase HPLC [Method B] to provide (2-(((tert-butyldimethylsilyl)oxy)methyl)-5-((2,4-dimethoxybenzyl)amino)-7-methoxy-[1,2,4]triazolo[1,5-c]quinazolin-10-yl)boronic acid. MS (ESI) m/z calc'd for C₂₆H₃₇BN₅O₆Si [M+H]⁺ 554.5. found 554.5.

### Step 3—Synthesis of Intermediate 15.3, 2-(((tert-butyldimethylsilyl)oxy)methyl)-10-(3,4-dihydro-2H-pyran-5-yl)-N-(2,4-dimethoxybenzyl)-7-methoxy-[1,2,4]triazolo[1,5-c]quinazolin-5-amine

K₂CO₃ (18 mg, 0.130 mmol), 4-bromo-3,6-dihydro-2H-pyran (12 mg, 0.074 mmol), and PdCl₂(dppf)·CH₂Cl₂ (10 mg, 0.012 mmol) was added to a stirred solution of (2-(((tert-butyldimethylsilyl)oxy)methyl)-5-((2,4-dimethoxybenzyl)amino)-7-methoxy-[1,2,4]triazolo[1,5-c]quinazolin-10-yl)boronic acid (20 mg, 0.036 mmol) in dioxane (2 mL) and water (0.4 mL). The reaction was stirred vigorously at 80° C. for 16 h. The reaction was cooled to room temperature, diluted with water (10 mL), and extracted with DCM (2×10 mL). The combined organic layers were concentrated under reduced pressure. The resulting crude residue was purified by reversed-phase HPLC [Method B] to provide 2-(((tert-butyldimethylsilyl)oxy)methyl)-10-(3,4-dihydro-2H-pyran-5-yl)-N-(2,4-dimethoxybenzyl)-7-methoxy-[1,2,4]triazolo[1,5-c]quinazolin-5-amine. MS (ESI) m/z calc'd for C₃₁H₄₁N₅O₅Si [M+H]⁺ 592.5. found 592.5.

### Step 4—Synthesis of Example 15.4, (5-amino-10-(3,4-dihydro-2H-pyran-5-yl)-7-methoxy-[1,2,4]triazolo[1,5-c]quinazolin-2-yl)methanol

TFA (1 mL) was added to a stirred solution of 2-(((tert-butyldimethylsilyl)oxy)methyl)-10-(3,4-dihydro-2H-pyran-5-yl)-N-(2,4-dimethoxybenzyl)-7-methoxy-[1,2,4]triazolo[1,5-c]quinazolin-5-amine (9 mg, 0.015 mmol) in DCM (1 mL). The reaction was stirred vigorously at 20° C. for 16 h. The reaction was concentrated under reduced pressure. The resulting crude residue was purified by reversed-phase HPLC [Method A] to provide (5-amino-10-(3,4-dihydro-2H-pyran-5-yl)-7-methoxy-[1,2,4]triazolo[1,5-c]quinazolin-2-yl)methanol (Example 15.4). MS (ESI) m/z: calc'd for C₁₆H₁₈N₅O₃ [M+H]⁺: 328.2. found 328.2. ¹H NMR (500 MHz, MeOD-d₄) δ 7.36 (br d, J=8.09 Hz, 1H), 7.24 (d, J=7.78 Hz, 1H), 5.71 (br s, 1H), 4.75-4.82 (m, 2H), 4.37 (br s, 2H), 4.05-4.16 (m, 5H), 3.21-3.31 (m, 2H), 2.50 (br s, 2H). A_{2A} IC₅₀ 2.7 nM (A).

### Preparation of Examples 16.3 and 16.4, ethyl 4-(4-((5-amino-9-fluoro-7-methoxy-[1,2,4]triazolo[1,5-c]quinazolin-2-yl)methyl)-1H-pyrazol-1-yl)butanoate and 5-(4-((5-amino-9-fluoro-7-methoxy-[1,2,4]triazolo[1,5-c]quinazolin-2-yl)methyl)-1H-pyrazol-1-yl)-2-methylpentan-2-ol

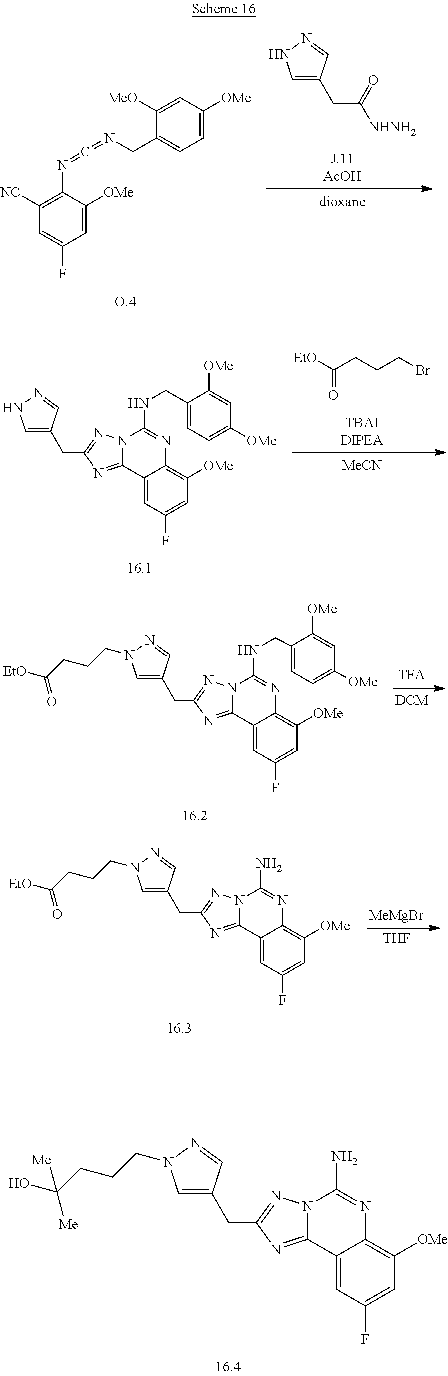

### Step 1—Synthesis of Intermediate 16.1, 2-((1H-pyrazol-4-yl)methyl)-N-(2,4-dimethoxybenzyl)-9-fluoro-7-methoxy-[1,2,4]triazolo[1,5-c]quinazolin-5-amine

AcOH (0.50 mL, 8.79 mmol) was added to a suspension of 2-((((2,4-dimethoxybenzyl)imino)methylene)amino)-5-fluoro-3-methoxybenzonitrile (6.0 g, 17.6 mmol) and 2-(1H-pyrazol-4-yl)acetohydrazide (2.48 g, 17.7 mmol) in dioxane (50 mL). The reaction was stirred vigorously at 60° C. for 20 h. The reaction was cooled to room temperature and purified by silica gel chromatography (gradient elution: 0-100% [3:1 EtOAc/EtOH]/hexanes) to provide 2-((1H-pyrazol-4-yl)methyl)-N-(2,4-dimethoxybenzyl)-9-fluoro-7-methoxy-[1,2,4]triazolo[1,5-c]quinazolin-5-amine. MS (ESI) m/z: calc'd for C₂₃H₂₃FN₇O₃ [M+H]⁺: 464.2. found 464.2.

### Step 2—Synthesis of Intermediate 16.2, ethyl 4-(4-((5-((2,4-dimethoxybenzyl)amino)-9-fluoro-7-methoxy-[1,2,4]triazolo[1,5-c]quinazolin-2-yl)methyl)-1H-pyrazol-1-yl)butanoate

Ethyl 4-bromobutanoate (0.41 mL, 2.86 mmol) and DIPEA (0.50 mL, 2.86 mmol) were added to a stirred solution of 2-((1H-pyrazol-4-yl)methyl)-N-(2,4-dimethoxybenzyl)-9-fluoro-7-methoxy-[1,2,4]triazolo[1,5-c]quinazolin-5-amine (530 mg, 1.14 mmol) and TBAI (21.1 mg, 0.057 mmol) in MeCN (3.0 mL). The reaction was stirred vigorously at 80° C. for 16 h. The reaction was cooled to room temperature and concentrated under reduced pressure. The resulting crude residue was purified by silica gel chromatography (gradient elution: 0-100% [3:1 EtOAc/EtOH]/hexanes) to provide ethyl 4-(4-((5-((2,4-dimethoxybenzyl)amino)-9-fluoro-7-methoxy-[1,2,4]triazolo[1,5-c]quinazolin-2-yl)methyl)-1H-pyrazol-1-yl)butanoate. MS (ESI) m/z: calc'd for C₂₉H₃₂FN₇O₅ [M+H]⁺: 578.3. found 578.3.

### Step 3—Synthesis of Example 16.3, ethyl 4-(4-((5-amino-9-fluoro-7-methoxy-[1,2,4]triazolo[1,5-c]quinazolin-2-yl)methyl)-1H-pyrazol-1-yl)butanoate

TFA (5 mL) was added to ethyl 4-(4-((5-((2,4-dimethoxybenzyl)amino)-9-fluoro-7-methoxy-[1,2,4]triazolo[1,5-c]quinazolin-2-yl)methyl)-1H-pyrazol-1-yl)butanoate from the previous step. The reaction was stirred vigorously at 60° C. for 2 h. The reaction was cooled to room temperature and concentrated under reduced pressure. The resulting crude residue was purified by reversed-phase HPLC [Method A] to provide ethyl 4-(4-((5-amino-9-fluoro-7-methoxy-[1,2,4]triazolo[1,5-c]quinazolin-2-yl)methyl)-1H-pyrazol-1-yl)butanoate (Example 16.3). MS (ESI) m/z: calc'd for C₂₀H₂₃FN₇O₃ [M+H]⁺: 428.2. found 428.2. ¹H NMR (500 MHz, DMSO-d₆) δ 7.86 (s, 2H), 7.68 (s, 1H), 7.49-7.36 (m, 2H), 7.18 (dd, J=11.1, 2.7 Hz, 1H), 4.16-3.99 (m, 6H), 3.94 (s, 3H), 2.25 (t, J=7.4 Hz, 2H), 1.98 (p, J=7.1 Hz, 2H), 1.15 (t, J=7.1 Hz, 3H). A_{2A} IC₅₀ 14.9 nM (A).

### Step 4—Synthesis of Example 16.4, 5-(4-((5-amino-9-fluoro-7-methoxy-[1,2,4]triazolo[1,5-c]quinazolin-2-yl)methyl)-1H-pyrazol-1-yl)-2-methylpentan-2-ol

MeMgBr (3 M, 0.16 mL, 0.47 mmol) was added to a stirred solution of ethyl 4-(4-((5-amino-9-fluoro-7-methoxy-[1,2,4]triazolo[1,5-c]quinazolin-2-yl)methyl)-1H-pyrazol-1-yl)butanoate (40 mg, 0.094 mmol) in THE (2 mL) at 0° C. The reaction was stirred vigorously at 0° C. for 2 h. The reaction was quenched with sat. aq. NH₄C₁ (0.25 mL), diluted with water, and extracted with EtOAc. The combined organic layers were dried over anhydrous Na₂SO₄, filtered, and concentrated under reduced pressure. The resulting crude residue was purified by reversed-phase HPLC [Method B] to provide 5-(4-((5-amino-9-fluoro-7-methoxy-[1,2,4]triazolo[1,5-c]quinazolin-2-yl)methyl)-1H-pyrazol-1-yl)-2-methylpentan-2-ol (Example 16.4). MS (ESI) m/z: calc'd for C₂₀H₂₅FN₇O₂ [M+H]⁺: 414.2. found 414.2. ¹H NMR (500 MHz, DMSO-d₆) δ 7.82 (s, 2H), 7.68 (s, 1H), 7.46-7.36 (m, 2H), 7.18 (dd, J=11.1, 2.7 Hz, 1H), 4.15 (s, 1H), 4.08 (s, 2H), 4.01 (t, J=7.2 Hz, 2H), 3.93 (s, 3H), 1.77 (dq, J=11.7, 7.5, 6.0 Hz, 2H), 1.34-1.25 (m, 2H), 1.04 (s, 6H). A_{2A} IC₅₀ 15.3 nM (A).

The following example in Table 17 was prepared according to Steps 2-4 in Scheme 16 and General Scheme 7, using Intermediate 16.1 and ethyl acrylate. For Step 2, the reaction was conducted in the absence of TBAI, and with DBU instead of DIPEA. Asterisk (*) indicates that A2B data is not available.

TABLE 17
Examples Prepared According to Scheme 16

| | | | A2a IC₅₀ |
|---|---|---|---|
| | | Observed | (nM) |
| | Structure | m/z | A2b IC₅₀ |
| Example | Name | [M + H]⁺ | (nM) |
| 16.5 | 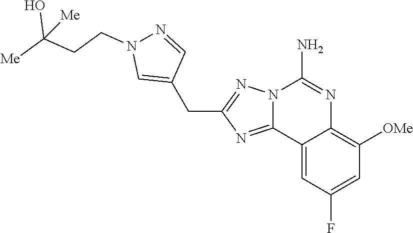 [SMILES: COc1cc(F)cc2c1nc(N)n1nc(Cc3cnn(CCC(C)(C)O)c3)nc21] | 400.2 | 43.4 (A) * |
| | 4-(4-((5-amino-9-fluoro-7-methoxy-[1,2,4]triazolo[1,5-c]- | | |
| | quinazolin-2-yl)methyl)-1H-pyrazol-1-yl)-2-methylbutan-2-ol | | |

### Preparation of Example 17.2, (R)-1-(5-amino-7-methoxy-[1,2,4]triazolo[1,5-c]quinazolin-2-yl)butan-2-ol

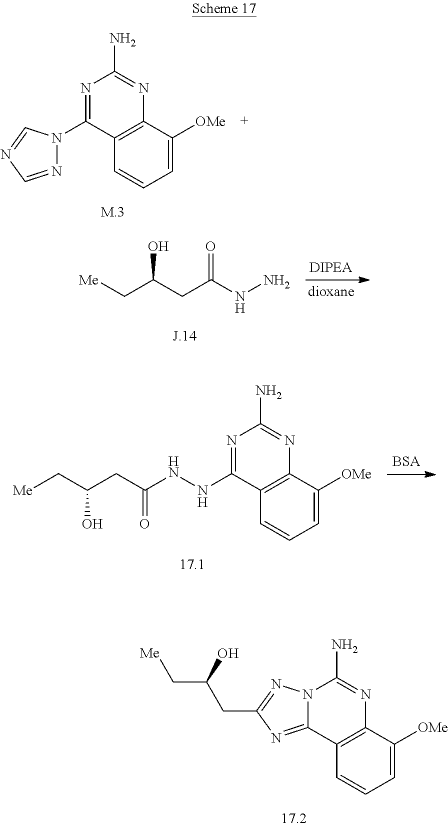

### Step 1—Synthesis of Intermediate 17.1, (R)—N′-(2-amino-8-methoxyquinazolin-4-yl)-3-hydroxypentanehydrazide

A mixture of (R)-3-hydroxypentanehydrazide (52.9 mg, 0.40 mmol), Fe ((65 mg, 0.268 mmol) and DIPEA (0.14 mL. 0.805 mmol) in dioxane (2 mL) was stirred vigorously at 80° C. for 16 h. The reaction was cooled to room temperature and concentrated under reduced pressure to provide (R)—N-(2-amino-8-methoxyquinazolin-4-yl)-3-hydroxypentanehydrazide, which was used in the subsequent step without further purification. MS (ESI) m/z: calc'd for C₁₄H₂₀N₅O₃ [M+H]⁺: 306.2. found 306.1.

### Step 2—Synthesis of Example 17.2, (R)-1-(5-amino-7-methoxy-[1,2,4]triazolo[1,5-c]quinazolin-2-yl)butan-2-ol

(R)—N′-(2-amino-8-methoxyquinazolin-4-yl)-3-hydroxypentanehydrazide (82 mg, 0.268 mmol) was added to BSA (2 mL, 8.16 mmol). The reaction was stirred vigorously at 120° C. for 2 h. The reaction was cooled to room temperature and concentrated under reduced pressure. The resulting crude residue was purified by reversed-phase HPLC [Method A] to provide (R)-1-(5-amino-7-methoxy-[1,2,4]triazolo[1,5-c]quinazolin-2-yl)butan-2-ol (Example 17.2). MS (ESI) m/z: calc'd for C₁₄H₁₈N₅O₂ [M+H]⁺: 288. ¹. found 288.1. ¹H NMR (600 Hz, DMSO-d₆) δ 7.96 (s, 2H), 7.75 (dd, J=7.9, 1.1 Hz, 1H), 7.32 (t, J=7.9 Hz, 1H), 7.25 (d, J=7.3 Hz, 1H), 4.02-3.95 (m, 1H), 3.92 (s, 3H), 2.99-2.88 (m, 2H), 1.61-1.50 (m, 1H), 1.50-1.40 (m, 1H), 0.93 (t, J=7.4 Hz, 3H). A_{2A} IC₅₀ 13.9 nM (A), A_{2B} IC₅₀ 307 nM (B).

The following examples in Table 18 were prepared according to Scheme 17 and General Scheme 2, using Intermediate M.3 and the appropriate hydrazide. Asterisk (*) indicates that A_{2B} data is not available.

TABLE 18
Examples Prepared According to Scheme 17

| | | | A2a IC₅₀ |
|---|---|---|---|
| | | Observed | (nM) |
| | Structure | m/z | A2b IC₅₀ |
| Example | Name | [M + H]⁺ | (nM) |
| 17.3 | 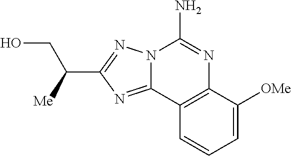 [SMILES: COc1cccc2c1nc(N)n1nc([C@@H](C)CO)nc21] | 274.2 | 264 (A) 2994 (B) |
| | (R)-2-(5-amino-7-methoxy-[1,2,4]triazolo[1,5- | | |
| | c]quinazolin-2-yl)propan-1-ol | | |
| 17.4 | 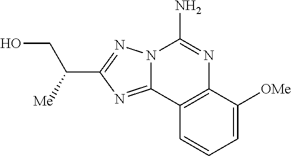 [SMILES: COc1cccc2c1nc(N)n1nc([C@H](C)CO)nc21] | 274.2 | 145 (A) 1074 (B) |
| | (S)-2-(5-amino-7-methoxy-[1,2,4]triazolo[1,5- | | |
| | c]quinazolin-2-yl)propan-1-ol | | |

### Preparation of Example 18.2, 1-(5-amino-7-methoxy-[1,2,4]triazolo[1,5-c]quinazolin-2-yl)-2-methylpropan-2-ol

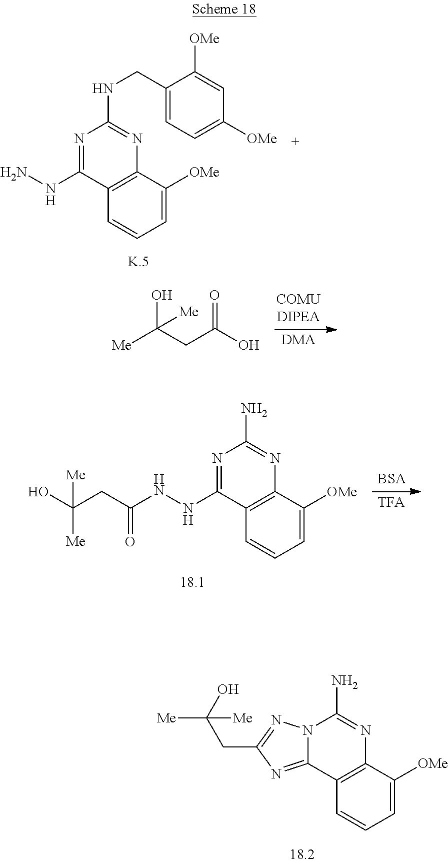

### Step 1—Synthesis of Intermediate 18.1, N′-(2-((2,4-dimethoxybenzyl)amino)-8-methoxyquinazolin-4-yl)-3-hydroxy-3-methylbutanehydrazide

A solution of COMU (169 mg, 0.394 mmol) in dimethylacetamide (DMA) (1 mL) and DIPEA (98 μL. 0.563 mmol) were sequentially added to 3-Hydroxy-3-methylbutyric acid (49.5 μL. 0.394). The reaction was stirred vigorously at room temperature for 10 min; at which point a suspension of N-(2,4-dimethoxybenzyl)-4-hydrazinyl-8-methoxyquinazolin-2-amine (100 mg, 0.281 mmol) in DMA (1 mL) was added. The reaction was stirred vigorously at room temperature for 4 h. The reaction was concentrated under reduced pressure, diluted with 3:1 CHCl₃/i-PrOH (5 mL), and washed with sat. aq. NaHCO₃ (5 mL). The combined organic layers were concentrated under reduced pressure to provide N-(2-((2,4-dimethoxybenzyl)amino)-8-methoxyquinazolin-4-yl)-3-hydroxy-3-methylbutanehydrazide, which was used in the subsequent step without further purification. MS (ESI) m/z: calc'd for C₂₃H₃₀N₅O₅ [M+H]⁺: 456.2. found 455.7.

### Step 2—Synthesis of Example 18.2, 1-(5-amino-7-methoxy-[1,2,4]triazolo[1,5-c]quinazolin-2-yl)-2-methylpropan-2-ol

BSA (2 mL, 8.16 mmol) was added to N-(2-((2,4-dimethoxybenzyl)amino)-8-methoxyquinazolin-4-yl)-3-hydroxy-3-methylbutanehydrazide (128 mg, 0.281 mmol). The reaction was stirred vigorously at 120° C. for 2 h. The reaction was cooled to room temperature and concentrated under reduced pressure, at which point TFA (2 mL) was added. The reaction was stirred vigorously at 50° C. for 2 h. The reaction was cooled to room temperature and concentrated under reduced pressure. The resulting crude residue was purified by reversed-phase HPLC [Method A] to provide 1-(5-amino-7-methoxy-[1,2,4]triazolo[1,5-c]quinazolin-2-yl)-2-methylpropan-2-ol (Example 18.2). MS (ESI) m/z: calc'd for C₁₄H₁₈N₅O₂ [M+H]: 288.1. found 287.9. ¹H NMR (600 MHz, DMSO-d₆) δ 7.98 (s, 2H), 7.75 (dd, J=7.9, 1.1 Hz, 1H), 7.33 (t, J=7.9 Hz, 1H), 7.26 (d, J=7.3 Hz, 1H), 3.93 (s, 3H), 3.00 (s, 2H), 1.26 (s, 6H). A_{2A} IC₅₀ 30.8 nM (A), A_{2B} IC₅₀ 128 nM (B).

The following examples in Table 19 were prepared according to Scheme 18 and General Scheme 4, using Intermediate K.5 and the appropriate acid. Asterisk (*) indicates that A2B data is not available.

TABLE 19
Examples Prepared According to Scheme 18

| | | | A2a IC₅₀ |
|---|---|---|---|
| | | Observed | (nM) |
| | | m/z | A2b IC₅₀ |
| Example | Name | [M + H]⁺ | (nM) |
| 18.3 | 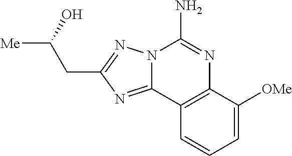 [SMILES: COc1cccc2c1nc(N)n1nc(C[C@H](C)O)nc21] | 274.2 | 6.7 (A) 214 (B) |
| | (S)-1-(5-amino-7-methoxy-[1,2,4]triazolo[1,5- | | |
| | c]quinazolin-2-yl)propan-2-ol | | |
| 18.4 | 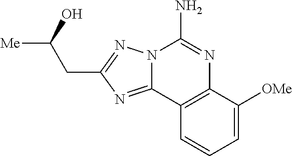 [SMILES: COc1cccc2c1nc(N)n1nc(C[C@@H](C)O)nc21] | 274.2 | 15.1 (A) 209 (B) |
| | (R)-1-(5-amino-7-methoxy-[1,2,4]triazolo[1,5- | | |
| | c]quinazolin-2-yl)propan-2-ol | | |

### Biological Assays

The IC₅₀ values reported for each of the compounds of the invention shown in the tables below were measured in accordance with the methods described below. Method (A) describes the procedure used to measure A2A binding affinity using radioligand binding. Method (B) describes the procedure used to measure A2A binding affinity using SPA technology. The method used to measure A2B binding affinity is also described below. The method used to determine the A_{2A} IC₅₀ value reported for each compound in the table is indicated next to the reported value. The A_{2B} IC₅₀ value measured using the A2B binding affinity assay is shown in the table next to the compound under the corresponding A2A value. An asterisk (*) indicates that the IC₅₀ value was not available.

The A_{2A} receptor affinity binding assay measured the amount of binding of a tritiated ligand with high affinity for the A_{2A} adenosine receptor to membranes made from HEK293 or CHO cells recombinantly expressing the human A_{2A} adenosine receptor, in the presence of varying concentrations of a compound of the invention. The data were generated using either filtration binding or a homogenous scintillation proximity assay (SPA). In both assay formats, the tested compounds of the invention were solubilized in 100% DMSO and further diluted in 100% DMSO to generate, typically, a 10-point titration at half-log intervals such that the final assay concentrations did not exceed 10 μM of compound or 1% DMSO.

### Method (A): Measurement of A_{2A} Binding Affinity Using Radioligand Binding

148 μL (5 μg/mL) membranes (Perkin Elmer, Cat. No. RBHA2aM400UA) and 2 μL compounds of the invention to be tested (test compound) were transferred to individual wells of a 96-well polypropylene assay plate and incubated for 15 to 30 min at room temperature. [³H] SCH58261 ((7-(2-phenylethyl)-5-amino-2-(2-furyl)-pyrazolo-[4,3-e]-1,2,4-triazolo[1,5-c]pyrimidine)) was diluted in assay buffer (50 mM Tris pH 7.4, 10 mM MgCl₂, 0.005% Tween20) to a concentration of 4 nM and 50 μL transferred to each well of the assay plate. To define total and non-specific binding, wells containing 1% DMSO and 1 μM ZM241385 (Tocris Bioscience, Cat. No. 1036) respectively, were also included. The assay plate was incubated at room temperature for 60 min with agitation. Using a FilterMate Harvester® (Perkin Elmer), the contents of the assay plate were filtered through a UniFilter-96® PEI coated plate (Perkin Elmer Cat. No. 6005274 or 6005277). Filtering was achieved by aspirating the contents of the assay plate for 5 sec, then washing and aspirating the contents three times with ice-cooled wash buffer (50 mM Tris-HCl pH 7.4, 150 mM NaCl) and allowing the vacuum manifold to dry the plate for 30 sec. The filter plate was incubated for at least 1 h at 55° C. and allowed to dry. The bottom of the filter plate was sealed with backing tape. 40 μL Ultima Gold™ (Perkin Elmer, Cat. No. 6013329) was added to each well of the filter plate and the top of the plate was sealed with TopSeal-A PLUS® clear plate seal (Perkin Elmer, Cat. No. 6050185). The plate was incubated for at least 20 min, and then the amount of radioactivity remaining in each well was determined using a TopCount® (Perkin Elmer) scintillation counter. After normalization to total and non-specific binding, the percent effect at each compound concentration was calculated. The plot of percent effect versus the log of compound concentration was analyzed electronically using a 4-parameter logistic fit based on the Levenberg-Marquardt algorithm to generate IC₅₀ values.

### Method (B): Measurement of A_{2A} Binding Affinity Using SPA

Binding affinity using SPA was conducted as follows. Test compounds (50 nL) were dispensed into individual wells of a 384-well OptiPlate™ well (Perkin Elmer) by Echo® acoustic liquid transfer (Labcyte). 20 μL of 1.25 nM [³H] SCH58261 ((7-(2-phenylethyl)-5-amino-2-(2-furyl)-pyrazolo-[4,3-e]-1,2,4-triazolo[1,5-c]pyrimidine)) in DPBS assay buffer (Dulbecco's phosphate buffered saline without calcium and magnesium, ThermoFisher Scientific, Cat. No. A1285601) supplemented with 10 mM MgCl₂ was added. A2A receptor-expressing membranes were incubated with 20 μg/mL adenosine deaminase (Roche, Cat. No. 10 102 105 001) for 15 min at room temperature. The receptor-expressing membranes were then combined with wheat germ agglutinin-coated yttrium silicate SPA beads (GE Healthcare, Cat. No. RPNQ0023) in a ratio of 1:1000 (w/w) and incubated for 30 min at room temperature. 30 μL of the membrane/bead mixture (0.25 μg and 25 μg per well respectively) were added to the 384-well OptiPlate™ well. To define total and non-specific binding, wells containing 1% DMSO or 1 μM CGS15943 (Tocris Bioscience, Cat. No. 1699) respectively were also included in the experiment. The plate was incubated for 1 h at room temperature with agitation. The assay plate was then incubated for an h to allow the beads to settle before data were collected using a TopCount® (Perkin Elmer) scintillation counter. After normalization to total and non-specific binding, the percent effect at each compound concentration was calculated. The plot of percent effect versus the log of compound concentration was analyzed electronically using a 4-parameter logistic fit based on the Levenberg-Marquardt algorithm to generate IC₅₀ values.

### Measurement of A_{2B} Binding Affinity

The reported affinity of the compounds of the invention for the human A_{2B} adenosine receptor was determined experimentally using a radioligand filtration binding assay. This assay measures the amount of binding of a tritiated proprietary A_{2B} receptor antagonist, in the presence and absence of a compound of the invention, to membranes made from HEK293 cells recombinantly expressing the human A_{2B} adenosine receptor (Perkin Elmer, Cat. No. ES-013-C).

To perform the assay, compounds of the invention to be tested were first solubilized in 100% DMSO and further diluted in 100% DMSO to generate, typically, a 10-point titration at half-log intervals such that the final assay concentrations did not exceed 10 μM of compound or 1% DMSO. 148 μL (135 μg/mL) membranes and 2 μL test compounds were transferred to individual wells of a 96-well polypropylene assay plate and incubated for 15 to 30 min at room temperature with agitation. Tritiated radioligand was diluted to a concentration of 14 nM in assay buffer (phosphate buffered saline without Magnesium and Calcium, pH 7.4; GE Healthcare Life Sciences, Cat. No. SH30256.01) and then 50 μL of the solution were transferred to each well of the assay plate. To define total and non-specific binding, wells containing 1% DMSO and 20 μM N-ethylcarboxamidoadenosine (Tocris Bioscience, Cat. No. 1691) respectively, were also included. The wells of the assay plate were incubated at room temperature for 60 min with agitation, then filtered using a FilterMate Harvester® (Perkin Elmer) or similar equipment through a UniFilter-96@ PEI coated plate (Perkin Elmer Cat. No. 6005274 or 6005277). Filtering was achieved by aspirating the contents of the assay plate for 5 sec, then washing and aspirating the contents three times with ice-cooled wash buffer (assay buffer supplemented with 0.0025% Brij58) and allowing the vacuum manifold to dry the plate for 30 sec. The filter plate was incubated for at least 1 h at 55° C. and allowed to dry. The bottom of the filter plate was then sealed with backing tape. 40 μL Ultima Gold™ (Perkin Elmer, Cat. No. 6013329) was added to each well of the filter plate and the top of the plate was sealed with TopSeal-A PLUS® clear plate seal (Perkin Elmer, Cat. No. 6050185). The plates were then incubated for at least 20 min, and then the amount of radioactivity remaining in each well was determined using a TopCount® (Perkin Elmer) scintillation counter. After normalization to total and non-specific binding, the percent effect at each compound concentration was calculated. The plot of percent effect versus the log of compound concentration was analyzed electronically using a 4-parameter logistic fit based on the Levenberg-Marquardt algorithm to generate IC₅₀ values.

1. A compound having a structural Formula (I): 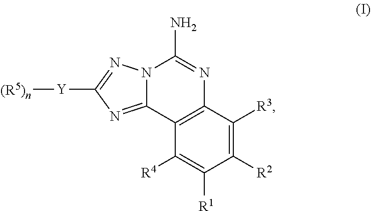
or a pharmaceutically acceptable salt thereof, wherein:
R¹ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl;
R² is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl;
R³ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl;
R⁴ is selected from the group consisting of hydrogen, halogen, (C₁-C₆)alkyl, O(C₁-C₆)alkyl, OH, O(C₁-C₆)haloalkyl, (C₁-C₆)haloalkyl, CN, (C₃-C₆)cycloalkyl and cycloheteroalkyl;
Y is a straight or branched (C₁-C₅)alkyl or (C₃-C₆)cycloalkyl(C₁-C₅)alkyl, wherein one or more —CH₂— groups in Y are optionally and independently replaced with a moiety selected from the group consisting of S, O and SO₂;
each occurrence of R⁵ is independently selected from hydrogen, halogen, aryl, cycloheteroalkyl, heteroaryl, (C₁-C₆)alkylOH, OH, (C₁-C₆)haloalkyl, (C₁-C₆)alkyl, (C₁-C₆)alkynyl, SO₂R⁶, SO(═NH)R⁶, SO(═NCH₃)R⁶ and COO(C₁-C₆)alkyl, wherein the aryl, cycloheteroalkyl or heteroaryl are optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH, (C₁-C₆)alkylC(O)O(C₁-C₆)alkyl and (C₁-C₆)alkylN(R⁷)₂;
R⁶ is selected from the group consisting of OH, NH₂, (C₁-C₆)alkyl, aryl, (C₁-C₆)haloalkyl, (C₃-C₆)cycloalkyl and haloaryl;
each occurrence of R⁷ is independently selected from the group consisting of H, (C₁-C₆)alkyl, and (C₃-C₆)cycloalkyl, or when two R⁷ substituents are taken together with the nitrogen to which they are attached, form a cycloheteroalkyl; and
n is 1, 2 or 3.

2. The compound of claim 1
or a pharmaceutically acceptable salt thereof, wherein:
R¹ is selected from the group consisting of hydrogen, halogen, (C₁-C₆)alkyl, O(C₁-C₆)alkyl and cycloheteroalkyl;
R² is selected from the group consisting of hydrogen, halogen, (C₁-C₆)alkyl, O(C₁-C₆)alkyl and cycloheteroalkyl;
R³ is selected from the group consisting of hydrogen, halogen, (C₁-C₆)alkyl, O(C₁-C₆)alkyl and cycloheteroalkyl;
R⁴ is selected from the group consisting of hydrogen, halogen, (C₁-C₆)alkyl, O(C₁-C₆)alkyl and cycloheteroalkyl;
Y is a straight or branched (C₁-C₅)alkyl or cycloalkyl(C₁-C₅)alkyl, wherein one or more —CH₂— groups in Y are optionally and independently replaced with a moiety selected from the group consisting of S, O, and SO₂;
each occurrence of R⁵ is independently selected from hydrogen, halogen, aryl, cycloheteroalkyl, heteroaryl, (C₁-C₆)alkylOH, OH, (C₁-C₆)haloalkyl, (C₁-C₆)alkyl, (C₁-C₆)alkynyl, SO₂R⁶, SO(═NH)R⁶, SO(═NCH₃)R⁶ and COO(C₁-C₆)alkyl, wherein the aryl, cycloheteroalkyl or heteroaryl are optionally substituted with one to three substituents selected from the group consisting of halogen, (C₁-C₆)alkyl, (C₁-C₆)haloalkyl, (C₃-C₆)cycloalkyl, (C₃-C₆)halocycloalkyl, (C₁-C₆)haloalkyl(C₃-C₆)cycloalkyl, (C₁-C₆)haloalkylOH, (C₁-C₆)alkylOH and (C₁-C₆)alkylNH₂; and
R⁶ is selected from the group consisting of NH₂, (C₁-C₆)alkyl, aryl, (C₁-C₆)haloalkyl, (C₃-C₆)cycloalkyl and haloaryl.

3. The compound of claim 2, or a pharmaceutically acceptable salt thereof, wherein R¹ is selected from the group consisting of hydrogen and fluorine.

4. The compound of claim 3, or a pharmaceutically acceptable salt thereof, wherein R² is selected from the group consisting of hydrogen and methoxy.

5. The compound of claim 4, or a pharmaceutically acceptable salt thereof, wherein R³ is selected from the group consisting of hydrogen, fluorine and methoxy.

6. The compound of claim 5, or a pharmaceutically acceptable salt thereof, wherein R⁴ is selected from the group consisting of hydrogen and

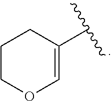 [SMILES: *C1=COCCC1]

7. The compound of claim 6, or a pharmaceutically acceptable salt thereof, wherein R¹, R², R³ and R⁴ are not simultaneously hydrogen.

8. The compound of claim 7, or a pharmaceutically acceptable salt thereof, wherein Y is a straight (C₁-C₅)alkyl.

9. The compound of claim 7, or a pharmaceutically acceptable salt thereof, wherein Y is a branched (C₁-C₅)alkyl.

10. The compound of claim 7, or a pharmaceutically acceptable salt thereof, wherein Y is a cycloalkyl(C₁-C₅)alkyl.

11. The compound of claim 7, or a pharmaceutically acceptable salt thereof, wherein Y is a straight or branched (C₁-C₅)alkyl, wherein one or more —CH₂— groups in Y are independently replaced with a moiety selected from the group consisting of O, S and SO₂.

12. The compound of claim 7, or a pharmaceutically acceptable salt thereof, wherein Y is

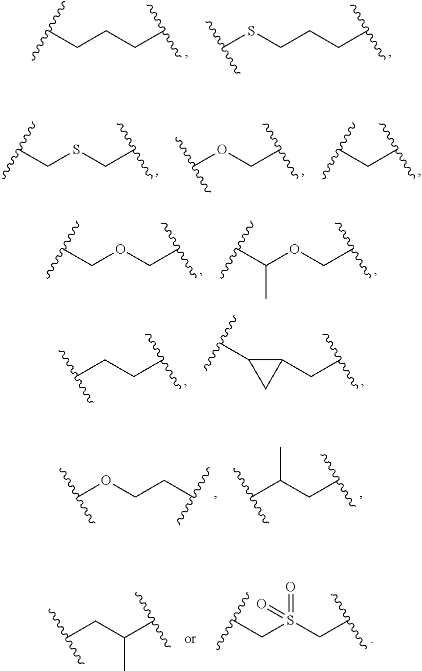

13. The compound of claim 12, or a pharmaceutically acceptable salt thereof, wherein R⁵ is chlorine, methyl, fluoromethyl, difluoromethyl, trifluoromethyl, OH, propyl, phenyl, SO₂R⁶, —COOCH₂CH₃,

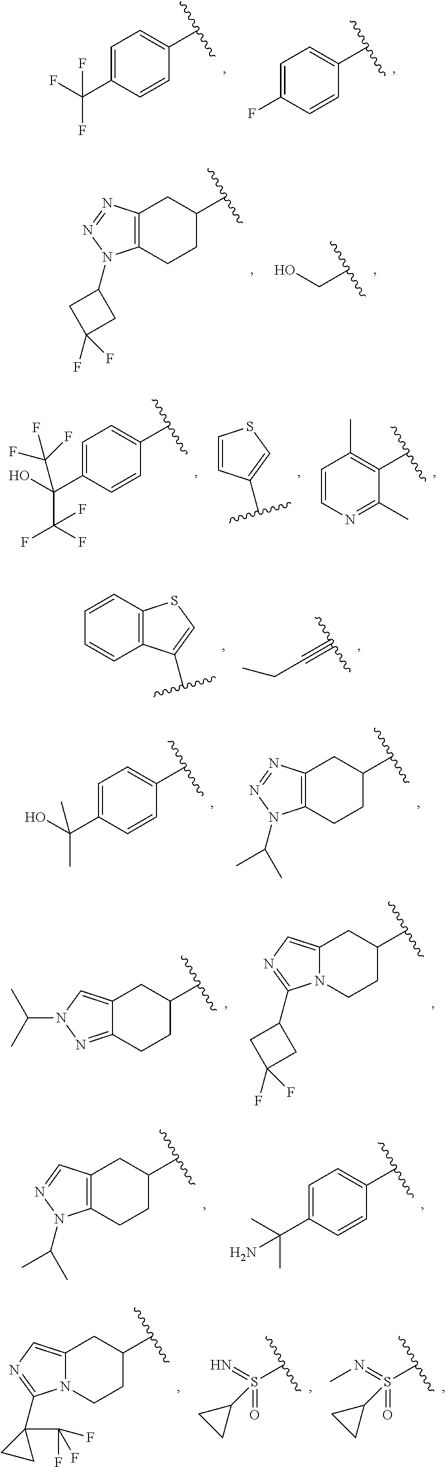

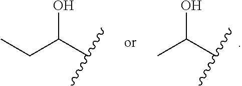

14. The compound of claim 13, or a pharmaceutically acceptable salt thereof, wherein R⁵ is SO₂R⁶, wherein R⁶ is methyl, NH₂, phenyl, cyclopropyl, fluorophenyl, trifluoromethyl, ethyl, iso-butyl or iso-propyl.

15. A compound, or pharmaceutically acceptable salt thereof, having the following structure:

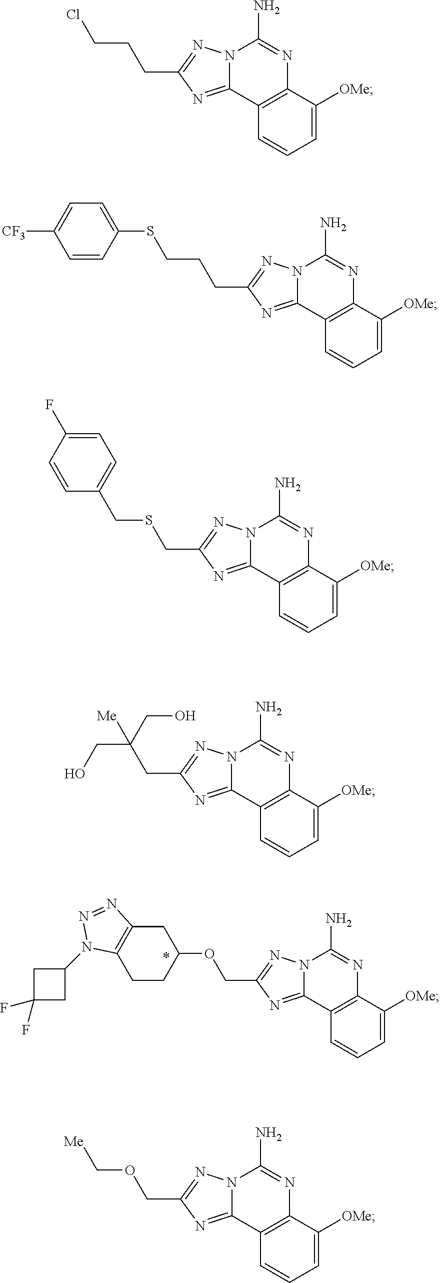 [SMILES: COc1cccc2c1nc(N)n1nc(CCCCl)nc21 ; COc1cccc2c1nc(N)n1nc(CCCSc3ccc(C(F)(F)F)cc3)nc21 ; COc1cccc2c1nc(N)n1nc(CSCc3ccc(F)cc3)nc21 ; COc1cccc2c1nc(N)n1nc(CC(C)(CO)CO)nc21 ; COc1cccc2c1nc(N)n1nc(COC3CCc4c(nnn4C4CC(F)(F)C4)C3)nc21 ; CCOCc1nc2c3cccc(OC)c3nc(N)n2n1]

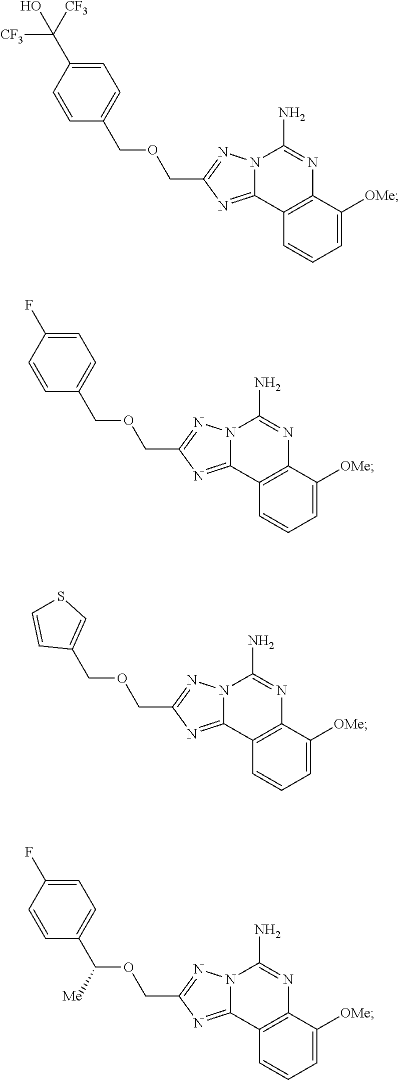 [SMILES: COc1cccc2c1nc(N)n1nc(COCc3ccc(C(O)(C(F)(F)F)C(F)(F)F)cc3)nc21 ; COc1cccc2c1nc(N)n1nc(COCc3ccc(F)cc3)nc21 ; COc1cccc2c1nc(N)n1nc(COCc3ccsc3)nc21 ; COc1cccc2c1nc(N)n1nc(CO[C@H](C)c3ccc(F)cc3)nc21]

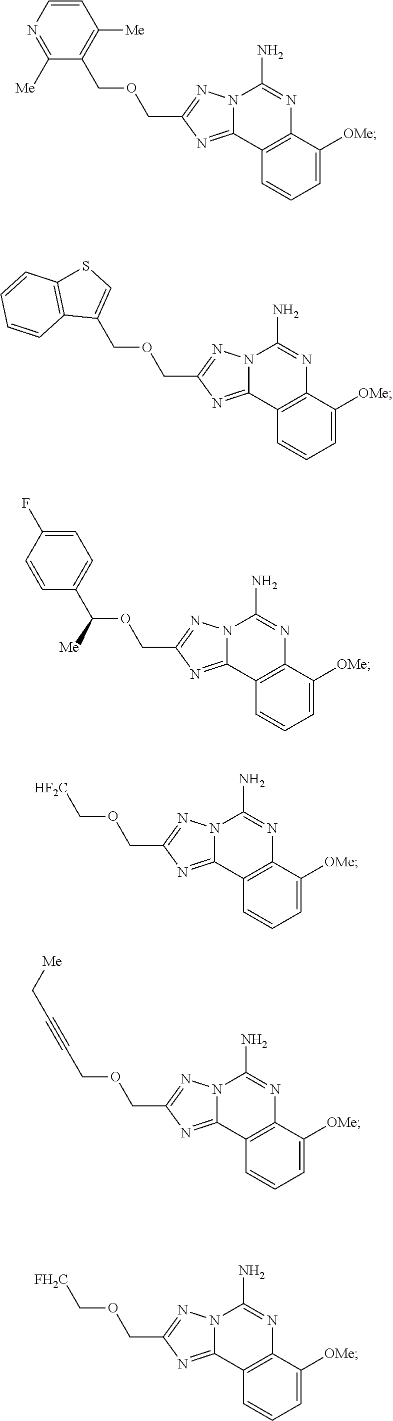

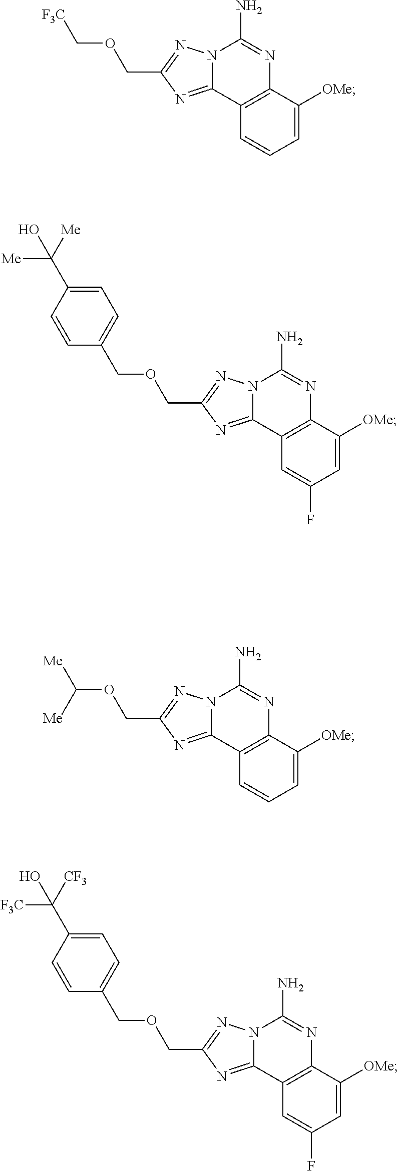 [SMILES: COc1cccc2c1nc(N)n1nc(COCC(F)(F)F)nc21 ; COc1cc(F)cc2c1nc(N)n1nc(COCc3ccc(C(C)(C)O)cc3)nc21 ; COc1cccc2c1nc(N)n1nc(COC(C)C)nc21 ; COc1cc(F)cc2c1nc(N)n1nc(COCc3ccc(C(O)(C(F)(F)F)C(F)(F)F)cc3)nc21]

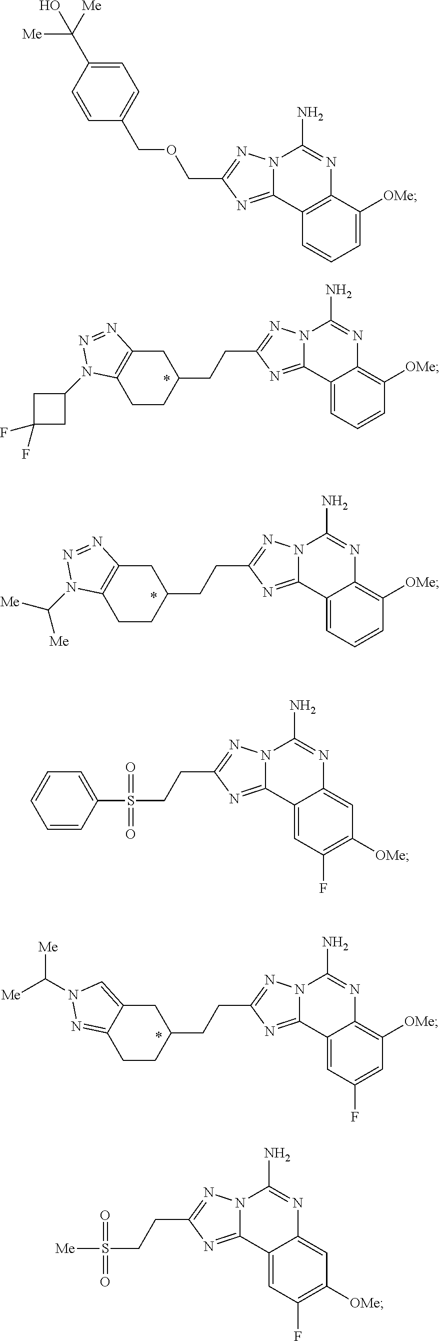

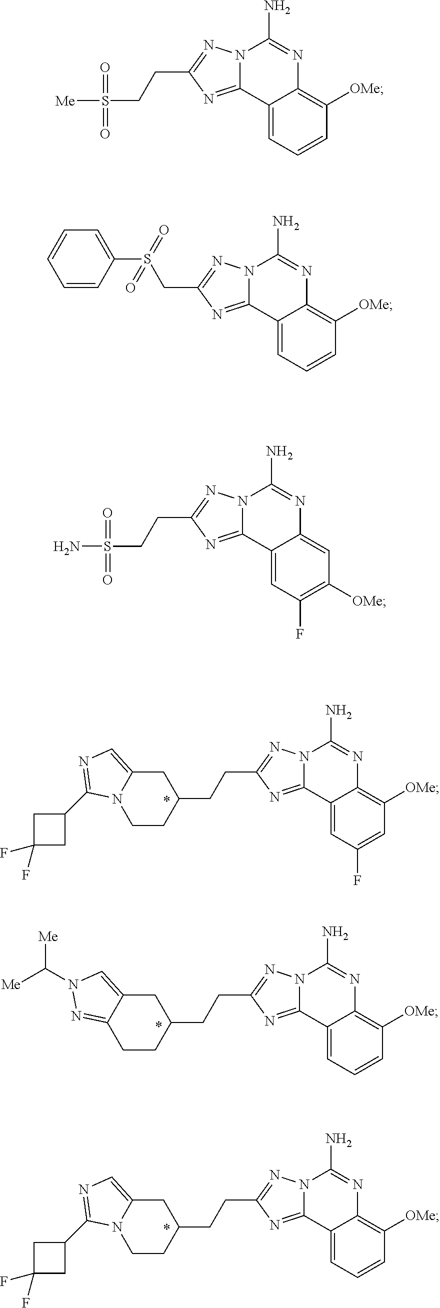 [SMILES: COc1cccc2c1nc(N)n1nc(CCS(C)(=O)=O)nc21 ; COc1cccc2c1nc(N)n1nc(CS(=O)(=O)c3ccccc3)nc21 ; COc1cc2nc(N)n3nc(CCS(N)(=O)=O)nc3c2cc1F ; COc1cc(F)cc2c1nc(N)n1nc(CCC3CCn4c(cnc4C4CC(F)(F)C4)C3)nc21 ; COc1cccc2c1nc(N)n1nc(CCC3CCc4nn(C(C)C)cc4C3)nc21 ; COc1cccc2c1nc(N)n1nc(CCC3CCn4c(cnc4C4CC(F)(F)C4)C3)nc21]

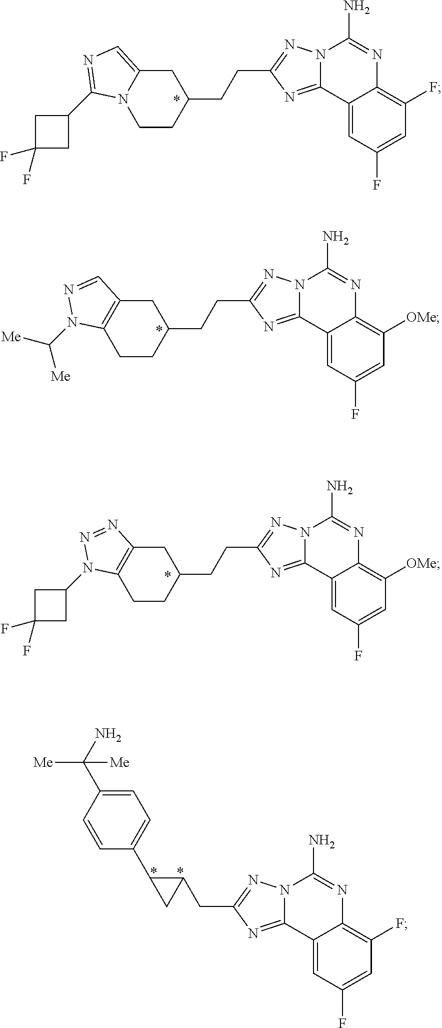 [SMILES: Nc1nc2c(F)cc(F)cc2c2nc(CCC3CCn4c(cnc4C4CC(F)(F)C4)C3)nn12 ; COc1cc(F)cc2c1nc(N)n1nc(CCC3CCc4c(cnn4C(C)C)C3)nc21 ; COc1cc(F)cc2c1nc(N)n1nc(CCC3CCc4c(nnn4C4CC(F)(F)C4)C3)nc21 ; CC(C)(N)c1ccc(C2CC2Cc2nc3c4cc(F)cc(F)c4nc(N)n3n2)cc1]

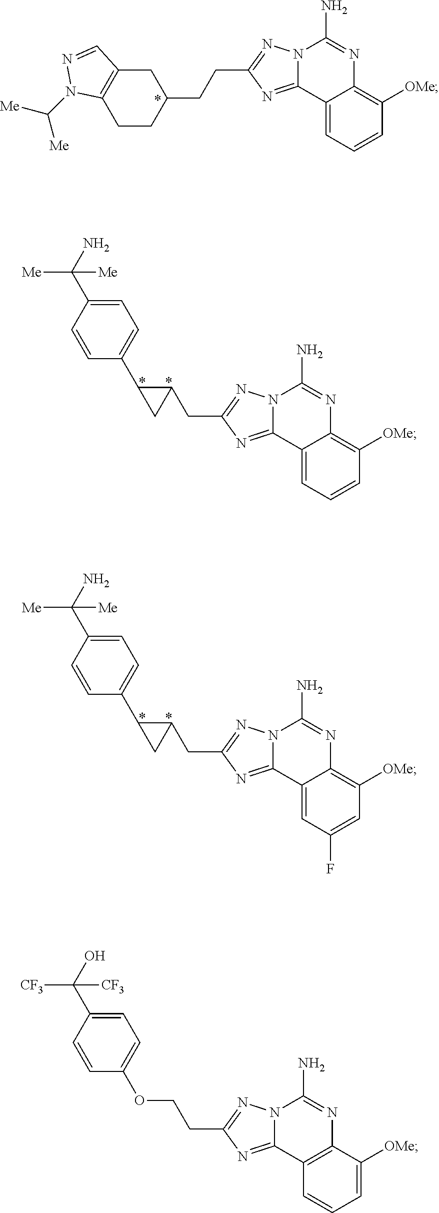 [SMILES: COc1cccc2c1nc(N)n1nc(CCC3CCc4c(cnn4C(C)C)C3)nc21 ; COc1cccc2c1nc(N)n1nc(CC3CC3c3ccc(C(C)(C)N)cc3)nc21 ; COc1cc(F)cc2c1nc(N)n1nc(CC3CC3c3ccc(C(C)(C)N)cc3)nc21 ; COc1cccc2c1nc(N)n1nc(CCOc3ccc(C(O)(C(F)(F)F)C(F)(F)F)cc3)nc21]

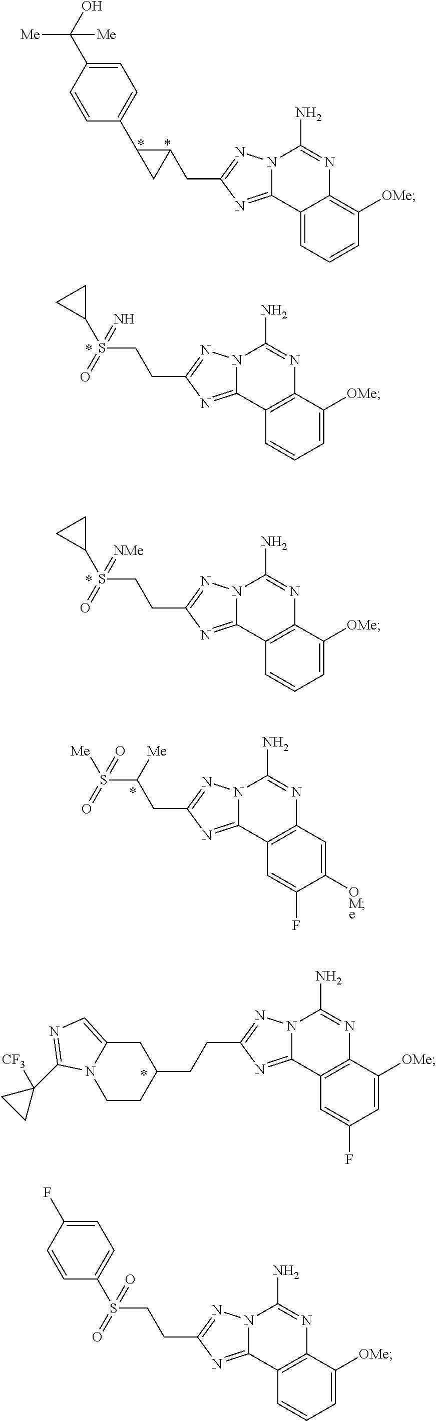

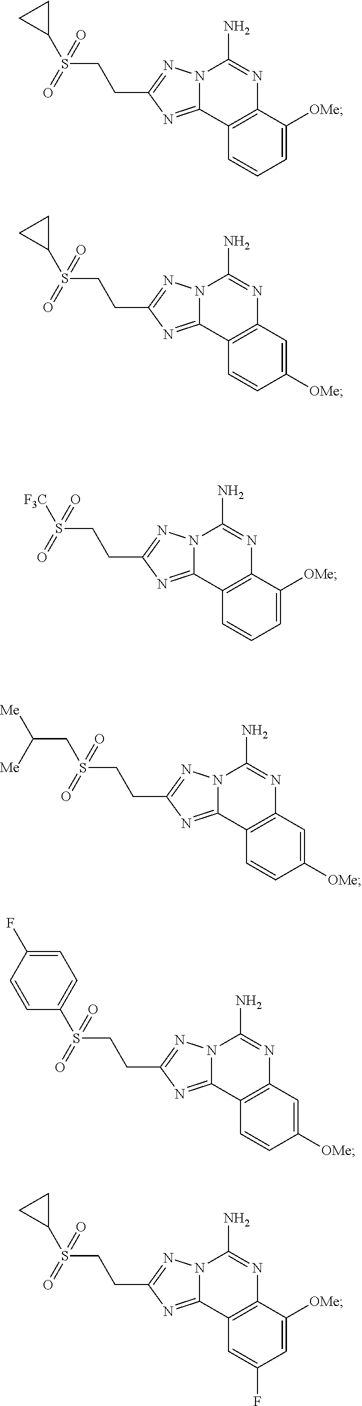 [SMILES: COc1cccc2c1nc(N)n1nc(CCS(=O)(=O)C3CC3)nc21 ; COc1ccc2c(c1)nc(N)n1nc(CCS(=O)(=O)C3CC3)nc21 ; COc1cccc2c1nc(N)n1nc(CCS(=O)(=O)C(F)(F)F)nc21 ; COc1ccc2c(c1)nc(N)n1nc(CCS(=O)(=O)CC(C)C)nc21 ; COc1ccc2c(c1)nc(N)n1nc(CCS(=O)(=O)c3ccc(F)cc3)nc21 ; COc1cc(F)cc2c1nc(N)n1nc(CCS(=O)(=O)C3CC3)nc21]

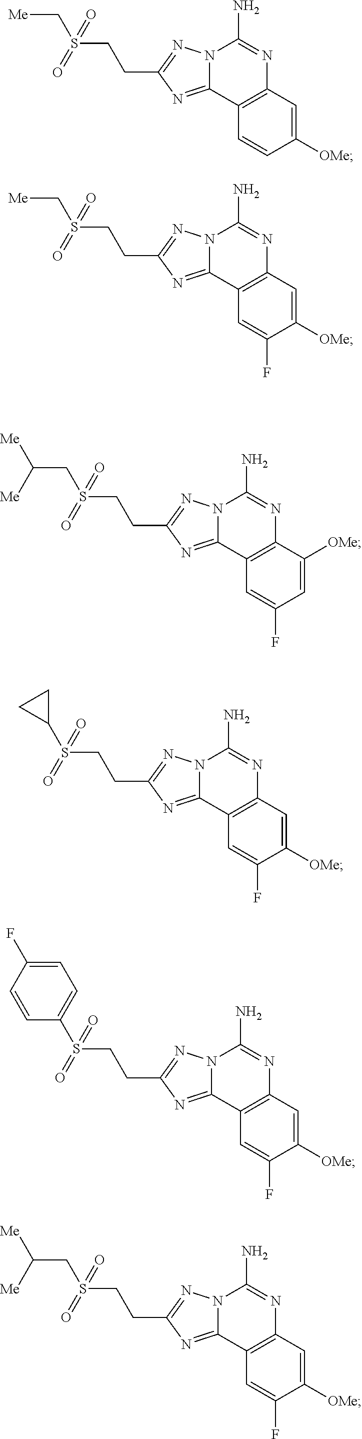

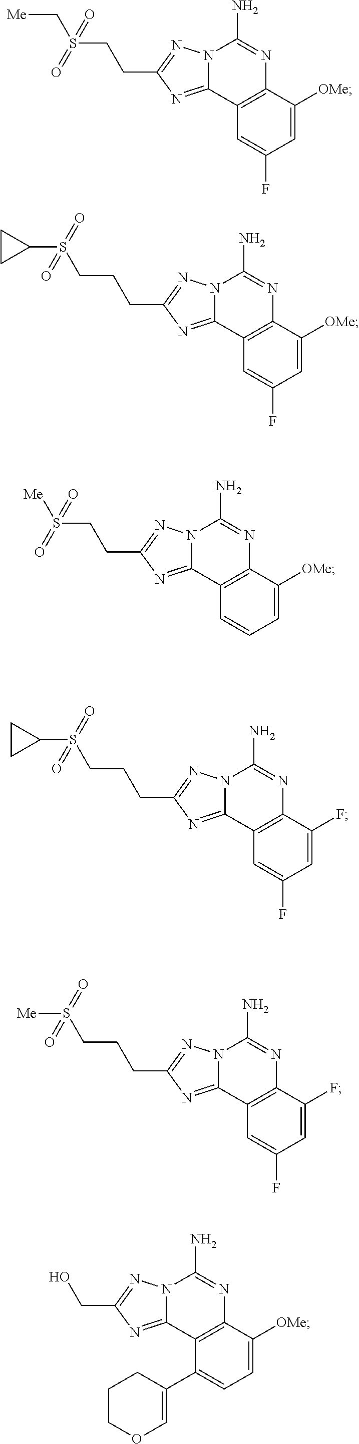 [SMILES: CCS(=O)(=O)CCc1nc2c3cc(F)cc(OC)c3nc(N)n2n1 ; COc1cc(F)cc2c1nc(N)n1nc(CCCS(=O)(=O)C3CC3)nc21 ; COc1cccc2c1nc(N)n1nc(CCS(C)(=O)=O)nc21 ; Nc1nc2c(F)cc(F)cc2c2nc(CCCS(=O)(=O)C3CC3)nn12 ; CS(=O)(=O)CCCc1nc2c3cc(F)cc(F)c3nc(N)n2n1 ; COc1ccc(C2=COCCC2)c2c1nc(N)n1nc(CO)nc21]

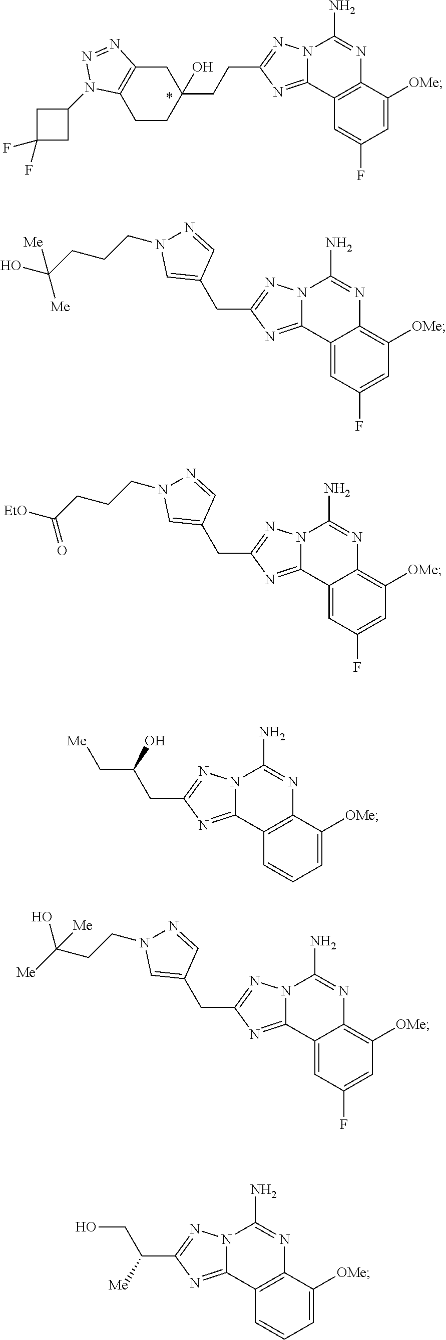 [SMILES: COc1cc(F)cc2c1nc(N)n1nc(CCC3(O)CCc4c(nnn4C4CC(F)(F)C4)C3)nc21 ; COc1cc(F)cc2c1nc(N)n1nc(Cc3cnn(CCCC(C)(C)O)c3)nc21 ; CCOC(=O)CCCn1cc(Cc2nc3c4cc(F)cc(OC)c4nc(N)n3n2)cn1 ; CC[C@@H](O)Cc1nc2c3cccc(OC)c3nc(N)n2n1 ; COc1cc(F)cc2c1nc(N)n1nc(Cc3cnn(CCC(C)(C)O)c3)nc21 ; COc1cccc2c1nc(N)n1nc([C@H](C)CO)nc21]

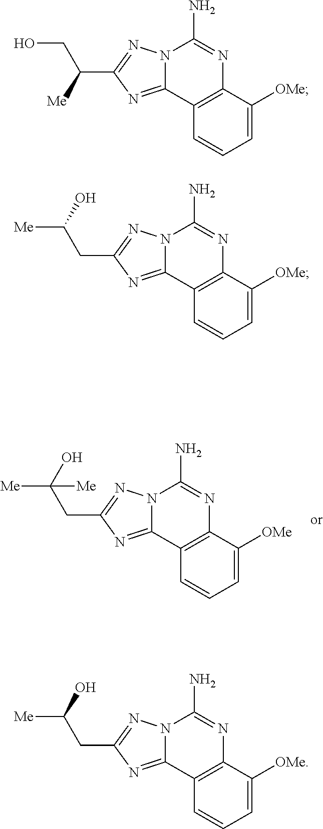

16. A pharmaceutical composition comprising a compound of claim 1, or a pharmaceutically acceptable salt thereof, and a pharmaceutically acceptable carrier.

17. A method of treating cancer comprising administering an effective amount of a compound of claim 1, or a pharmaceutically acceptable salt thereof, to a person in need thereof.

18. The method of claim 17, wherein said cancer is selected from melanoma, head and neck cancer, classical Hodgkin lymphoma, urothelial carcinoma, gastric cancer, cervical cancer, primary mediastinal large-B-cell lymphoma, microsatellite instability-high cancer, non-small cell lung cancer, hepatocellular carcinoma, clear cell kidney cancer, colorectal cancer, breast cancer, squamous cell lung cancer, basal carcinoma, sarcoma, bladder cancer, endometrial cancer, pancreatic cancer, liver cancer, gastrointestinal cancer, multiple myeloma, renal cancer, mesothelioma, ovarian cancer, anal cancer, biliary tract cancer, esophageal cancer, salivary cancer, and prostate cancer, and metastatic castration resistant prostate cancer.

19. The method of claim 18, wherein said compound, or a pharmaceutically acceptable salt thereof, is administered in combination with another therapeutic agent.

20. The method of claim 19, wherein said additional therapeutic agent is a PD-1 antagonist.

21. The method of claim 20, wherein said additional therapeutic agent is selected from pembrolizumab nivolumab, atezolizumab, durvalumab, and avelumab.

22. The method of claim 21, wherein said additional therapeutic agent is pembrolizumab.
